SEQUENCE LISTING (115 sequences)

SEQ ID NO: 1 - protein, 10 aa, synthetic construct
  REGION 1..10: note = Synthetic Construct
YSISSGYYWG

SEQ ID NO: 2 - protein, 16 aa, synthetic construct
  REGION 1..16: note = Synthetic Construct
TIYHSGSTYYNPSLKS

SEQ ID NO: 3 - protein, 16 aa, synthetic construct
  REGION 1..16: note = Synthetic Construct
TIYHSGSTYYNPSLES

SEQ ID NO: 4 - protein, 16 aa, synthetic construct
  REGION 1..16: note = Synthetic Construct
  VARIANT 15: note = Xaa = Lys or Glu
TIYHSGSTYYNPSLXS

SEQ ID NO: 5 - protein, 15 aa, synthetic construct
  REGION 1..15: note = Synthetic Construct
ARQGSIQQGYYGMDV

SEQ ID NO: 6 - protein, 15 aa, synthetic construct
  REGION 1..15: note = Synthetic Construct
ARQGSIKQGYYGMDV

SEQ ID NO: 7 - protein, 15 aa, synthetic construct
  REGION 1..15: note = Synthetic Construct
  VARIANT 7: note = Xaa = Gln or Lys
ARQGSIXQGYYGMDV

SEQ ID NO: 8 - protein, 16 aa, synthetic construct
  REGION 1..16: note = Synthetic Construct
RSSQSLLRSNGYNYLD

SEQ ID NO: 9 - protein, 16 aa, synthetic construct
  REGION 1..16: note = Synthetic Construct
RSSQSLLRSTGYNYLD

SEQ ID NO: 10 - protein, 16 aa, synthetic construct
  REGION 1..16: note = Synthetic Construct
RSSQSLLRSSGYNYLD

SEQ ID NO: 11 - protein, 16 aa, synthetic construct
  REGION 1..16: note = Synthetic Construct
RSSQSLLRSGGYNYLD

SEQ ID NO: 12 - protein, 16 aa, synthetic construct
  REGION 1..16: note = Synthetic Construct
RSSQSLLRSRGYNYLD

SEQ ID NO: 13 - protein, 16 aa, synthetic construct
  REGION 1..16: note = Synthetic Construct
RSSQSLLRSDGYNYLD

SEQ ID NO: 14 - protein, 16 aa, synthetic construct
  REGION 1..16: note = Synthetic Construct
RSSQSLLRSHGYNYLD

SEQ ID NO: 15 - protein, 16 aa, synthetic construct
  REGION 1..16: note = Synthetic Construct
RSSQSLLRSKGYNYLD

SEQ ID NO: 16 - protein, 16 aa, synthetic construct
  REGION 1..16: note = Synthetic Construct
RSSQSLLRSQGYNYLD

SEQ ID NO: 17 - protein, 16 aa, synthetic construct
  REGION 1..16: note = Synthetic Construct
RSSQSLLRSYGYNYLD

SEQ ID NO: 18 - protein, 16 aa, synthetic construct
  REGION 1..16: note = Synthetic Construct
RSSQSLLRSEGYNYLD

SEQ ID NO: 19 - protein, 16 aa, synthetic construct
  REGION 1..16: note = Synthetic Construct
RSSQSLLRSWGYNYLD

SEQ ID NO: 20 - protein, 16 aa, synthetic construct
  REGION 1..16: note = Synthetic Construct
RSSQSLLRSFGYNYLD

SEQ ID NO: 21 - protein, 16 aa, synthetic construct
  REGION 1..16: note = Synthetic Construct
RSSQSLLRSIGYNYLD

SEQ ID NO: 22 - protein, 16 aa, synthetic construct
  REGION 1..16: note = Synthetic Construct
RSSQSLLRSVGYNYLD

SEQ ID NO: 23 - protein, 16 aa, synthetic construct
  REGION 1..16: note = Synthetic Construct
RSSQSLLRSAGYNYLD

SEQ ID NO: 24 - protein, 16 aa, synthetic construct
  REGION 1..16: note = Synthetic Construct
RSSQSLLRSMGYNYLD

SEQ ID NO: 25 - protein, 16 aa, synthetic construct
  REGION 1..16: note = Synthetic Construct
RSSQSLLRSLGYNYLD

SEQ ID NO: 26 - protein, 16 aa, synthetic construct
  REGION 1..16: note = Synthetic Construct
RSSQSLLHSNGYNYLD

SEQ ID NO: 27 - protein, 16 aa, synthetic construct
  REGION 1..16: note = Synthetic Construct
RSSQGLLRSNGYNYLD

SEQ ID NO: 28 - protein, 16 aa, synthetic construct
  REGION 1..16: note = Synthetic Construct
  VARIANT 5: note = Xaa = Ser or Gly
  VARIANT 8: note = Xaa = Arg or His
  VARIANT 10: note = Xaa = Asn, Thr, Ser, Gly, Arg, Asp, His, Lys, Gln, Tyr, Glu, Trp, Phe, Ile, Val, Ala, Met, or Leu
RSSQXLLXSXGYNYLD

SEQ ID NO: 29 - protein, 7 aa, synthetic construct
  REGION 1..7: note = Synthetic Construct
LGSNRAS

SEQ ID NO: 30 - protein, 7 aa, synthetic construct
  REGION 1..7: note = Synthetic Construct
LGSNRVS

SEQ ID NO: 31 - protein, 7 aa, synthetic construct
  REGION 1..7: note = Synthetic Construct
  VARIANT 6: note = Xaa = Ala or Val
LGSNRXS

SEQ ID NO: 32 - protein, 9 aa, synthetic construct
  REGION 1..9: note = Synthetic Construct
MQQQEAPLT

SEQ ID NO: 33 - protein, 9 aa, synthetic construct
  REGION 1..9: note = Synthetic Construct
MQQQETPLT

SEQ ID NO: 34 - protein, 9 aa, synthetic construct
  REGION 1..9: note = Synthetic Construct
  VARIANT 6: note = Xaa = Ala or Thr
MQQQEXPLT

SEQ ID NO: 35 - protein, 26 aa, synthetic construct
  REGION 1..26: note = Synthetic Construct
QVQLQESGPGLVKPSETLSLTCAVSG

SEQ ID NO: 36 - protein, 14 aa, synthetic construct
  REGION 1..14: note = Synthetic Construct
WIRQPPGKGLEWIG

SEQ ID NO: 37 - protein, 30 aa, synthetic construct
  REGION 1..30: note = Synthetic Construct
QVTISVDTSKNQFSLELSSVTAADTAVYYC

SEQ ID NO: 38 - protein, 30 aa, synthetic construct
  REGION 1..30: note = Synthetic Construct
RVTISVDTSKNQFSLKLSSVTAADTAVYYC

SEQ ID NO: 39 - protein, 30 aa, synthetic construct
  REGION 1..30: note = Synthetic Construct
  VARIANT 1: note = Xaa = Gln or Arg
  VARIANT 16: note = Xaa = Glu or Lys
XVTISVDTSKNQFSLXLSSVTAADTAVYYC

SEQ ID NO: 40 - protein, 11 aa, synthetic construct
  REGION 1..11: note = Synthetic Construct
WGQGTTVTVSS

SEQ ID NO: 41 - protein, 23 aa, synthetic construct
  REGION 1..23: note = Synthetic Construct
DIVMTQSPLSLPVTPGEPASISC

SEQ ID NO: 42 - protein, 23 aa, synthetic construct
  REGION 1..23: note = Synthetic Construct
DIVMTQSPLSLPVTPGGPASISC

SEQ ID NO: 43 - protein, 23 aa, synthetic construct
  REGION 1..23: note = Synthetic Construct
DIVMTQSPLSLPVTPGESASISC

SEQ ID NO: 44 - protein, 23 aa, synthetic construct
  REGION 1..23: note = Synthetic Construct
  VARIANT 17: note = Xaa = Glu or Gly
  VARIANT 18: note = Xaa = Pro or Ser
DIVMTQSPLSLPVTPGXXASISC

SEQ ID NO: 45 - protein, 15 aa, synthetic construct
  REGION 1..15: note = Synthetic Construct
WYLQKPGQSPQLLIY

SEQ ID NO: 46 - protein, 15 aa, synthetic construct
  REGION 1..15: note = Synthetic Construct
WYLQKPGQPPQLLIY

SEQ ID NO: 47 - protein, 15 aa, synthetic construct
  REGION 1..15: note = Synthetic Construct
  VARIANT 9: note = Xaa = Ser or Pro
WYLQKPGQXPQLLIY

SEQ ID NO: 48 - protein, 32 aa, synthetic construct
  REGION 1..32: note = Synthetic Construct
GVPDRFSGSGSGTDFTLKISRAEAEDVGVYYC

SEQ ID NO: 49 - protein, 32 aa, synthetic construct
  REGION 1..32: note = Synthetic Construct
GVPDRFSGSGSGTDFTLKISRVEAEDVGVYYC

SEQ ID NO: 50 - protein, 32 aa, synthetic construct
  REGION 1..32: note = Synthetic Construct
GVPDRFSGSGSGTDFTLKISRVEAEDVGAYYC

SEQ ID NO: 51 - protein, 32 aa, synthetic construct
  REGION 1..32: note = Synthetic Construct
GVPDRLSGSGSGTDFTLKISRVEAEDVGVYYC

SEQ ID NO: 52 - protein, 32 aa, synthetic construct
  REGION 1..32: note = Synthetic Construct
  VARIANT 6: note = Xaa = Phe or Leu
  VARIANT 22: note = Xaa = Ala or Val
  VARIANT 29: note = Xaa = Val or Ala
GVPDRXSGSGSGTDFTLKISRXEAEDVGXYYC

SEQ ID NO: 53 - protein, 10 aa, synthetic construct
  REGION 1..10: note = Synthetic Construct
FGGGTKVEIK

SEQ ID NO: 54 - protein, 122 aa, synthetic construct
  REGION 1..122: note = Synthetic Construct
QVQLQESGPGLVKPSETLSLTCAVSGYSISSGYYWGWIRQPPGKGLEWIGTIYHSGSTYYNPSLKSQVTISVDTSKNQFSLELSSVTAADTAVYYCARQGSIQQGYYGMDVWGQGTTVTVSS

SEQ ID NO: 55 - protein, 122 aa, synthetic construct
  REGION 1..122: note = Synthetic Construct
QVQLQESGPGLVKPSETLSLTCAVSGYSISSGYYWGWIRQPPGKGLEWIGTIYHSGSTYYNPSLESRVTISVDTSKNQFSLKLSSVTAADTAVYYCARQGSIQQGYYGMDVWGQGTTVTVSS

SEQ ID NO: 56 - protein, 122 aa, synthetic construct
  REGION 1..122: note = Synthetic Construct
QVQLQESGPGLVKPSETLSLTCAVSGYSISSGYYWGWIRQPPGKGLEWIGTIYHSGSTYYNPSLKSRVTISVDTSKNQFSLKLSSVTAADTAVYYCARQGSIKQGYYGMDVWGQGTTVTVSS

SEQ ID NO: 57 - protein, 112 aa, synthetic construct
  REGION 1..112: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRAEAEDVGVYYCMQQQEAPLTFGGGTKVEIK

SEQ ID NO: 58 - protein, 112 aa, synthetic construct
  REGION 1..112: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRVEAEDVGVYYCMQQQEAPLTFGGGTKVEIK

SEQ ID NO: 59 - protein, 112 aa, synthetic construct
  REGION 1..112: note = Synthetic Construct
DIVMTQSPLSLPVTPGEPASISCRSSQSLLRSNGYNYLDWYLQKPGQPPQLLIYLGSNRVSGVPDRFSGSGSGTDFTLKISRVEAEDVGVYYCMQQQETPLTFGGGTKVEIK

SEQ ID NO: 60 - protein, 112 aa, synthetic construct
  REGION 1..112: note = Synthetic Construct
DIVMTQSPLSLPVTPGEPASISCRSSQSLLRSTGYNYLDWYLQKPGQSPQLLIYLGSNRASGVPDRFSGSGSGTDFTLKISRAEAEDVGVYYCMQQQEAPLTFGGGTKVEIK

SEQ ID NO: 61 - protein, 112 aa, synthetic construct
  REGION 1..112: note = Synthetic Construct
DIVMTQSPLSLPVTPGEPASISCRSSQSLLRSSGYNYLDWYLQKPGQSPQLLIYLGSNRASGVPDRFSGSGSGTDFTLKISRAEAEDVGVYYCMQQQEAPLTFGGGTKVEIK

SEQ ID NO: 62 - protein, 112 aa, synthetic construct
  REGION 1..112: note = Synthetic Construct
DIVMTQSPLSLPVTPGEPASISCRSSQSLLRSGGYNYLDWYLQKPGQSPQLLIYLGSNRASGVPDRFSGSGSGTDFTLKISRAEAEDVGVYYCMQQQEAPLTFGGGTKVEIK

SEQ ID NO: 63 - protein, 112 aa, synthetic construct
  REGION 1..112: note = Synthetic Construct
DIVMTQSPLSLPVTPGEPASISCRSSQSLLRSRGYNYLDWYLQKPGQSPQLLIYLGSNRASGVPDRFSGSGSGTDFTLKISRAEAEDVGVYYCMQQQEAPLTFGGGTKVEIK

SEQ ID NO: 64 - protein, 112 aa, synthetic construct
  REGION 1..112: note = Synthetic Construct
DIVMTQSPLSLPVTPGEPASISCRSSQSLLRSDGYNYLDWYLQKPGQSPQLLIYLGSNRASGVPDRFSGSGSGTDFTLKISRAEAEDVGVYYCMQQQEAPLTFGGGTKVEIK

SEQ ID NO: 65 - protein, 112 aa, synthetic construct
  REGION 1..112: note = Synthetic Construct
DIVMTQSPLSLPVTPGEPASISCRSSQSLLRSHGYNYLDWYLQKPGQSPQLLIYLGSNRASGVPDRFSGSGSGTDFTLKISRAEAEDVGVYYCMQQQEAPLTFGGGTKVEIK

SEQ ID NO: 66 - protein, 112 aa, synthetic construct
  REGION 1..112: note = Synthetic Construct
DIVMTQSPLSLPVTPGEPASISCRSSQSLLRSKGYNYLDWYLQKPGQSPQLLIYLGSNRASGVPDRFSGSGSGTDFTLKISRAEAEDVGVYYCMQQQEAPLTFGGGTKVEIK

SEQ ID NO: 67 - protein, 112 aa, synthetic construct
  REGION 1..112: note = Synthetic Construct
DIVMTQSPLSLPVTPGEPASISCRSSQSLLRSQGYNYLDWYLQKPGQSPQLLIYLGSNRASGVPDRFSGSGSGTDFTLKISRAEAEDVGVYYCMQQQEAPLTFGGGTKVEIK

SEQ ID NO: 68 - protein, 112 aa, synthetic construct
  REGION 1..112: note = Synthetic Construct
DIVMTQSPLSLPVTPGEPASISCRSSQSLLRSYGYNYLDWYLQKPGQSPQLLIYLGSNRASGVPDRFSGSGSGTDFTLKISRAEAEDVGVYYCMQQQEAPLTFGGGTKVEIK

SEQ ID NO: 69 - protein, 112 aa, synthetic construct
  REGION 1..112: note = Synthetic Construct
DIVMTQSPLSLPVTPGEPASISCRSSQSLLRSEGYNYLDWYLQKPGQSPQLLIYLGSNRASGVPDRFSGSGSGTDFTLKISRAEAEDVGVYYCMQQQEAPLTFGGGTKVEIK

SEQ ID NO: 70 - protein, 112 aa, synthetic construct
  REGION 1..112: note = Synthetic Construct
DIVMTQSPLSLPVTPGEPASISCRSSQSLLRSWGYNYLDWYLQKPGQSPQLLIYLGSNRASGVPDRFSGSGSGTDFTLKISRAEAEDVGVYYCMQQQEAPLTFGGGTKVEIK

SEQ ID NO: 71 - protein, 112 aa, synthetic construct
  REGION 1..112: note = Synthetic Construct
DIVMTQSPLSLPVTPGEPASISCRSSQSLLRSFGYNYLDWYLQKPGQSPQLLIYLGSNRASGVPDRFSGSGSGTDFTLKISRAEAEDVGVYYCMQQQEAPLTFGGGTKVEIK

SEQ ID NO: 72 - protein, 112 aa, synthetic construct
  REGION 1..112: note = Synthetic Construct
DIVMTQSPLSLPVTPGEPASISCRSSQSLLRSIGYNYLDWYLQKPGQSPQLLIYLGSNRASGVPDRFSGSGSGTDFTLKISRAEAEDVGVYYCMQQQEAPLTFGGGTKVEIK

SEQ ID NO: 73 - protein, 112 aa, synthetic construct
  REGION 1..112: note = Synthetic Construct
DIVMTQSPLSLPVTPGEPASISCRSSQSLLRSVGYNYLDWYLQKPGQSPQLLIYLGSNRASGVPDRFSGSGSGTDFTLKISRAEAEDVGVYYCMQQQEAPLTFGGGTKVEIK

SEQ ID NO: 74 - protein, 112 aa, synthetic construct
  REGION 1..112: note = Synthetic Construct
DIVMTQSPLSLPVTPGEPASISCRSSQSLLRSAGYNYLDWYLQKPGQSPQLLIYLGSNRASGVPDRFSGSGSGTDFTLKISRAEAEDVGVYYCMQQQEAPLTFGGGTKVEIK

SEQ ID NO: 75 - protein, 112 aa, synthetic construct
  REGION 1..112: note = Synthetic Construct
DIVMTQSPLSLPVTPGEPASISCRSSQSLLRSMGYNYLDWYLQKPGQSPQLLIYLGSNRASGVPDRFSGSGSGTDFTLKISRAEAEDVGVYYCMQQQEAPLTFGGGTKVEIK

SEQ ID NO: 76 - protein, 112 aa, synthetic construct
  REGION 1..112: note = Synthetic Construct
DIVMTQSPLSLPVTPGEPASISCRSSQSLLRSLGYNYLDWYLQKPGQSPQLLIYLGSNRASGVPDRFSGSGSGTDFTLKISRAEAEDVGVYYCMQQQEAPLTFGGGTKVEIK

SEQ ID NO: 77 - protein, 112 aa, synthetic construct
  REGION 1..112: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRVEAEDVGAYYCMQQQEAPLTFGGGTKVEIK

SEQ ID NO: 78 - protein, 112 aa, synthetic construct
  REGION 1..112: note = Synthetic Construct
DIVMTQSPLSLPVTPGGPASISCRSSQSLLRSNGYNYLDWYLQKPGQSPQLLIYLGSNRASGVPDRLSGSGSGTDFTLKISRVEAEDVGVYYCMQQQETPLTFGGGTKVEIK

SEQ ID NO: 79 - protein, 112 aa, synthetic construct
  REGION 1..112: note = Synthetic Construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQQQETPLTFGGGTKVEIK

SEQ ID NO: 80 - protein, 112 aa, synthetic construct
  REGION 1..112: note = Synthetic Construct
DIVMTQSPLSLPVTPGESASISCRSSQGLLRSNGYNYLDWYLQKPGQSPQLLIYLGSNRASGVPDRFSGSGSGTDFTLKISRVEAEDVGVYYCMQQQEAPLTFGGGTKVEIK

SEQ ID NO: 81 - protein, 831 aa, Homo sapiens
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 82 - protein, 825 aa, Mus musculus
MERPRGAADGLLRWPLGLLLLLQLLPPAAVGQDRLDAPPPPAPPLLRWAGPVGVSWGLRAAAPGGPVPRAGRWRRGAPAEDQDCGRLPDFIAKLTNNTHQHVFDDLSGSVSLSWVGDSTGVILVLTTFQVPLVIVSFGQSKLYRSEDYGKNFKDITNLINNTFIRTEFGMAIGPENSGKVILTAEVSGGSRGGRVFRSSDFAKNFVQTDLPFHPLTQMMYSPQNSDYLLALSTENGLWVSKNFGEKWEEIHKAVCLAKWGPNNIIFFTTHVNGSCKADLGALELWRTSDLGKTFKTIGVKIYSFGLGGRFLFASVMADKDTTRRIHVSTDQGDTWSMAQLPSVGQEQFYSILAANEDMVFMHVDEPGDTGFGTIFTSDDRGIVYSKSLDRHLYTTTGGETDFTNVTSLRGVYITSTLSEDNSIQSMITFDQGGRWEHLRKPENSKCDATAKNKNECSLHIHASYSISQKLNVPMAPLSEPNAVGIVIAHGSVGDAISVMVPDVYISDDGGYSWAKMLEGPHYYTILDSGGIIVAIEHSNRPINVIKFSTDEGQCWQSYVFTQEPIYFTGLASEPGARSMNISIWGFTESFITRQWVSYTVDFKDILERNCEEDDYTTWLAHSTDPGDYKDGCILGYKEQFLRLRKSSVCQNGRDYVVAKQPSVCPCSLEDFLCDFGYFRPENASECVEQPELKGHELEFCLYGKEEHLTTNGYRKIPGDKCQGGMNPAREVKDLKKKCTSNFLNPTKQNSKSNSVPIILAIVGLMLVTVVAGVLIVKKYVCGGRFLVHRYSVLQQHAEADGVEALDSTSHAKSGYHDDSDEDLLE

SEQ ID NO: 83 - protein, 825 aa, Rattus norvegicus
MERPRGAADGLLRWPLGLLLLLQLLPPAAVGQDRLDAPPPPAPPLLRWAGPVGVSWGLRAAAPGGPVPRAGRWRRGAPAEDQDCGRLPDFIAKLTNNTHQHVFDDLSGSVSLSWVGDSTGVILVLTTFQVPLVIVSFGQSKLYRSEDYGKNFKDITNLINNTFIRTEFGMAIGPENSGKVILTAEVSGGSRGGRVFRSSDFAKNFVQTDLPFHPLTQMMYSPQNSDYLLALSTENGLWVSKNFGEKWEEIHKAVCLAKWGPNNIIFFTTHVNGSCKADLGALELWRTSDLGKTFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSTLSEDNSIQSMITFDQGGRWEHLQKPENSKCDATAKNKNECSLHIHASYSISQKLNVPMAPLSEPNAVGIVIAHGSVGDAISVMVPDVYISDDGGYSWAKMLEGPHYYTILDSGGIIVAIEHSNRPINVIKFSTDEGQCWQSYVFSQEPVYFTGLASEPGARSMNISIWGFTESFLTRQWVSYTIDFKDILERNCEENDYTTWLAHSTDPGDYKDGCILGYKEQFLRLRKSSVCQNGRDYVVAKQPSICPCSLEDFLCDFGYFRPENASECVEQPELKGHELEFCLYGKEEHLTTNGYRKIPGDRCQGGMNPAREVKDLKKKCTSNFLNPKKQNSKSSSVPIILAIVGLMLVTVVAGVLIVKKYVCGGRFLVHRYSVLQQHAEADGVEALDTASHAKSGYHDDSDEDLLE

SEQ ID NO: 84 - protein, 330 aa, synthetic construct
  REGION 1..330: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 329 aa, synthetic construct
  REGION 1..329: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 452 aa, synthetic construct
  REGION 1..452: note = Synthetic Construct
QVQLQESGPGLVKPSETLSLTCAVSGYSISSGYYWGWIRQPPGKGLEWIGTIYHSGSTYYNPSLKSQVTISVDTSKNQFSLELSSVTAADTAVYYCARQGSIQQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 451 aa, synthetic construct
  REGION 1..451: note = Synthetic Construct
QVQLQESGPGLVKPSETLSLTCAVSGYSISSGYYWGWIRQPPGKGLEWIGTIYHSGSTYYNPSLKSQVTISVDTSKNQFSLELSSVTAADTAVYYCARQGSIQQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 452 aa, synthetic construct
  REGION 1..452: note = Synthetic Construct
QVQLQESGPGLVKPSETLSLTCAVSGYSISSGYYWGWIRQPPGKGLEWIGTIYHSGSTYYNPSLESRVTISVDTSKNQFSLKLSSVTAADTAVYYCARQGSIQQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 451 aa, synthetic construct
  REGION 1..451: note = Synthetic Construct
QVQLQESGPGLVKPSETLSLTCAVSGYSISSGYYWGWIRQPPGKGLEWIGTIYHSGSTYYNPSLESRVTISVDTSKNQFSLKLSSVTAADTAVYYCARQGSIQQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 452 aa, synthetic construct
  REGION 1..452: note = Synthetic Construct
QVQLQESGPGLVKPSETLSLTCAVSGYSISSGYYWGWIRQPPGKGLEWIGTIYHSGSTYYNPSLKSRVTISVDTSKNQFSLKLSSVTAADTAVYYCARQGSIKQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 451 aa, synthetic construct
  REGION 1..451: note = Synthetic Construct
QVQLQESGPGLVKPSETLSLTCAVSGYSISSGYYWGWIRQPPGKGLEWIGTIYHSGSTYYNPSLKSRVTISVDTSKNQFSLKLSSVTAADTAVYYCARQGSIKQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 219 aa, synthetic construct
  REGION 1..219: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 219 aa, synthetic construct
  REGION 1..219: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRVEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 219 aa, synthetic construct
  REGION 1..219: note = Synthetic Construct
DIVMTQSPLSLPVTPGEPASISCRSSQSLLRSNGYNYLDWYLQKPGQPPQLLIYLGSNRVSGVPDRFSGSGSGTDFTLKISRVEAEDVGVYYCMQQQETPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 219 aa, synthetic construct
  REGION 1..219: note = Synthetic Construct
DIVMTQSPLSLPVTPGEPASISCRSSQSLLRSTGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 219 aa, synthetic construct
  REGION 1..219: note = Synthetic Construct
DIVMTQSPLSLPVTPGEPASISCRSSQSLLRSSGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 219 aa, synthetic construct
  REGION 1..219: note = Synthetic Construct
DIVMTQSPLSLPVTPGEPASISCRSSQSLLRSGGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 219 aa, synthetic construct
  REGION 1..219: note = Synthetic Construct
DIVMTQSPLSLPVTPGEPASISCRSSQSLLRSRGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 219 aa, synthetic construct
  REGION 1..219: note = Synthetic Construct
DIVMTQSPLSLPVTPGEPASISCRSSQSLLRSDGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 219 aa, synthetic construct
  REGION 1..219: note = Synthetic Construct
DIVMTQSPLSLPVTPGEPASISCRSSQSLLRSHGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 219 aa, synthetic construct
  REGION 1..219: note = Synthetic Construct
DIVMTQSPLSLPVTPGEPASISCRSSQSLLRSKGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 219 aa, synthetic construct
  REGION 1..219: note = Synthetic Construct
DIVMTQSPLSLPVTPGEPASISCRSSQSLLRSQGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 219 aa, synthetic construct
  REGION 1..219: note = Synthetic Construct
DIVMTQSPLSLPVTPGEPASISCRSSQSLLRSYGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 219 aa, synthetic construct
  REGION 1..219: note = Synthetic Construct
DIVMTQSPLSLPVTPGEPASISCRSSQSLLRSEGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 219 aa, synthetic construct
  REGION 1..219: note = Synthetic Construct
DIVMTQSPLSLPVTPGEPASISCRSSQSLLRSWGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 219 aa, synthetic construct
  REGION 1..219: note = Synthetic Construct
DIVMTQSPLSLPVTPGEPASISCRSSQSLLRSFGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 219 aa, synthetic construct
  REGION 1..219: note = Synthetic Construct
DIVMTQSPLSLPVTPGEPASISCRSSQSLLRSIGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 219 aa, synthetic construct
  REGION 1..219: note = Synthetic Construct
DIVMTQSPLSLPVTPGEPASISCRSSQSLLRSVGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 219 aa, synthetic construct
  REGION 1..219: note = Synthetic Construct
DIVMTQSPLSLPVTPGEPASISCRSSQSLLRSAGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 219 aa, synthetic construct
  REGION 1..219: note = Synthetic Construct
DIVMTQSPLSLPVTPGEPASISCRSSQSLLRSMGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 219 aa, synthetic construct
  REGION 1..219: note = Synthetic Construct
DIVMTQSPLSLPVTPGEPASISCRSSQSLLRSLGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 219 aa, synthetic construct
  REGION 1..219: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRVEAEDVGAYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 219 aa, synthetic construct
  REGION 1..219: note = Synthetic Construct
DIVMTQSPLSLPVTPGGPASISCRSSQSLLRSNGYNYLDWYLQKPGQSPQLLIYLGSNRASGVPDRLSGSGSGTDFTLKISRVEAEDVGVYYCMQQQETPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 219 aa, synthetic construct
  REGION 1..219: note = Synthetic Construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQQQETPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 219 aa, synthetic construct
  REGION 1..219: note = Synthetic Construct
DIVMTQSPLSLPVTPGESASISCRSSQGLLRSNGYNYLDWYLQKPGQSPQLLIYLGSNRASGVPDRFSGSGSGTDFTLKISRVEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0223365 A1
(43) Pub. Date: Jul. 10, 2025

(54) METHODS OF USE OF ANTI-SORTILIN ANTIBODIES

(71) Applicant: Alector LLC, South San Francisco, CA (US)

(72) Inventors: Robert PAUL, San Francisco, CA (US); Michael F. WARD, San Francisco, CA (US); Hua LONG, San Carlos, CA (US); Shiao-Ping LU, Los Altos, CA (US); Omer Rizwan SIDDIQUI, Redwood City, CA (US); Arnon ROSENTHAL, Woodside, CA (US); Herve RHINN, Paris (FR)

(73) Assignee: Alector LLC, South San Francisco, CA (US)

(21) Appl. No.: 18/776,646

(22) Filed: Jul. 18, 2024

Related U.S. Application Data
(63) Continuation of application No. 16/898,307, filed on Jun. 10, 2020, now abandoned.
(60) Provisional application No. 62/961,591, filed on Jan. 15, 2020.
(60) Provisional application No. 62/947,503, filed on Dec. 12, 2019.
(60) Provisional application No. 62/874,475, filed on Jul. 15, 2019.
(60) Provisional application No. 62/868,850, filed on Jun. 28, 2019.
(60) Provisional application No. 62/860,207, filed on Jun. 11, 2019.

(51) Int. Cl. C07K 16/28; A61K 9/00; A61K 39/00; G01N 33/68

(52) U.S. Cl. CPC C07K 16/286; G01N 33/6896; A61K 9/0019; A61K 2039/545; C07K 2317/24; C07K 2317/40; C07K 2317/56; G01N 2333/70571

(57) ABSTRACT

The present disclosure is generally directed to the use of compositions that include antibodies, e.g., monoclonal, chimeric, affinity-matured, humanized antibodies, antibody fragments, etc., that specifically bind one or more epitopes within a Sortilin protein, e.g., human Sortilin or mammalian Sortilin, and have improved and/or enhanced functional characteristics, in treating and/or delaying progression of a disease or injury in an individual in need thereof.

### CROSS-REFERENCE TO RELATED APPLICATION

This application is a continuation of U.S. application Ser. No. 16/898,307, filed Jun. 10, 2020, which claims the benefit of U.S. Provisional Application No. 62/860,207, filed Jun. 11, 2019, U.S. Provisional Application No. 62/868,850, filed Jun. 28, 2019, U.S. Provisional Application No. 62/874,475, filed Jul. 15, 2019, U.S. Provisional Application No. 62/947,503, filed Dec. 12, 2019, and U.S. Provisional Application No. 62/961,591, filed Jan. 15, 2020, each of which is hereby incorporated by reference in its entirety.

### REFERENCE TO SEQUENCE LISTING SUBMITTED ELECTRONICALLY

The content of the electronic sequence listing (735022003001seqlist.xml; Size: 162,846 bytes; and Date of Creation: May 1, 2024) is herein incorporated by reference in its entirety.

### FIELD

This present disclosure relates to therapeutic uses of anti-Sortilin antibodies.

### BACKGROUND

Sortilin is a Type I transmembrane protein that acts both as a receptor of several ligands, and in the sorting of select cargo from the trans-Golgi network (TGN) to late endosomes and lysosomes for degradation. Sortilin binds the secreted protein Progranulin (PGRN) and targets it for lysosomal degradation, thus negatively regulating extracellular levels of PGRN (Hu, F et al. (2010) Neuron 68, 654-667). In line with this, deficiency of Sortilin significantly increases plasma PGRN levels both in mouse models in vivo and human cells in vitro (Carrasquillo, M. M et al., (2010) Am J Hum Genet 87, 890-897; Lee, W. C et al., (2014) Hum Mol Genet 23, 1467-1478). Moreover, a polymorphism in Sortilin was shown to be strongly associated with PGRN serum levels in humans (Carrasquillo M M e al., (2010), Am J Hum Genet. 10; 87(6):890-7).

Progranulin (PGRN) is a secreted, growth factor-like, trophic, and anti-inflammatory protein, which also plays a role as an adipokine involved in diet-induced obesity and insulin resistance (Nguyen D A et al., (2013). Trends in Endocrinology and Metabolism, 24, 597-606). Progranulin deficiency accounts for roughly 25% of all heritable forms of frontotemporal dementia (FTD), an early-onset neurodegenerative disease. Patients with heterozygous loss-of-function mutations in PGRN have ˜50% reduced extracellular levels of the protein and they will invariably develop FTD, making PGRN a causal gene for the disease (Baker, M et al., (2006) Nature 442, 916-919; Carecchio M et al., (2011) J Alzheimers Dis 27, 781-790; Cruts, M et al., (2008) Trends Genet 24, 186-194; Galimberti, D et al., (2010) J Alzheimers Dis 19, 171-177). In addition, PGRN mutant alleles have been identified in Alzheimer's disease patients (Seelaar, H et al., (2011). Journal of neurology, neurosurgery, and psychiatry 82, 476-486). Importantly, PGRN acts protectively in several disease models, with increased PGRN levels accelerating behavioral recovery from ischemia (Tao, J et al., (2012) Brain Res 1436, 130-136; Egashira, Y. et al., (2013) J Neuroinflammation 10, 105), suppressing locomotor deficits in a Parkinson's disease model (Van Kampen, J. M et al. (2014). PLoS One 9, e97032), attenuating pathology in a model of amyotrophic lateral sclerosis (Laird, A. S et al., (2010). PLoS One 5, e13368.) and arthritis (Tang, W et al., (2011). Science 332, 478-484), and preventing memory deficits in an Alzheimer's disease model (Minami, S. S et al., (2014). Nat Med 20, 1157-1164).

Through its various interactions with proteins, such as Progranulin, Sortilin and its multiple ligands have been shown to be involved in various diseases, disorders, and conditions, such as frontotemporal dementia (FTD), amyotrophic lateral sclerosis (ALS), amyotrophic lateral sclerosis-frontotemporal dementia phenotypes, Alzheimer's disease, Parkinson's disease, depression, neuropsychiatric disorders, vascular dementia, seizures, retinal dystrophy, age related macular degeneration, glaucoma, traumatic brain injury, aging, seizures, wound healing, stroke, arthritis, and atherosclerotic vascular diseases.

Novel therapeutic antibodies targeting Sortilin are one solution to treating diseases associated with Sortilin activity. Systemically administered monoclonal antibodies normally exhibit a biphasic pharmacokinetic profile, being first distributed relatively quickly and then eliminated more slowly (Ovacik, M and Lin, L, (2018) Clin Transl Sci 11, 540-552). Circulation of systemically administered antibodies is typically confined to the vasculature and interstitial space (Ovacik, M and Lin, L, (2018) Clin Transl Sci 11, 540-552). This is because of their size, polarity, recycling and clearance kinetics, and typically relatively long half-lives, which are often 11-30 days in humans (Ovacik, M and Lin, L, (2018) Clin Transl Sci 11, 540-552).

Administration of monoclonal antibodies presents a challenge for therapeutic use. Monoclonal antibodies have limited oral bioavailability, so they are typically administered intravenously, subcutaneously, or intramuscularly (Ovacik, M and Lin, L, (2018) Clin Transl Sci 11, 540-552). Of those options, subcutaneous administration is the most convenient because it can be done at home and often by the patient himself, but intravenous administration delivers higher systemic exposures. Delivery to the cerebrospinal fluid (CSF) requires high systemic doses. Thus, when treatment requires impacting the CSF, intravenous administration is usually required because subcutaneous administration cannot deliver sufficiently high doses.

However, intravenous administration is particularly challenging for patients with neurodegenerative diseases, such as FTD and ALS. These diseases affect patients for long periods of time and thus require regular treatment over the course of many years. As intravenous administration cannot be done at home, patients must be transported to infusion centers on a regular basis, which is a burden on both the patient and caregiver. Finally, the memory loss, mood swings, aggression, and other behavioral symptoms of these diseases make patient compliance difficult.

Accordingly, there is a need for therapeutic antibodies that specifically bind Sortilin proteins and block the binding of Sortilin to its ligands, such as Progranulin, or otherwise modulate the effective concentration of the ligands, in order to treat one or more diseases, disorders, and conditions associated with Sortilin activity. Furthermore, due to the limitations on modes of administration and dosing, there are additional needs for identifying methods of treating patients with the correct dose and of administering that dose in ways that ease patient compliance. All references cited herein, including patents, patent applications and publications, are hereby incorporated by reference in their entirety.

### SUMMARY

The present disclosure is generally directed to methods of using compositions that include antibodies, e.g., monoclonal, chimeric, humanized antibodies, antibody fragments, etc., that specifically bind human Sortilin.

In certain aspects, provided herein is a method of treating and/or delaying progression of a disease or injury in an individual, comprising administering to the individual an anti-Sortilin antibody intravenously at a dose of at least about 30 mg/kg once every four weeks or more frequently, where the antibody comprises: (i) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32); (ii) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRVS (SEQ ID NO: 30), and the HVR-L3 comprising the amino acid sequence MQQQETPLT (SEQ ID NO: 33); (iii) a heavy chain variable region comprising the HVR-H1 comprising the amino acid sequence YSISSGYYWG (SEQ ID NO: 1), the HVR-H2 comprising the amino acid sequence TIYHSGSTYYNPSLES (SEQ ID NO: 3), the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32); (iv) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32); (v) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSTGYNYLD (SEQ ID NO: 9), the HVR-L2 comprising the amino acid sequence LGSNRAS (SEQ ID NO: 29), and the HVR-L3 comprising the amino acid sequence MQQQEAPLT (SEQ ID NO: 32); (vi) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQETPLT (SEQ ID NO: 33); (vii) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIQQGYYGMDV (SEQ ID NO: 5); and a light chain variable region comprising the HVR-L1 comprising the amino acid sequence RSSQSLLHSNGYNYLD (SEQ ID NO: 26), the HVR-L2 comprising the amino acid sequence LGSNRAS (SEQ ID NO: 29), and the HVR-L3 comprising the amino acid sequence MQQQETPLT (SEQ ID NO: 33); or (viii) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQGLLRSNGYNYLD (SEQ ID NO: 27), the HVR-L2 comprising the amino acid sequence LGSNRAS (SEQ ID NO: 29), and the HVR-L3 comprising the amino acid sequence MQQQEAPLT (SEQ ID NO: 32).

In some embodiments, the anti-Sortilin antibody comprises a heavy chain variable region and a light chain variable region, wherein the heavy chain variable region comprises an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and the light chain variable region comprises an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32).

In some embodiments, the anti-Sortilin antibody comprises a heavy chain variable region and a light chain variable region, wherein the antibody comprises a heavy chain variable region with an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and the light chain variable region comprises an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

In some embodiments, the anti-Sortilin antibody comprises a heavy chain variable region comprising the amino acid sequence of SEQ ID NO: 54, and a light chain variable region comprising the amino acid sequence of SEQ ID NO: 57.

In some embodiments, the anti-Sortilin antibody comprises a heavy chain variable region comprising the amino acid sequence of SEQ ID NO: 54, and a light chain variable region comprising the amino acid sequence of SEQ ID NO: 58.

In some embodiments, the anti-Sortilin antibody comprises a heavy chain variable region comprising the amino acid sequence of SEQ ID NO: 54, and a light chain variable region comprising the amino acid sequence of SEQ ID NO: 59.

In some embodiments, the anti-Sortilin antibody comprises a heavy chain variable region comprising the amino acid sequence of SEQ ID NO: 55, and a light chain variable region comprising the amino acid sequence of SEQ ID NO: 57.

In some embodiments, the anti-Sortilin antibody comprises a heavy chain variable region comprising the amino acid sequence of SEQ ID NO: 55, and a light chain variable region comprising the amino acid sequence of SEQ ID NO: 58.

In some embodiments, the anti-Sortilin antibody comprises a heavy chain variable region comprising the amino acid sequence of SEQ ID NO: 56, and a light chain variable region comprising the amino acid sequence of SEQ ID NO: 57.

In some embodiments, the anti-Sortilin antibody comprises a heavy chain variable region comprising the amino acid sequence of SEQ ID NO: 56, and a light chain variable region comprising the amino acid sequence of SEQ ID NO: 77.

In some embodiments, the anti-Sortilin antibody comprises a heavy chain variable region comprising the amino acid sequence of SEQ ID NO: 56, and a light chain variable region comprising the amino acid sequence of SEQ ID NO: 78.

In some embodiments, the anti-Sortilin antibody comprises a heavy chain variable region comprising the amino acid sequence of SEQ ID NO: 54, and a light chain variable region comprising the amino acid sequence of SEQ ID NO: 79.

In some embodiments, the anti-Sortilin antibody comprises a heavy chain variable region comprising the amino acid sequence of SEQ ID NO: 56, and a light chain variable region comprising the amino acid sequence of SEQ ID NO: 80.

In some embodiments, the anti-Sortilin antibody comprises a heavy chain variable region comprising the amino acid sequence of SEQ ID NO: 56 and a light chain variable region comprising the amino acid sequence of SEQ ID NO: 57. In some embodiments, the anti-Sortilin antibody comprises a heavy chain variable region comprising the amino acid sequence of SEQ ID NO: 56 and a light chain variable region comprising the amino acid sequence of SEQ ID NO: 60.

In some embodiments, the antibody is an IgG1 isotype and the Fc region comprises amino acid substitutions at positions L234A, L235A, and P331S, wherein the numbering of the residue position is according to EU numbering.

In some embodiments, the dose is at least about 35 mg/kg, at least about 40 mg/kg, at least about 45 mg/kg, at least about 50 mg/kg, at least about 55 mg/kg, or at least about 60 mg/kg. In some embodiments, the dose is between about 30 mg/kg and about 60 mg/kg. In some embodiments, the dose is about 60 mg/kg.

In some embodiments, the anti-Sortilin antibody is administered once every two weeks. In some embodiments, the anti-Sortilin antibody is administered once every three weeks. In some embodiments, the anti-Sortilin antibody is administered once every four weeks.

In some embodiments, the anti-Sortilin antibody is administered once every four weeks at a dose of about 60 mg/kg.

In some embodiments, the disease or injury is selected from the group consisting of frontotemporal dementia, progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, and osteoarthritis. In some embodiments, the disease or injury is frontotemporal dementia. In some embodiments, the disease or injury is amytrophic lateral sclerosis.

In some embodiments, the individual is heterozygous for a mutation in GRN. In some embodiments, the mutation in GRN is a loss-of-function mutation. In some embodiments, the individual is heterozygous for a C9orf72 hexanucleotide repeat expansion. In some embodiments, the individual shows symptoms of frontotemporal dementia. In some embodiments, the individual does not show symptoms of frontotemporal dementia.

In some embodiments, the level of PGRN protein in the plasma of the individual after administration of the anti-Sortilin antibody is at least one-fold higher than the level of PGRN protein in the plasma of the individual before administration of the anti-Sortilin antibody. In some embodiments, the level of PGRN protein in the plasma of the individual after administration of the anti-Sortilin antibody is at least two-fold higher than the level of PGRN protein in the plasma of the individual before administration of the anti-Sortilin antibody. In some embodiments, the fold increase in the level of PGRN protein in the plasma of the individual is present at about five days after administration of the anti-Sortilin antibody. In some embodiments, the fold increase in the level of PGRN protein in the plasma of the individual is present at about 42 days after administration of the anti-Sortilin antibody. In some embodiments, the fold increase in the level of PGRN protein in the plasma of the individual is present at about 56 days after administration of the anti-Sortilin antibody. In some embodiments, the level of PGRN protein in the plasma of the individual after administration of the anti-Sortilin antibody is at least 0.25-fold higher than the level of PGRN protein in the plasma of the individual before administration of the anti-Sortilin antibody at about forty days after administration of the anti-Sortilin antibody.

In some embodiments, the level of PGRN protein in the plasma of the individual after administration of the anti-Sortilin antibody is at least two-fold, three-fold, or four-fold higher than the level of PGRN protein in the plasma of the individual before administration of the anti-Sortilin antibody. In some embodiments, the fold increase in the level of PGRN protein in the plasma of the individual is present at about five days after the last administration of the anti-Sortilin antibody. In some embodiments, the fold increase in the level of PGRN protein in the plasma of the individual is present at about 28 days, 35 days, 42 days, 49 days, or 56 days after the last administration of the anti-Sortilin antibody.

In some embodiments, the level of PGRN protein in the cerebrospinal fluid of the individual after administration of the anti-Sortilin antibody is at least 0.8-fold higher than the level of PGRN protein in the cerebrospinal fluid of the individual before administration of the anti-Sortilin antibody. In some embodiments, the level of PGRN protein in the cerebrospinal fluid of the individual after administration of the anti-Sortilin antibody is at least one-fold higher than the level of PGRN protein in the cerebrospinal fluid of the individual before administration of the anti-Sortilin antibody. In some embodiments, the fold increase in the level of PGRN protein in the cerebrospinal fluid of the individual is present at about twelve days after administration of the anti-Sortilin antibody. In some embodiments, the fold increase in the level of PGRN protein in the cerebrospinal fluid of the individual is present at about 24 days after administration of the anti-Sortilin antibody. In some embodiments, the fold increase in the level of PGRN protein in the cerebrospinal fluid of the individual is present at about 56 days after administration of the anti-Sortilin antibody. In some embodiments, the level of PGRN protein in the cerebrospinal fluid of the individual after administration of the anti-Sortilin antibody is at least 0.2-fold higher than the level of PGRN protein in the cerebrospinal fluid of the individual before administration of the anti-Sortilin antibody at about 42 days after administration of the anti-Sortilin antibody.

In some embodiments, the level of PGRN protein in the cerebrospinal fluid of the individual after administration of the anti-Sortilin antibody is at least two-fold higher than the level of PGRN protein in the cerebrospinal fluid of the individual before administration of the anti-Sortilin antibody. In some embodiments, the fold increase in the level of PGRN protein in the cerebrospinal fluid of the individual is present at about twelve days after the last administration of the anti-Sortilin antibody. In some embodiments, the fold increase in the level of PGRN protein in the cerebrospinal fluid of the individual is present at about 24 days after the last administration of the anti-Sortilin antibody. In some embodiments, the fold increase in the level of PGRN protein in the cerebrospinal fluid of the individual is present at about 28, 35, 42, 49, or 56 days after the last administration of the anti-Sortilin antibody. In some embodiments, the fold increase in the level of PGRN protein in the cerebrospinal fluid of the individual is present at about 28 days, 35 days, 42 days, 49 days, or 56 days after the last administration of the anti-Sortilin antibody.

In some embodiments, the expression level of SORT1 protein on peripheral white blood cells of the individual after administration of the anti-Sortilin antibody is reduced by at least 50% compared to the expression level of SORT1 protein on peripheral white blood cells of the individual before administration of the anti-Sortilin antibody. In some embodiments, the expression level of SORT1 protein on peripheral white blood cells of the individual after administration of the anti-Sortilin antibody is reduced by at least 70% compared to the expression level of SORT1 protein on peripheral white blood cells of the individual before administration of the anti-Sortilin antibody. In some embodiments, the reduction in the expression level of SORT1 in peripheral white blood cells of the individual is present at about twelve days or more after administration of the anti-Sortilin antibody. In some embodiments, the reduction in the expression level of SORT1 in peripheral white blood cells of the individual is present at about seventeen days or more after administration of the anti-Sortilin antibody. In some embodiments, the reduction in the expression level of SORT1 in peripheral white blood cells of the individual is present at about forty days or more after administration of the anti-Sortilin antibody. In some embodiments, the reduction in the expression level of SORT1 in peripheral white blood cells of the individual is present at about twelve days or more after the last administration of the anti-Sortilin antibody. In some embodiments, the reduction in the expression level of SORT1 in peripheral white blood cells of the individual is present at about seventeen days or more after the last administration of the anti-Sortilin antibody. In some embodiments, the reduction in the expression level of SORT1 in peripheral white blood cells of the individual is present at about forty days or more after the last administration of the anti-Sortilin antibody.

In some embodiments, the half-life of the anti-Sortilin antibody in plasma is around 5 days. In some embodiments, the half-life of the anti-Sortilin antibody in plasma is around 8 days.

In some embodiments, the individual is treated for a treatment period of up to 48 weeks in length. In some embodiments, the individual is treated for a treatment period of 48 weeks in length. In some embodiments, administration of the anti-Sortilin antibody occurs on the first day of the treatment period and every four weeks thereafter. In some embodiments, the anti-Sortilin antibody is administered a total of 13 times during the treatment period.

In some embodiments, the disease or injury is frontotemporal dementia (FTD), and plasma neurofilament light chain (NfL) levels are reduced by at least 10%. In some embodiments, the disease or injury is frontotemporal dementia (FTD), and plasma neurofilament light chain (NfL) levels are reduced by at least 10% after administration of the anti-Sortilin antibody compared to the plasma neurofilament light chain (NfL) levels before administration of the anti-Sortilin antibody.

In some embodiments, the protein levels of CTSB in the CSF of the individual are increased by at least about 20% compared to the protein levels of CTSB in the CSF of the individual before administration of the anti-Sortilin antibody. In some embodiments, the protein levels of SPP1 in the CSF of the individual are decreased by at least about 10% compared to the protein levels of SPP1 in the CSF of the individual before administration of the anti-Sortilin antibody. In some embodiments, the protein levels of CTSB in the CSF of the individual are increased by at least about 20% after administration of the anti-Sortilin antibody compared to the protein levels of CTSB in the CSF of the individual before administration of the anti-Sortilin antibody. In some embodiments, the protein levels of SPP1 in the CSF of the individual are decreased by at least about 10% after administration of the anti-Sortilin antibody compared to the protein levels of SPP1 in the CSF of the individual before administration of the anti-Sortilin antibody. In some embodiments, the protein levels of N-acetylglucosamine kinase (NAGK) in the CSF of the individual are increased after administration of the anti-Sortilin antibody compared to the protein levels of NAGK in the CSF of the individual before administration of the anti-Sortilin antibody. In some embodiments, the protein levels of one or more inflammatory proteins in the CSF of the individual are decreased after administration of the anti-Sortilin antibody compared to the protein levels of the one or more inflammatory proteins in the CSF of the individual before administration of the anti-Sortilin antibody, wherein the one or more inflammatory proteins are selected from the group consisting of 14-3-3 protein epsilon (YWHAE), allograft inflammatory factor 1 (AIF1), colony stimulating factor 1 (CSF1), chitinase 1 (CHIT1), lymphocyte antigen 86 (LY86), and CD86.

In another aspect, provided herein is a method of monitoring the treatment of an individual being administered an anti-Sortilin antibody comprising measuring the level of one or more proteins in a sample from the individual before and after the individual has received one or more doses of an anti-Sortilin antibody, wherein the one or more proteins are CTSB and/or SPP1. In some embodiments, the method of monitoring the treatment of an individual being administered an anti-Sortilin antibody further comprises a step of assessing the activity of the anti-Sortilin antibody in the individual based on the level of the one or more proteins in the sample. In some embodiments, the sample is from the cerebrospinal fluid of the individual or the blood of the individual. In some embodiments, the sample is from the cerebrospinal fluid of the individual.

In another aspect, provided herein is a method of monitoring the treatment of an individual being administered an anti-Sortilin antibody, comprising measuring the level of one or more proteins in a sample from the individual before and after the individual has received one or more doses of an anti-Sortilin antibody, wherein the one or more proteins are selected from the group consisting of CTSB, SPP1, NAGK, YWHAE, AIF1, CSF1, CHIT1, LY86, and CD86. In some embodiments, the method further comprises assessing the activity of the anti-Sortilin antibody in the individual based on the level of the one or more proteins in the sample. In some embodiments, the sample is from the cerebrospinal fluid of the individual. In some embodiments, the anti-Sortilin antibody is determined to be active in the individual if the level of CTSB in the cerebrospinal fluid after the individual has received one or more doses of the anti-Sortilin antibody is increased compared to the level of CTSB in the cerebrospinal fluid before the individual received one or more doses of the anti-Sortilin antibody. In some embodiments, the anti-Sortilin antibody is determined to be active in the individual if the level of CTSB in the cerebrospinal fluid after the individual has received one or more doses of the anti-Sortilin antibody is increased by at least about 20% compared to the level of CTSB in the cerebrospinal fluid before the individual received one or more doses of the anti-Sortilin antibody. In some embodiments, the anti-Sortilin antibody is determined to be active in the individual if the level of SPP1 in the cerebrospinal fluid after the individual has received one or more doses of the anti-Sortilin antibody is decreased compared to the level of SPP1 in the cerebrospinal fluid before the individual has received one or more doses of the anti-Sortilin antibody. In some embodiments, the anti-Sortilin antibody is determined to be active in the individual if the level of SPP1 in the cerebrospinal fluid after the individual has received one or more doses of the anti-Sortilin antibody is decreased by at least about 10% compared to the level of SPP1 in the cerebrospinal fluid before the individual has received one or more doses of the anti-Sortilin antibody. In some embodiments, the anti-Sortilin antibody is determined to be active in the individual if the level of NAGK in the cerebrospinal fluid after the individual has received one or more doses of the anti-Sortilin antibody is increased compared to the level of NAGK in the cerebrospinal fluid before the individual has received one or more doses of the anti-Sortilin antibody. In some embodiments, the anti-Sortilin antibody is determined to be active in the individual if the levels of one or more inflammatory proteins in the cerebrospinal fluid after the individual has received one or more doses of the anti-Sortilin antibody are decreased compared to the levels of the one or more inflammatory proteins in the cerebrospinal fluid before the individual has received one or more doses of the anti-Sortilin antibody, wherein the one or more inflammatory proteins are selected from the group consisting of 14-3-3 protein epsilon (YWHAE), allograft inflammatory factor 1 (AIF1), colony stimulating factor 1 (CSF1), chitinase 1 (CHIT1), lymphocyte antigen 86 (LY86), and CD86. In some embodiments, the sample is from the blood of the individual.

### BRIEF DESCRIPTION OF THE DRAWINGS

FIGS. 1A-1C provide pharmacokinetic and pharmacodynamic studies of non-human primates administered single doses of anti-Sortilin antibody S-60-15.1 [N33T] LALAPS. FIG. 1A provides the level of SORT1 in peripheral white blood cells as a percentage from baseline at the indicated times after treatment (hours) with the specified anti-Sortilin antibody doses. SORT1 expression decreased with all of the anti-Sortilin antibody doses tested. Higher antibody doses (60 mg/kg, 200 mg/kg) resulted in both an earlier and more prolonged decrease of SORT1 levels compared to lower anti-Sortilin antibody doses (5 mg/kg, 20 mg/kg). FIG. 1B provides the levels of PGRN in the plasma as a percentage from baseline at the indicated times after treatment (hours) with the specified anti-Sortilin antibody doses. The levels of PGRN increased in a time- and dose-dependent manner. In particular, plasma PGRN levels increased 3- to 4-fold at Cₘₐₓ, compared to baseline levels, for all anti-Sortilin antibody doses tested and remained elevated for longer periods of time at the higher antibody doses. FIG. 1C provides the levels of PGRN in CSF as a percentage from baseline at the indicated times after treatment (hours) with the specified anti-Sortilin antibody doses. CSF PGRN levels increased 2- to 3-fold above baseline in animals administered either 20 mg/kg, 60 mg/kg, or 200 mg/kg. As observed with plasma PGRN levels (FIG. 1B), CSF PGRN levels remained elevated over time in the higher antibody dose groups. For FIGS. 1A-1C, n=3 animals per dose.

FIGS. 2A-2C provide pharmacokinetic and pharmacodynamic studies of non-human primates administered repeat doses of anti-Sortilin antibody S-60-15.1 [N33T] LALAPS. Animals (2 males and 2 females) were administered anti-Sortilin antibody S-60-15.1 [N33T] LALAPS at a dose of 60 mg/kg once per week for four. The days on which dosing occurred are represented by the vertical dashed lines. FIG. 2A provides the mean (+/−standard deviation) of the concentration of SORT1 in peripheral white blood cells (WBCs) as a percentage of baseline at the indicated times (days). SORT1 levels in peripheral white blood cells remained decreased throughout the duration of the study. FIG. 2B provides the mean (+/−standard deviation) of the concentration of PGRN in plasma as a percentage of baseline (normalized) at the indicated times (days). Plasma PGRN levels increased to 5- to 6-fold above baseline at peak levels. A decrease in plasma PGRN was observed following the fourth and final administration of anti-Sortilin antibody; however, the plasma PGRN levels remained elevated by 2-fold above baseline. FIG. 2C provides the mean (+/−standard deviation) of the concentration of PGRN in CSF as a percentage of baseline (normalized) at the indicated times (days). CSF PGRN levels were increased 3- to 4-fold above baseline (FIG. 2C).

FIGS. 3A-3C show the effect of anti-Sortilin antibody 5-60-15.1 [N33T] LALAPS on SORT1 levels in white blood cells and on plasma PGRN levels. In FIG. 3A, dashed lines represent SORT1 expression levels on peripheral white blood cells (wbc) as percent change from baseline at the indicated times in 5 healthy volunteer cohorts treated with the specified doses of anti-Sortilin antibody 5-60-15.1 [N33T] LALAPS; solid lines represent plasma (PL) PGRN levels as percent change from baseline at the indicated times in 5 healthy volunteer cohorts treated with the specified doses of anti-Sortilin antibody 5-60-15.1 [N33T] LALAPS. Administration of anti-Sortilin antibody 5-60-15.1 [N33T] LALAPS to human subjects resulted in a decrease in SORT1 expression levels on peripheral white blood cells and an increase in plasma PGRN levels. A further analysis of SORT1 levels on peripheral white blood cells at the indicated times (days post dose) in human subjects administered anti-Sortilin antibody 5-60-15.1 [N33T] LALAPS is provided in FIG. 3B. A further analysis of PGRN levels relative to baseline at the indicated times (days post dose) in human subjects administered anti-Sortilin antibody S-60-15.1 [N33T] LALAPS is provided in FIG. 3C. The horizontal dashed line indicates a 2-fold increase over baseline.

FIGS. 4A-4C show the effect of anti-Sortilin antibody S-60-15.1 [N33T] LALAPS on PGRN levels in CSF. Pharmacodynamic data for CSF PGRN levels were obtained from healthy volunteer cohorts dosed at 0 mg/kg (placebo), 15 mg/kg, 30 mg/kg, or 60 mg/kg. CSF samples were collected at pre-dose, and then at approximately 30-hours, 12 days, 24 days, and 42 days after antibody administration. As shown in FIG. 4A, statistically significant increases in CSF PGRN levels (compared to PGRN levels observed at baseline) were seen at 30-hours and 12—days for all cohorts. In addition, administration of the antibody at 60 mg/kg led to increased levels of CSF PGRN that were sustained for at least 24—days after a single IV dose of anti-Sortilin antibody. FIG. 4B shows the percent change from baseline of CSF PGRN levels in healthy volunteers dosed at 0 mg/kg (Placebo), 15 mg/kg (“Cohort 3”), 30 mg/kg (“Cohort 4”), or 60 mg/kg (“Cohort 5”) on study day 13 (12—days post dose). Asterisks indicate statistical significance. (****: P<0.0001, adjusted for multiplicity.) FIG. 4C shows the percent change from baseline of CSF PGRN levels at the indicated days post-dosing in healthy volunteers dosed at 60 mg/kg (“Cohort 5” and “Cohort 6” combined).

FIGS. 5A-5C show the effect of anti-Sortilin antibody S-60-15.1 [N33T] LALAPS on PGRN levels in plasma and CSF of aFTD-GRN and FTD-GRN subjects. FIG. 5A provides the mean percent change in plasma PGRN levels at the indicated days post-dosing in one aFTD-GRN subject and three FTD-GRN subjects. FIG. 5B provides the mean percent change from baseline in CSF PGRN levels in one aFTD-GRN subject (study day 13) and three FTD-GRN patients (study day 57). FIG. 5C provides the concentration of PGRN in CSF (ng/mL) from normal healthy volunteers and from three FTD-GRN patients at pre-dose and on study day 57.

FIG. 6 provides a schematic depiction of the Phase 2 study described in Example 3. CSF=cerebrospinal fluid; GRN=Granulin; IV=intravenous; MRI=magnetic resonance imaging; PD=pharmacodynamic; PET=positron emission tomography; q4w=every 4 weeks; TSPO=translocator protein.

FIG. 7 shows the effect of anti-Sortilin antibody S-60-15.1 [N33T] LALAPS on PGRN concentration (ng/mL) in the plasma of aFTD-GRN and FTD-GRN subjects at the indicated times after administration of the antibody as described in Example 5. SD=single dose; MD=multiple dose. The median baseline concentrations of PGRN in the plasma of healthy volunteers (HV) and FTD patients are indicated by horizontal lines.

FIG. 8 shows the effect of anti-Sortilin antibody S-60-15.1 [N33T] LALAPS on PGRN concentration (ng/mL) in the CSF of aFTD-GRN (asymptomatic) and FTD-GRN (symptomatic) subjects at the indicated times after administration of the antibody. The concentrations of PGRN in the CSF of healthy volunteers (HV) are provided. One symptomatic subject did not have a reportable CSF PGRN result at baseline.

FIG. 9 shows the effect of anti-Sortilin antibody S-60-15.1 [N33T] LALAPS on the CSF protein signature in FTD-GRN patients by SOMASCAN analysis of >1000 proteins, as described in Example 5. The Y-axis provides Z-scores of the ratio of the levels of each protein in FTD-GRN patients and in healthy volunteers. The X-axis provides Z-scores of the ratio of the levels of each protein in FTD-GRN patients at 57 days after administration of anti-Sortilin antibody S-60-15.1 [N33T] LALAPS and at baseline (before administration of anti-Sortilin antibody S-60-15.1 [N33T] LALAPS). Proteins that are upregulated in FTD-GRN patients and were normalized after administration of anti-Sortilin antibody S-60-15.1 [N33T] LALAPS are shown in the upper left quadrant in the scatterplot. Proteins that are downregulated in FTD-GRN patients and were restored after administration of anti-Sortilin antibody 5-60-15.1 [N33T] LALAPS are shown in the lower right quadrant in the scatterplot.

FIGS. 10A-10B show NfL plasma levels in FTD-GRN patients. In FIG. 10A, NfL plasma levels were measured using the SIMOA Nf-Light Advantage assay by Quinterix. In FIG. 10A, NfL plasma levels are indicated at various time points as a ratio to baseline level for each of five patients. FIG. 10B shows the geometric mean of the data of FIG. 10A.

FIGS. 11A-11B show the effect of anti-Sortilin antibody S-60-15.1 [N33T] LALAPS on SPP1, a biomarker that is upregulated in FTD patients, and CTSB, a biomarker that is downregulated in FTD patients. FIG. 11A shows that the biomarker SPP1 is upregulated in FTD patients relative to healthy volunteers, and treatment of FTD patients with 5-60-15.1 [N33T] LALAPS reduces SPP1 closer to normal levels. Conversely, FIG. 11B shows that the biomarker CTSB is downregulated in FTD patients relative to healthy volunteers, and treatment of FTD patients with S-60-15.1 [N33T] LALAPS increases CTSB levels closer to normal levels.

### DETAILED DESCRIPTION

### Definitions

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. Preferably, the individual is human.

The terms “Sortilin” or “Sortilin polypeptide” are used interchangeably herein refer herein to any native Sortilin from any mammalian source, including primates (e.g., humans and cynos) and rodents (e.g., mice and rats), unless otherwise indicated. In some embodiments, the term encompasses both wild-type sequences and naturally occurring variant sequences, e.g., splice variants or allelic variants. In some embodiments, the term encompasses “full-length,” unprocessed Sortilin as well as any form of Sortilin that results from processing in the cell. In some embodiments, the Sortilin is human Sortilin. In some embodiments, the amino acid sequence of an exemplary human Sortilin is SEQ ID NO: 81.

The terms “anti-Sortilin antibody,” an “antibody that binds to Sortilin,” and “antibody that specifically binds Sortilin” refer to an antibody that is capable of binding Sortilin with sufficient affinity such that the antibody is useful as a diagnostic and/or therapeutic agent in targeting Sortilin. In one embodiment, the extent of binding of an anti-Sortilin antibody to an unrelated, non-Sortilin polypeptide is less than about 10% of the binding of the antibody to Sortilin as measured, e.g., by a radioimmunoassay (RIA). In certain embodiments, an antibody that binds to Sortilin has a dissociation constant (KD) of <1 μM, <100 nM, <10 nM, <1 nM, <0.1 nM, <0.01 nM, or <0.001 nM (e.g., 10-8 M or less, e.g. from 10-8 M to 10-13 M, e.g., from 10-9 M to 10-13 M). In certain embodiments, an anti-Sortilin antibody binds to an epitope of Sortilin that is conserved among Sortilin from different species.

The term “immunoglobulin” (Ig) is used interchangeably with “antibody” herein. The term “antibody” herein is used in the broadest sense and specially covers monoclonal antibodies, polyclonal antibodies, multispecific antibodies (e.g., bispecific antibodies) including those formed from at least two intact antibodies, and antibody fragments so long as they exhibit the desired biological activity.

“Native antibodies” are usually heterotetrameric glycoproteins of about 150,000 Daltons, composed of two identical Light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6.

The light chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4^{th} ed. (W. B. Saunders Co., 2000).

The “variable region” or “variable domain” of an antibody, such as an anti-Sortilin antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-Sortilin antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

An “isolated” antibody, such as an anti-Sortilin antibody of the present disclosure, is one that has been identified, separated and/or recovered from a component of its production environment (e.g., naturally or recombinantly). Preferably, the isolated polypeptide is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T cells since at least one component of the antibody's natural environment will not be present. Ordinarily, however, an isolated polypeptide or antibody will be prepared by at least one purification step.

The term “monoclonal antibody” as used herein refers to an antibody, such as a monoclonal anti-Sortilin antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, but not limited to one or more of the following methods, immunization methods of animals including, but not limited to rats, mice, rabbits, guinea pigs, hamsters and/or chickens with one or more of DNA(s), virus-like particles, polypetide(s), and/or cell(s), the hybridoma methods, B-cell cloning methods, recombinant DNA methods, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody, such as an anti-Sortilin antibody of the present disclosure, in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-Sortilin antibodies of the present disclosure, produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of antibodies, such as anti-Sortilin antibodies of the present disclosure, comprise a portion of an intact antibody, generally including the antigen binding or variable region of the intact antibody or the F region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

As used herein, a “chimeric antibody” refers to an antibody (immunoglobulin), such as a chimeric anti-Sortilin antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-Sortilin antibodies of the present disclosure, are chimeric antibodies comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino-acid sequence corresponding to that of an antibody, such as an anti-Sortilin antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries and yeast-based platform technologies. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody-variable domain, such as that of an anti-Sortilin antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat timbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the VL acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino-acid modification” at a specified position, e.g., of an anti-Sortilin antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody, such as an anti-Sortilin antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically recognizes” or “specifically binds” refers to measurable and reproducible interactions such as attraction or binding between a target and an antibody, such as an anti-Sortilin antibody of the present disclosure, that is determinative of the presence of the target in the presence of a heterogeneous population of molecules including biological molecules. For example, an antibody, such as an anti-Sortilin antibody of the present disclosure, that specifically or preferentially binds to a target or an epitope is an antibody that binds this target or epitope with greater affinity, avidity, more readily, and/or with greater duration than it binds to other targets or other epitopes of the target. It is also understood by reading this definition that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³M⁻¹ or 10⁴M⁻¹, sometimes about 10⁵ M⁻¹ or 10⁶M⁻¹, in other instances about 10⁶M⁻¹ or 10⁷M⁻¹, about 10⁸M⁻¹ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, an “interaction” between a Sortilin protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

An “agonist” antibody or an “activating” antibody is an antibody, such as an agonist anti-Sortilin antibody of the present disclosure, that induces (e.g., increases) one or more activities or functions of the antigen after the antibody binds the antigen.

A “blocking” antibody, an “antagonist” antibody, or an “inhibitory” antibody is an antibody, such as an anti-Sortilin antibody of the present disclosure, that inhibits or reduces (e.g., decreases) antigen binding to one or more ligand after the antibody binds the antigen, and/or that inhibits or reduces (e.g., decreases) one or more activities or functions of the antigen after the antibody binds the antigen. In some embodiments, blocking antibodies, antagonist antibodies, or inhibitory antibodies substantially or completely inhibit antigen binding to one or more ligand and/or one or more activities or functions of the antigen.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fe region or to the Fe region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies. As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

An “isolated” cell is a molecule or a cell that is identified and separated from at least one contaminant cell with which it is ordinarily associated in the environment in which it was produced. In some embodiments, the isolated cell is free of association with all components associated with the production environment. The isolated cell is in a form other than in the form or setting in which it is found in nature. Isolated cells are distinguished from cells existing naturally in tissues, organs, or individuals. In some embodiments, the isolated cell is a host cell of the present disclosure.

An “isolated” nucleic acid molecule encoding an antibody, such as an anti-Sortilin antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the present disclosure.

“Carriers” as used herein include pharmaceutically acceptable carriers, excipients, or stabilizers that are nontoxic to the cell or mammal being exposed thereto at the dosages and concentrations employed.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising,” “consisting,” and “consisting essentially of” aspects and embodiments.

### Overview

The present disclosure relates to methods of treating and/or delaying the progression of a disease or injury in an individual by administering an anti-Sortilin antibody to the individual. Non-limiting examples of diseases that may be treated or delayed include Frontotemporal Dementia (FTD) and Amyotrophic Lateral Sclerosis (ALS). As described below, the methods of the present disclosure meet the need in the art for identifying methods of treating patients with the correct dose and of administering that dose in ways that ease patient compliance.

Advantageously, intravenous administration of a single or repeated doses of an anti-Sortilin antibody of the present disclosure to non-human primates (see, e.g., Example 1) leads to a decrease of SORT1 protein on white blood cells in a dose-dependent manner and an increase in PGRN protein levels in plasma (e.g., 2- to 6-fold increase) and cerebrospinal fluid (CSF) (e.g., 2- to 4-fold increase). Moreover, while the half-life of the anti-Sortilin antibody is relatively short (e.g., up to 73.6 hours), unexpectedly, the decrease of SORT1 protein on white blood cells and the increase in PGRN protein levels in plasma and CSF persist over time (e.g., up to 14 days after the last dose of anti-Sortilin antibody) antibody. Furthermore, advantageously, exposure increases over time (e.g., day 1 versus day 22), indicating accumulation of the anti-Sortilin antibody.

Similarly, intravenous administration of a single dose of an anti-Sortilin antibody of the present disclosure to healthy humans (see, e.g., Example 2) leads to a decrease of SORT1 protein on white blood cells in a dose-dependent manner (e.g., 50% or 70% decrease) and an increase in PGRN protein levels in plasma (e.g., 1.29- to 2.14-fold increase) and in CSF (e.g., 0.57- to 1.13-fold increase). Moreover, while the half-life of the anti-Sortilin antibody is relatively short (e.g., up to 190 hours), unexpectedly, the decrease of SORT1 protein on white blood cells (e.g., 40 days or more) and the increase in PGRN protein levels in plasma (e.g., 40 days to 42 days or more) and CSF persist over time (e.g., at least 24 days).

Patients with neurodegenerative diseases, such as FTD and ALS, are affected by the diseases for long periods of time and thus require regular treatment over the course of many years. As intravenous administration of therapeutics cannot be done at home, patients must be transported to infusion centers, which is a burden on both the patient and caregiver. Finally, the memory loss, mood swings, aggression, and other behavioral symptoms of these diseases make patient compliance difficult.

Advantageously, while the anti-Sortilin antibody of the present disclosure exhibits a relatively short half-life and thus may not be expected to be useful therapeutically, when administered according to the methods provided herein, the antibody unexpectedly exhibits long-lasting pharmacodynamic (PD) effects (e.g., increase of PGRN levels in plasma and CSF, and decrease of SORT1 levels on WBCs and in CSF). Thus, methods provided herein permit relatively infrequent administration of the anti-Sortilin antibody, which is particularly beneficial for patients with neurodegenerative diseases, such as FTD and ALS.

Accordingly, in some embodiments, the present disclosure further relates to methods of treating and/or delaying the progression of FTD (see, e.g., Example 3) or ALS (see, e.g., Example 4) in an individual by administering to the individual an anti-Sortilin antibody intravenously at a dose of at least about 30 mg/kg at least once every four weeks. In some embodiments, the anti-Sortilin antibody is administered once every four weeks at dose of about 60 mg/kg.

All references cited herein, including patents, patent applications and publications, are hereby incorporated by reference in their entirety.

### Therapeutic Uses

The present disclosure provides methods of treating and/or delaying the progression of a disease or injury in an individual, comprising administering to the individual an anti-Sortilin antibody, where the antibody comprises a heavy chain variable region and a light chain variable region, wherein the heavy chain variable region comprises an HVR-H1 comprising the amino acid sequence of SEQ ID NO: 1; an HVR-H2 comprising an amino acid sequence selected from the group consisting of SEQ ID NOs: 2-3; and an HVR-H3 comprising an amino acid sequence selected from the group consisting of SEQ ID NOs: 5-6; and the light chain variable region comprises: an HVR-L1 comprising an amino acid sequence selected from the group consisting of SEQ ID NOs: 8-27; an HVR-L2 comprising an amino acid sequence selected from the group consisting of SEQ ID NOs: 29-30; and an HVR-L3 comprising the amino acid sequence of SEQ ID NO: 32.

As disclosed herein, anti-Sortilin antibodies of the present disclosure may be used for treating and/or delaying progression of frontotemporal dementia, progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, limbic-predominant age-related TDP43 encephalopathy (LATE), acute disseminated encephalomyelitis, retinal degeneration, age related macular degeneration, glaucoma, multiple sclerosis, septic shock, bacterial infection, arthritis, or osteoarthritis. In some embodiments, the disease or injury is frontotemporal dementia or amyotrophic lateral sclerosis. In some embodiments, anti-Sortilin antibodies of the present disclosure may be used for treating or alleviating TDP43 pathologies, including but not limited to TDP43 pathologies associated with dementia, C9orf72 associated diseases, FTD, Alzheimer's disease, ALS, LATE, and Parkinson's disease.

In some embodiments, a method of the present disclosure includes an anti-Sortilin antibody comprising two or more anti-Sortilin antibodies.

### Dementia

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia.

Exemplary forms of dementia include, without limitation, frontotemporal dementia, Alzheimer's disease, vascular dementia, semantic dementia, and dementia with Lewy bodies.

Without wishing to be bound by theory, it is believed that administering an anti-Sortilin antibody of the present disclosure can treat and/or delay the progression of dementia. In some embodiments, administering an anti-Sortilin antibody may induce one or more Progranulin activities in an individual having dementia (e.g., neurotrophic and/or survival activity on neurons, and anti-inflammatory activity.

### Frontotemporal Dementia

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. The clinical features of FTD include memory deficits, behavioral abnormalities, personality changes, and language impairments (Cruts, M. & Van Broeckhoven, C., Trends Genet. 24:186-194 (2008); Neary, D., et al., Neurology 51:1546-1554 (1998); Ratnavalli, E., Brayne, C., Dawson, K. & Hodges, J. R., Neurology 58:1615-1621 (2002)).

A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule associated protein Tau was supported by the identification of mutations in the gene encoding the Tau protein in several families (Hutton, M., et al., Nature 393:702-705 (1998). However, the majority of FTD brains show no accumulation of hyperphosphorylated Tau but do exhibit immunoreactivity to ubiquitin (Ub) and TAR DNA binding protein (TDP43) (Neumann, M., et al., Arch. Neurol. 64:1388-1394 (2007)). A majority of those FTD cases with Ub inclusions (FTD-U) were shown to carry mutations in the Progranulin gene.

Progranulin mutations result in haploinsufficiency and are known to be present in nearly 50% of familial FTD cases, making Progranulin mutation a major genetic contributor to FTD. Without wishing to be bound by theory, it is believed that the loss-of-function heterozygous character of Progranulin mutations indicates that in healthy individuals, Progranulin expression plays a dose-dependent, critical role in protecting healthy individuals from the development of FTD. Accordingly, increasing levels of Progranulin by inhibiting the interaction between Sortilin and Progranulin, can treat and/or delay the progression of FTD.

In some embodiments, administering an anti-Sortilin antibody of the present disclosure, can treat and/or delay the progression of FTD. In some embodiments, administering an anti-Sortilin antibody may modulate one or more Sortilin activities in an individual having FTD.

In some embodiments, treatment and/or delay of FTD progression is determined by a change from baseline in neurocognitive and/or functional tests or assessments (i.e., clinical outcome assessments). Non-limiting examples of neurocognitive and functional tests that may be used to evaluate the treatment and/or delay of FTD progression include the Frontotemporal Dementia Clinical Rating Scale (FCRS), the Frontotemporal Dementia Rating Scale (FRS), the Clinical Global Impression-Improvement (CGI-I) assessment, the Neuropsychiatric Inventory (NPI) assessment, the Color Trails Test (CTT) Part 2, the Repeatable Battery for the Assessment of Neuropsychological Status (RBANS), the Delis-Kaplan Executive Function System Color-Word Interference Test, the Interpersonal Reactivity Index, the Winterlight Lab Speech Assessment (WLA), and the Summerlight Lab Speech Assessment (SLA). In some embodiments, treatment and/or delay of FTD progression is determined by a change from baseline in one neurocognitive and/or functional test or assessment. In some embodiments, treatment and/or delay of FTD progression is determined by a change from baseline in more than one neurocognitive and/or functional tests or assessments (e.g., 2, 3, 4, 5, 6, 7, 8, 9 or more neurocognitive and/or functional tests or assessments).

In some embodiments, treatment and/or delay of FTD progression is determined by a change from baseline in global and/or regional brain volumes, volume of white matter hyperintensities, brain perfusion, fractional anisotropy, mean diffusivity, axial diffusivity, and radial diffusivity, and/or functional brain activity. In certain embodiments, brain perfusion is measured by arterial spin labeling MRI. In certain embodiments, radial diffusivity is measured by diffusion tensor imaging. In certain embodiments, functional brain activity is measured by functional MRI.

In some embodiments, treatment and/or delay of FTD progression is determined by a change from baseline in markers of neurodegeneration in whole blood, plasma, and CSF. Markers of neurodegeneration may include, without limitation, neurofilament light chain [NfL], Tau, and/or pTau. Neurofilament light chain may be measured by methods including, without limitation, assays from Quanterix and/or Roche Diagnostics. In some embodiments, treatment with an anti-Sortilin antibody of the present disclosure reduces NfL levels by at least 10%, 12%, 15%, 20%, 25%, 30%, 35%, 40%, 45%, or 50%. In some embodiments, treatment and/or delay of FTD progression is determined by a change (e.g., an increase) from baseline in markers of lysosomal function. Markers of lysosomal function may be, without limitation, Cathepsins, such as Cathepsin B (CTSB). In some embodiments, treatment with an anti-Sortilin antibody of the present disclosure increases the level of one or more lysosomal markers, such as CTSB, by any of at least 10%, at least 20%, at least 30%, at least 40%, at least 50%, at least 60%, at least 70%, at least 80%, at least 90%, at least 100%, or more, compared to the baseline level of the one or more lysosomal markers, such as CTSB. In some embodiments, treatment with an anti-Sortilin antibody of the present disclosure increases the level of CTSB by at least about 20% compared to the baseline level of CTSB. Another non-limiting example of a lysosomal marker is N-acetylglucosamine kinase (NAGK). In some embodiments, treatment with an anti-Sortilin antibody of the present disclosure increases the level of NAGK by any of at least 10%, at least 20%, at least 30%, at least 40%, at least 50%, at least 60%, at least 70%, at least 80%, at least 90%, at least 100%, or more, compared to the baseline level of NAGK.

In some embodiments, treatment and/or delay of FTD progression is determined by a change (e.g., a decrease) from baseline in the levels of inflammatory markers, such as Osteopontin (SPP1). In some embodiments, treatment with an anti-Sortilin antibody of the present disclosure decreases the level of one or more inflammatory markers, such as SPP1, by any of at least 10%, at least 20%, at least 30%, at least 40%, at least 50%, at least 60%, at least 70%, at least 80%, at least 90%, at least 100%, or more, compared to the baseline level of the one or more inflammatory markers, such as SPP1. In some embodiments, treatment with an anti-Sortilin antibody of the present disclosure decreases the level of one or more inflammatory markers, such as SPP1, by any of at least 10%, at least 20%, at least 30%, at least 40%, at least 50%, at least 60%, at least 70%, at least 80%, at least 90%, or 100% compared to the baseline level of the one or more inflammatory markers, such as SPP1. In some embodiments, treatment with an anti-Sortilin antibody of the present disclosure decreases the level of SPP1 by at least about 10% compared to the baseline level of SPP1. Other examples of inflammatory markers include, without limitation, YWHAE (14-3-3 protein epsilon), allograft inflammatory factor 1 (AIF1), colony stimulating factor 1 (CSF1), chitinase 1 (CHIT1), lymphocyte antigen 86 (LY86), and CD86. In some embodiments, treatment with an anti-Sortilin antibody of the present disclosure decreases the level of one or more inflammatory markers, such as YWHAE (14-3-3 protein epsilon), allograft inflammatory factor 1 (AIF1), colony stimulating factor 1 (CSF1), chitinase 1 (CHIT1), lymphocyte antigen 86 (LY86), or CD86, by any of at least 10%, at least 20%, at least 30%, at least 40%, at least 50%, at least 60%, at least 70%, at least 80%, at least 90%, or 100% compared to the baseline level of the one or more inflammatory markers, such as YWHAE (14-3-3 protein epsilon), allograft inflammatory factor 1 (AIF1), colony stimulating factor 1 (CSF1), chitinase 1 (CHIT1), lymphocyte antigen 86 (LY86), or CD86.

In some embodiments, treatment and/or delay of FTD progression is determined by a change from baseline in markers of microglial activity. Markers of microglial activity may be, without limitation, YKL-40 and/or Interleukin-6. In some embodiments, treatment and/or delay of FTD progression is determined by a change from baseline of messenger ribonucleic acid (mRNA) expression in peripheral cells. In some embodiments, treatment and/or delay of FTD progression is determined by a change from baseline in analytes relevant to FTD disease biology and/or response to anti-Sortilin antibody.

In some embodiments, the levels of one or more proteins (e.g., one or more of YKL-40, IL-6, CTSB, SPP1, NAGK, YWHAE, AIF1, CSF1, CHIT1, LY86, or CD86) may be measured in a sample obtained from the individual, such as a sample of whole blood, plasma, and/or CSF. Non-limiting examples of methods that may be used to measure the levels of one or more proteins (e.g., one or more of YKL-40, IL-6, CTSB, SPP1, NAGK, YWHAE, AIF1, CSF1, CHIT1, LY86, or CD86) in a sample obtained from the individual include SOMASCAN assay (see, e.g., Candia et al. (2017) Sci Rep 7, 14248), Western blots, mass spectrometry, flow cytometry, and enzyme-linked immunosorbent assay (ELISA) assays.

In some embodiments, treatment and/or delay of FTD progression is determined by a change from baseline in neuroinflammation and/or microglial activation. Neuroinflammation and/or microglial activation may be measured by any known method in the art. In certain embodiments, Neuroinflammation and/or microglial activation may be measured using Translocator Protein-Positron Emission (TSPO-PET) imaging. In certain embodiments, [¹⁸F]PBR06 and/or [¹¹C]PBR28 PET are used as radiotracers in TSPO-PET imaging. In certain embodiments, [¹⁸F]PBR06 is used as a radiotracer in TSPO-PET imaging. In certain embodiments, [¹¹C]PBR28 PET is used as a radiotracer in TSPO-PET imaging.

In some embodiments, the individual is heterozygous for a mutation in GRN (the Granulin gene). In some embodiments, the mutation in GRN is a loss-of-function mutation. In some embodiments, the individual is heterozygous for a C9orf72 hexanucleotide repeat expansion. In some embodiments, the individual shows symptoms of FTD. In some embodiments, the individual does not show symptoms of FTD.

In some embodiments, the individual shows symptoms of FTD if the individual meets diagnostic criteria for possible behavioral variant FTD (bvFTD) or probable bvFTD or primary progressive aphasia (PPA). In some embodiments, the individual has one or more of the behavioral/cognitive symptoms required for a diagnosis of possible bvFTD (Rascovsky et al., (2011) Brain 134(9):2456-2477). In some embodiments, the individual has mild symptomatology not significantly affecting activities of daily living (e.g., mild cognitive impairment, mild behavioral impairment). In certain embodiments, the individual has bvFTD or PPa with concomitant motor neuron disease. In some embodiments, the individual has FTD of mild severity as defined by a Clinical Dementia Rating Scale (CDR) global score of 1 or less and a box score of 1 or less on both the Language domain, and the Behavior, Comportment and Personality domain of the Frontotemporal Dementia Clinical Rating Scale (FCRS).

### Alzheimer's Disease

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include, behavioral symptoms, such as difficulty in remembering recent events; cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

It has been shown that Sortilin binds to amyloid precursor protein (APP) and the APP processing enzyme BACE1. Without wishing to be bound by theory, it is believed that these interactions are involved in Alzheimer's disease. Accordingly, and without wishing to be bound by theory, it is believed that anti-Sortilin antibodies of the present disclosure can be utilized to inhibit such interactions and prevent, reduce the risk of, or treat Alzheimer's disease in individuals in need thereof.

In some embodiments, and without wishing to be bound by theory, it is believed that anti-Sortilin antibodies of the present disclosure that inhibit the interaction between Sortilin and neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), p75, amyloid precursor protein (APP), and/or the A beta peptide, or that inhibit one or more activities of Sortilin can be utilized to treat and/or delay the progression of Alzheimer's disease in individuals in need thereof.

In some embodiments, administering an anti-Sortilin antibody of the present disclosure can treat and/or delay the progression of Alzheimer's disease. In some embodiments, administering an anti-Sortilin antibody may modulate one or more Sortilin activities in an individual having Alzheimer's disease.

### Vascular Dementia

Vascular dementia (VaD) is a subtly progressive worsening of memory and other cognitive functions that is believed to be due to cerebrovascular disease (vascular disease within the brain). Cerebrovascular disease is the progressive change in our blood vessels (vasculature) in the brain (cerebrum). The most common vascular change associated with age is the accumulation of cholesterol and other substances in the blood vessel walls. This results in the thickening and hardening of the walls, as well as narrowing of the vessels, which can result in a reduction or even a complete stopping of blood flow to brain regions supplied by the affected artery. Vascular dementia patients often present with similar symptoms to Alzheimer's disease (AD) patients. However, the related changes in the brain are not due to AD pathology but to chronic reduced blood flow in the brain, eventually resulting in dementia. VaD is considered one of the most common types of dementia in older adults. Symptoms of VaD include difficulties with memory, difficulty with organization and solving complex problems, slowed thinking, distraction or “absent mindedness,” difficulty retrieving words from memory, changes in mood or behavior such as depression, irritability, or apathy, and hallucinations or delusions.

Without wishing to be bound by theory, it is believed that one or more activities of Sortilin, or one or more interactions between Sortilin and Progranulin, neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, lipoprotein lipase, apolipoprotein AV, and/or receptor-associated protein are involved in vascular dementia. Accordingly, and without wishing to be bound by theory, it is believed that anti-Sortilin antibodies of the present disclosure that inhibit the interaction between Sortilin and neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, p75, Sortilin propeptide (Sort-pro), amyloid precursor protein (APP), the A beta peptide, lipoprotein lipase (LpL), apolipoprotein AV (APOA5), apolipoprotein E (APOE), and/or receptor associated protein (RAP); or that inhibit one or more activities of Sortilin can be utilized to prevent, reduce the risk of, or treat vascular dementia in individuals in need thereof.

In some embodiments, administering an anti-Sortilin antibody of the present disclosure can treat and/or delay the progression of VaD. In some embodiments, administering an anti-Sortilin antibody may modulate one or more Sortilin activities in an individual having VaD.

### Seizures, Retinal Dystrophy, Traumatic Brain Injuries, and Spinal Cord Injuries

As used herein, retinal dystrophy refers to any disease or condition that involves the degeneration of the retinal. Such diseases or conditions may lead to loss of vision or complete blindness.

As used herein, seizures also include epileptic seizures, and refer to a transient symptom of abnormal excessive or synchronous neuronal activity in the brain. The outward effect can be as dramatic as a wild thrashing movement or as mild as a brief loss of awareness. Seizures can manifest as an alteration in mental state, tonic or clonic movements, convulsions, and various other psychic symptoms.

Traumatic brain injuries (TBI), may also be known as intracranial injuries. Traumatic brain injuries occur when an external force traumatically injures the brain. Traumatic brain injuries can be classified based on severity, mechanism (closed or penetrating head injury), or other features (e.g., occurring in a specific location or over a widespread area).

Spinal cord injuries (SCI) include any injury to the spinal cord that is caused by trauma instead of disease. Depending on where the spinal cord and nerve roots are damaged, the symptoms can vary widely, from pain to paralysis to incontinence. Spinal cord injuries are described at various levels of “incomplete”, which can vary from having no effect on the patient to a “complete” injury which means a total loss of function.

It has been shown that pro-neurotrophins (e.g., pro-neurotrophin-4/5, neurotrophin-4/5, pro-NGF, pro-BDNF, etc.) play a role in seizures, retinal dystrophy, traumatic brain injury, and spinal cord injury.

Accordingly, and without wishing to be bound by theory, it is believed that anti-Sortilin antibodies of the present disclosure that inhibit the interaction between Sortilin and neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.); or that inhibit one or more activities of Sortilin can be utilized to prevent, reduce the risk of, or treat seizures, retinal dystrophy, traumatic brain injuries, and/or spinal cord injuries in individuals in need thereof.

In some embodiments, administering an anti-Sortilin antibody of the present disclosure can treat and/or delay the progression of seizures, retinal dystrophy, traumatic brain injuries, and/or spinal cord injuries. In some embodiments, administering an anti-Sortilin antibody may modulate one or more Sortilin activities in an individual having seizures, retinal dystrophy, traumatic brain injuries, and/or spinal cord injuries.

### Undesirable Symptoms of Aging

As used herein, undesirable symptoms of aging include, without limitation, memory loss, behavioral changes, dementia, Alzheimer's disease, retinal degeneration, atherosclerotic vascular diseases, hearing loss, and cellular break-down.

In some embodiments, and without wishing to be bound by theory, it is believed that anti-Sortilin antibodies of the present disclosure that inhibit the interaction between Sortilin and Progranulin, neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, p75, lipoprotein lipase (LpL), apolipoprotein AV (APOA5), and/or receptor associated protein (RAP); or that inhibit one or more activities of Sortilin can be utilized to prevent, reduce the risk of, or treat one or more undesirable symptoms of aging.

In some embodiments, administering an anti-Sortilin antibody of the present disclosure can treat and/or delay the progression of one or more undesirable symptoms of aging. In some embodiments, administering an anti-Sortilin antibody may modulate one or more Sortilin activities in an individual having one or more undesirable symptoms of aging.

### Amyotrophic Lateral Sclerosis (ALS)

As used herein, amyotrophic lateral sclerosis (ALS) or, motor neuron disease or, Lou Gehrig's disease are used interchangeably and refer to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

PGRN haploinsufficiency due to heterozygous loss-of-function mutations in the GRN gene results in a reduction of CSF PGRN levels and is causal for the development of frontotemporal dementia (FTD) with TDP-43 pathology (Sleegers et al., (2009) Ann Neurol 65:603; Smith et al., (2012) Am J Hum Genet 90:1102). TDP-43 has also been identified as a major pathological protein in ALS, suggesting a similarity between ALS and FTD.

For example, over twenty dominant mutations in TDP-43 have been identified in sporadic and familial ALS patients (Lagier-Tourenne et al., (2009) Cell 136:1001) and TDP-43 positive aggregates are found in approximately 95% of ALS cases (Prasad et al., (2019) Front Mol Neurosci 12:25). Furthermore, ALS risk genes, such as MOBP, C90RF72, MOBKL2B, NSF and FUS, can also cause FTD (Karch et al., (2018) JAMA Neurol 75:860). In addition, both PGRN and C90RF72 mutations are associated with abnormal microglial activation, which appears to be another common pathology of FTD and ALS (Haukedal et al., (2019) J Mol Biol 431:1818). Other evidence also suggests that ALS and FTD are closely related conditions with overlapping genetic, neuropathological, and clinical features (Weishaupt et al., (2016) Trends Mol Med 22:769; McCauley et al., (2018) Acta Neuropathol 137:715). Taken together, these results suggest that both diseases could benefit from shared treatments and that PGRN genetic variability acts as a modifier of the course of ALS.

Moreover, aside from demonstrations that loss of PGRN is detrimental in multiple models of acute and chronic neurodegeneration (Boddaert et al., (2018) Methods Mol Biol 1806:233), overexpression of PGRN has been found to be protective in many animal models of ALS (Laird et al., (2010) PLoS One 5:e13368; Tauffenberger et al., (2013) Hum Mol Genet 22:782; Beel et al., (2018) Mol Neurodegener 13:55; Chang et al., (2017) J Exp Med 214:2611). In addition, common variants in GRN are significantly associated with a reduction in age at onset and a shorter survival after onset in ALS patients (Sleegers et al., (2008) Neurology 71:253).

In summary, both human genetics and data from disease models support a protective function for PGRN in reducing pathology in ALS patients that are associated with TDP-43 pathology.

In some embodiments, and without wishing to be bound by theory, it is believed that anti-Sortilin antibodies of the present disclosure that inhibit the interaction between Sortilin and Progranulin, neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, p75, lipoprotein lipase (LpL), apolipoprotein AV (APOA5), and/or receptor associated protein (RAP); or that inhibit one or more activities of Sortilin can be utilized to prevent, or treat one or more undesirable symptoms of ALS.

In some embodiments, administering an anti-Sortilin antibody of the present disclosure can treat and/or delay the progression of ALS. In some embodiments, administering an anti-Sortilin antibody may modulate one or more Sortilin activities in an individual having ALS. In some embodiments, the individual is heterozygous for a C9orf72 hexanucleotide repeat expansion.

In some embodiments, treatment and/or delay of ALS progression is determined by a change from baseline in brain atrophy, brain connectivity, brain free water and/or brain inflammation. Any method known in the art including, without limitation, MRI, may be used to measure brain atrophy, brain connectivity, brain free water and/or brain inflammation. In certain embodiments, brain atrophy is measured using structural MRI. In certain embodiments, brain free water and/or brain inflammation are measured using diffusion tensor imaging (DTI).

In some embodiments, treatment and/or delay of ALS progression is determined by a change from baseline in Progranulin, markers of neurodegeneration, markers of glial activation, and/or markers of TDP-43 pathology. In certain embodiments, Proganulin is measured using an Adipogen immunoassay. In certain embodiments, markers of neurodegeneration include, without limitation, neurofilament light chain. Neurofilament light chain may be measured by any known methods in the art including, without limitation, assays from Quanterix and/or Roche Diagnostics. In certain embodiments, markers of glial activation include, without limitation, YKL-40 (CHI3L), IL-6, and/or GFAP. GFAP may be measured using any methods known in the art including, without limitation, assays from Roche Diagnostics.

### Parkinson's Disease

Parkinson's disease, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include, without limitation, tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

In some embodiments, administering an anti-Sortilin antibody of the present disclosure can treat and/or delay the progression of Parkinson's disease. In some embodiments, administering an anti-Sortilin antibody may induce one or more Progranulin activities in an individual having Parkinson's disease. In some embodiments, administering an anti-Sortilin antibody may modulate one or more Sortilin activities in an individual having Parkinson's disease.

### Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. See, e.g., www.ninds.nih.gov/Disorders/Patient-Caregiver-Education/Hope-Through-Research/Multiple-Sclerosis-Hope-Through-Research.

Symptoms of MS include, without limitation, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

In some embodiments, administering an anti-Sortilin antibody of the present disclosure can treat and/or delay the progression of multiple sclerosis. In some embodiments, administering an anti-Sortilin antibody may induce one or more Progranulin activities in an individual having multiple sclerosis. In some embodiments, administering an anti-Sortilin antibody may modulate one or more Sortilin activities in an individual having multiple sclerosis.

### Glaucoma and Macular Degeneration

Glaucoma describes, without limitation, a group of diseases that are characterized by a damaged optic nerve, resulting in vision loss and blindness. Glaucoma is usually caused by increased fluid pressure (=intraocular pressure) in the anterior chamber underneath the cornea. Glaucoma results in the successive loss of retinal ganglion cells that are important for vision. Age-related macular degeneration usually affects older people and primarily causes loss of vision in the macula, the central field of vision. Macular degeneration causes, without limitation, drusen, pigmentary changes, distorted vision, hemorrhages of the eye, atrophy, reduced visual acuity, blurred vision, central scotomas, reduced color vision and reduced contrast sensitivity.

Without wishing to be bound by theory, it is believed that administering an anti-Sortilin antibody of the present disclosure can treat and/or delay the progression of glaucoma and macular degeneration. In some embodiments, administering an anti-Sortilin antibody may induce one or more Progranulin activities in an individual having glaucoma or macular degeneration. In some embodiments, administering an anti-Sortilin antibody may modulate one or more Sortilin activities in an individual having glaucoma or macular degeneration.

### Granulin Mutations

In some embodiments, the individual is heterozygous for a mutation in GRN (the Granulin gene). In some embodiments, the mutation in GRN is a loss-of-function mutation.

In some embodiments, the presence of mutations in GRN is determined by any known method in the art. Non-limiting examples of methods that may be used to determine the presence of mutations in GRN include DNA sequencing, DNA hybridization, polymerase chain reaction (PCR), multiplex PCR, nested PCR, real-time PCR, quantitative PCR, semi-quantitative PCR, DNA microarrays, multiplex ligation-dependent probe amplification, single strand conformation polymorphism analysis, denaturing gradient gel electrophoresis, heteroduplex analysis, Southern blotting, genetic linkage analysis (e.g., using short tandem repeats and/or variable number tandem repeats), fluorescence in situ hybridization, comparative genomic hybridization, allele-specific amplification, and/or restriction enzyme digestion methods (e.g., restriction-fragment length polymorphism analysis) (Mahdieh et al., Iran J Pediatr (2013) 23(4):375-388).

In some embodiments, the presence of mutations in GRN is determined by DNA sequencing (Chang et al., (2010) Arch Neurol 67(2):161-170). In some embodiments, the presence of mutations in GRN is determined by DNA sequencing and genotyping (Chang et al., (2010) Arch Neurol 67(2):161-170).

In some embodiments, low serum progranulin predicts the presence of mutations in GRN (Schofield et al., (2010) J Alzheimers Dis 22(3):981-4). The level of PGRN may be determined as discussed in the “PGRN Levels” section, below.

### C9orf72 Mutations

In some embodiments, the individual is heterozygous for a C9orf72 hexanucleotide repeat expansion.

In some embodiments, the presence of a C9orf72 hexanucleotide repeat expansion is determined by any known method in the art. Non-limiting examples of methods that may be used to determine the presence of a C9orf72 hexanucleotide repeat expansion include DNA sequencing, long-read DNA sequencing, DNA hybridization, polymerase chain reaction (PCR), multiplex PCR, nested PCR, real-time PCR, quantitative PCR, semi-quantitative PCR, DNA microarrays, Southern blotting, multiplex ligation-dependent probe amplification, single strand conformation polymorphism analysis, denaturing gradient gel electrophoresis, heteroduplex analysis, genetic linkage analysis (e.g., using short tandem repeats and/or variable number tandem repeats), fluorescence in situ hybridization, comparative genomic hybridization, allele-specific amplification, and/or restriction enzyme digestion methods (e.g., restriction-fragment length polymorphism analysis) (Mahdieh et al., Iran J Pediatr (2013) 23(4):375-388).

In some embodiments, the presence of a C9orf72 hexanucleotide repeat expansion is determined by DNA sequencing (Ebbert et al., Mol Neurodegener (2018) 13(1):46). In some embodiments, the presence of a C9orf72 hexanucleotide repeat expansion is determined by long-read sequencing (Ebbert et al., Mol Neurodegener (2018) 13(1):46). In some embodiments, the presence of a C9orf72 hexanucleotide repeat expansion is determined using a Pacific Biosciences sequencing platform or an Oxford Nanopore Technologies sequencing platform (Ebbert et al., Mol Neurodegener (2018) 13(1):46). In some embodiments, the presence of a C9orc72 hexanucleotide repeat expansion is determined using a commercially available test. Non-limiting examples of commercially available tests include tests from GeneDx (available at the website www[dot]genedx[dot]com/wp-content/uploads/2017/06/info_sheet_C9orf72.pdf), Fulgent (available at the website www[dot]fulgentgenetics[dot]com/repeatexpansion-c9orf72), Prevention Genetics (available at the website www[dot]preventiongenetics.[dot]com/testlnfo.php?sel=test&val=C9orf72+Gene+Hexanucleotide+Repeat+Expansion), and/or Athena Diagnostics (available at the website www[dot]athenadiagnostics[dot]com/view-full-catalog/c/c9orf72-dna-test).

### Pharmaceutical Dosages

An antibody provided herein (and any additional therapeutic agent) can be administered by any suitable means, including parenteral, intrapulmonary, intranasal, intralesional administration, intracerobrospinal, intracranial, intraspinal, intrasynovial, intrathecal, oral, topical, or inhalation routes. Parenteral infusions include intramuscular, intravenous administration as a bolus or by continuous infusion over a period of time, intraarterial, intra-articular, intraperitoneal, or subcutaneous administration. In some embodiments, the administration is intravenous administration. In some embodiments, the administration is subcutaneous. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies provided herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

Dosages for a particular anti-Sortilin antibody may be determined empirically in individuals who have been given one or more administrations of the anti-Sortilin antibody. Individuals are given incremental doses of an anti-Sortilin antibody. To assess efficacy of an anti-Sortilin antibody, a clinical symptom of any of the diseases, disorders, or conditions of the present disclosure (e.g., frontotemporal dementia, Alzheimer's disease, vascular dementia, seizures, retinal dystrophy, a traumatic brain injury, a spinal cord injury, long-term depression, atherosclerotic vascular diseases, and undesirable symptoms of normal aging) can be monitored.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 g/kg to 15 mg/kg (e.g., 0.1 mg/kg-10 mg/kg) of antibody can be an initial candidate dosage for administration to the individual, whether, for example, by one or more separate administrations, or by continuous infusion. One typical daily dosage might range from about 1 g/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 15 mg/kg to about 70 mg/kg. Thus, one or more doses of about 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, or 70 mg/kg (or any combination thereof) may be administered to the individual. Another exemplary dosage of the antibody would be in the range from about 30 mg/kg to about 60 mg/kg. Thus, one or more doses of about 30 mg/kg, 35 mg/kg, 40 mg/kg, 45 mg/kg, 50 mg/kg, 55 mg/kg, or 60 mg/kg (or any combination thereof) may be administered to the individual.

In some aspects, methods of the present disclosure comprise administering to the individual an anti-Sortilin antibody intravenously at a dose of at least about 30 mg/kg. In some embodiments, the dose is at least about 35 mg/kg, at least about 40 mg/kg, at least about 45 mg/kg, at least about 50 mg/kg, at least about 55 mg/kg, or at least about 60 mg/kg. In some embodiments, the dose is between about 30 mg/kg and about 60 mg/kg. In some embodiments, the dose is about 60 mg/kg.

Such doses may be administered intermittently, e.g., every week or every three weeks (e.g., such that the individual receives from about two to about twenty, or e.g., about six doses of the antibody). In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. In some embodiments, doses are administered about once a month. In some embodiments, the dosing frequency is equal to or greater than q2w (i.e., doses are administered once every two weeks or less frequently than once every two weeks), equal to or greater than q3w, equal to or greater than q4w, equal to or greater than q5w, equal to or greater than q6w, equal to or greater than q7w, or equal to or greater than q8w.

In some aspects, methods of the present disclosure comprise administering an anti-Sortilin antibody to the individual intravenously at a dose of at least about 30 mg/kg once every four weeks or more frequently. In some embodiments, the anti-Sortilin antibody is administered to the individual intravenously at a dose of at least about 30 mg/kg once every two weeks. In some embodiments, the anti-Sortilin antibody is administered to the individual intravenously at a dose of at least about 30 mg/kg once every three weeks. In some embodiments, the anti-Sortilin antibody is administered to the individual intravenously at a dose of at least about 30 mg/kg once every four weeks.

In some aspects, methods of the present disclosure comprise administering an anti-Sortilin antibody to the individual intravenously at a dose of at least about 35 mg/kg once every four weeks or more frequently. In some embodiments, the anti-Sortilin antibody is administered to the individual intravenously at a dose of at least about 35 mg/kg once every two weeks. In some embodiments, the anti-Sortilin antibody is administered to the individual intravenously at a dose of at least about 35 mg/kg once every three weeks. In some embodiments, the anti-Sortilin antibody is administered to the individual intravenously at a dose of at least about 35 mg/kg once every four weeks.

In some aspects, methods of the present disclosure comprise administering an anti-Sortilin antibody to the individual intravenously at a dose of at least about 40 mg/kg once every four weeks or more frequently. In some embodiments, the anti-Sortilin antibody is administered to the individual intravenously at a dose of at least about 40 mg/kg once every two weeks. In some embodiments, the anti-Sortilin antibody is administered to the individual intravenously at a dose of at least about 40 mg/kg once every three weeks. In some embodiments, the anti-Sortilin antibody is administered to the individual intravenously at a dose of at least about 40 mg/kg once every four weeks.

In some aspects, methods of the present disclosure comprise administering an anti-Sortilin antibody to the individual intravenously at a dose of at least about 45 mg/kg once every four weeks or more frequently. In some embodiments, the anti-Sortilin antibody is administered to the individual intravenously at a dose of at least about 45 mg/kg once every two weeks. In some embodiments, the anti-Sortilin antibody is administered to the individual intravenously at a dose of at least about 45 mg/kg once every three weeks. In some embodiments, the anti-Sortilin antibody is administered to the individual intravenously at a dose of at least about 45 mg/kg once every four weeks.

In some aspects, methods of the present disclosure comprise administering an anti-Sortilin antibody to the individual intravenously at a dose of at least about 50 mg/kg once every four weeks or more frequently. In some embodiments, the anti-Sortilin antibody is administered to the individual intravenously at a dose of at least about 50 mg/kg once every two weeks. In some embodiments, the anti-Sortilin antibody is administered to the individual intravenously at a dose of at least about 50 mg/kg once every three weeks. In some embodiments, the anti-Sortilin antibody is administered to the individual intravenously at a dose of at least about 50 mg/kg once every four weeks.

In some aspects, methods of the present disclosure comprise administering an anti-Sortilin antibody to the individual intravenously at a dose of at least about 55 mg/kg once every four weeks or more frequently. In some embodiments, the anti-Sortilin antibody is administered to the individual intravenously at a dose of at least about 55 mg/kg once every two weeks. In some embodiments, the anti-Sortilin antibody is administered to the individual intravenously at a dose of at least about 55 mg/kg once every three weeks. In some embodiments, the anti-Sortilin antibody is administered to the individual intravenously at a dose of at least about 55 mg/kg once every four weeks.

In some embodiments, the anti-Sortilin antibody is administered to the individual intravenously at a dose of about 60 mg/kg once every four weeks or more frequently. In some embodiments, the anti-Sortilin antibody is administered to the individual intravenously at a dose of about 60 mg/kg once every two weeks. In some embodiments, the anti-Sortilin antibody is administered to the individual intravenously at a dose of about 60 mg/kg once every three weeks. In some embodiments, the anti-Sortilin antibody is administered to the individual intravenously at a dose of about 60 mg/kg once every four weeks.

In some aspects, methods of the present disclosure comprise administering an anti-Sortilin antibody to the individual intravenously at a dose of at least 30 mg/kg once every four weeks or more frequently. In some embodiments, the anti-Sortilin antibody is administered to the individual intravenously at a dose of at least 30 mg/kg once every two weeks. In some embodiments, the anti-Sortilin antibody is administered to the individual intravenously at a dose of at least 30 mg/kg once every three weeks. In some embodiments, the anti-Sortilin antibody is administered to the individual intravenously at a dose of at least 30 mg/kg once every four weeks.

In some aspects, methods of the present disclosure comprise administering an anti-Sortilin antibody to the individual intravenously at a dose of at least 35 mg/kg once every four weeks or more frequently. In some embodiments, the anti-Sortilin antibody is administered to the individual intravenously at a dose of at least 35 mg/kg once every two weeks. In some embodiments, the anti-Sortilin antibody is administered to the individual intravenously at a dose of at least 35 mg/kg once every three weeks. In some embodiments, the anti-Sortilin antibody is administered to the individual intravenously at a dose of at least 35 mg/kg once every four weeks.

In some aspects, methods of the present disclosure comprise administering an anti-Sortilin antibody to the individual intravenously at a dose of at least 40 mg/kg once every four weeks or more frequently. In some embodiments, the anti-Sortilin antibody is administered to the individual intravenously at a dose of at least 40 mg/kg once every two weeks. In some embodiments, the anti-Sortilin antibody is administered to the individual intravenously at a dose of at least 40 mg/kg once every three weeks. In some embodiments, the anti-Sortilin antibody is administered to the individual intravenously at a dose of at least 40 mg/kg once every four weeks.

In some aspects, methods of the present disclosure comprise administering an anti-Sortilin antibody to the individual intravenously at a dose of at least 45 mg/kg once every four weeks or more frequently. In some embodiments, the anti-Sortilin antibody is administered to the individual intravenously at a dose of at least 45 mg/kg once every two weeks. In some embodiments, the anti-Sortilin antibody is administered to the individual intravenously at a dose of at least 45 mg/kg once every three weeks. In some embodiments, the anti-Sortilin antibody is administered to the individual intravenously at a dose of at least 45 mg/kg once every four weeks.

In some aspects, methods of the present disclosure comprise administering an anti-Sortilin antibody to the individual intravenously at a dose of at least 50 mg/kg once every four weeks or more frequently. In some embodiments, the anti-Sortilin antibody is administered to the individual intravenously at a dose of at least 50 mg/kg once every two weeks. In some embodiments, the anti-Sortilin antibody is administered to the individual intravenously at a dose of at least 50 mg/kg once every three weeks. In some embodiments, the anti-Sortilin antibody is administered to the individual intravenously at a dose of at least 50 mg/kg once every four weeks.

In some aspects, methods of the present disclosure comprise administering an anti-Sortilin antibody to the individual intravenously at a dose of at least 55 mg/kg once every four weeks or more frequently. In some embodiments, the anti-Sortilin antibody is administered to the individual intravenously at a dose of at least 55 mg/kg once every two weeks. In some embodiments, the anti-Sortilin antibody is administered to the individual intravenously at a dose of at least 55 mg/kg once every three weeks. In some embodiments, the anti-Sortilin antibody is administered to the individual intravenously at a dose of at least 55 mg/kg once every four weeks.

In some embodiments, the anti-Sortilin antibody is administered to the individual intravenously at a dose of 60 mg/kg once every four weeks or more frequently. In some embodiments, the anti-Sortilin antibody is administered to the individual intravenously at a dose of 60 mg/kg once every two weeks. In some embodiments, the anti-Sortilin antibody is administered to the individual intravenously at a dose of 60 mg/kg once every three weeks. In some embodiments, the anti-Sortilin antibody is administered to the individual intravenously at a dose of 60 mg/kg once every four weeks.

In certain embodiments, the anti-Sortilin antibody is administered to the individual intravenously over about 60 minutes.

In certain embodiments, the anti-Sortilin antibody is administered to the individual intravenously at a dose of at least about 30 mg/kg over about 60 minutes. In certain embodiments, the anti-Sortilin antibody is administered to the individual intravenously at a dose of at least about 30 mg/kg over at least 60 minutes. In certain embodiments, the anti-Sortilin antibody is administered to the individual intravenously at a dose of at least about 35 mg/kg over about 60 minutes. In certain embodiments, the anti-Sortilin antibody is administered to the individual intravenously at a dose of at least about 35 mg/kg over at least 60 minutes. In certain embodiments, the anti-Sortilin antibody is administered to the individual intravenously at a dose of at least about 40 mg/kg over about 60 minutes. In certain embodiments, the anti-Sortilin antibody is administered to the individual intravenously at a dose of at least about 40 mg/kg over at least 60 minutes. In certain embodiments, the anti-Sortilin antibody is administered to the individual intravenously at a dose of at least about 45 mg/kg over about 60 minutes. In certain embodiments, the anti-Sortilin antibody is administered to the individual intravenously at a dose of at least about 45 mg/kg over at least 60 minutes. In certain embodiments, the anti-Sortilin antibody is administered to the individual intravenously at a dose of at least about 50 mg/kg over about 60 minutes. In certain embodiments, the anti-Sortilin antibody is administered to the individual intravenously at a dose of at least about 50 mg/kg over at least 60 minutes. In certain embodiments, the anti-Sortilin antibody is administered to the individual intravenously at a dose of at least about 55 mg/kg over about 60 minutes. In certain embodiments, the anti-Sortilin antibody is administered to the individual intravenously at a dose of at least about 55 mg/kg over at least 60 minutes. In certain embodiments, the anti-Sortilin antibody is administered to the individual intravenously at a dose of about 60 mg/kg over about 60 minutes. In certain embodiments, the anti-Sortilin antibody is administered to the individual intravenously at a dose of about 60 mg/kg over at least 60 minutes.

In certain embodiments, the anti-Sortilin antibody is administered to the individual intravenously at a dose of at least 30 mg/kg over about 60 minutes. In certain embodiments, the anti-Sortilin antibody is administered to the individual intravenously at a dose of at least 30 mg/kg over at least 60 minutes. In certain embodiments, the anti-Sortilin antibody is administered to the individual intravenously at a dose of at least 35 mg/kg over about 60 minutes. In certain embodiments, the anti-Sortilin antibody is administered to the individual intravenously at a dose of at least 35 mg/kg over at least 60 minutes. In certain embodiments, the anti-Sortilin antibody is administered to the individual intravenously at a dose of at least 40 mg/kg over about 60 minutes. In certain embodiments, the anti-Sortilin antibody is administered to the individual intravenously at a dose of at least 40 mg/kg over at least 60 minutes. In certain embodiments, the anti-Sortilin antibody is administered to the individual intravenously at a dose of at least 45 mg/kg over about 60 minutes. In certain embodiments, the anti-Sortilin antibody is administered to the individual intravenously at a dose of at least 45 mg/kg over at least 60 minutes. In certain embodiments, the anti-Sortilin antibody is administered to the individual intravenously at a dose of at least 50 mg/kg over about 60 minutes. In certain embodiments, the anti-Sortilin antibody is administered to the individual intravenously at a dose of at least 50 mg/kg over at least 60 minutes. In certain embodiments, the anti-Sortilin antibody is administered to the individual intravenously at a dose of at least 55 mg/kg over about 60 minutes. In certain embodiments, the anti-Sortilin antibody is administered to the individual intravenously at a dose of at least 55 mg/kg over at least 60 minutes. In certain embodiments, the anti-Sortilin antibody is administered to the individual intravenously at a dose of 60 mg/kg over about 60 minutes. In certain embodiments, the anti-Sortilin antibody is administered to the individual intravenously at a dose of 60 mg/kg over at least 60 minutes.

In certain embodiments, at least 2 doses, at least 4 doses, at least 6 doses, at least 8 doses, at least 10 doses, at least 12 doses, at least 14 doses, at least 16 doses, at least 18 doses, or at least 20 doses of the anti-Sortilin antibody are administered to the individual intravenously. In certain embodiments, a total of 13 doses of the anti-Sortilin antibody are administered to the individual.

In some embodiments, the individual is treated for a treatment period of up to 24 weeks, up to 25 weeks, up to 26 weeks, up to 27 weeks, up to 28 weeks, up to 29 weeks, up to 30 weeks, up to 31 weeks, up to 32 weeks, up to 33 weeks, up to 34 weeks, up to 35 weeks, up to 36 weeks, up to 37 weeks, up to 38 weeks, up to 39 weeks, up to 40 weeks, up to 41 weeks, up to 42 weeks, up to 43 weeks, up to 44 weeks, up to 45 weeks, up to 46 weeks, up to 47 weeks, or up to 48 weeks in length. In some embodiments, the individual is treated for a treatment period of up to 48 weeks in length. In some embodiments, the individual is treated for a treatment period of 48 weeks in length.

In some embodiments, administration of the anti-Sortilin antibody occurs on the first day of the treatment period and every four weeks thereafter.

In some embodiments, the anti-Sortilin antibody is administered a total of 13 times during the treatment period.

An initial higher loading dose, followed by one or more lower doses may be administered. However, other dosage regimens may be useful. The progress of this therapy is easily monitored by conventional techniques and assays.

### PGRN Levels

In some aspects, methods of the present disclosure comprise administering an anti-Sortilin antibody to the individual intravenously where the level of PGRN protein in the plasma of the individual after administration of the anti-Sortilin antibody is higher than the level of PGRN protein in the plasma of the individual before administration of the anti-Sortilin antibody. In some embodiments, a 1-fold increase in the level of PGRN protein in the plasma of the individual corresponds to a 100% increase in the level of PGRN protein in the plasma of the individual. In some embodiments, the level of PGRN protein in the plasma of the individual after administration of the anti-Sortilin antibody is at least 1-fold higher, at least 1.25-fold higher, at least 1.5-fold higher, at least 1.75-fold higher, at least 2-fold higher, at least 2.25-fold higher, at least 2.5-fold higher, at least 2.75-fold higher, or at least 3-fold higher, than the level of PGRN protein in the plasma of the individual before administration of the anti-Sortilin antibody. In some embodiments, the level of PGRN protein in the plasma of the individual after administration of the anti-Sortilin antibody is at least 1-fold higher than the level of PGRN protein in the plasma of the individual before administration of the anti-Sortilin antibody. In some embodiments, the level of PGRN protein in the plasma of the individual after administration of the anti-Sortilin antibody is at least 2-fold higher than the level of PGRN protein in the plasma of the individual before administration of the anti-Sortilin antibody.

In some embodiments, a 2-fold increase in the level of PGRN protein in the plasma of the individual after administration of the anti-Sortilin antibody corresponds to a 100% increase in the level of PGRN protein in the plasma of the individual compared to the level of PGRN protein in the plasma of the individual before administration of the anti-Sortilin antibody. In some embodiments, the level of PGRN protein in the plasma of the individual after administration of the anti-Sortilin antibody is at least two-fold, at least three-fold, or at least four-fold higher than the level of PGRN protein in the plasma of the individual before administration of the anti-Sortilin antibody. In some embodiments, the level of PGRN protein in the plasma of the individual after administration of the anti-Sortilin antibody is at least two-fold higher than the level of PGRN protein in the plasma of the individual before administration of the anti-Sortilin antibody.

In some embodiments, the fold increase in the level of PGRN protein in the plasma of the individual is present at about 5 days, at about 6 days, at about 7 days, at about 8 days, at about 9 days, at about 10 days, at about 11 days, or at about 12 days after administration of the anti-Sortilin antibody. In some embodiments, the fold increase in the level of PGRN protein in the plasma of the individual is present at about five days after administration of the anti-Sortilin antibody. In some embodiments, the fold increase in the level of PGRN protein in the plasma of the individual is present at about 42 days after administration of the anti-Sortilin antibody. In some embodiments, the fold increase in the level of PGRN protein in the plasma of the individual is present at about 56 days after administration of the anti-Sortilin antibody.

In some embodiments, the fold increase in the level of PGRN protein in the plasma of the individual is present at about 5 days, at about 6 days, at about 7 days, at about 8 days, at about 9 days, at about 10 days, at about 11 days, or at about 12 days after the last administration of the anti-Sortilin antibody. In some embodiments, the fold increase in the level of PGRN protein in the plasma of the individual is present at about 28 days, 35 days, 42 days, 49 days, or 56 days after the last administration of the anti-Sortilin antibody. In some embodiments, the fold increase in the level of PGRN protein in the plasma of the individual is present at about five days after the last administration of the anti-Sortilin antibody. In some embodiments, the fold increase in the level of PGRN protein in the plasma of the individual is present at about 28 days after the last administration of the anti-Sortilin antibody. In some embodiments, the fold increase in the level of PGRN protein in the plasma of the individual is present at about 35 days after the last administration of the anti-Sortilin antibody. In some embodiments, the fold increase in the level of PGRN protein in the plasma of the individual is present at about 42 days after the last administration of the anti-Sortilin antibody. In some embodiments, the fold increase in the level of PGRN protein in the plasma of the individual is present at about 49 days after the last administration of the anti-Sortilin antibody. In some embodiments, the fold increase in the level of PGRN protein in the plasma of the individual is present at about 56 days after the last administration of the anti-Sortilin antibody.

In some embodiments, the level of PGRN protein in the plasma of the individual after administration of the anti-Sortilin antibody is at least 0.25-fold higher, at least 0.3-fold higher, at least 0.35-fold higher, at least 0.4-fold higher, at least 0.45-fold higher, at least 0.5-fold higher, at least 0.55-fold higher, at least 0.6-fold higher, at least 0.65-fold higher, at least 0.7-fold higher, at least 0.75-fold higher, at least 0.8-fold higher, at least 0.85-fold higher, at least 0.9-fold higher, at least 0.95-fold higher, at least 1-fold higher, or at least 1.5-fold higher than the level of PGRN protein in the plasma of the individual before administration of the anti-Sortilin antibody at about forty days, about 41 days, or about 42 days after administration of the anti-Sortilin antibody. In some embodiments, the level of PGRN protein in the plasma of the individual after administration of the anti-Sortilin antibody is at least 0.25-fold higher than the level of PGRN protein in the plasma of the individual before administration of the anti-Sortilin antibody at about forty days after administration of the anti-Sortilin antibody.

In some embodiments, the level of PGRN protein in the plasma of the individual is determined by drawing blood at multiple time-points. In certain embodiments, the level of PGRN protein in the plasma of the individual is determined by drawing blood 8, 5, 3, 2, 1 and/or 0 days before administration of the anti-Sortilin antibody and 1, 2, 3, 4, 5, 6, 7, 8, 9, 10, 11, 12, 13, 18, 30, 42, 43, 57, 85, and/or 113 days after administration of the anti-Sortilin antibody. In certain embodiments, the level of PGRN protein in the plasma of the individual is determined by drawing blood 8, 5, 3, 2, 1 and/or 0 days before administration of the anti-Sortilin antibody and 1, 2, 3, 6, 8, 13, 30, 43, 57, 85, and 113 days after administration of the anti-Sortilin antibody. In certain embodiments, the level of PGRN protein in the plasma of the individual is determined by drawing blood up to 6 weeks, up to 5 weeks, up to 4 weeks, up to 3 weeks, up to 2 weeks, up to 1 week, up to 7 days, up to 6 days, up to 5 days, up to 4 days, up to 3 days, up to 2 days, up to 1 day, and/or 0 days before administration of the first dose of anti-Sortilin antibody, on the same day of each administration of the anti-Sortilin antibody, and 10 weeks, 20 weeks, 30 weeks, 40 weeks, 50 weeks, 60 weeks, and/or 70 weeks after administration of the first dose of anti-Sortilin antibody. In certain embodiments, the level of PGRN protein in the plasma of the individual is determined by drawing blood up to 6 weeks, up to 5 weeks, up to 4 weeks, up to 3 weeks, up to 2 weeks, up to 1 week, up to 7 days, up to 6 days, up to 5 days, up to 4 days, up to 3 days, up to 2 days, up to 1 day, and/or 0 days before administration of the first dose of anti-Sortilin antibody, on the same day of each administration of the anti-Sortilin antibody, and 61 weeks after administration of the first dose of anti-Sortilin antibody.

In some aspects, methods of the present disclosure comprise administering an anti-Sortilin antibody to the individual intravenously where the level of PGRN protein in the cerebrospinal fluid of the individual after administration of the anti-Sortilin antibody is higher than the level of PGRN protein in the cerebrospinal fluid of the individual before administration of the anti-Sortilin antibody. In some embodiments, a 1-fold increase in the level of PGRN protein in the cerebrospinal fluid of the individual corresponds to a 100% increase in the level of PGRN protein in the cerebrospinal fluid of the individual. In some embodiments, the level of PGRN protein in the cerebrospinal fluid of the individual after administration of the anti-Sortilin antibody is at least 0.8-fold higher, at least 0.85-fold higher, at least 0.9-fold higher, at least 0.95-fold higher, at least 1-fold higher, or at least 1.2-fold higher than the level of PGRN protein in the cerebrospinal fluid of the individual before administration of the anti-Sortilin antibody. In some embodiments, the level of PGRN protein in the cerebrospinal fluid of the individual after administration of the anti-Sortilin antibody is at least 0.8-fold higher than the level of PGRN protein in the cerebrospinal fluid of the individual before administration of the anti-Sortilin antibody. In some embodiments, the level of PGRN protein in the cerebrospinal fluid of the individual after administration of the anti-Sortilin antibody is at least 1-fold higher than the level of PGRN protein in the cerebrospinal fluid of the individual before administration of the anti-Sortilin antibody.

In some embodiments, the fold increase in the level of PGRN protein in the cerebrospinal fluid of the individual is present at about 1 day, at about 2 days, at about 3 days, at about 4 days, at about 5 days, at about 6 days, at about 7 days, at about 8 days, at about 9 days, at about 10 days, at about 11 days, at about 12 days, at about 13 days, at about 14 days, at about 15 days, at about 16 days, at about 17 days, at about 18 days, at about 19 days, at about 20 days, at about 21 days, at about 22 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, or at about 42 days after administration of the anti-Sortilin antibody. In some embodiments, the fold increase in the level of PGRN protein in the cerebrospinal fluid of the individual is present at about twelve days after administration of the anti-Sortilin antibody. In some embodiments, the fold increase in the level of PGRN protein in the cerebrospinal fluid of the individual is present at about 24 days after administration of the anti-Sortilin antibody. In some embodiments, the fold increase in the level of PGRN protein in the cerebrospinal fluid of the individual is present at about 56 days after administration of the anti-Sortilin antibody.

In some embodiments, a two-fold increase in the level of PGRN protein in the cerebrospinal fluid of the individual corresponds to a 100% increase in the level of PGRN protein in the cerebrospinal fluid of the individual. In some embodiments, the level of PGRN protein in the cerebrospinal fluid of the individual after administration of the anti-Sortilin antibody is any of at least 2-fold higher, at least 2.5-fold higher, at least 3-fold higher, at least 3.5-fold higher, at least 4-fold higher, at least 4.5-fold higher, at least 5-fold higher than the level of PGRN protein in the cerebrospinal fluid of the individual before administration of the anti-Sortilin antibody. In some embodiments, the level of PGRN protein in the cerebrospinal fluid of the individual after administration of the anti-Sortilin antibody is at least two-fold higher than the level of PGRN protein in the cerebrospinal fluid of the individual before administration of the anti-Sortilin antibody.

In some embodiments, the fold increase in the level of PGRN protein in the cerebrospinal fluid of the individual is present at about 1 day, at about 2 days, at about 3 days, at about 4 days, at about 5 days, at about 6 days, at about 7 days, at about 8 days, at about 9 days, at about 10 days, at about 11 days, at about 12 days, at about 13 days, at about 14 days, at about 15 days, at about 16 days, at about 17 days, at about 18 days, at about 19 days, at about 20 days, at about 21 days, at about 22 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, or at about 42 days after the last administration of the anti-Sortilin antibody. In some embodiments, the fold increase in the level of PGRN protein in the cerebrospinal fluid of the individual is present at any of about 28 days, 35 days, 42 days, 49 days, or 56 days after the last administration of the anti-Sortilin antibody. In some embodiments, the fold increase in the level of PGRN protein in the cerebrospinal fluid of the individual is present at about twelve days after the last administration of the anti-Sortilin antibody. In some embodiments, the fold increase in the level of PGRN protein in the cerebrospinal fluid of the individual is present at about 24 days after the last administration of the anti-Sortilin antibody. In some embodiments, the fold increase in the level of PGRN protein in the cerebrospinal fluid of the individual is present at about 28 days after the last administration of the anti-Sortilin antibody. In some embodiments, the fold increase in the level of PGRN protein in the cerebrospinal fluid of the individual is present at about 35 days after the last administration of the anti-Sortilin antibody. In some embodiments, the fold increase in the level of PGRN protein in the cerebrospinal fluid of the individual is present at about 42 days after the last administration of the anti-Sortilin antibody. In some embodiments, the fold increase in the level of PGRN protein in the cerebrospinal fluid of the individual is present at about 49 days after the last administration of the anti-Sortilin antibody. In some embodiments, the fold increase in the level of PGRN protein in the cerebrospinal fluid of the individual is present at about 56 days after the last administration of the anti-Sortilin antibody.

In some embodiments, the level of PGRN protein in the cerebrospinal fluid of the individual after administration of the anti-Sortilin antibody is at least 0.2-fold higher than the level of PGRN protein in the cerebrospinal fluid of the individual before administration of the anti-Sortilin antibody at about 1 day, at about 2 days, at about 3 days, at about 4 days, at about 5 days, at about 6 days, at about 7 days, at about 8 days, at about 9 days, at about 10 days, at about 11 days, at about 12 days, at about 13 days, at about 14 days, at about 15 days, at about 16 days, at about 17 days, at about 18 days, at about 19 days, at about 20 days, at about 21 days, at about 22 days, at about 23 days, at about 24 days, at about 25 days, at about 26 days, at about 27 days, at about 28 days, at about 29 days, at about 30 days, at about 31 days, at about 32 days, at about 33 days, at about 34 days, at about 35 days, at about 36 days, at about 37 days, at about 38 days, at about 39 days, at about 40 days, at about 41 days, or at about 42 days after administration of the anti-Sortilin antibody. In some embodiments, the level of PGRN protein in the cerebrospinal fluid of the individual after administration of the anti-Sortilin antibody is at least 0.2-fold higher than the level of PGRN protein in the cerebrospinal fluid of the individual before administration of the anti-Sortilin antibody at about 42 days after administration of the anti-Sortilin antibody.

In some embodiments, the level of PGRN protein in the cerebrospinal fluid of the individual is determined by performing a lumbar puncture at multiple time-points. In certain embodiments, the level of PGRN protein in the cerebrospinal fluid of the individual is determined by performing a lumbar puncture 8, 5, 3, 2, 1, and/or 0 days before administration of the anti-Sortilin antibody and 1 day, 30 hours, 2 days, 12 days, 24 days, and/or 42 days after administration of the anti-Sortilin antibody. In certain embodiments, the level of PGRN protein in the cerebrospinal fluid of the individual is determined by performing a lumbar puncture up to 6 weeks, up to 5 weeks, up to 4 weeks, up to 3 weeks, up to 2 weeks, up to 1 week, up to 7 days, up to 6 days, up to 5 days, up to 4 days, up to 3 days, up to 2 days, up to 1 day, and/or 0 days before administration of the first dose of anti-Sortilin antibody and at least 10 weeks, at least 15 weeks, at least 20 weeks, at least 25 weeks, at least 30 weeks, at least 40 weeks, at least 50 weeks, and/or at least 60 weeks after administration of the first dose of anti-Sortilin antibody. In certain embodiments, the level of PGRN protein in the cerebrospinal fluid of the individual is determined by performing a lumbar puncture up to 6 weeks, up to 5 weeks, up to 4 weeks, up to 3 weeks, up to 2 weeks, up to 1 week, up to 7 days, up to 6 days, up to 5 days, up to 4 days, up to 3 days, up to 2 days, up to 1 day, and/or 0 days before administration of the first dose of anti-Sortilin antibody and during week 25 and during week 61 after administration of the first dose of anti-Sortilin antibody.

In some embodiments, the level of PGRN protein in the plasma or the cerebrospinal fluid of the individual is determined using any method of quantifying proteins known in the art. Non-limiting examples of methods that may be used to quantify PGRN protein include SOMASCAN assay (see, e.g., Candia et al. (2017) Sci Rep 7, 14248), Western blots, mass spectrometry, flow cytometry, and enzyme-linked immunosorbent assay (ELISA) assays. In certain embodiments, the level of PGRN protein in the plasma or the cerebrospinal fluid of the individual is determined using ELISA assays.

### SORT1 Levels

In some aspects, methods of the present disclosure comprise administering an anti-Sortilin antibody to the individual intravenously where the expression level of SORT1 protein on peripheral white blood cells of the individual after administration of the anti-Sortilin antibody is reduced compared to the expression level of SORT1 protein on peripheral white blood cells of the individual before administration of the anti-Sortilin antibody. In some embodiments, the expression level of SORT1 protein on peripheral white blood cells of the individual after administration of the anti-Sortilin antibody is reduced by at least 40%, at least 45%, at least 50%, at least 55%, at least 60%, at least 65%, at least 70%, at least 75%, or at least 80% compared to the expression level of SORT1 protein on peripheral white blood cells of the individual before administration of the anti-Sortilin antibody. In some embodiments, the expression level of SORT1 protein on peripheral white blood cells of the individual after administration of the anti-Sortilin antibody is reduced by at least 50% compared to the expression level of SORT1 protein on peripheral white blood cells of the individual before administration of the anti-Sortilin antibody. In some embodiments, the expression level of SORT1 protein on peripheral white blood cells of the individual after administration of the anti-Sortilin antibody is reduced by at least 70% compared to the expression level of SORT1 protein on peripheral white blood cells of the individual before administration of the anti-Sortilin antibody.

In some embodiments, the reduction in the expression level of SORT1 in peripheral white blood cells of the individual is present at about 10 days or more, 11 days or more, 12 days or more, 13 days or more, 14 days or more, 15 days or more, 16 days or more, 17 days or more, 18 days or more, 19 days or more, 20 days or more, 21 days or more, 22 days or more, 23 days or more, 24 days or more, 25 days or more, 26 days or more, 27 days or more, 28 days or more, 29 days or more, 30 days or more, 31 days or more, 32 days or more, 33 days or more, 34 days or more, 35 days or more, 36 days or more, 37 days or more, 38 days or more, 39 days or more, 40 days or more, 41 days or more, 42 days or more, 43 days or more, 44 days or more, or 45 days or more after administration of the anti-Sortilin antibody. In some embodiments, the reduction in the expression level of SORT1 in peripheral white blood cells of the individual is present at about twelve days or more after administration of the anti-Sortilin antibody. In some embodiments, the reduction in the expression level of SORT1 in peripheral white blood cells of the individual is present at seventeen days or more after administration of the anti-Sortilin antibody. In some embodiments, the reduction in the expression level of SORT1 in peripheral white blood cells of the individual is present at about forty days or more after administration of the anti-Sortilin antibody.

In some embodiments, the reduction in the expression level of SORT1 in peripheral white blood cells of the individual is present at about 10 days or more, 11 days or more, 12 days or more, 13 days or more, 14 days or more, 15 days or more, 16 days or more, 17 days or more, 18 days or more, 19 days or more, 20 days or more, 21 days or more, 22 days or more, 23 days or more, 24 days or more, 25 days or more, 26 days or more, 27 days or more, 28 days or more, 29 days or more, 30 days or more, 31 days or more, 32 days or more, 33 days or more, 34 days or more, 35 days or more, 36 days or more, 37 days or more, 38 days or more, 39 days or more, 40 days or more, 41 days or more, 42 days or more, 43 days or more, 44 days or more, or 45 days or more after the last administration of the anti-Sortilin antibody. In some embodiments, the reduction in the expression level of SORT1 in peripheral white blood cells of the individual is present at about twelve days or more after the last administration of the anti-Sortilin antibody. In some embodiments, the reduction in the expression level of SORT1 in peripheral white blood cells of the individual is present at about seventeen days or more after the last administration of the anti-Sortilin antibody. In some embodiments, the reduction in the expression level of SORT1 in peripheral white blood cells of the individual is present at about forty days or more after the last administration of the anti-Sortilin antibody.

In some aspects, methods of the present disclosure comprise administering an anti-Sortilin antibody to the individual intravenously where the level of SORT1 protein in the cerebrospinal fluid of the individual after administration of the anti-Sortilin antibody is reduced compared to the level of SORT1 protein in the cerebrospinal fluid of the individual before administration of the anti-Sortilin antibody. In some embodiments, the level of SORT1 protein in the cerebrospinal fluid of the individual after administration of the anti-Sortilin antibody is reduced by least 10%, at least 20%, at least 30%, at least 40%, at least 50%, at least 60%, at least 70% at least 80%, or at least 90% compared to the level of SORT1 protein in the cerebrospinal fluid of the individual before administration of the anti-Sortilin antibody. In some embodiments, the level of SORT1 protein in the cerebrospinal fluid of the individual after administration of the anti-Sortilin antibody is reduced by at least 50% compared to the level of SORT1 protein in the cerebrospinal fluid of the individual before administration of the anti-Sortilin antibody. In some embodiments, the level of SORT1 protein in the cerebrospinal fluid of the individual after administration of the anti-Sortilin antibody is reduced by at least 70% compared to the level of SORT1 protein in the cerebrospinal fluid of the individual before administration of the anti-Sortilin antibody.

In some embodiments, the level of SORT1 protein on peripheral white blood cells of the individual is determined by drawing blood at multiple time-points. In certain embodiments, the level of SORT1 on peripheral white blood cells of the individual is determined by drawing blood 8, 5, 3, 2, 1 and/or 0 days before administration of the anti-Sortilin antibody and 1, 2, 3, 4, 5, 6, 7, 8, 9, 10, 11, 12, 13, 18, 30, 42, 43, 57, 85, and/or 113 days after administration of the anti-Sortilin antibody. In certain embodiments, the level of SORT1 on peripheral white blood cells of the individual is determined by drawing blood 8, 5, 3, 2, 1 and/or 0 days before administration of the anti-Sortilin antibody and 1, 2, 3, 6, 8, 9, 13, 18, 30, 43, 57, 85, and 113 days after administration of the anti-Sortilin antibody. In certain embodiments, the level of SORT1 protein on peripheral white blood cells of the individual is determined by drawing blood up to 6 weeks, up to 5 weeks, up to 4 weeks, up to 3 weeks, up to 2 weeks, up to 1 week, up to 7 days, up to 6 days, up to 5 days, up to 4 days, up to 3 days, up to 2 days, up to 1 day, and/or 0 days before administration of the first dose of anti-Sortilin antibody, on the same day of each administration of the anti-Sortilin antibody, and 10 weeks, 20 weeks, 30 weeks, 40 weeks, 50 weeks, 60 weeks, and/or 70 weeks after administration of the first dose of anti-Sortilin antibody. In certain embodiments, the level of SORT1 protein on peripheral white blood cells of the individual is determined by drawing blood up to 6 weeks, up to 5 weeks, up to 4 weeks, up to 3 weeks, up to 2 weeks, up to 1 week, up to 7 days, up to 6 days, up to 5 days, up to 4 days, up to 3 days, up to 2 days, up to 1 day, and/or 0 days before administration of the first dose of anti-Sortilin antibody, on the same day of each administration of the anti-Sortilin antibody, and during week 61 after administration of the first dose of anti-Sortilin antibody.

In some embodiments, the level of SORT1 protein in the cerebrospinal fluid of the individual is determined by performing a lumbar puncture at multiple time-points. In certain embodiments, the level of SORT1 protein in the cerebrospinal fluid of the individual is determined by performing a lumbar puncture 8, 5, 3, 2, 1, and/or 0 days before administration of the anti-Sortilin antibody and 1 day, 30 hours, 12 days, 24 days, and/or 42 days after administration of the anti-Sortilin antibody. In certain embodiments, the level of SORT1 protein in the cerebrospinal fluid of the individual is determined by performing a lumbar puncture up to 6 weeks, up to 5 weeks, up to 4 weeks, up to 3 weeks, up to 2 weeks, up to 1 week, up to 7 days, up to 6 days, up to 5 days, up to 4 days, up to 3 days, up to 2 days, up to 1 day, and/or 0 days before administration of the first dose of anti-Sortilin antibody and at least 10 weeks, at least 15 weeks, at least 20 weeks, at least 25 weeks, at least 30 weeks, at least 40 weeks, at least 50 weeks, and/or at least 60 weeks after administration of the first dose of anti-Sortilin antibody. In certain embodiments, the level of SORT1 protein in the cerebrospinal fluid of the individual is determined by performing a lumbar puncture up to 6 weeks, up to 5 weeks, up to 4 weeks, up to 3 weeks, up to 2 weeks, up to 1 week, up to 7 days, up to 6 days, up to 5 days, up to 4 days, up to 3 days, up to 2 days, up to 1 day, and/or 0 days before administration of the first dose of anti-Sortilin antibody and during week 25 and during week 61 after administration of the first dose of anti-Sortilin antibody.

In some embodiments, the level of SORT1 protein on peripheral white blood cells or the level of soluble SORT1 protein in the cerebrospinal fluid of the individual is determined using any method of quantifying proteins known in the art. Non-limiting examples of methods that may be used to quantify SORT1 protein include SOMASCAN assay (see, e.g., Candia et al. (2017) Sci Rep 7, 14248), Western blots, mass spectrometry, flow cytometry, and enzyme-linked immunosorbent assay (ELISA) assays. In certain embodiments, the level of SORT1 protein on peripheral white blood cells or in the cerebrospinal fluid of the individual is determined using ELISA assays. In certain embodiments, the level of SORT1 protein on peripheral white blood cells or in the cerebrospinal fluid of the individual is determined using ELISA assays with anti-Sortilin antibody-specific anti-idiotypic antibodies.

### Pharmacokinetics of Anti-Sortilin Antibodies

In some embodiments, the half-life of the anti-Sortilin antibody in plasma is around 5 days, around 6 days, around 7 days, around 8 days, or around 9 days. In some embodiments, the half-life of the anti-Sortilin antibody in plasma is around 5 days. In some embodiments, the half-life of the anti-Sortilin antibody in plasma is around 8 days.

### Diagnostic Uses

The isolated antibodies of the present disclosure (e.g., an anti-Sortilin antibody described herein) also have diagnostic utility. This disclosure therefore provides for methods of using the antibodies of this disclosure, or functional fragments thereof, for diagnostic purposes, such as the detection of a Sortilin protein in an individual or in tissue samples derived from an individual.

In some embodiments, the individual is a human. In some embodiments, the individual is a human patient suffering from, or at risk for developing a disease, disorder, or injury of the present disclosure. In some embodiments, the diagnostic methods involve detecting a Sortilin protein in a biological sample, such as a biopsy specimen, a tissue, or a cell. An anti-Sortilin antibody described herein is contacted with the biological sample and antigen-bound antibody is detected. For example, a biopsy specimen may be stained with an anti-Sortilin antibody described herein in order to detect and/or quantify disease-associated cells. The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, or micro-positron emission tomography. In certain embodiments, the antibody is radiolabeled, for example with ¹⁸F and subsequently detected utilizing micro-positron emission tomography analysis. Antibody-binding may also be quantified in a individual by non-invasive techniques such as positron emission tomography (PET), X-ray computed tomography, single-photon emission computed tomography (SPECT), computed tomography (CT), and computed axial tomography (CAT).

In other embodiments, an isolated antibody of the present disclosure (e.g., an anti-Sortilin antibody described herein) may be used to detect and/or quantify, for example, microglia in a brain specimen taken from a preclinical disease model (e.g., a non-human disease model). As such, an isolated antibody of the present disclosure (e.g., an anti-Sortilin antibody described herein) may be useful in evaluating therapeutic response after treatment in a model for a nervous system disease or injury such as frontotemporal dementia, Alzheimer's disease, vascular dementia, seizures, retinal dystrophy, atherosclerotic vascular diseases, Nasu-Hakola disease, or multiple sclerosis, as compared to a control.

### Sortilin Antibodies

Certain aspects of the present disclosure relate to anti-Sortilin antibodies comprising one or more improved and/or enhanced functional characteristics. In some embodiments, anti-Sortilin antibodies of the present disclosure comprise one or more improved and/or enhanced functional characteristics relative to an anti-Sortilin antibody, 5-60, having a heavy chain variable region and a light chain variable region as described in WO2016164637. In some embodiments, anti-Sortilin antibodies of the present disclosure have an affinity for Sortilin (e.g., human Sortilin) that is higher than that of a control anti-Sortilin antibody (e.g., a control anti-Sortilin antibody comprising a heavy chain variable region and a light chain variable region corresponding to S-60). In some embodiments, anti-Sortilin antibodies of the present disclosure decrease cellular levels (e.g., cell surface levels) of Sortilin to a greater degree and with a half-maximal effective concentration (EC₅₀) that is lower than that of a control antibody (e.g., a control anti-Sortilin antibody comprising a heavy chain variable region and a light chain variable region corresponding to S-60). In some embodiments, anti-Sortilin antibodies of the present disclosure improve the maximal reduction of cell surface levels of Sortilin relative to an anti-Sortilin antibody comprising a heavy chain variable region and a light chain variable region corresponding to S-60. In some embodiments, anti-Sortilin antibodies of the present disclosure increase the secretion of extracellular Progranulin (PGRN) relative to an anti-Sortilin antibody comprising a heavy chain variable region and a light chain variable region corresponding to S-60. In some embodiments, anti-Sortilin antibodies of the present disclosure blocking binding of PGRN to Sortilin to a greater degree and with a half-maximal effective concentration (EC₅₀) that is lower than that of a control antibody (e.g., a control anti-Sortilin antibody comprising a heavy chain variable region and a light chain variable region corresponding to S-60). In some embodiments, anti-Sortilin antibodies of the present disclosure improve the maximal blocking of PGRN binding to Sortilin relative to an anti-Sortilin antibody comprising a heavy chain variable region and a light chain variable region corresponding to S-60.

Also contemplated herein are anti-Sortilin antibodies with different Fe variants that exhibit one or more improved and/or enhanced functional characteristics relative to an anti-Sortilin antibody comprising a heavy chain variable region and a light chain variable region corresponding to S-60, including decreasing the half-maximal effective concentration (EC₅₀) to reduce cell surface levels of Sortilin, improving the maximal reduction of cell surface levels of Sortilin, increasing extracellular secretion of PGRN, decreasing the half-maximal effective concentration (EC₅₀) to block PGRN binding to Sortilin, and improving the maximal blocking of PGRN binding to Sortilin.

In some embodiments, an anti-Sortilin antibody of the present disclosure is a human antibody, a bispecific antibody, a monoclonal antibody, a multivalent antibody, a conjugated antibody, or a chimeric antibody

In a preferred embodiment, an anti-Sortilin antibody of the present disclosure is a monoclonal antibody.

### Anti-Sortilin Antibody Heavy Chain and Light Chain Variable Regions

### A. Heavy Chain HVRs

In some embodiments, anti-Sortilin antibodies of the present disclosure include a heavy chain variable region comprising one or more (e.g., one or more, two or more, or all three) HVRs selected from HVR-H1, HVR-H2, and HVR-H3 (as shown in Tables 11-13). In some embodiments, the heavy chain variable region comprises an HVR-H1, an HVR-H2, and an HVR-H3 (as shown in Tables 11-13).

In some embodiments, the HVR-H1 comprises a sequence of YSISSGYYWG (SEQ ID NO: 1). In some embodiments, the HVR-H2 comprises a sequence according to Formula I: TIYHSGSTYYNPSLXiS (SEQ ID NO: 4), wherein X₁ is K or E. In some embodiments, the HVR-H2 comprises a sequence selected from SEQ ID NOs: 2-3. In some embodiments, the HVR-H3 comprises a sequence according to Formula II: ARQGSIXiQGYYGMDV (SEQ ID NO: 7). In some embodiments, the HVR-H3 comprises a sequence selected from SEQ ID NOs: 5-6.

In some embodiments, the HVR-H1 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence of SEQ ID NO: 1. In some embodiments, the HVR-H1 comprises an amino acid sequence containing substitutions (e.g., conservative substitutions, insertions, or deletions relative to an amino acid sequence of SEQ ID NO: 1), but retains the ability to bind to Sortilin. In certain embodiments, up to 1, up to 2, up to 3, up to 4, or up to 5 amino acids been substituted, inserted, and/or deleted in the HVR-H1 amino acid sequence of SEQ ID NO: 1. In some embodiments, the HVR-H2 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 2-3. In some embodiments, the HVR-H2 comprises an amino acid sequence containing substitutions (e.g., conservative substitutions, insertions, or deletions relative to an amino acid sequence selected from SEQ ID NOs: 2-3), but retains the ability to bind to Sortilin. In certain embodiments, up to 1, up to 2, up to 3, up to 4, or up to 5 amino acids been substituted, inserted, and/or deleted in the HVR-H2 amino acid sequence selected from SEQ ID NOs: 2-3. In some embodiments, the HVR-H3 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 5-6. In some embodiments, the HVR-H3 comprises an amino acid sequence containing substitutions (e.g., conservative substitutions, insertions, or deletions relative to an amino acid sequence selected from SEQ ID NOs: 5-6), but retains the ability to bind to Sortilin. In certain embodiments, up to 1, up to 2, up to 3, up to 4, or up to 5 amino acids been substituted, inserted, and/or deleted in the HVR-H3 amino acid sequence selected from SEQ ID NOs: 5-6.

In some embodiments, the heavy chain variable region comprises an HVR-H1 comprising a sequence of YSISSGYYWG (SEQ ID NO: 1), an HVR-H2 comprising a sequence according to Formula I, and an HVR-H3 comprising a sequence according to Formula II.

In some embodiments, the heavy chain variable region comprises an HVR-H1 comprising a sequence of SEQ ID NO: 1, an HVR-H2 comprising a sequence selected from SEQ ID NOs: 2-3, and an HVR-H3 comprising a sequence selected from SEQ ID NOs: 5-6.

In some embodiments, the heavy chain variable region comprises the HVR-H1, HVR-H2, and HVR-H3 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, S-60-24, or any combination thereof (as shown in Tables 11-13).

In some embodiments, anti-Sortilin antibodies of the present disclosure include a heavy chain variable region, wherein the heavy chain variable region comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H1 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H2 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H3 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], 5-60-15.7 [N33K], 5-60-15.8 [N33Q], 5-60-15.9 [N33Y], 5-60-15.10 [N33E], 5-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24.

In some embodiments, anti-Sortilin antibodies of the present disclosure comprise an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6).

### B. Light Chain HVRs

In some embodiments, anti-Sortilin antibodies of the present disclosure comprise a light chain variable region comprising one or more (e.g., one or more, two or more, or all three) HVRs selected from HVR-L1, HVR-L2, and HVR-L3 (as shown in Tables 14-16). In some embodiments, the light chain variable region comprises an HVR-L1, an HVR-L2, and an HVR-L3 (as shown in Tables 14-16).

In some embodiments, the HVR-L1 comprises a sequence according to Formula III: RSSQX₁LLX₂SX₃GYNYLD (SEQ ID NO: 28), wherein X₁ is S or G, X₂ is R or H, and X₃ is N, T, S, G, R, D, H, K, Q, Y, E, W, F, I, V, A, M, or L. In some embodiments, the HVR-L1 comprises a sequence selected from SEQ ID NOs: 8-27. In some embodiments, the HVR-L1 comprises a sequence of RSSQSLLRSNGYNYLD (SEQ ID NO:8), RSSQSLLRSTGYNYLD (SEQ ID NO:9), RSSQS LLRSSGYNYLD (SEQ ID NO:10), RSSQSLLRSGGYNYLD (SEQ ID NO:11), RSSQSLLRSRG YNYLD (SEQ ID NO:12), RSSQSLLRSDGYNYLD (SEQ ID NO:13), RSSQSLLRSHGYNYLD (SEQ ID NO:14), RSSQSLLRSKGYNYLD (SEQ ID NO:15), RSSQSLLRSQGYNYLD (SEQ ID NO:16), RSSQSLLRSYGYNYLD (SEQ ID NO:17), RSSQSLLRSEGYNYLD (SEQ ID NO:18), RSSQSLLRSWGYNYLD (SEQ ID NO:19), RSSQSLLRSFGYNYLD (SEQ ID NO:20), RSSQSL LRSIGYNYLD (SEQ ID NO:21), RSSQSLLRSVGYNYLD (SEQ ID NO:22), RSSQSLLRSAG YNYLD (SEQ ID NO:23), RSSQSLLRSMGYNYLD (SEQ ID NO:24), RSSQSLLRSLGYNYLD (SEQ ID NO:25), RSSQSLLHSNGYNYLD (SEQ ID NO:26), or RSSQGLLRSNGYNYLD (SEQ ID NO:27). In one specific embodiment, the HVR-L1 comprises a sequence of RSSQSLLRSNGYNYLD (SEQ ID NO:8). In another specific embodiment, the HVR-L1 comprises a sequence of RSSQSLLRSTGYNYLD (SEQ ID NO:9) (as shown in Table 14).

In some embodiments, the HVR-L2 comprises a sequence according to Formula IV: LGSNRX1S (SEQ ID NO: 31), wherein X₁ is A or V. In some embodiments, the HVR-L2 comprises a sequence selected from SEQ ID NOs: 29-30.

In some embodiments, the HVR-L3 comprises a sequence according to Formula V: MQQQEX1PLT (SEQ ID NO: 34), wherein X₁ is A or T. In some embodiments, the HVR-L3 comprises a sequence selected from SEQ ID NOs: 32-33.

In some embodiments, the HVR-L1 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 8-27. In some embodiments, the HVR-L1 comprises an amino acid sequence containing substitutions (e.g., conservative substitutions, insertions, or deletions relative to an amino acid sequence selected from SEQ ID NOs: 8-27), but retains the ability to bind to Sortilin. In certain embodiments, up to 1, up to 2, up to 3, up to 4, or up to 5 amino acids been substituted, inserted, and/or deleted in the HVR-L1 amino acid sequence selected from SEQ ID NOs: 8-27. In some embodiments, the HVR-L2 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 29-30. In some embodiments, the HVR-L2 comprises an amino acid sequence containing substitutions (e.g., conservative substitutions, insertions, or deletions relative to an amino acid sequence selected from SEQ ID NOs: 29-30), but retains the ability to bind to Sortilin. In certain embodiments, up to 1, up to 2, up to 3, up to 4, or up to 5 amino acids been substituted, inserted, and/or deleted in the HVR-L2 amino acid sequence selected from SEQ ID NOs: 29-30. In some embodiments, the HVR-L3 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 32-33. In some embodiments, the HVR-L3 comprises an amino acid sequence containing substitutions (e.g., conservative substitutions, insertions, or deletions relative to an amino acid sequence selected from SEQ ID NOs: 32-33), but retains the ability to bind to Sortilin. In certain embodiments, up to 1, up to 2, up to 3, up to 4, or up to 5 amino acids been substituted, inserted, and/or deleted in the HVR-L3 amino acid sequence selected from SEQ ID NOs: 32-33.

In some embodiments, the light chain variable region comprises an HVR-L1 comprising a sequence according to Formula III, an HVR-L2 comprising a sequence according to Formula IV, and an HVR-L3 comprising a sequence according to Formula V. In some embodiments, the light chain variable region comprises an HVR-L1 comprising a sequence selected from SEQ ID NOs: 8-27, an HVR-L2 comprising a sequence selected from SEQ ID NOs: 29-30, and an HVR-L3 comprising a sequence selected from SEQ ID NOs: 32-33.

In some embodiments, the light chain variable region comprises the HVR-L1, HVR-L2, and HVR-L3 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, S-60-24, or any combination thereof (as shown in Tables 14-16).

In some embodiments, anti-Sortilin antibodies of the present disclosure include a light chain variable region, wherein the light chain variable region comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L1 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L2 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L3 amino acid sequence of antibody S-60-10, S-60-11, S-60-12, S-60-13, S-60-14, S-60-15 [N33 (wt)], S-60-15.1 [N33T], 5-60-15.2 [N33S], 5-60-15.3 [N33G], 5-60-15.4 [N33R], 5-60-15.5 [N33D], 5-60-15.6 [N33H], 5-60-15.7 [N33K], 5-60-15.8 [N33Q], 5-60-15.9 [N33Y], 5-60-15.10 [N33E], 5-60-15.11 [N33W], 5-60-15.12 [N33F], 5-60-15.13 [N331], 5-60-15.14 [N33V], 5-60-15.15 [N33A], 5-60-15.16 [N33M], 5-60-15.17 [N33L], 5-60-16; 5-60-18, 5-60-19, or 5-60-24.

In some embodiments, anti-Sortilin antibodies of the present disclosure comprise an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32).

In some embodiments, anti-Sortilin antibodies of the present disclosure comprise an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

### C. Heavy Chain HVRs and Light Chain HVRs

In some embodiments, anti-Sortilin antibodies of the present disclosure include a heavy chain variable region comprising one or more (e.g., one or more, two or more, or all three) HVRs selected from HVR-H1, HVR-H2, and HVR-H3 (as shown in Tables 11-13), and a light chain variable region comprising one or more (e.g., one or more, two or more, or all three) HVRs selected from HVR-L1, HVR-L2, and HVR-L3 (as shown in Tables 14-16). In some embodiments, the heavy chain variable region comprises an HVR-H1, an HVR-H2, and an HVR-H3 (as shown in Tables 11-13), and the light chain variable region comprises an HVR-L1, an HVR-L2, and an HVR-L3 (as shown in Tables 14-16).

In some embodiments, the heavy chain variable region comprises an HVR-H1 comprising a sequence of YSISSGYYWG (SEQ ID NO: 1), an HVR-H2 comprising a sequence according to Formula I, and an HVR-H3 comprising a sequence according to Formula II, and the light chain variable region comprises an HVR-L1 comprising a sequence according to Formula III, an HVR-L2 comprising a sequence according to Formula IV, and an HVR-L3 comprising a sequence according to Formula V. In some embodiments, the heavy chain variable region comprises an HVR-H1 comprising a sequence of SEQ ID NO: 1, an HVR-H2 comprising a sequence selected from SEQ ID NOs: 2-3, and an HVR-H3 comprising a sequence selected from SEQ ID NOs: 5-6, and the light chain variable region comprises an HVR-L1 comprising a sequence selected from SEQ ID NOs: 8-27, an HVR-L2 comprising a sequence selected from SEQ ID NOs: 29-30, and an HVR-L3 comprising a sequence selected from SEQ ID NOs: 32-33.

In some aspects, the heavy chain variable region comprises an HVR-H1 comprising a sequence of SEQ ID NO: 1, an HVR-H2 comprising a sequence selected from SEQ ID NOs: 2-3, and an HVR-H3 comprising a sequence selected from SEQ ID NOs: 5-6, and the light chain variable region comprises an HVR-L1 comprising a sequence selected from SEQ ID NOs: 8-27, an HVR-L2 comprising a sequence selected from SEQ ID NOs: 29-30, and an HVR-L3 comprising a sequence of SEQ ID NO: 32.

In some embodiments, anti-Sortilin antibodies of the present disclosure comprise a heavy chain variable region comprising the HVR-H1, HVR-H2, and HVR-H3 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, S-60-24, or any combination thereof (as shown in Tables 11-13); and a light chain variable region comprising the HVR-L1, HVR-L2, and HVR-L3 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, S-60-24, or any combination thereof (as shown in Tables 14-16).

In some embodiments, anti-Sortilin antibodies of the present disclosure comprise a heavy chain variable region comprising an HVR-H1, HVR-H2, and HVR-H3 and a light chain variable region comprising an HVR-L1, HVR-L2, and HVR-L3, wherein the antibody comprises the HVR-H1, HVR-H2, HVR-H3, HVR-L1, HVR-L2, and HVR-L3 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24 (as shown in Tables 11-16).

In some embodiments, anti-Sortilin antibodies of the present disclosure comprise a heavy chain variable region and a light chain variable region, wherein the heavy chain variable region comprises one or more of: (a) an HVR-H1 comprising an amino acid sequence with at least 90%, at least 91%, at least 92%, at least 93%, at least 94%, at least 95%, at least 96%, at least 97%, at least 98%, at least 99%, or 100% identity to an HVR-H1 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24; (b) an HVR-H2 comprising an amino acid sequence with at least 90%, at least 91%, at least 92%, at least 93%, at least 94%, at least 95%, at least 96%, at least 97%, at least 98%, at least 99%, or 100% identity to an HVR-H2 amino acid sequence of antibody S-60-10, S-60-11, S-60-12, S-60-13, S-60-14, S-60-15 [N33 (wt)], S-60-15.1 [N33T], S-60-15.2 [N33S], 5-60-15.3 [N33G], 5-60-15.4 [N33R], 5-60-15.5 [N33D], 5-60-15.6 [N33H], 5-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, 5-60-19, or 5-60-24; and (c) an HVR-H3 comprising an amino acid sequence with at least 90%, at least 91%, at least 92%, at least 93%, at least 94%, at least 95%, at least 96%, at least 97%, at least 98%, at least 99%, or 100% identity to an HVR-H3 amino acid sequence of antibody S-60-10, S-60-11, S-60-12, S-60-13, 5-60-14, 5-60-15 [N33 (wt)], 5-60-15.1 [N33T], 5-60-15.2 [N33S], 5-60-15.3 [N33G], 5-60-15.4 [N33R], 5-60-15.5 [N33D], 5-60-15.6 [N33H], 5-60-15.7 [N33K], 5-60-15.8 [N33Q], 5-60-15.9 [N33Y], S-60-15.10 [N33E], 5-60-15.11 [N33W], 5-60-15.12 [N33F], 5-60-15.13 [N331], 5-60-15.14 [N33V], 5-60-15.15 [N33A], 5-60-15.16 [N33M], 5-60-15.17 [N33L], 5-60-16; 5-60-18, 5-60-19, or 5-60-24; and wherein the light chain variable region comprises one or more of: (a) an HVR-L1 comprising an amino acid sequence with at least 90%, at least 91%, at least 92%, at least 93%, at least 94%, at least 95%, at least 96%, at least 97%, at least 98%, at least 99%, or 100% identity to an HVR-L1 amino acid sequence of antibody 5-60-10, 5-60-11, 5-60-12, 5-60-13, 5-60-14, 5-60-15 [N33 (wt)], 5-60-15.1 [N33T], 5-60-15.2 [N33S], 5- 60-15.3 [N33G], 5-60-15.4 [N33R], 5-60-15.5 [N33D], 5-60-15.6 [N33H], 5-60-15.7 [N33K], 5-60-15.8 [N33Q], S-60-15.9 [N33Y], 5-60-15.10 [N33E], 5-60-15.11 [N33W], 5-60-15.12 [N33F], 5-60-15.13 [N331], 5-60-15.14 [N33V], 5-60-15.15 [N33A], 5-60-15.16 [N33M], 5-60-15.17 [N33L], 5-60-16; 5-60-18, 5-60-19, or 5-60-24; (b) an HVR-L2 comprising an amino acid sequence with at least 90%, at least 91%, at least 92%, at least 93%, at least 94%, at least 95%, at least 96%, at least 97%, at least 98%, at least 99%, or 100% identity to an HVR-L2 amino acid sequence of antibody 5-60-10, 5-60-11, 5-60-12, 5-60-13, 5-60-14, 5-60-15 [N33 (wt)], 5-60-15.1 [N33T], 5-60-15.2 [N33S], 5-60-15.3 [N33G], 5-60-15.4 [N33R], 5-60-15.5 [N33D], 5-60-15.6 [N33H], 5-60-15.7 [N33K], 5-60-15.8 [N33Q], 5-60-15.9 [N33Y], 5-60-15.10 [N33E], 5-60- 15.11 [N33W], 5-60-15.12 [N33F], 5-60-15.13 [N331], 5-60-15.14 [N33V], 5-60-15.15 [N33A], 5-60-15.16 [N33M], 5-60-15.17 [N33L], 5-60-16; 5-60-18, 5-60-19, or 5-60-24; and (c) an HVR-L3 comprising an amino acid sequence with at least 90%, at least 91%, at least 92%, at least 93%, at least 94%, at least 95%, at least 96%, at least 97%, at least 98%, at least 99%, or 100% identity to an HVR-L3 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24.

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32).

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRVS (SEQ ID NO: 30), and the HVR-L3 comprising the amino acid sequence MQQQETPLT (SEQ ID NO: 33).

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLES (SEQ ID NO: 3), the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32).

In some aspects, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32).

In some aspects, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSTGYNYLD (SEQ ID NO: 9), the HVR-L2 comprising the amino acid sequence LGSNRAS (SEQ ID NO: 29), and the HVR-L3 comprising the amino acid sequence MQQQEAPLT (SEQ ID NO: 32).

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQETPLT (SEQ ID NO: 33).

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIQQGYYGMDV (SEQ ID NO: 5); and a light chain variable region comprising the HVR-L1 comprising the amino acid sequence RSSQSLLHSNGYNYLD (SEQ ID NO: 26), the HVR-L2 comprising the amino acid sequence LGSNRAS (SEQ ID NO: 29), and the HVR-L3 comprising the amino acid sequence MQQQETPLT (SEQ ID NO: 33).

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQGLLRSNGYNYLD (SEQ ID NO: 27), the HVR-L2 comprising the amino acid sequence LGSNRAS (SEQ ID NO: 29), and the HVR-L3 comprising the amino acid sequence MQQQEAPLT (SEQ ID NO: 32).

### D. Heavy Chain Variable Region

In some embodiments, anti-Sortilin antibodies of the present disclosure include a heavy chain variable region comprising an amino acid sequence selected from SEQ ID NOs: 54-56. In some embodiments, the heavy chain variable region comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 54-56. In some embodiments, the heavy chain variable region comprises an amino acid sequence containing substitutions (e.g., conservative substitutions, insertions, or deletions relative to an amino acid sequence selected from SEQ ID NOs: 54-56), but retains the ability to bind to Sortilin. In certain embodiments, up to 1, up to 2, up to 3, up to 4, up to 5, up to 6, up to 7, up to 8, up to 9, or up to 10 amino acids been substituted, inserted, and/or deleted in the heavy chain variable region amino acid sequence selected from SEQ ID NOs: 54-56.

In some embodiments, the heavy chain variable region comprises the amino acid sequence of SEQ ID NO: 56.

In some embodiments, anti-Sortilin antibodies of the present disclosure include a heavy chain variable region of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24 (as shown in Table 25).

In some embodiments, anti-Sortilin antibodies of the present disclosure include a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6).

### E. Light Chain Variable Region

In some embodiments, anti-Sortilin antibodies of the present disclosure include a light chain variable region comprising an amino acid sequence selected from SEQ ID NOs: 57-80. In some embodiments, the light chain variable region comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 57-80. In some embodiments, the light chain variable region comprises an amino acid sequence containing substitutions (e.g., conservative substitutions, insertions, or deletions relative to an amino acid sequence selected from SEQ ID NOs: 57-80), but retains the ability to bind to Sortilin. In certain embodiments, up to 1, up to 2, up to 3, up to 4, up to 5, up to 6, up to 7, up to 8, up to 9, or up to 10 amino acids been substituted, inserted, and/or deleted in the light chain variable region amino acid sequence selected from SEQ ID NOs: 57-80.

In some embodiments, the light chain variable region includes the amino acid sequence of SEQ ID NO: 57. In some embodiments, the light chain variable region includes the amino acid sequence of SEQ ID NO: 60.

In some embodiments, anti-Sortilin antibodies of the present disclosure comprise a light chain variable region of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24 (as shown in Table 26).

In some embodiments, anti-Sortilin antibodies of the present disclosure include a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32).

In some embodiments, anti-Sortilin antibodies of the present disclosure include a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

### F. Heavy Chain Variable Region and Light Chain Variable Region

In some aspects, an anti-Sortilin antibody of the present disclosure includes a heavy chain variable region comprising an amino acid sequence selected from the group consisting of SEQ ID NOs: 54-56; and/or a light chain variable region comprises an amino acid sequence selected from the group consisting of SEQ ID NOs: 57-80. In some embodiments, the heavy chain variable region comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 54-56, and the light chain variable region comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 57-80. In some embodiments, an anti-Sortilin antibody of the present disclosure includes a heavy chain variable region comprising an amino acid sequence containing substitutions (e.g., conservative substitutions, insertions, or deletions relative to an amino acid sequence selected from SEQ ID NOs: 54-56), and a light chain variable region comprising an amino acid sequence containing substitutions (e.g., conservative substitutions, insertions, or deletions relative to an amino acid sequence selected from SEQ ID NOs: 57-80), but retains the ability to bind to Sortilin. In certain embodiments, up to 1, up to 2, up to 3, up to 4, up to 5, up to 6, up to 7, up to 8, up to 9, or up to 10 amino acids been substituted, inserted, and/or deleted in the heavy chain variable region amino acid sequence selected from SEQ ID NOs: 54-56; and up to 1, up to 2, up to 3, up to 4, up to 5, up to 6, up to 7, up to 8, up to 9, or up to 10 amino acids been substituted, inserted, and/or deleted in the light chain variable region amino acid sequence selected from SEQ ID NOs: 57-80.

In some aspects, an anti-Sortilin antibody of the present disclosure includes a heavy chain variable region comprising an amino acid sequence selected from the group consisting of SEQ ID NOs: 54-56; and/or a light chain variable region comprises an amino acid sequence selected from the group consisting of SEQ ID NOs: 57-58, 60-78, and 80.

In some embodiments, an anti-Sortilin antibody of the present disclosure binds to a Sortilin protein, wherein the antibody includes a heavy chain variable region comprising the amino acid sequence of SEQ ID NO: 54, and a light chain variable region comprising the amino acid sequence of SEQ ID NO: 57; a heavy chain variable region comprising the amino acid sequence of SEQ ID NO: 54, and a light chain variable region comprising the amino acid sequence of SEQ ID NO: 58; a heavy chain variable region comprising the amino acid sequence of SEQ ID NO: 54, and a light chain variable region comprising the amino acid sequence of SEQ ID NO: 59; a heavy chain variable region comprising the amino acid sequence of SEQ ID NO: 55, and a light chain variable region comprising the amino acid sequence of SEQ ID NO: 57; a heavy chain variable region comprising the amino acid sequence of SEQ ID NO: 55, and a light chain variable region comprising the amino acid sequence of SEQ ID NO: 58; a heavy chain variable region comprising the amino acid sequence of SEQ ID NO: 56, and a light chain variable region comprising the amino acid sequence of SEQ ID NO: 57; a heavy chain variable region comprising the amino acid sequence of SEQ ID NO: 56, and a light chain variable region comprising the amino acid sequence of SEQ ID NO: 77; a heavy chain variable region comprising the amino acid sequence of SEQ ID NO: 56, and a light chain variable region comprising the amino acid sequence of SEQ ID NO: 78; a heavy chain variable region comprising the amino acid sequence of SEQ ID NO: 54, and a light chain variable region comprising the amino acid sequence of SEQ ID NO: 79; or a heavy chain variable region comprising the amino acid sequence of SEQ ID NO: 56, and a light chain variable region comprising the amino acid sequence of SEQ ID NO: 80.

In one aspect, an anti-Sortilin antibody of the present disclosure includes a heavy chain variable region having the amino acid sequence of SEQ ID NO: 56, and a light chain variable region having the amino acid sequence of SEQ ID NO: 57.

In one aspect, an anti-Sortilin antibody of the present disclosure includes a heavy chain variable region having the amino acid sequence of SEQ ID NO: 56, and a light chain variable region having the amino acid sequence of SEQ ID NO: 60.

In some embodiments, anti-Sortilin antibodies of the present disclosure comprise a heavy chain variable region of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16; S-60-18, S-60-19, or S-60-24 (as shown in Table 25), and a light chain variable region of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16; S-60-18, S-60-19, or S-60-24 (as shown in Table 26).

In some embodiments, anti-Sortilin antibodies of the present disclosure comprise a heavy chain variable region comprising the amino acid sequence of SEQ ID NO: 56, and a light chain variable region comprising an amino acid sequence selected from SEQ ID NOs: 57 and 60. In some embodiments, the antibody comprises a heavy chain variable region of S-60-15 [N33 (wt)](as shown in Table 25), and a light chain variable region of antibody 5-60-15 [N33 (wt)](as shown in Table 26). In some embodiments, the antibody comprises a heavy chain variable region of S-60-15.1 [N33T](as shown in Table 25), and a light chain variable region of antibody 5-60-15.1 [N33T](as shown in Table 26).

### Exemplary Anti-Sortilin Antibodies

In some embodiments, the anti-Sortilin antibody is an anti-Sortilin monoclonal antibody comprising the heavy chain variable region and the light chain variable region of an antibody selected from S-60-10, S-60-11, S-60-12, S-60-13, S-60-14, S-60-15 [N33 (wt)], S-60-15.1 [N33T], S-60-15.2 [N33S], S- 60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16; S-60-18, S-60-19, or S-60-24. In some embodiments, the anti-Sortilin antibody is an anti-Sortilin monoclonal antibody comprising the heavy chain and the light chain of an antibody selected from S-60-10, 5-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16; S-60-18, S-60-19, or S-60-24.

### (1) S-60-10

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-10 or to the amino acid sequence of SEQ ID NO: 54; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-10 or to the amino acid sequence of SEQ ID NO: 57. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-10 or to the amino acid sequence of SEQ ID NO: 54, wherein the heavy chain variable domain comprises the HVR-H1, HVR-H2, and HVR-H3 amino acid sequences of antibody S-60-10. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-10 or to the amino acid sequence of SEQ ID NO: 57, wherein the light chain variable domain comprises the HVR-L1, HVR-L2, and HVR-L3 amino acid sequences of antibody S-60-10. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-10 or to the amino acid sequence of SEQ ID NO: 54 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-10 or the amino acid sequence of SEQ ID NO: 54. In certain embodiments, a total of 1 to 5 amino acids have been substituted, inserted and/or deleted in the heavy chain variable domain amino acid sequence of antibody S-60-10 or the amino acid sequence of SEQ ID NO: 54. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VH sequence of antibody S-60-10 or of SEQ ID NO: 54, including post-translational modifications of that sequence. In a particular embodiment, the VH comprises one, two or three HVRs selected from: (a) the HVR-H1 amino acid sequence of antibody S-60-10, (b) the HVR-H2 amino acid sequence of antibody S-60-10, and (c) the HVR-H3 amino acid sequence of antibody S-60-10. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-10 or to the amino acid sequence of SEQ ID NO: 57 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-10 or the amino acid sequence of SEQ ID NO: 57. In certain embodiments, a total of 1 to 5 amino acids have been substituted, inserted and/or deleted in the light chain variable domain amino acid sequence of antibody S-60-10 or the amino acid sequence of SEQ ID NO: 57. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VL sequence of antibody S-60-10 or of SEQ ID NO: 57, including post-translational modifications of that sequence. In a particular embodiment, the VL comprises one, two or three HVRs selected from: (a) the HVR-L1 amino acid sequence of antibody S-60-10, (b) the HVR-L2 amino acid sequence of antibody S-60-10, and (c) the HVR-L3 amino acid sequence of antibody S-60-10.

In some embodiments, anti-Sortilin antibodies of the present disclosure comprise a heavy chain comprising the amino acid sequence of SEQ ID NO: 86 or SEQ ID NO: 87. In some embodiments, anti-Sortilin antibodies of the present disclosure comprise a light chain comprising the amino acid sequence of SEQ ID NO: 92. In some embodiments, anti-Sortilin antibodies of the present disclosure comprise a heavy chain comprising the amino acid sequence of SEQ ID NO: 86 or SEQ ID NO: 87 and a light chain comprising the amino acid sequence of SEQ ID NO: 92.

### (2) S-60-11

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-11 or to the amino acid sequence of SEQ ID NO: 54; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-11 or to the amino acid sequence of SEQ ID NO: 58. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-11 or to the amino acid sequence of SEQ ID NO: 54, wherein the heavy chain variable domain comprises the HVR-H1, HVR-H2, and HVR-H3 amino acid sequences of antibody S-60-11. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-11 or to the amino acid sequence of SEQ ID NO: 58, wherein the light chain variable domain comprises the HVR-L1, HVR-L2, and HVR-L3 amino acid sequences of antibody S-60-11. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-11 or to the amino acid sequence of SEQ ID NO: 54 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-11 or the amino acid sequence of SEQ ID NO: 54. In certain embodiments, a total of 1 to 5 amino acids have been substituted, inserted and/or deleted in the heavy chain variable domain amino acid sequence of antibody S-60-11 or the amino acid sequence of SEQ ID NO: 54. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VH sequence of antibody S-60-11 or of SEQ ID NO: 54, including post-translational modifications of that sequence. In a particular embodiment, the VH comprises one, two or three HVRs selected from: (a) the HVR-H1 amino acid sequence of antibody S-60-11, (b) the HVR-H2 amino acid sequence of antibody S-60-11, and (c) the HVR-H3 amino acid sequence of antibody S-60-11. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-11 or to the amino acid sequence of SEQ ID NO: 58 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-11 or the amino acid sequence of SEQ ID NO: 58. In certain embodiments, a total of 1 to 5 amino acids have been substituted, inserted and/or deleted in the light chain variable domain amino acid sequence of antibody S-60-11 or the amino acid sequence of SEQ ID NO: 58. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VL sequence of antibody S-60-11 or of SEQ ID NO: 58, including post-translational modifications of that sequence. In a particular embodiment, the VL comprises one, two or three HVRs selected from: (a) the HVR-L1 amino acid sequence of antibody S-60-11, (b) the HVR-L2 amino acid sequence of antibody S-60-11, and (c) the HVR-L3 amino acid sequence of antibody S-60-11.

In some embodiments, anti-Sortilin antibodies of the present disclosure comprise a heavy chain comprising the amino acid sequence of SEQ ID NO: 86 or SEQ ID NO: 87. In some embodiments, anti-Sortilin antibodies of the present disclosure comprise a light chain comprising the amino acid sequence of SEQ ID NO: 93. In some embodiments, anti-Sortilin antibodies of the present disclosure comprise a heavy chain comprising the amino acid sequence of SEQ ID NO: 86 or SEQ ID NO: 87 and a light chain comprising the amino acid sequence of SEQ ID NO: 93.

### (3) S-60-12

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-12 or to the amino acid sequence of SEQ ID NO: 54; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-12 or to the amino acid sequence of SEQ ID NO: 59. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-12 or to the amino acid sequence of SEQ ID NO: 54, wherein the heavy chain variable domain comprises the HVR-H1, HVR-H2, and HVR-H3 amino acid sequences of antibody S-60-12. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-12 or to the amino acid sequence of SEQ ID NO: 59, wherein the light chain variable domain comprises the HVR-L1, HVR-L2, and HVR-L3 amino acid sequences of antibody S-60-12. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-12 or to the amino acid sequence of SEQ ID NO: 54 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-12 or the amino acid sequence of SEQ ID NO: 54. In certain embodiments, a total of 1 to 5 amino acids have been substituted, inserted and/or deleted in the heavy chain variable domain amino acid sequence of antibody S-60-12 or the amino acid sequence of SEQ ID NO: 54. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VH sequence of antibody S-60-12 or of SEQ ID NO: 54, including post-translational modifications of that sequence. In a particular embodiment, the VH comprises one, two or three HVRs selected from: (a) the HVR-H1 amino acid sequence of antibody S-60-12, (b) the HVR-H2 amino acid sequence of antibody S-60-12, and (c) the HVR-H3 amino acid sequence of antibody S-60-12. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-12 or to the amino acid sequence of SEQ ID NO: 59 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-12 or the amino acid sequence of SEQ ID NO: 59. In certain embodiments, a total of 1 to 5 amino acids have been substituted, inserted and/or deleted in the light chain variable domain amino acid sequence of antibody S-60-12 or the amino acid sequence of SEQ ID NO: 59. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VL sequence of antibody S-60-12 or of SEQ ID NO: 59, including post-translational modifications of that sequence. In a particular embodiment, the VL comprises one, two or three HVRs selected from: (a) the HVR-L1 amino acid sequence of antibody S-60-12, (b) the HVR-L2 amino acid sequence of antibody S-60-12, and (c) the HVR-L3 amino acid sequence of antibody S-60-12.

In some embodiments, anti-Sortilin antibodies of the present disclosure comprise a heavy chain comprising the amino acid sequence of SEQ ID NO: 86 or SEQ ID NO: 87. In some embodiments, anti-Sortilin antibodies of the present disclosure comprise a light chain comprising the amino acid sequence of SEQ ID NO: 94. In some embodiments, anti-Sortilin antibodies of the present disclosure comprise a heavy chain comprising the amino acid sequence of SEQ ID NO: 86 or SEQ ID NO: 87 and a light chain comprising the amino acid sequence of SEQ ID NO: 94.

### (4) S-60-13

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-13 or to the amino acid sequence of SEQ ID NO: 55; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-13 or to the amino acid sequence of SEQ ID NO: 57. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-13 or to the amino acid sequence of SEQ ID NO: 55, wherein the heavy chain variable domain comprises the HVR-H1, HVR-H2, and HVR-H3 amino acid sequences of antibody S-60-13. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-13 or to the amino acid sequence of SEQ ID NO: 57, wherein the light chain variable domain comprises the HVR-L1, HVR-L2, and HVR-L3 amino acid sequences of antibody S-60-13. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-13 or to the amino acid sequence of SEQ ID NO: 55 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-13 or the amino acid sequence of SEQ ID NO: 55. In certain embodiments, a total of 1 to 5 amino acids have been substituted, inserted and/or deleted in the heavy chain variable domain amino acid sequence of antibody S-60-13 or the amino acid sequence of SEQ ID NO: 55. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VH sequence of antibody S-60-13 or of SEQ ID NO: 55, including post-translational modifications of that sequence. In a particular embodiment, the VH comprises one, two or three HVRs selected from: (a) the HVR-H1 amino acid sequence of antibody S-60-13, (b) the HVR-H2 amino acid sequence of antibody S-60-13, and (c) the HVR-H3 amino acid sequence of antibody S-60-13. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-13 or to the amino acid sequence of SEQ ID NO: 57 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-13 or the amino acid sequence of SEQ ID NO: 57. In certain embodiments, a total of 1 to 5 amino acids have been substituted, inserted and/or deleted in the light chain variable domain amino acid sequence of antibody S-60-13 or the amino acid sequence of SEQ ID NO: 57. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VL sequence of antibody S-60-13 or of SEQ ID NO: 57, including post-translational modifications of that sequence. In a particular embodiment, the VL comprises one, two or three HVRs selected from: (a) the HVR-L1 amino acid sequence of antibody S-60-13, (b) the HVR-L2 amino acid sequence of antibody S-60-13, and (c) the HVR-L3 amino acid sequence of antibody S-60-13.

In some embodiments, anti-Sortilin antibodies of the present disclosure comprise a heavy chain comprising the amino acid sequence of SEQ ID NO: 88 or SEQ ID NO: 89. In some embodiments, anti-Sortilin antibodies of the present disclosure comprise a light chain comprising the amino acid sequence of SEQ ID NO: 92. In some embodiments, anti-Sortilin antibodies of the present disclosure comprise a heavy chain comprising the amino acid sequence of SEQ ID NO: 88 or SEQ ID NO: 89 and a light chain comprising the amino acid sequence of SEQ ID NO: 92.

### (5) S-60-14

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-14 or to the amino acid sequence of SEQ ID NO: 55; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-14 or to the amino acid sequence of SEQ ID NO: 58. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-14 or to the amino acid sequence of SEQ ID NO: 55, wherein the heavy chain variable domain comprises the HVR-H1, HVR-H2, and HVR-H3 amino acid sequences of antibody S-60-14. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-14 or to the amino acid sequence of SEQ ID NO: 58, wherein the light chain variable domain comprises the HVR-L1, HVR-L2, and HVR-L3 amino acid sequences of antibody S-60-14. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-14 or to the amino acid sequence of SEQ ID NO: 55 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-14 or the amino acid sequence of SEQ ID NO: 55. In certain embodiments, a total of 1 to 5 amino acids have been substituted, inserted and/or deleted in the heavy chain variable domain amino acid sequence of antibody S-60-14 or the amino acid sequence of SEQ ID NO: 55. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VH sequence of antibody S-60-14 or of SEQ ID NO: 55, including post-translational modifications of that sequence. In a particular embodiment, the VH comprises one, two or three HVRs selected from: (a) the HVR-H1 amino acid sequence of antibody S-60-14, (b) the HVR-H2 amino acid sequence of antibody S-60-14, and (c) the HVR-H3 amino acid sequence of antibody S-60-14. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-14 or to the amino acid sequence of SEQ ID NO: 58 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-14 or the amino acid sequence of SEQ ID NO: 58. In certain embodiments, a total of 1 to 5 amino acids have been substituted, inserted and/or deleted in the light chain variable domain amino acid sequence of antibody S-60-14 or the amino acid sequence of SEQ ID NO: 58. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VL sequence of antibody S-60-14 or of SEQ ID NO: 58, including post-translational modifications of that sequence. In a particular embodiment, the VL comprises one, two or three HVRs selected from: (a) the HVR-L1 amino acid sequence of antibody S-60-14, (b) the HVR-L2 amino acid sequence of antibody S-60-14, and (c) the HVR-L3 amino acid sequence of antibody S-60-14.

In some embodiments, anti-Sortilin antibodies of the present disclosure comprise a heavy chain comprising the amino acid sequence of SEQ ID NO: 88 or SEQ ID NO: 89. In some embodiments, anti-Sortilin antibodies of the present disclosure comprise a light chain comprising the amino acid sequence of SEQ ID NO: 93. In some embodiments, anti-Sortilin antibodies of the present disclosure comprise a heavy chain comprising the amino acid sequence of SEQ ID NO: 88 or SEQ ID NO: 89 and a light chain comprising the amino acid sequence of SEQ ID NO: 93.

### (6) S-60-15

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15 or to the amino acid sequence of SEQ ID NO: 56; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15 or to the amino acid sequence of SEQ ID NO: 57. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15 or to the amino acid sequence of SEQ ID NO: 56, wherein the heavy chain variable domain comprises the HVR-H1, HVR-H2, and HVR-H3 amino acid sequences of antibody S-60-15. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15 or to the amino acid sequence of SEQ ID NO: 57, wherein the light chain variable domain comprises the HVR-L1, HVR-L2, and HVR-L3 amino acid sequences of antibody S-60-15. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15 or to the amino acid sequence of SEQ ID NO: 56 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-15 or the amino acid sequence of SEQ ID NO: 56. In certain embodiments, a total of 1 to 5 amino acids have been substituted, inserted and/or deleted in the heavy chain variable domain amino acid sequence of antibody S-60-15 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VH sequence of antibody S-60-15 or of SEQ ID NO: 56, including post-translational modifications of that sequence. In a particular embodiment, the VH comprises one, two or three HVRs selected from: (a) the HVR-H1 amino acid sequence of antibody S-60-15, (b) the HVR-H2 amino acid sequence of antibody S-60-15, and (c) the HVR-H3 amino acid sequence of antibody S-60-15. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15 or to the amino acid sequence of SEQ ID NO: 57 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-15 or the amino acid sequence of SEQ ID NO: 57. In certain embodiments, a total of 1 to 5 amino acids have been substituted, inserted and/or deleted in the light chain variable domain amino acid sequence of antibody S-60-15 or the amino acid sequence of SEQ ID NO: 57. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VL sequence of antibody S-60-15 or of SEQ ID NO: 57, including post-translational modifications of that sequence. In a particular embodiment, the VL comprises one, two or three HVRs selected from: (a) the HVR-L1 amino acid sequence of antibody S-60-15, (b) the HVR-L2 amino acid sequence of antibody S-60-15, and (c) the HVR-L3 amino acid sequence of antibody S-60-15.

In some embodiments, anti-Sortilin antibodies of the present disclosure comprise a heavy chain comprising the amino acid sequence of SEQ ID NO: 90 or SEQ ID NO: 91. In some embodiments, anti-Sortilin antibodies of the present disclosure comprise a light chain comprising the amino acid sequence of SEQ ID NO: 92. In some embodiments, anti-Sortilin antibodies of the present disclosure comprise a heavy chain comprising the amino acid sequence of SEQ ID NO: 90 or SEQ ID NO: 91 and a light chain comprising the amino acid sequence of SEQ ID NO: 92.

### (7) S-60-15.1

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15.1 or to the amino acid sequence of SEQ ID NO: 56; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15.1 or to the amino acid sequence of SEQ ID NO: 60. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15.1 or to the amino acid sequence of SEQ ID NO: 56, wherein the heavy chain variable domain comprises the HVR-H1, HVR-H2, and HVR-H3 amino acid sequences of antibody S-60-15.1. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15.1 or to the amino acid sequence of SEQ ID NO: 60, wherein the light chain variable domain comprises the HVR-L1, HVR-L2, and HVR-L3 amino acid sequences of antibody S-60-15.1. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15.1 or to the amino acid sequence of SEQ ID NO: 56 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-15.1 or the amino acid sequence of SEQ ID NO: 56. In certain embodiments, a total of 1 to 5 amino acids have been substituted, inserted and/or deleted in the heavy chain variable domain amino acid sequence of antibody S-60-15.1 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VH sequence of antibody S-60-15.1 or of SEQ ID NO: 56, including post-translational modifications of that sequence. In a particular embodiment, the VH comprises one, two or three HVRs selected from: (a) the HVR-H1 amino acid sequence of antibody S-60-15.1, (b) the HVR-H2 amino acid sequence of antibody S-60-15.1, and (c) the HVR-H3 amino acid sequence of antibody S-60-15.1. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15.1 or to the amino acid sequence of SEQ ID NO: 60 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-15.1 or the amino acid sequence of SEQ ID NO: 60. In certain embodiments, a total of 1 to 5 amino acids have been substituted, inserted and/or deleted in the light chain variable domain amino acid sequence of antibody S-60-15.1 or the amino acid sequence of SEQ ID NO: 60. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VL sequence of antibody S-60-15.1 or of SEQ ID NO: 60, including post-translational modifications of that sequence. In a particular embodiment, the VL comprises one, two or three HVRs selected from: (a) the HVR-L1 amino acid sequence of antibody S-60-15.1, (b) the HVR-L2 amino acid sequence of antibody S-60-15.1, and (c) the HVR-L3 amino acid sequence of antibody S-60-15.1.

In some embodiments, anti-Sortilin antibodies of the present disclosure comprise a heavy chain comprising the amino acid sequence of SEQ ID NO: 90 or SEQ ID NO: 91. In some embodiments, anti-Sortilin antibodies of the present disclosure comprise a light chain comprising the amino acid sequence of SEQ ID NO: 95. In some embodiments, anti-Sortilin antibodies of the present disclosure comprise a heavy chain comprising the amino acid sequence of SEQ ID NO: 90 or SEQ ID NO: 91 and a light chain comprising the amino acid sequence of SEQ ID NO: 95.

### (8) S-60-16

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-16 or to the amino acid sequence of SEQ ID NO: 56; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-16 or to the amino acid sequence of SEQ ID NO: 77. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-16 or to the amino acid sequence of SEQ ID NO: 56, wherein the heavy chain variable domain comprises the HVR-H1, HVR-H2, and HVR-H3 amino acid sequences of antibody S-60-16. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-16 or to the amino acid sequence of SEQ ID NO: 77, wherein the light chain variable domain comprises the HVR-L1, HVR-L2, and HVR-L3 amino acid sequences of antibody S-60-16. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-16 or to the amino acid sequence of SEQ ID NO: 56 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-16 or the amino acid sequence of SEQ ID NO: 56. In certain embodiments, a total of 1 to 5 amino acids have been substituted, inserted and/or deleted in the heavy chain variable domain amino acid sequence of antibody S-60-16 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VH sequence of antibody S-60-16 or of SEQ ID NO: 56, including post-translational modifications of that sequence. In a particular embodiment, the VH comprises one, two or three HVRs selected from: (a) the HVR-H1 amino acid sequence of antibody S-60-16, (b) the HVR-H2 amino acid sequence of antibody S-60-16, and (c) the HVR-H3 amino acid sequence of antibody S-60-16. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-16 or to the amino acid sequence of SEQ ID NO: 77 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-16 or the amino acid sequence of SEQ ID NO: 77. In certain embodiments, a total of 1 to 5 amino acids have been substituted, inserted and/or deleted in the light chain variable domain amino acid sequence of antibody S-60-16 or the amino acid sequence of SEQ ID NO: 77. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VL sequence of antibody S-60-16 or of SEQ ID NO: 77, including post-translational modifications of that sequence. In a particular embodiment, the VL comprises one, two or three HVRs selected from: (a) the HVR-L1 amino acid sequence of antibody S-60-16, (b) the HVR-L2 amino acid sequence of antibody S-60-16, and (c) the HVR-L3 amino acid sequence of antibody S-60-16.

In some embodiments, anti-Sortilin antibodies of the present disclosure comprise a heavy chain comprising the amino acid sequence of SEQ ID NO: 90 or SEQ ID NO: 91. In some embodiments, anti-Sortilin antibodies of the present disclosure comprise a light chain comprising the amino acid sequence of SEQ ID NO: 112. In some embodiments, anti-Sortilin antibodies of the present disclosure comprise a heavy chain comprising the amino acid sequence of SEQ ID NO: 90 or SEQ ID NO: 91 and a light chain comprising the amino acid sequence of SEQ ID NO: 112.

### (9) S-60-18

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-18 or to the amino acid sequence of SEQ ID NO: 56; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-18 or to the amino acid sequence of SEQ ID NO: 78. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-18 or to the amino acid sequence of SEQ ID NO: 56, wherein the heavy chain variable domain comprises the HVR-H1, HVR-H2, and HVR-H3 amino acid sequences of antibody S-60-18. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-18 or to the amino acid sequence of SEQ ID NO: 78, wherein the light chain variable domain comprises the HVR-L1, HVR-L2, and HVR-L3 amino acid sequences of antibody S-60-18. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-18 or to the amino acid sequence of SEQ ID NO: 56 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-18 or the amino acid sequence of SEQ ID NO: 56. In certain embodiments, a total of 1 to 5 amino acids have been substituted, inserted and/or deleted in the heavy chain variable domain amino acid sequence of antibody S-60-18 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VH sequence of antibody S-60-18 or of SEQ ID NO: 56, including post-translational modifications of that sequence. In a particular embodiment, the VH comprises one, two or three HVRs selected from: (a) the HVR-H1 amino acid sequence of antibody S-60-18, (b) the HVR-H2 amino acid sequence of antibody S-60-18, and (c) the HVR-H3 amino acid sequence of antibody S-60-18. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-18 or to the amino acid sequence of SEQ ID NO: 78 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-18 or the amino acid sequence of SEQ ID NO: 78. In certain embodiments, a total of 1 to 5 amino acids have been substituted, inserted and/or deleted in the light chain variable domain amino acid sequence of antibody S-60-18 or the amino acid sequence of SEQ ID NO: 78. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VL sequence of antibody S-60-18 or of SEQ ID NO: 78, including post-translational modifications of that sequence. In a particular embodiment, the VL comprises one, two or three HVRs selected from: (a) the HVR-L1 amino acid sequence of antibody S-60-18, (b) the HVR-L2 amino acid sequence of antibody S-60-18, and (c) the HVR-L3 amino acid sequence of antibody S-60-18.

In some embodiments, anti-Sortilin antibodies of the present disclosure comprise a heavy chain comprising the amino acid sequence of SEQ ID NO: 90 or SEQ ID NO: 91. In some embodiments, anti-Sortilin antibodies of the present disclosure comprise a light chain comprising the amino acid sequence of SEQ ID NO: 113. In some embodiments, anti-Sortilin antibodies of the present disclosure comprise a heavy chain comprising the amino acid sequence of SEQ ID NO: 90 or SEQ ID NO: 91 and a light chain comprising the amino acid sequence of SEQ ID NO: 113.

### (10) S-60-19

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-19 or to the amino acid sequence of SEQ ID NO: 54; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-19 or to the amino acid sequence of SEQ ID NO: 79. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-19 or to the amino acid sequence of SEQ ID NO: 54, wherein the heavy chain variable domain comprises the HVR-H1, HVR-H2, and HVR-H3 amino acid sequences of antibody S-60-19. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-19 or to the amino acid sequence of SEQ ID NO: 79, wherein the light chain variable domain comprises the HVR-L1, HVR-L2, and HVR-L3 amino acid sequences of antibody S-60-19. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-19 or to the amino acid sequence of SEQ ID NO: 54 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-19 or the amino acid sequence of SEQ ID NO: 54. In certain embodiments, a total of 1 to 5 amino acids have been substituted, inserted and/or deleted in the heavy chain variable domain amino acid sequence of antibody S-60-19 or the amino acid sequence of SEQ ID NO: 54. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VH sequence of antibody S-60-19 or of SEQ ID NO: 54, including post-translational modifications of that sequence. In a particular embodiment, the VH comprises one, two or three HVRs selected from: (a) the HVR-H1 amino acid sequence of antibody S-60-19, (b) the HVR-H2 amino acid sequence of antibody S-60-19, and (c) the HVR-H3 amino acid sequence of antibody S-60-19. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-19 or to the amino acid sequence of SEQ ID NO: 79 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-19 or the amino acid sequence of SEQ ID NO: 79. In certain embodiments, a total of 1 to 5 amino acids have been substituted, inserted and/or deleted in the light chain variable domain amino acid sequence of antibody S-60-19 or the amino acid sequence of SEQ ID NO: 79. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VL sequence of antibody S-60-19 or of SEQ ID NO: 79, including post-translational modifications of that sequence. In a particular embodiment, the VL comprises one, two or three HVRs selected from: (a) the HVR-L1 amino acid sequence of antibody S-60-19, (b) the HVR-L2 amino acid sequence of antibody S-60-19, and (c) the HVR-L3 amino acid sequence of antibody S-60-19.

In some embodiments, anti-Sortilin antibodies of the present disclosure comprise a heavy chain comprising the amino acid sequence of SEQ ID NO: 86 or SEQ ID NO: 87. In some embodiments, anti-Sortilin antibodies of the present disclosure comprise a light chain comprising the amino acid sequence of SEQ ID NO: 114. In some embodiments, anti-Sortilin antibodies of the present disclosure comprise a heavy chain comprising the amino acid sequence of SEQ ID NO: 86 or SEQ ID NO: 87 and a light chain comprising the amino acid sequence of SEQ ID NO: 114.

### (11) S-60-24

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-24 or to the amino acid sequence of SEQ ID NO: 56; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-24 or to the amino acid sequence of SEQ ID NO: 80. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-24 or to the amino acid sequence of SEQ ID NO: 56, wherein the heavy chain variable domain comprises the HVR-H1, HVR-H2, and HVR-H3 amino acid sequences of antibody S-60-24. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-24 or to the amino acid sequence of SEQ ID NO: 80, wherein the light chain variable domain comprises the HVR-L1, HVR-L2, and HVR-L3 amino acid sequences of antibody S-60-24. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-24 or to the amino acid sequence of SEQ ID NO: 56 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-24 or the amino acid sequence of SEQ ID NO: 56. In certain embodiments, a total of 1 to 5 amino acids have been substituted, inserted and/or deleted in the heavy chain variable domain amino acid sequence of antibody S-60-24 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VH sequence of antibody S-60-24 or of SEQ ID NO: 56, including post-translational modifications of that sequence. In a particular embodiment, the VH comprises one, two or three HVRs selected from: (a) the HVR-H1 amino acid sequence of antibody S-60-24, (b) the HVR-H2 amino acid sequence of antibody S-60-24, and (c) the HVR-H3 amino acid sequence of antibody S-60-24. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-24 or to the amino acid sequence of SEQ ID NO: 80 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-24 or the amino acid sequence of SEQ ID NO: 80. In certain embodiments, a total of 1 to 5 amino acids have been substituted, inserted and/or deleted in the light chain variable domain amino acid sequence of antibody S-60-24 or the amino acid sequence of SEQ ID NO: 80. In certain embodiments, substitutions, insertions, or deletions occur in regions outside the HVRs (i.e., in the FR regions). In some embodiments, the substitutions, insertions, or deletions occur in in the FR regions. Optionally, the anti-Sortilin antibody comprises the VL sequence of antibody S-60-24 or of SEQ ID NO: 80, including post-translational modifications of that sequence. In a particular embodiment, the VL comprises one, two or three HVRs selected from: (a) the HVR-L1 amino acid sequence of antibody S-60-24, (b) the HVR-L2 amino acid sequence of antibody S-60-24, and (c) the HVR-L3 amino acid sequence of antibody S-60-24.

In some embodiments, anti-Sortilin antibodies of the present disclosure comprise a heavy chain comprising the amino acid sequence of SEQ ID NO: 90 or SEQ ID NO: 91. In some embodiments, anti-Sortilin antibodies of the present disclosure comprise a light chain comprising the amino acid sequence of SEQ ID NO: 115. In some embodiments, anti-Sortilin antibodies of the present disclosure comprise a heavy chain comprising the amino acid sequence of SEQ ID NO: 90 or SEQ ID NO: 91 and a light chain comprising the amino acid sequence of SEQ ID NO: 115.

In some embodiments, an anti-Sortilin antibody of the present disclosure binds essentially the same Sortilin epitope as an antibody comprising the heavy chain variable domain and the light chain variable domain of an antibody selected from the group consisting of S-60-10, S-60-11, S-60-12, S-60-13, S-60-14, S-60-15 [N33 (wt)], S-60-15.1 [N33T], S-60-16, S-60-18, S-60-19, and S-60-24.

In some embodiments, the anti-Sortilin antibody is anti-Sortilin monoclonal antibody S-60-10. In some embodiments, the anti-Sortilin antibody is an isolated antibody which binds essentially the same Sortilin epitope as S-60-10. In some embodiments, the anti-Sortilin antibody is an isolated antibody comprising the heavy chain variable region of monoclonal antibody S-60-10. In some embodiments, the anti-Sortilin antibody is an isolated antibody comprising the light chain variable region of monoclonal antibody S-60-10. In some embodiments, the anti-Sortilin antibody is an isolated antibody comprising the heavy chain variable region and the light chain variable region of monoclonal antibody S-60-10.

In some embodiments, the anti-Sortilin antibody is anti-Sortilin monoclonal antibody S-60-11. In some embodiments, the anti-Sortilin antibody is an isolated antibody which binds essentially the same Sortilin epitope as S-60-11. In some embodiments, the anti-Sortilin antibody is an isolated antibody comprising the heavy chain variable region of monoclonal antibody S-60-11. In some embodiments, the anti-Sortilin antibody is an isolated antibody comprising the light chain variable region of monoclonal antibody S-60-11. In some embodiments, the anti-Sortilin antibody is an isolated antibody comprising the heavy chain variable region and the light chain variable region of monoclonal antibody S-60-11.

In some embodiments, the anti-Sortilin antibody is anti-Sortilin monoclonal antibody S-60-12. In some embodiments, the anti-Sortilin antibody is an isolated antibody which binds essentially the same Sortilin epitope as S-60-12. In some embodiments, the anti-Sortilin antibody is an isolated antibody comprising the heavy chain variable region of monoclonal antibody S-60-12. In some embodiments, the anti-Sortilin antibody is an isolated antibody comprising the light chain variable region of monoclonal antibody S-60-12. In some embodiments, the anti-Sortilin antibody is an isolated antibody comprising the heavy chain variable region and the light chain variable region of monoclonal antibody S-60-12.

In some embodiments, the anti-Sortilin antibody is anti-Sortilin monoclonal antibody S-60-13. In some embodiments, the anti-Sortilin antibody is an isolated antibody which binds essentially the same Sortilin epitope as S-60-13. In some embodiments, the anti-Sortilin antibody is an isolated antibody comprising the heavy chain variable region of monoclonal antibody S-60-13. In some embodiments, the anti-Sortilin antibody is an isolated antibody comprising the light chain variable region of monoclonal antibody S-60-13. In some embodiments, the anti-Sortilin antibody is an isolated antibody comprising the heavy chain variable region and the light chain variable region of monoclonal antibody S-60-13.

In some embodiments, the anti-Sortilin antibody is anti-Sortilin monoclonal antibody S-60-14. In some embodiments, the anti-Sortilin antibody is an isolated antibody which binds essentially the same Sortilin epitope as S-60-14. In some embodiments, the anti-Sortilin antibody is an isolated antibody comprising the heavy chain variable region of monoclonal antibody S-60-14. In some embodiments, the anti-Sortilin antibody is an isolated antibody comprising the light chain variable region of monoclonal antibody S-60-14. In some embodiments, the anti-Sortilin antibody is an isolated antibody comprising the heavy chain variable region and the light chain variable region of monoclonal antibody S-60-14.

In some embodiments, the anti-Sortilin antibody is anti-Sortilin monoclonal antibody S-60-15. In some embodiments, the anti-Sortilin antibody is an isolated antibody which binds essentially the same Sortilin epitope as S-60-15. In some embodiments, the anti-Sortilin antibody is an isolated antibody comprising the heavy chain variable region of monoclonal antibody S-60-15. In some embodiments, the anti-Sortilin antibody is an isolated antibody comprising the light chain variable region of monoclonal antibody S-60-15. In some embodiments, the anti-Sortilin antibody is an isolated antibody comprising the heavy chain variable region and the light chain variable region of monoclonal antibody S-60-15.

In some embodiments, the anti-Sortilin antibody is anti-Sortilin monoclonal antibody S-60-15.1. In some embodiments, the anti-Sortilin antibody is an isolated antibody which binds essentially the same Sortilin epitope as S-60-15.1. In some embodiments, the anti-Sortilin antibody is an isolated antibody comprising the heavy chain variable region of monoclonal antibody S-60-15.1. In some embodiments, the anti-Sortilin antibody is an isolated antibody comprising the light chain variable region of monoclonal antibody S-60-15.1. In some embodiments, the anti-Sortilin antibody is an isolated antibody comprising the heavy chain variable region and the light chain variable region of monoclonal antibody S-60-15.1.

In some embodiments, the anti-Sortilin antibody is anti-Sortilin monoclonal antibody S-60-16. In some embodiments, the anti-Sortilin antibody is an isolated antibody which binds essentially the same Sortilin epitope as S-60-16. In some embodiments, the anti-Sortilin antibody is an isolated antibody comprising the heavy chain variable region of monoclonal antibody S-60-16. In some embodiments, the anti-Sortilin antibody is an isolated antibody comprising the light chain variable region of monoclonal antibody S-60-16. In some embodiments, the anti-Sortilin antibody is an isolated antibody comprising the heavy chain variable region and the light chain variable region of monoclonal antibody S-60-16.

In some embodiments, the anti-Sortilin antibody is anti-Sortilin monoclonal antibody S-60-18. In some embodiments, the anti-Sortilin antibody is an isolated antibody which binds essentially the same Sortilin epitope as S-60-18. In some embodiments, the anti-Sortilin antibody is an isolated antibody comprising the heavy chain variable region of monoclonal antibody S-60-18. In some embodiments, the anti-Sortilin antibody is an isolated antibody comprising the light chain variable region of monoclonal antibody S-60-18. In some embodiments, the anti-Sortilin antibody is an isolated antibody comprising the heavy chain variable region and the light chain variable region of monoclonal antibody S-60-18.

In some embodiments, the anti-Sortilin antibody is anti-Sortilin monoclonal antibody S-60-19. In some embodiments, the anti-Sortilin antibody is an isolated antibody which binds essentially the same Sortilin epitope as S-60-19. In some embodiments, the anti-Sortilin antibody is an isolated antibody comprising the heavy chain variable region of monoclonal antibody S-60-19. In some embodiments, the anti-Sortilin antibody is an isolated antibody comprising the light chain variable region of monoclonal antibody S-60-19. In some embodiments, the anti-Sortilin antibody is an isolated antibody comprising the heavy chain variable region and the light chain variable region of monoclonal antibody S-60-19.

In some embodiments, the anti-Sortilin antibody is anti-Sortilin monoclonal antibody S-60-24. In some embodiments, the anti-Sortilin antibody is an isolated antibody which binds essentially the same Sortilin epitope as S-60-24. In some embodiments, the anti-Sortilin antibody is an isolated antibody comprising the heavy chain variable region of monoclonal antibody S-60-24. In some embodiments, the anti-Sortilin antibody is an isolated antibody comprising the light chain variable region of monoclonal antibody S-60-24. In some embodiments, the anti-Sortilin antibody is an isolated antibody comprising the heavy chain variable region and the light chain variable region of monoclonal antibody S-60-24.

In certain embodiments, the anti-Sortilin antibody is an antagonist antibody. In certain embodiments, the anti-Sortilin antibody is an agonist antibody. In some embodiments, anti-Sortilin antibodies of the present disclosure are of the IgG class the IgM class, or the IgA class. In some embodiments, anti-Sortilin antibodies of the present disclosure are of the IgG class and have an IgG1, IgG2, IgG3, or IgG4 isotype.

Additional anti-Sortilin antibodies, e.g., antibodies that specifically bind to a Sortilin protein of the present disclosure, may be identified, screened, and/or characterized for their physical/chemical properties and/or biological activities by various assays known in the art.

Certain aspects of the present disclosure relate to the use of two or more anti-Sortilin antibodies that when utilized together display additive or synergistic effects, as compared to utilization of a corresponding single anti-Sortilin antibody.

In some embodiments, an anti-Sortilin antibody of the present disclosure is an antibody fragment that binds to a human Sortilin protein.

In some embodiments, an anti-Sortilin antibody of the present disclosure is an antibody fragment that binds to one or more human proteins selected from the group consisting of human Sortilin, a naturally occurring variant of human Sortilin, and a disease variant of human Sortilin.

In some embodiments, an anti-Sortilin antibody of the present disclosure is antibody fragment, wherein the antibody fragment is an Fab, Fab′, Fab′-SH, F(ab′)2, Fv, or scFv fragment.

### Antibody Frameworks

In some embodiments, anti-Sortilin antibodies of the present disclosure comprise a heavy chain variable region comprising one or more (e.g., one or more, two or more, three or more, or all four) framework regions selected from VH FR1, VH FR2, VH FR3, and VH FR4 (as shown in Tables 17-20). In some embodiments, the VH FR1 comprises a sequence of QVQLQESGPGLVKPSETLSL TCAVSG (SEQ ID NO: 35). In some embodiments, the VH FR2 comprises a sequence of WIRQPPGKGLEWIG (SEQ ID NO: 36). In some embodiments, the VH FR3 comprises the sequence according to Formula VI: X₁VTISVDTSKNQFSLX2LSSVTAADTAVYYC (SEQ ID NO: 39), wherein X₁ is Q or R, and X₂ is E or K. In some embodiments, VH FR3 comprises a sequence selected from the group consisting of SEQ ID NOs: 37-38. In some embodiments, VH FR4 comprises a sequence of WGQGTTVTVSS (SEQ ID NO: 40). In some embodiments, an antibody comprises a heavy chain variable region comprising a VH FR1 comprising the sequence of SEQ ID NO: 35, a VH FR2 comprising the sequence of SEQ ID NO: 36, a VH FR3 according to Formula VI, and a VH FR4 comprising the sequence of SEQ ID NO: 40.

In some embodiments, an antibody comprises a heavy chain variable region comprising a VH FR1 comprising the sequence of SEQ ID NO: 35, a VH FR2 comprising the sequence of SEQ ID NO: 36, a VH FR3 comprising the sequence selected from SEQ ID NOs: 37-38, and a VH FR4 comprising the sequence of SEQ ID NO: 40.

In some embodiments, anti-Sortilin antibodies of the present disclosure comprise a heavy chain variable region comprising a VH FR1, a VH FR2, a VH FR3, and VH FR4 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24 (as shown in Tables 17-20).

In some embodiments, anti-Sortilin antibodies of the present disclosure comprise a light chain variable region comprising one or more (e.g., one or more, two or more, three or more, or all four) framework regions selected from VL FR1, VL FR2, VL FR3, and VL FR4 (as shown in Tables 21-24). In some embodiments, the VL FR1 comprises a sequence according to Formula VII: DIVMTQSPLSLPVTPGX₁X₂ASISC (SEQ ID NO: 44), wherein X₁ is E or G, and X₂ is P or S. In some embodiments, VL FR1 comprises a sequence selected from the group consisting of SEQ ID NOs: 41-43. In some embodiments, the VL FR2 comprises a sequence according to Formula VIII: WYLQKPGQXiPQLLIY (SEQ ID NO: 47), wherein X₁ is S or P. In some embodiments, VL FR2 comprises a sequence selected from the group consisting of SEQ ID NOs: 45-46. In some embodiments, the VL FR3 comprises a sequence according to Formula IX: GVPDRXiSGSGSGT DFTLKISRX2EAEDVGX3YYC (SEQ ID NO: 52), wherein X₁ is F or L, X₂ is A or V, and X₃ is V or A. In some embodiments, VL FR3 comprises a sequence selected from the group consisting of SEQ ID NOs: 48-51. In some embodiments, the VL FR4 comprises a sequence of FGGGTKVEIK (SEQ ID NO: 53). In some embodiments, anti-Sortilin antibodies of the present disclosure comprise a light chain variable region comprising a VL FR1 comprising the sequence according to Formula VII, a VL FR2 comprising the sequence according to Formula VIII, a VL FR3 comprising the sequence according to Formula IX, and a VL FR4 comprising the sequence of SEQ ID NO: 53.

In some embodiments, anti-Sortilin antibodies of the present disclosure comprise a light chain variable region comprising a VL FR1 comprising the sequence selected from SEQ ID NOs: 41-43, a VL FR2 comprising the sequence selected from SEQ ID NOs: 45-46, a VL FR3 comprising the sequence selected from SEQ ID NOs: 48-51, and a VL FR4 comprising the sequence of SEQ ID NO: 53.

In some embodiments, anti-Sortilin antibodies of the present disclosure comprise a light chain variable region comprising a VL FR1, a VL FR2, a VL FR3, and VL FR4 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24 (as shown in Tables 21-24).

In some embodiments, anti-Sortilin antibodies of the present disclosure comprise a heavy chain variable region comprising one or more (e.g., one or more, two or more, three or more, or all four) framework regions selected from VH FR1, VH FR2, VH FR3, and VH FR4 (as shown in Tables 17-20), and a light chain variable region comprising one or more (e.g., one or more, two or more, three or more, or all four) framework regions selected from VL FR1, VL FR2, VL FR3, and VL FR4 (as shown in Tables 21-24). In some embodiments, anti-Sortilin antibodies of the present disclosure comprise a heavy chain variable region comprising a

VH FR1 comprising the sequence of SEQ ID NO: 35, a VH FR2 comprising the sequence of SEQ ID NO: 36, a VH FR3 according to Formula VI, and a VH FR4 comprising the sequence of SEQ ID NO: 40; and a light chain variable region comprising a VL FR1 comprising the sequence according to Formula VII, a VL FR2 comprising the sequence according to Formula VIII, a VL FR3 comprising the sequence according to Formula IX, and a VL FR4 comprising the sequence of SEQ ID NO: 53. In some embodiments, anti-Sortilin antibodies of the present disclosure comprise a heavy chain variable region comprising a VH FR1 comprising the sequence of SEQ ID NO: 35, a VH FR2 comprising the sequence of SEQ ID NO: 36, a VH FR3 comprising the sequence selected from SEQ ID NOs: 37-38, and a VH FR4 comprising the sequence of SEQ ID NO: 40; a light chain variable region comprising a VL FR1 comprising the sequence selected from SEQ ID NOs: 41-43, a VL FR2 comprising the sequence selected from SEQ ID NOs: 45-46, a VL FR3 comprising the sequence selected from SEQ ID NOs: 48-51, and a VL FR4 comprising the sequence of SEQ ID NO: 53.

In some embodiments, anti-Sortilin antibodies of the present disclosure comprise a heavy chain variable region comprising a VH FR1, a VH FR2, a VH FR3, and VH FR4 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24 (as shown in Tables 17-20), and a light chain variable region comprising a VL FR1, a VL FR2, a VL FR3, and VL FR4 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16; S-60-18, S-60-19, or S-60-24 (as shown in Tables 21-24).

### Anti-Sortilin Antibody Activities

In certain aspects of any of the anti-Sortilin antibodies, anti-Sortilin antibodies of the present disclosure can inhibit one or more activities of a Sortilin protein, including, but not limited to, decreasing cellular levels of Sortilin (e.g., cell surface levels of Sortilin, intracellular levels of Sortilin, and/or total levels of Sortilin); increasing Progranulin levels (e.g., extracellular levels of Progranulin and/or cellular levels of Progranulin); and inhibiting the interaction (e.g., binding) between Progranulin and Sortilin. As contemplated herein, anti-Sortilin antibodies of the present disclosure may inhibit additional activities of a Sortilin protein, including but not limited to inhibiting interaction (e.g., binding) with one or more of pro-neurotrophins of the present disclosure (pro-neurotrophin-3, pro-neurotrophin-4/5, pro-NGF, pro-BDNF, etc.), neurotrophins of the present disclosure (neurotrophin-3, neurotrophin-4/5, NGF, BDNF, etc.), neurotensin, p75, Sortilin propeptide (Sort-pro), amyloid precursor protein (APP), the A beta peptide, lipoprotein lipase (LpL), apolipoprotein AV (APOA5), apolipoprotein E (APOE), and receptor associated protein (RAP), decreasing secretion of PCSK9, decreasing production of beta amyloid peptide.

In certain embodiments, the present disclosure provides an anti-Sortilin antibody, wherein (a) the anti-Sortilin antibody increases extracellular levels of Progranulin, decreases cellular levels of Sortilin, inhibits interaction between Sortilin and Progranulin, or any combination thereof; (b) the anti-Sortilin antibody decreases cell surface levels of Sortilin, increases extracellular levels of Progranulin, inhibits interaction between Sortilin and Progranulin, or any combination thereof; (c) the anti-Sortilin antibody decreases cell surface levels of Sortilin, decreases intracellular levels of Sortilin, decreases total levels of Sortilin, or any combination thereof; (d) the anti-Sortilin antibody induces Sortilin degradation, Sortilin cleavage, Sortilin internalization, Sortilin down regulation, or any combination thereof; (e) the anti-Sortilin antibody decreases cellular levels of Sortilin and inhibits the interaction between Sortilin and Progranulin; (f) the anti-Sortilin antibody decreases cellular levels of Sortilin and increases cellular levels of Progranulin; and/or (g) the anti-Sortilin antibody increases the effective concentration of Progranulin.

In certain embodiments, the present disclosure provides an anti-Sortilin antibody, wherein the anti-Sortilin antibody decreases cell surface levels of Sortilin, increases extracellular levels of Progranulin, inhibits interaction between Sortilin and Progranulin, or any combination thereof.

In some embodiments, an anti-Sortilin antibody of the present disclosure (a) reduces cell surface levels of Sortilin with a half maximal effective concentration (EC₅₀) that is less than 150 μM, as measured by flow cytometry; (b) reduces cell surface levels of Sortilin by more than about 50% at 1.25 nM IgG, by more than about 80% at 0.63 nM IgG, or by more than about 69% at 150 nM IgG relative to control, as measured by flow cytometry; increases Progranulin secretion by more than about 1.13 fold over control at 0.63 nM IgG, or by more than about 1.22 fold over control at 50 nM IgG, as measured by standard ELISA; blocks binding of Progranulin to Sortilin with a half maximal effective concentration (EC₅₀) that is less than 0.325 nM, as measured by flow cytometry; (e) blocks binding of Progranulin to Sortilin by more than about 88% at 50 nM IgG, or by more than about 27.5% at 150 nM IgG relative to control, as measured by flow cytometry; or (f) any combination thereof.

In some embodiments, an anti-Sortilin antibody of the present disclosure (a) reduces cell surface levels of Sortilin with a half maximal effective concentration (EC₅₀) that is less than 681 μM, as measured by flow cytometry; (b) reduces cell surface levels of Sortilin by more than about 40% at 1.25 nM IgG, by more than about 29% at 0.6 nM IgG, or by more than about 62% at 150 nM IgG relative to control, as measured by flow cytometry; (c) increases Progranulin secretion by more than about 1.11 fold over control at 0.63 nM IgG, or by more than about 1.75 fold over control at 50 nM IgG, as measured by standard ELISA; (d) blocks binding of Progranulin to Sortilin with a half maximal effective concentration (EC₅₀) that is less than 0.751 nM, as measured by flow cytometry; (e) blocks binding of Progranulin to Sortilin by more than about 90% at 50 nM IgG, or by more than about 95% at 150 nM IgG relative to control, as measured by flow cytometry; or (f) any combination thereof.

### Decreasing Sortilin Levels

In some embodiments, anti-Sortilin antibodies of the present disclosure bind to a Sortilin protein of the present disclosure expressed on the surface of a cell and modulate (e.g., induce or inhibit) one or more Sortilin activities of the present disclosure after binding to the surface-expressed Sortilin protein.

In some embodiments, anti-Sortilin antibodies of the present disclosure decrease cellular levels of Sortilin in vitro. In some embodiments, anti-Sortilin antibodies of the present disclosure may decrease cellular levels of Sortilin in vivo (e.g., in the brain, and/or peripheral organs of an individual). In some embodiments, a decrease in cellular levels of Sortilin comprises a decrease in cell surface levels of Sortilin. As used herein, an anti-Sortilin antibody decreases cell surface levels of Sortilin if it induces a decrease at saturating antibody concentrations (e.g., 0.6 nM, 0.63 nM, 1.25 nM, 50 nM or 150 nM) and/or relative to a control antibody (e.g. an anti-Sortilin antibody having a heavy chain variable region and a light chain variable region corresponding to S-60) in cell surface levels of Sortilin as measured by any in vitro cell-based assays or suitable in vivo model described herein or known in the art. In some embodiments, a decrease in cellular levels of Sortilin comprises a decrease in intracellular levels of Sortilin. As contemplated herein, an anti-Sortilin antibody decreases intracellular levels of Sortilin if it induces a decrease at saturating antibody concentrations and/or relative to a control antibody (e.g. an anti-Sortilin antibody having a heavy chain variable region and a light chain variable region corresponding to S-60) in intracellular levels of Sortilin as measured by any in vitro cell-based assays or suitable in vivo model described herein or known in the art. In some embodiments, a decrease in cellular levels of Sortilin comprises a decrease in total levels of Sortilin. As contemplated herein, an anti-Sortilin antibody decreases total levels of Sortilin if it induces a decrease at saturating antibody concentrations and/or relative to a control antibody (e.g. an anti-Sortilin antibody having a heavy chain variable region and a light chain variable region corresponding to S-60) in total levels of Sortilin as measured by any in vitro cell-based assays or suitable in vivo model described herein or known in the art.

As used herein, levels of Sortilin may refer to expression levels of the gene encoding Sortilin; to expression levels of one or more transcripts encoding Sortilin; to expression levels of Sortilin protein; and/or to the amount of Sortilin protein present within cells and/or on the cell surface. Any methods known in the art for measuring levels of gene expression, transcription, translation, and/or protein abundance or localization may be used to determine the levels of Sortilin.

Cellular levels of Sortilin may refer to, without limitation, cell surface levels of Sortilin, intracellular levels of Sortilin, and total levels of Sortilin. In some embodiments, a decrease in cellular levels of Sortilin comprises decrease in cell surface levels of Sortilin. In some embodiments, anti-Sortilin antibodies of the present disclosure that decrease cellular levels of Sortilin (e.g., cell surface levels of Sortilin) have one or more of the following characteristics: (1) inhibits or reduces one or more Sortilin activities; (2) the ability to inhibit or reduce binding of a Sortilin to one or more of its ligands; (3) the ability to reduce Sortilin expression in Sortilin-expressing cells; (4) the ability to interact, bind, or recognize a Sortilin protein; (5) the ability to specifically interact with or bind to a Sortilin protein; and (6) the ability to treat, ameliorate, or prevent any aspect of a disease or disorder described or contemplated herein.

In some embodiments, an isolated anti-Sortilin antibody of the present disclosure induces downregulation of Sortilin. In some embodiments, an isolated anti-Sortilin antibody of the present disclosure induces cleavage of Sortilin. In some embodiments, an isolated anti-Sortilin antibody of the present disclosure induces internalization of Sortilin. In some embodiments, an isolated anti-Sortilin antibody of the present disclosure induces shedding of Sortilin. In some embodiments, an isolated anti-Sortilin antibody of the present disclosure induces degradation of Sortilin. In some embodiments, an isolated anti-Sortilin antibody of the present disclosure induces desensitization of Sortilin. In some embodiments, an isolated anti-Sortilin antibody of the present disclosure acts as a ligand mimetic to transiently activate Sortilin. In some embodiments, an isolated anti-Sortilin antibody of the present disclosure acts as a ligand mimetic and transiently activates Sortilin before inducing a decrease in cellular levels of Sortilin and/or inhibition of interaction (e.g., binding) between Sortilin and one or more Sortilin ligands. In some embodiments, an isolated anti-Sortilin antibody of the present disclosure acts as a ligand mimetic and transiently activates Sortilin before inducing degradation of Sortilin. In some embodiments, an isolated anti-Sortilin antibody of the present disclosure acts as a ligand mimetic and transiently activates Sortilin before inducing cleavage of Sortilin. In some embodiments, an isolated anti-Sortilin antibody of the present disclosure acts as a ligand mimetic and transiently activates Sortilin before inducing internalization of Sortilin. In some embodiments, an isolated anti-Sortilin antibody of the present disclosure acts as a ligand mimetic and transiently activates Sortilin before inducing shedding of Sortilin. In some embodiments, an isolated anti-Sortilin antibody of the present disclosure acts as a ligand mimetic and transiently activates Sortilin before inducing downregulation of Sortilin expression. In some embodiments, an isolated anti-Sortilin antibody of the present disclosure acts as a ligand mimetic and transiently activates Sortilin before inducing desensitization of Sortilin.

In certain embodiments, anti-Sortilin antibodies of the present disclosure may decrease cellular levels of Sortilin (e.g., cell surface levels of Sortilin, intracellular levels of Sortilin, and/or total levels of Sortilin) by inducing Sortilin degradation. Accordingly, in some embodiments, anti-Sortilin antibodies of the present disclosure induce Sortilin degradation.

Anti-Sortilin antibodies of the present disclosure may decrease cellular levels (e.g., cell surface levels) of Sortilin with a half-maximal effective concentration (EC₅₀) (e.g., when measured in vitro) in the picomolar range. In certain embodiments, the EC₅₀ of the antibody is less than about 680.9 μM. In certain embodiments, the EC₅₀ of the antibody is about 72.58 μM to about 680.9 nM. In certain embodiments, the EC₅₀ of the antibody is about 103.6 μM to about 680.9 nM. In certain embodiments, the EC₅₀ of the antibody is less than about 600 μM, 500 μM, 400 μM, 300 μM, 200 μM, 100 μM, 50 μM, 40 μM, 30 μM, 20 μM, 10 μM, 1 μM, or 0.5 μM.

In some embodiments, the EC₅₀ of the antibody is less than about or equal to about 675 μM, 650 μM, 625 μM, 600 μM, 575 μM, 550 μM, 525 μM, 500 μM, 475 μM, 450 μM, 425 μM, 400 μM, 375 μM, 350 μM, 325 μM, 300 μM, 275 μM, 250 μM, 225 μM, 200 μM, 175 μM, 150 μM, 125 μM, 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, or 0.5 μM.

In some embodiments, the EC₅₀ of the antibody is less than about 680.9 μM. In some embodiments, the EC₅₀ of the antibody is greater than about or equal to about 0.1 μM, 0.5 μM, 1 μM, 10 μM, 20 μM, 30 μM, 40 μM, 50 μM, 60 μM, 70 μM, 80 μM, 90 μM, 100 μM, 125 μM, 150 μM, 175 μM, 200 μM, 225 μM, 250 μM, 275 μM, 300 μM, 325 μM, 350 μM, 375 μM, 400 μM, 425 μM, 450 μM, 475 μM, 500 μM, 525 μM, 550 μM, 575 μM, 600 μM, 625 μM, 650 μM, 675 μM. That is, the EC₅₀ of the antibody can be any of a range having an upper limit of about 675 μM, 650 nM, 650 μM, 625 μM, 600 μM, 575 μM, 550 μM, 525 μM, 500 μM, 475 μM, 450 μM, 425 μM, 400 μM, 375 μM, 350 μM, 325 μM, 300 μM, 275 μM, 250 μM, 225 μM, 200 μM, 175 μM, 150 μM, 125 μM, 100 μM, 90 μM, 80 μM, 70 μM, 60 μM, 50 μM, 40 μM, 30 μM, 20 μM, 10 μM, 1 μM, or 0.5 μM, and an independently selected lower limit of about 0.1 μM, 0.5 μM, 1 μM, 10 μM, 20 μM, 30 μM, 40 μM, 50 μM, 60 μM, 70 μM, 80 μM, 90 μM, 100 μM, 125 μM, 150 μM, 175 μM, 200 μM, 225 μM, 250 μM, 275 μM, 300 μM, 325 μM, 350 μM, 375 μM, 400 μM, 425 μM, 450 μM, 475 μM, 500 μM, 525 μM, 550 μM, 575 μM, 600 μM, 625 μM, 650 μM, or 675 μM, wherein the lower limit is less than the upper limit. In some embodiments, the EC₅₀ of the antibody is any of about 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 105 μM, 110 μM, 115 μM, 120 μM, 125 μM, 130 μM, 135 μM, 140 μM, 145 μM, 150 μM, 155 μM, 160 μM, 165 μM, 170 μM, 175 μM, 180 μM, 185 μM, 190 μM, 195 μM, or 200 μM.

In some embodiments, an anti-Sortilin antibody of the present disclosure reduces cell surface levels of Sortilin with a half maximal effective concentration (EC₅₀) that is less than 150 μM, as measured by flow cytometry. In some embodiments, the EC₅₀ of an anti-Sortilin antibody of the present disclosure is about 103.6 μM. In some embodiments, the EC₅₀ of an anti-Sortilin antibody of the present disclosure is about 72.58 μM.

In some embodiments, an anti-Sortilin antibody of the present disclosure reduces cell surface levels of Sortilin by more than about 40% at 1.25 nM IgG or by more than about 80% at 0.63 nM IgG, as measured by flow cytometry. In some embodiments, an anti-Sortilin antibody of the present disclosure reduces cell surface levels of Sortilin by about 60.92% at 1.25 nM IgG, as measured by flow cytometry. In some embodiments, an anti-Sortilin antibody of the present disclosure reduces cell surface levels of Sortilin by about 69.3% at 150 nM IgG, as measured by flow cytometry. In some embodiments, an anti-Sortilin antibody of the present disclosure reduces cell surface levels of Sortilin by about 70.3% at 150 nM IgG, as measured by flow cytometry.

Various methods of measuring antibody EC₅₀ values are known in the art, including, for example, by flow cytometry. In some embodiments, the EC₅₀ is measured in vitro using cells engineered to express human Sortilin. In some embodiments, the EC₅₀ is measured at a temperature of approximately 4° C. In some embodiments, the EC₅₀ is measured at a temperature of approximately 25° C. In some embodiments, the EC₅₀ is measured at a temperature of approximately 35° C. In some embodiments, the EC₅₀ is measured at a temperature of approximately 37° C. In some embodiments, the EC₅₀ is determined using a monovalent antibody (e.g., a Fab) or a full-length antibody in a monovalent form. In some embodiments, the EC₅₀ is determined using antibodies containing constant regions that demonstrate enhanced Fc receptor binding. In some embodiments, the EC₅₀ is determined using antibodies containing constant regions that demonstrate reduced Fc receptor binding.

In some embodiments, anti-Sortilin antibodies of the present disclosure have higher potencies in reducing cell surface levels of Sortilin relative to a control antibody (e.g. an anti-Sortilin antibody having a heavy chain variable region and a light chain variable region corresponding to S-60). In some embodiments, anti-Sortilin antibodies of the present disclosure decrease cellular levels (e.g., cell surface levels) of Sortilin with a lower EC₅₀ (e.g., as measured in vitro) than a control antibody (e.g. an anti-Sortilin antibody having a heavy chain variable region and a light chain variable region corresponding to S-60). In some embodiments, anti-Sortilin antibodies of the present disclosure decrease cellular levels (e.g., cell surface levels) of Sortilin with an EC₅₀ that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% lower than the EC₅₀ of a control antibody (e.g. an anti-Sortilin antibody having a heavy chain variable region and a light chain variable region corresponding to S-60). In some embodiments, anti-Sortilin antibodies of the present disclosure decrease cellular levels (e.g., cell surface levels) of Sortilin with an EC₅₀ that is at least about 1-fold, at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 17.5-fold, at least about 20-fold, at least about 22.5-fold, at least about 25-fold, at least about 27.5-fold, at least about 30-fold, at least about 50-fold, or at least about 100-fold lower than the EC₅₀ of a control antibody (e.g. an anti-Sortilin antibody having a heavy chain variable region and a light chain variable region corresponding to S-60).

In some embodiments, anti-Sortilin antibodies of the present disclosure have an EC₅₀ that is at least 1.5-fold lower than control antibody (e.g. an anti-Sortilin antibody having a heavy chain variable region and a light chain variable region corresponding to S-60). In some embodiments, anti-Sortilin antibodies of the present disclosure have an EC₅₀ that is at least 1.1-fold lower than control antibody (e.g. an anti-Sortilin antibody having a heavy chain variable region and a light chain variable region corresponding to S-60).

In some embodiments, an anti-Sortilin antibody of the present disclosure (a) reduces cell surface levels of Sortilin with a half maximal effective concentration (EC₅₀) that is less than 681 μM, as measured by flow cytometry; (b) reduces cell surface levels of Sortilin by more than about 40% at 1.25 nM IgG, by more than about 29% at 0.6 nM IgG, or by more than about 62% at 150 nM IgG relative to control, as measured by flow cytometry; (c) increases Progranulin secretion by more than about 1.11 fold over control at 0.63 nM IgG, or by more than about 1.75 fold over control at 50 nM IgG, as measured by standard ELISA; (d) blocks binding of Progranulin to Sortilin with a half maximal effective concentration (EC₅₀) that is less than 0.751 nM, as measured by flow cytometry; (e) blocks binding of Progranulin to Sortilin by more than about 90% at 50 nM IgG, or by more than about 95% at 150 nM IgG relative to control, as measured by flow cytometry; or (f) any combination thereof.

### Increasing Progranulin Levels

In some embodiments, anti-Sortilin antibodies of the present disclosure increase extracellular levels of Progranulin in vitro. In some embodiments, anti-Sortilin antibodies of the present disclosure may increase cellular levels of Progranulin or in vivo (e.g., in the brain, blood, and/or peripheral organs of an individual). As used herein, an anti-Sortilin antibody increases extracellular levels of Progranulin if it induces an increase at saturating antibody concentrations (e.g., 0.6 nM, 0.63 nM, 1.25 nM, 50 nM or 150 nM) and/or relative to a control antibody (e.g. an anti-Sortilin antibody having a heavy chain variable region and a light chain variable region corresponding to S-60) in extracellular levels of Progranulin as measured by any in vitro cell-based assays or in tissue-based (such as brain tissue-based) assays described herein or known in the art. As contemplated herein, an anti-Sortilin antibody increases cellular levels of Progranulin if it induces an increase at saturating antibody concentrations (e.g., 0.6 nM, 0.63 nM, 1.25 nM, 50 nM or 150 nM) and/or relative to a control antibody (e.g. an anti-Sortilin antibody having a heavy chain variable region and a light chain variable region corresponding to S-60) in cellular levels of Progranulin as measured by any in vitro cell-based assays or in tissue-based (such as brain tissue-based) assays described herein or known in the art.

As used herein, levels of Progranulin may refer to expression levels of the gene encoding Progranulin; to expression levels of one or more transcripts encoding Progranulin; to expression levels of Progranulin protein; and/or to the amount of Progranulin protein secreted from cells and/or present within cells. Any methods known in the art for measuring levels of gene expression, transcription, translation, protein abundance, protein secretion, and/or protein localization may used to determine the levels of Progranulin.

As used herein, Progranulin levels may refer to, without limitation, extracellular levels of Progranulin, intracellular levels of Progranulin, and total levels of Progranulin. In some embodiments, an increase in levels of Progranulin comprises an increase in extracellular levels of Progranulin.

In some embodiments, an anti-Sortilin antibody of the present disclosure increases Progranulin secretion by more than about 1.11 fold over control at 0.63 nM IgG, as measured by standard ELISA. In some embodiments, an anti-Sortilin antibody of the present disclosure increases Progranulin secretion by about 1.42 fold over control at 0.63 nM IgG, as measured by standard ELISA. In some embodiments, an anti-Sortilin antibody of the present disclosure increases Progranulin secretion by more than about 1.75 fold over control at 50 nM IgG, as measured by standard ELISA. In some embodiments, an anti-Sortilin antibody of the present disclosure increases Progranulin secretion by about 1.97 fold over control at 50 nM IgG, as measured by standard ELISA. In some embodiments, an anti-Sortilin antibody of the present disclosure increases Progranulin secretion by about 2.29 fold over control at 50 nM IgG, as measured by standard ELISA.

Various methods of measuring Progranulin secretion are known in the art, including, for example, by ELISA. In some embodiments, the EC₅₀ is measured in vitro using cells expressing human Sortilin. In some embodiments, Progranulin secretion is determined using a monovalent antibody (e.g., a Fab) or a full-length antibody in a monovalent form. In some embodiments, Progranulin secretion is determined using antibodies containing constant regions that demonstrate enhanced Fc receptor binding. In some embodiments, Progranulin secretion is determined using antibodies containing constant regions that demonstrate reduced Fc receptor binding.

In some embodiments, anti-Sortilin antibodies of the present disclosure have higher potencies in increasing levels of Progranulin relative to a control antibody (e.g. an anti-Sortilin antibody having a heavy chain variable region and a light chain variable region corresponding to S-60). In some embodiments, anti-Sortilin antibodies of the present disclosure increase levels (e.g., extracellular levels) of Progranulin with a lower EC₅₀ (e.g., as measured in vitro) than a control antibody (e.g. an anti-Sortilin antibody having a heavy chain variable region and a light chain variable region corresponding to S-60). In some embodiments, anti-Sortilin antibodies of the present disclosure increase levels (e.g., extracellular levels) of Progranulin by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% than a control antibody (e.g. an anti-Sortilin antibody having a heavy chain variable region and a light chain variable region corresponding to S-60). In some embodiments, anti-Sortilin antibodies of the present disclosure increase levels (e.g., extracellular levels) of Progranulin by about 1-fold, at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 17.5-fold, at least about 20-fold, at least about 22.5-fold, at least about 25-fold, at least about 27.5-fold, at least about 30-fold, at least about 50-fold, or at least about 100-fold higher than a control antibody (e.g. an anti-Sortilin antibody having a heavy chain variable region and a light chain variable region corresponding to S-60).

In some embodiments, anti-Sortilin antibodies of the present disclosure increase Progranulin levels by about 1.1-fold higher than a control antibody (e.g. an anti-Sortilin antibody having a heavy chain variable region and a light chain variable region corresponding to S-60). In some embodiments, anti-Sortilin antibodies of the present disclosure increase Progranulin levels by about 1.3-fold higher than a control antibody (e.g. an anti-Sortilin antibody having a heavy chain variable region and a light chain variable region corresponding to S-60).

In some embodiments, anti-Sortilin antibodies of the present disclosure increase the effective concentration of Progranulin. The effective concentration of Progranulin refers to the concentration of Progranulin in plasma or cerebrospinal fluid. In some embodiments, an increase in the effective concentration of Progranulin is an increase of greater than 1.5 fold. In some embodiments, the effective concentration of Progranulin is increased for 7-28 days.

### Decreasing Interaction Between Sortilin and Progranulin

In some embodiments, anti-Sortilin antibodies of the present disclosure increase Progranulin levels and/or decrease cellular levels of Sortilin while blocking (e.g. inhibiting) the interaction (e.g., binding) between Sortilin and Progranulin. Accordingly, in some embodiments, anti-Sortilin antibodies of the present disclosure block the interaction (e.g., binding) between Sortilin and Progranulin. As used herein, an anti-Sortilin antibody blocks the interaction (e.g., binding) between Sortilin and Progranulin if it decreases Progranulin binding to Sortilin relative to a control antibody (e.g. an anti-Sortilin antibody having a heavy chain variable region and a light chain variable region corresponding to S-60) at saturating antibody concentrations (e.g., 0.6 nM, 0.63 nM, 1.25 nM, 50 nM or 150 nM) in any in vitro assay or cell-based culture assay described herein or known in the art.

Anti-Sortilin antibodies of the present disclosure may decrease Progranulin binding to Sortilin with a half-maximal effective concentration (EC₅₀) (e.g., when measured in vitro) in the picomolar range. In certain embodiments, the EC₅₀ of the antibody is less than about 2.2 nM. In certain embodiments, the EC₅₀ of the antibody is less than about 1.22 nM. In certain embodiments, the EC₅₀ of the antibody is less than about 751 μM. In certain embodiments, the EC₅₀ of the antibody is about 325 μM to about 751 nM. In certain embodiments, the EC₅₀ of the antibody is about 405 μM to about 751 nM. In certain embodiments, the EC₅₀ of the antibody is about 588 μM to about 751 nM. In certain embodiments, the EC₅₀ of the antibody is less than about 2.2 nM, 2.1 nM, 2.0 nM, 1.9 nM, 1.8 nM, 1.7 nM, 1.6 nM, 1.5 nM, 1.4 nM, 1.3 nM, 1.2 nM, 1.1 nM, 1.0 nM, 900 μM, 800 μM, 700 μM, 600 μM, 500 μM, 400 μM, 300 μM, 200 μM, 100 μM, 50 μM, 40 μM, 30 μM, 20 μM, 10 μM, 1 μM, or 0.5 μM.

In some embodiments, the EC₅₀ of the antibody for decreasing Progranulin binding to Sortilin is less than about or equal to about 2.2 nM, 2.1 nM, 2.0 nM, 1.9 nM, 1.8 nM, 1.7 nM, 1.6 nM, 1.5 nM, 1.4 nM, 1.3 nM, 1.2 nM, 1.1 nM, 1.0 nM, 900 μM, 800 μM, 700 μM, 600 μM, 500 μM, 475 μM, 450 μM, 425 μM, 400 μM, 375 μM, 350 μM, 325 μM, 300 μM, 275 μM, 250 μM, 225 μM, 200 μM, 175 μM, 150 μM, 125 μM, 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, or 0.5 μM.

In some embodiments, the EC₅₀ of an anti-Sortilin antibody of the present disclosure is about 1.22 nM. In some embodiments, the EC₅₀ of an anti-Sortilin antibody of the present disclosure is about 588 μM. In some embodiments, the EC₅₀ of an anti-Sortilin antibody of the present disclosure is about 405 μM. In some embodiments, the EC₅₀ of an anti-Sortilin antibody of the present disclosure is about 325 μM.

Various methods of measuring antibody EC₅₀ values are known in the art, including, for example, by flow cytometry. In some embodiments, the EC₅₀ for decreasing Progranulin binding to Sortlin is measured in vitro using cells expressing human Sortilin. In some embodiments, the EC₅₀ is measured at a temperature of approximately 4° C. In some embodiments, the EC₅₀ is measured at a temperature of approximately 25° C. In some embodiments, the EC₅₀ is measured at a temperature of approximately 35° C. In some embodiments, the EC₅₀ is measured at a temperature of approximately 37° C. In some embodiments, the EC₅₀ for decreasing Progranulin binding to Sortlin is determined using a monovalent antibody (e.g., a Fab) or a full-length antibody in a monovalent form. In some embodiments, the EC₅₀ is determined using antibodies containing constant regions that demonstrate enhanced Fc receptor binding. In some embodiments, the EC₅₀ for decreasing Progranulin binding to Sortlin is determined using antibodies containing constant regions that demonstrate reduced Fc receptor binding.

In some embodiments, anti-Sortilin antibodies of the present disclosure have higher potencies in reducing Progranulin binding to Sortlin relative to a control antibody (e.g. an anti-Sortilin antibody having a heavy chain variable region and a light chain variable region corresponding to S-60). In some embodiments, anti-Sortilin antibodies of the present disclosure decrease Progranulin binding to Sortlin with a lower EC₅₀ (e.g., as measured in vitro) than a control antibody (e.g. an anti-Sortilin antibody having a heavy chain variable region and a light chain variable region corresponding to S-60). In some embodiments, anti-Sortilin antibodies of the present disclosure decrease Progranulin binding to Sortlin with an EC₅₀ that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% lower than the EC₅₀ of a control antibody (e.g. an anti-Sortilin antibody having a heavy chain variable region and a light chain variable region corresponding to S-60). In some embodiments, anti-Sortilin antibodies of the present disclosure decrease Progranulin binding to Sortlin with an EC₅₀ that is at least about 1-fold, at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 17.5-fold, at least about 20-fold, at least about 22.5-fold, at least about 25-fold, at least about 27.5-fold, at least about 30-fold, at least about 50-fold, or at least about 100-fold lower than the EC₅₀ of a control antibody (e.g. an anti-Sortilin antibody having a heavy chain variable region and a light chain variable region corresponding to S-60).

In some embodiments, anti-Sortilin antibodies of the present disclosure have an EC₅₀ that is at least 1.3-fold lower than control antibody (e.g. an anti-Sortilin antibody having a heavy chain variable region and a light chain variable region corresponding to S-60). In some embodiments, anti-Sortilin antibodies of the present disclosure have an EC₅₀ that is at least 1.8-fold lower than control antibody (e.g. an anti-Sortilin antibody having a heavy chain variable region and a light chain variable region corresponding to S-60). In some embodiments, anti-Sortilin antibodies of the present disclosure have an EC₅₀ that is at least 1.9-fold lower than control antibody (e.g. an anti-Sortilin antibody having a heavy chain variable region and a light chain variable region corresponding to S-60). In some embodiments, anti-Sortilin antibodies of the present disclosure have an EC₅₀ that is at least 2.3-fold lower than control antibody (e.g. an anti-Sortilin antibody having a heavy chain variable region and a light chain variable region corresponding to S-60).

Any in vitro cell-based assays or suitable in vivo model described herein or known in the art may be used to measure inhibition or reduction of interaction (e.g., binding) between Sortilin and one or more Sortilin ligands. In some embodiments, anti-Sortilin antibodies of the present disclosure inhibit or reduce interaction (e.g., binding) between Sortilin and one or more Sortilin ligands by reducing Sortilin expression (e.g., by reducing cell surface levels of Sortilin). In some embodiments, anti-Sortilin antibodies of the present disclosure inhibit or reduce interaction (e.g., binding) between Sortilin and one or more Sortilin ligands by at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more at saturating antibody concentrations utilizing any in vitro assay or cell-based culture assay described herein or known in the art.

In some embodiments, an anti-Sortilin antibody of the present disclosure blocks Progranulin binding to Sortlin by more than about 90% at 50 nM IgG or by more than about 96% at 150 nM IgG, as measured by flow cytometry. In some embodiments, an anti-Sortilin antibody of the present disclosure blocks Progranulin binding to Sortlin by about 90.74% at 50 nM IgG, as measured by flow cytometry. In some embodiments, an anti-Sortilin antibody of the present disclosure blocks Progranulin binding to Sortlin by about 96.5% at 150 nM IgG, as measured by flow cytometry. In some embodiments, an anti-Sortilin antibody of the present disclosure blocks Progranulin binding to Sortlin by about 96.9% at 150 nM IgG, as measured by flow cytometry.

### Decreasing Expression of Pro-Inflammatory Mediators

In some embodiments, anti-Sortilin antibodies of the present disclosure may decrease the expression of pro-inflammatory mediators after binding to a Sortilin protein expressed in a cell.

As used herein, pro-inflammatory mediators are proteins involved either directly or indirectly (e.g., by way of pro-inflammatory signaling pathways) in a mechanism that induces, activates, promotes, or otherwise decreases an inflammatory response. Any method known in the art for identifying and characterizing pro-inflammatory mediators may be used.

Examples of pro-inflammatory mediators include, without limitation, cytokines, such as type I and II interferons, IL-6, IL12p70, IL12p40, IL-10, TNF-α, IL-8, CRP, IL-20 family members, IL-33, LIF, OSM, CNTF, GM-CSF, IL-11, IL-12, IL-17, IL-18, and CRP. Further examples of pro-inflammatory mediators include, without limitation, chemokines, such as CXCL1, CCL2, CCL3, CCL4, and CCL5.

In some embodiments, the anti-Sortilin antibodies of the present disclosure may decrease functional expression and/or secretion of pro-inflammatory mediators, IL-6, IL12p70, IL12p40, IL-10, TNF-α, CXCL1, CCL2, CCL3, CCL4, and CCL5. In certain embodiments, decreased expression of the pro-inflammatory mediators occurs in macrophages, dendritic cells, monocytes, osteoclasts, Langerhans cells of skin, Kupffer cells, T cells, and/or microglial cells. Decreased expression may include, without limitation, a decrease in gene expression, a decrease in transcriptional expression, or a decrease in protein expression. Any method known in the art for determining gene, transcript (e.g., mRNA), and/or protein expression may be used. For example, Northern blot analysis may be used to determine pro-inflammatory mediator gene expression levels, RT-PCR may be used to determine the level of pro-inflammatory mediator transcription, and Western blot analysis may be used to determine pro-inflammatory mediator protein levels.

As used herein, a pro-inflammatory mediator may have decreased expression if its expression in one or more cells of a subject treated with a Sortilin agent, such as an agonist anti-Sortilin antibody of the present disclosure is more than the expression of the same pro-inflammatory mediator expressed in one or more cells of a corresponding subject that is not treated with the agonist anti-Sortilin antibody. In some embodiments, the anti-Sortilin antibody of the present disclosure may decrease pro-inflammatory mediator expression in one or more cells of a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to pro-inflammatory mediator expression in one or more cells of a corresponding subject that is not treated with the anti-Sortilin antibody. In other embodiments, the anti-Sortilin antibody may decrease pro-inflammatory mediator expression in one or more cells of a subject by at least at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to pro-inflammatory mediator expression in one or more cells of a corresponding subject that is not treated with the anti-Sortilin antibody.

In some embodiments, an anti-Sortilin antibody according to any of the above embodiments may incorporate any of the features, singly or in combination, as described in Sections 1-7 below:

### (1) Anti-Sortilin Antibody Binding Affinity

In some embodiments of any of the antibodies provided herein, the antibody has a dissociation constant (Kd) of <1 M, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹ M).

Anti-Sortilin antibodies of the present disclosure may have nanomolar or even picomolar affinities for the target antigen (e.g., human Sortilin or mammalian Sortilin). In certain embodiments, the binding affinity of an anti-Sortilin antibody of the present disclosure for target antigen (e.g., human Sortilin or mammalian Sortilin) is measured by the dissociation constant, K_{D}. Dissociation constants may be determined through any analytical technique, including any biochemical or biophysical technique such as fluorescent activated cell sorting (FACS), flow cytometry, enzyme-linked immunosorbent assay (ELISA), surface plasmon resonance (SPR), BioLayer interferometry (see, e.g., Octet System by ForteBio), meso scale discover (see, e.g., MSD-SET), isothermal titration calorimetry (ITC), differential scanning calorimetry (DSC), circular dichroism (CD), stopped-flow analysis, and colorimetric or fluorescent protein melting analyses; or a cell binding assay. In some embodiments, the K_{D} for Sortilin is determined at a temperature of approximately 25° C. In some embodiments, the dissociation constant (K_{D}) may be measured at 4° C. or room temperature utilizing, for example, FACS or BioLlayer interferometry assay.

In some embodiments, the K_{D} for Sortilin is determined at a temperature of approximately 4° C. In some embodiments, the K_{D} is determined using a monovalent antibody (e.g., a Fab) or a full-length antibody in a monovalent form. In some embodiments, the K_{D} is determined using a bivalent antibody and monomeric recombinant Sortilin protein.

In certain embodiments, the K_{D} of an anti-Sortilin antibody of the present disclosure for human Sortilin, mammalian Sortilin, or both, is measured using FACS as described herein. In certain embodiments, the K_{D} of an anti-Sortilin antibody of the present disclosure for human Sortilin, mammalian Sortilin, or both, is measured using BioLayer Interferometry as described herein.

In some embodiments, the anti-Sortilin antibody has a dissociation constant (K_{D}) for human Sortilin that is up to 2.5-fold lower than an anti-Sortilin antibody comprising a heavy chain variable region comprising the sequence of SEQ ID NO: 56 and a light chain variable region comprising the sequence of SEQ ID NO: 79, wherein the K_{D} is determined by FACS. In some embodiments, the anti-Sortilin antibody has a dissociation constant (K_{D}) for human Sortilin that ranges from about 1.10E-8 M to about 4.68E-10 M wherein the K_{D} is determined by FACS, or about 270 to about 2910 pM wherein the K_{D} is determined by Bio-layer interferometry.

In certain embodiments, the K_{D} of an anti-Sortilin antibody of the present disclosure for human Sortilin, mammalian Sortilin, or both, may be less than than 100 nM, less than 90 nM, less than 80 nM, less than 70 nM, less than 60 nM, less than 50 nM, less than 40 nM, less than 30 nM, less than 20 nM, less than 10 nM, less than 9 nM, less than 8 nM, less than 7 nM, less than 6 nM, less than 5 nM, less than 4 nM, less than 3 nM, less than 2 nM, less than 1 nM, less than 0.5 nM, less than 0.1 nM, less than 0.09 nM, less than 0.08 nM, less than 0.07 nM, less than 0.06 nM, less than 0.05 nM, less than 0.04 nM, less than 0.03 nM, less than 0.02 nM, less than 0.01 nM, less than 0.009 nM, less than 0.008 nM, less than 0.007 nM, less than 0.006 nM, less than 0.005 nM, less than 0.004 nM, less than 0.003 nM, less than 0.002 nM, less than 0.001 nM, or less than 0.001 nM.

The dissociation constants (K_{D}) of anti-Sortilin antibodies for human Sortilin, mammalian Sortilin, or both, may be less than 10 nM, less than 9.5 nM, less than 9 nM, less than 8.5 nM, less than 8 nM, less than 7.5 nM, less than 7 nM, less than 6.9 nM, less than 6.8 nM, less than 6.7 nM, less than 6.6 nM, less than 6.5 nM, less than 6.4 nM, less than 6.3 nM, less than 6.2 nM, less than 6.1 nM, less than 6 nM, less than 5.5 nM, less than 5 nM, less than 4.5 nM, less than 4 nM, less than 3.5 nM, less than 3 nM, less than 2.5 nM, less than 2 nM, less than 1.5 nM, less than 1 nM, less than 0.95 nM, less than 0.9 nM, less than 0.89 nM, less than 0.88 nM, less than 0.87 nM, less than 0.86 nM, less than 0.85 nM, less than 0.84 nM, less than 0.83 nM, less than 0.82 nM, less than 0.81 nM, less than 0.8 nM, less than 0.75 nM, less than 0.7 nM, less than 0.65 nM, less than 0.64 nM, less than 0.63 nM, less than 0.62 nM, less than 0.61 nM, less than 0.6 nM, less than 0.55 nM, less than 0.5 nM, less than 0.45 nM, less than 0.4 nM, less than 0.35 nM, less than 0.3 nM, less than 0.29 nM, less than 0.28 nM, less than 0.27 nM, less than 0.26 nM, less than 0.25 nM, less than 0.24 nM, less than 0.23 nM, less than 0.22 nM, less than 0.21 nM, less than 0.2 nM, less than 0.15 nM, less than 0.1 nM, less that 0.09 nM, less than 0.08 nM, less than 0.07 nM, less than 0.06 nM, less than 0.05 nM, less than 0.04 nM, less than 0.03 nM, less than 0.02 nM, less than 0.01 nM, less that 0.009 nM, less than 0.008 nM, less than 0.007 nM, less than 0.006 nM, less than 0.005 nM, less than 0.004 nM, less than 0.003 nM, less than 0.002 nM, or less than 0.001 nM.

In certain embodiments, the dissociation constant (K_{D}) of the antibody for Sortilin is from about 0.560 nM to about 1.63 nM, for example when the K_{D} is determined by FACS. In certain embodiments, the dissociation constant (K_{D}) of the antibody for Sortilin is from about 0.270 nM to about 2.910 nM, for example when the K_{D} is determined by BioLayer Interferometry. In some embodiments, the antibody has a dissociation constant (K_{D}) for human Sortilin, mouse Sortilin, or both, that ranges from about 0.36 nM to about 0.43 nM, or less than 1.02 nM. In some embodiments, the dissociation constant is less than 1.02 nM. In some embodiments, an anti-Sortilin antibody of the present disclosure has a dissociation constant for human Sortilin of 0.560 nM or less.

In one specific embodiment, an anti-Sortilin antibody of the present disclosure has a dissociation constant for human Sortilin of about 0.560 nM. In one specific embodiment, an anti-Sortilin antibody of the present disclosure has a dissociation constant for human Sortilin of about 0.423 nM. In one specific embodiment, an anti-Sortilin antibody of the present disclosure has a dissociation constant for human Sortilin of about 0.365 nM. In one specific embodiment, an anti-Sortilin antibody of the present disclosure has a dissociation constant for human Sortilin of about 0.344 nM. In one specific embodiment, an anti-Sortilin antibody of the present disclosure has a dissociation constant for human Sortilin of about 0.298 nM. In one specific embodiment, an anti-Sortilin antibody of the present disclosure has a dissociation constant for human Sortilin of about 0.270 nM. In another specific embodiment, an anti-Sortilin antibody of the present disclosure has a dissociation constant for human Sortilin of about 0.260 nM.

In some embodiments, anti-Sortilin antibodies of the present disclosure have a lower dissociation constant (K_{D}) for Sortilin than a control anti-Sortilin antibody (e.g., a control anti-Sortilin antibody comprising a heavy chain variable region and a light chain variable region corresponding to S-60. In some embodiments, anti-Sortilin antibodies of the present disclosure have a K_{D} for a target (e.g., human Sortilin) that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% lower than the K_{D} of a control anti-Sortilin antibody for the target (e.g., a control anti-Sortilin antibody comprising a heavy chain variable region and a light chain variable region corresponding to S-60. In some embodiments, anti-Sortilin antibodies of the present disclosure have a K_{D} for a target (e.g., human Sortilin) that is at least about 1-fold, at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 17.5-fold, at least about 20-fold, at least about 22.5-fold, at least about 25-fold, at least about 27.5-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, or at least about 1000-fold lower than the K_{D} of a control anti-Sortilin antibody for the target (e.g., a control anti-Sortilin antibody comprising a heavy chain variable region and a light chain variable region corresponding to S-60.

In some embodiments, anti-Sortilin antibodies of the present disclosure have a K_{D} for human Sortilin that is at least 100-fold lower than an anti-Sortilin antibody having a heavy chain variable region and a light chain variable region corresponding to S-60. In some embodiments, anti-Sortilin antibodies of the present disclosure have a K_{D} for human Sortilin that is at least 50-fold lower than an anti-Sortilin antibody having a heavy chain variable region and a light chain variable region corresponding to S-60. In some embodiments, anti-Sortilin antibodies of the present disclosure have a K_{D} for human Sortilin that is at least 10-fold lower than an anti-Sortilin antibody having a heavy chain variable region and a light chain variable region corresponding to S-60. In some embodiments, anti-Sortilin antibodies of the present disclosure have a K_{D} for human Sortilin that is at least 5-fold lower than an anti-Sortilin antibody having a heavy chain variable region and a light chain variable region corresponding to S-60. In some embodiments, anti-Sortilin antibodies of the present disclosure have a K_{D} for human Sortilin that is at least 2-fold lower than an anti-Sortilin antibody having a heavy chain variable region and a light chain variable region corresponding to S-60.

In a specific embodiment, an anti-Sortilin antibody of the present disclosure has a K_{D} for human Sortilin that is about 2.79-fold lower than an anti-Sortilin antibody having a heavy chain variable region and a light chain variable region corresponding to S-60. In another specific embodiment, an anti-Sortilin antibody of the present disclosure has a K_{D} for human Sortilin that is about 2.05-fold lower than an anti-Sortilin antibody having a heavy chain variable region and a light chain variable region corresponding to S-60.

### (2) Antibody Fragments

In some embodiments of any of the antibodies provided herein, the antibody antibodies is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

In some embodiments, the antibody fragment is used in combination with a second Sortilin antibody and/or with one or more antibodies that specifically bind a disease-causing protein selected from: amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, proline-arginine (PR) repeat peptides, and any combination thereof.

### (3) Chimeric and Humanized Antibodies

In some embodiments of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments of any of the antibodies provided herein, the antibody is a humanized antibody. Typically, a non-human antibody is humanized to reduce immunogenicity to humans, while retaining the specificity and affinity of the parental non-human antibody. In certain embodiments, a humanized antibody is substantially non-immunogenic in humans. In certain embodiments, a humanized antibody has substantially the same affinity for a target as an antibody from another species from which the humanized antibody is derived. See, e.g., U.S. Pat. Nos. 5,530,101, 5,693,761; 5,693,762; and 5,585,089. In certain embodiments, amino acids of an antibody variable domain that can be modified without diminishing the native affinity of the antigen binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

### (4) Human Antibodies

In some embodiments of any of the antibodies provided herein, the antibody is a human antibody. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al. Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3):927-937 (2005) and Vollmers et al. Methods and Findings in Experimental and Clinical Pharmacology 27(3):185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some embodiments of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast-based platforms (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J. Mol. Biol. 338(2): 299-310, 2004; Lee et al. J. Mol. Biol. 340(5): 1073-1093, 2004; Fellouse Proc. Natl. Acad. Sci. USA 101(34):12467-12472 (2004); and Lee et al. J. Immunol. Methods 284(−2):1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12: 725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

### (5) Constant Regions Including Fc Regions

In some embodiments of any of the antibodies provided herein, the antibody comprises an Fe. In some embodiments, the Fe is a human IgG1, IgG2, IgG3, and/or IgG4 isotype. In some embodiments, the antibody is of the IgG class, the IgM class, or the IgA class.

In certain embodiments of any of the antibodies provided herein, the antibody has an IgG2 isotype. In some embodiments, the antibody contains a human IgG2 constant region. In some embodiments, the human IgG2 constant region includes an Fc region. In some embodiments, the antibody induces the one or more Sortilin activities or independently of binding to an Fc receptor. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has a hybrid IgG2/4 isotype. In some embodiments, the antibody includes an amino acid sequence comprising amino acids 118 to 260 according to EU numbering of human IgG2 and amino acids 261-447 according to EU numbering of human IgG4 (WO 1997/11971; WO 2007/106585).

In some embodiments, the Fc region increases clustering without activating complement as compared to a corresponding antibody comprising an Fc region that does not comprise the amino acid substitutions. In some embodiments, the antibody induces one or more activities of a target specifically bound by the antibody. In some embodiments, the antibody binds to Sortilin.

It may also be desirable to modify an anti-Sortilin antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some embodiments, the effector function is impaired by removing N-glycosylation of the Fc region (e.g., in the CH2 domain of IgG) of the antibody. In some embodiments, the effector function is impaired by modifying regions such as 233-236, 297, and/or 327-331 of human IgG as described in WO 99/58572 and Armour et al. Molecular Immunology 40: 585-593 (2003); Reddy et al. J. Immunology 164:1925-1933 (2000). In other embodiments, it may also be desirable to modify an anti-Sortilin antibody of the present disclosure to modify effector function to increase finding selectivity toward the ITIM-containing FcgRIIb (CD32b) to increase clustering of Sortilin antibodies on adjacent cells without activating humoral responses including Antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Other amino acid sequence modifications.

### (6) Multispecific Antibodies

Multispecific are antibodies that have binding specificities for at least two different epitopes, including those on the same or another polypeptide (e.g., one or more Sortilin polypeptides of the present disclosure). In some embodiments, the multispecific antibody can be a bispecific antibody. In some embodiments, the multispecific antibody can be a trispecific antibody. In some embodiments, the multispecific antibody can be a tetraspecific antibody. Such antibodies can be derived from full-length antibodies or antibody fragments (e.g., F(ab′)2bispecific antibodies). In some embodiments, the multispecific antibody comprises a first antigen binding region which binds to first site on Sortilin and comprises a second antigen binding region which binds to a second site on Sortilin. In some embodiment, the multispecific antibodies comprises a first antigen binding region which binds to Sortilin and a second antigen binding region that binds to a second polypeptide.

Provided herein are multispecific antibodies comprises a first antigen binding region, wherein the first antigen binding region comprises the six HVRs of an antibody described herein, which binds to Sortilin and a second antigen binding region that binds to a second polypeptide. In some embodiments, the first antigen binding region comprises the V_{H} or V_{L} of an antibody described herein.

In some embodiments of any of the multispecific antibodies, the second polypeptide is a) an antigen facilitating transport across the blood-brain-barrier; (b) an antigen facilitating transport across the blood-brain-barrier selected from transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, and ANG1005; (c) a disease-causing protein selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; (d) ligands and/or proteins expressed on immune cells, wherein the ligands and/or proteins selected from CD40, OX40, ICOS, CD28, CD137/4-1BB, CD27, GITR, PD-L1, CTLA-4, PD-L2, PD-1, B7-H3, B7-H4, HVEM, BTLA, KIR, GAL9, TIM3, A2AR, LAG-3, and phosphatidylserine; and/or (e) a protein, lipid, polysaccharide, or glycolipid expressed on one or more tumor cells and any combination thereof.

Numerous antigens are known in the art that facilitate transport across the blood-brain barrier (see, e.g., Gabathuler R. Neurobiol. Dis. 37:48-57 (2010)). Such second antigens include, without limitation, transferrin receptor (TR), insulin receptor (HIR), Insulin-like growth factor receptor (IGFR), low-density lipoprotein receptor related proteins 1 and 2 (LPR-1 and 2), diphtheria toxin receptor, including CRM197 (a non-toxic mutant of diphtheria toxin), llama single domain antibodies such as TMEM 30(A) (Flippase), protein transduction domains such as TAT, Syn-B, or penetratin, poly-arginine or generally positively charged peptides, Angiopep peptides such as ANG1005 (see, e.g., Gabathuler, 2010), and other cell surface proteins that are enriched on blood-brain barrier endothelial cells (see, e.g., Daneman et al. PLoS One 5(10):e13741 (2010)).

The multivalent antibodies may recognize the Sortilin antigen as well as without limitation additional antigens Aβ peptide, antigen or an α-synuclein protein antigen or, Tau protein antigen or, TDP-43 protein antigen or, prion protein antigen or, huntingtin protein antigen, or RAN, translation Products antigen, including the DiPeptide Repeats, (DPRs peptides) composed of glycine-alanine (GA), glycine-proline (GP), glycine-arginine (GR), proline-alanine (PA), or proline-arginine (PR), Insulin receptor, insulin like growth factor receptor. Transferrin receptor or any other antigen that facilitate antibody transfer across the blood brain barrier. In some embodiments, the second polypeptide is transferrin. In some embodiments, the second polypeptide is Tau. In some embodiments, the second polypeptide is Aβ. In some embodiments, the second polypeptide is TREM2. In some embodiments, the second polypeptide is α-synuclein.

The multivalent antibody contains at least one polypeptide chain (and preferably two polypeptide chains), wherein the polypeptide chain or chains comprise two or more variable domains. For instance, the polypeptide chain or chains may comprise VD1-(X1)ₙ-VD2-(X2)ₙ-Fc, wherein VD1 is a first variable domain, VD2 is a second variable domain, Fe is one polypeptide chain of an Fc region, X1 and X2 represent an amino acid or polypeptide, and n is 0 or 1. Similarly, the polypeptide chain or chains may comprise V_{H}-C_{H}1-flexible linker-V_{H}-C_{H}1-Fc region chain; or V_{H}-C_{H}1-V_{H}-C_{H}1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

Techniques for making multispecific antibodies include, but are not limited to, recombinant co-expression of two immunoglobulin heavy chain-light chain pairs having different specificities (see Milstein and Cuello Nature 305: 537 (1983), WO 93/08829, and Traunecker et al. EMBO J. 10:3655 (1991)), and “knob-in-hole” engineering (see, e.g., U.S. Pat. No. 5,731,168). See also WO 2013/026833 (CrossMab). Multi-specific antibodies may also be made by engineering electrostatic steering effects for making antibody Fc-heterodimeric molecules (WO 2009/089004A1); cross-linking two or more antibodies (see, e.g., U.S. Pat. No. 4,676,980); using leucine; using “diabody” technology for making bispecific antibody fragments (see, e.g., Hollinger et al. Proc. Natl. Acad. Sci. USA 90:6444-6448 (1993)); and using single-chain Fv (scFv) dimers (see, e.g., Gruber et al. J. Immunol. 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576). The antibody herein also includes a “Dual Acting FAb” or “DAF” comprising an antigen binding site that binds to multiple Sortilin (see, US 2008/0069820, for example).

(7) Antibodies with Improved Stability

Amino acid sequence modifications of anti-Sortilin antibodies of the present disclosure, or antibody fragments thereof to improve stability during manufacturing, storage, and in vivo administration, are also contemplated. For example, it may be desirable to reduce degradation of the antibodies or antibody fragments of the present disclosure through multiple pathways, including without limitation, oxidation and deamidation. Amino acid sequence variants of the antibodies or antibody fragments are prepared by introducing appropriate nucleotide changes into the nucleic acid encoding the antibodies or antibody fragments, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics (i.e., reduced susceptibility to degradation).

In some embodiments, the asparagine (N33) site in the HVR-L1 region of an anti-Sortilin antibody of the present disclosure may be susceptible to degradation by means of deamidation. In certain embodiments, the asparagine (N33) site in the HVR-L1 region of S-60-15 (SEQ ID NO:8) may be susceptible to deamidation. Upon deamidation, the asparagine (N33) site in the HVR-L1 region of S-60-15 results in an Asn to Asp/IsoAsp change. In certain embodiments, the asparagine (N33) site in the HVR-L1 region of S-60-15 may be substituted to prevent or reduce deamidation. Non-limiting exemplary amino acid sequence variants of S-60-15 having amino acid substitutions in the asparagine (N33) site of the HVR-L1 region include S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], or S-60-15.17 [N33L].

### (8) Antibody Variants

In some embodiments of any of the antibodies provided herein, amino acid sequence variants of the antibodies are contemplated. For example, it may be desirable to improve the binding affinity and/or other biological properties of the antibody.

### (i) Substitution, Insertion, and Deletion Variants

In some embodiments ofany of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE 1
Amino Acid Substitutions

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions”.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

### (ii) Glycosylation Variants

In some embodiments of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. See, e.g., US Patent Publication Nos. 2003/0157108 and 2004/0093621. Examples of publications related to “defucosylated” or “fucose-deficient” antibody variants include: US 2003/0157108; US 2003/0115614; US 2002/0164328; US 2004/0093621; US 2004/0132140; US 2004/0110704; US 2004/0110282; US 2004/0109865; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Led 3 CHO cells deficient in protein fucosylation (Ripka et al. Arch. Biochem. Biophys. 249:533-545 (1986); US 2003/0157108), and knockout cell lines, such as alpha-1,6-fucosyltransferase gene, FUT8, knockout CHO cells (see, e.g., Yamane-Ohnuki et al. Biotech. Bioeng. 87: 614 (2004) and Kanda et al. Biotechnol. Bioeng. 94(4):680-688 (2006)).

(iii) Modified Constant Regions

In some embodiments of any of the antibodies provided herein, the antibody Fe is an antibody Fe isotypes and/or modifications. In some embodiments, the antibody Fe isotype and/or modification is capable of binding to Fe gamma receptor.

In some embodiments of any of the antibodies provided herein, the modified antibody Fe is an IgG1 modified Fe. In some embodiments, the IgG1 modified Fc comprises one or more modifications. For example, in some embodiments, the IgG1 modified Fc comprises one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention. In some embodiments of any of the antibodies provided herein, the antibody is an IgG1 isotype and the Fc region comprises amino acid substitutions at positions L234A, L235A, and P331S, wherein the numbering of the residue position is according to EU numbering.

In some embodiments of any of the IgG1 modified Fe, the Fc comprises N297A mutation according to EU numbering. In some embodiments of any of the IgG1 modified Fe, the Fc comprises D265A and N297A mutations according to EU numbering. In some embodiments of any of the IgG1 modified Fe, the Fc comprises D270A mutations according to EU numbering. In some embodiments, the IgG1 modified Fc comprises L234A and L235A mutations according to EU numbering. In some embodiments of any of the IgG1 modified Fe, the Fc comprises L234A and G237A mutations according to EU numbering. In some embodiments of any of the IgG1 modified Fe, the Fc comprises L234A, L235A and G237A mutations according to EU numbering. In some embodiments of any of the IgG1 modified Fe, the Fc comprises one or more (including all) of P238D, L328E, E233, G237D, H268D, P271G and A330R mutations according to EU numbering. In some embodiments of any of the IgG1 modified Fe, the Fc comprises one or more of S267E/L328F mutations according to EU numbering. In some embodiments of any of the IgG1 modified Fe, the Fc comprises P238D, L328E, E233D, G237D, H268D, P271G and A330R mutations according to EU numbering. In some embodiments of any of the IgG1 modified Fe, the Fc comprises P238D, L328E, G237D, H268D, P271G and A330R mutations according to EU numbering. In some embodiments of any of the IgG1 modified Fe, the Fc comprises P238D, S267E, L328E, E233D, G237D, H268D, P271G and A330R mutations according to EU numbering. In some embodiments of any of the IgG1 modified Fe, the Fc comprises P238D, S267E, L328E, G237D, H268D, P271G and A330R mutations according to EU numbering. In some embodiments of any of the IgG1 modified Fe, the Fc comprises C226S, C229S, E233P, L234V, and L235A mutations according to EU numbering. In some embodiments of any of the IgG1 modified Fe, the Fc comprises L234F, L235E, and P331S mutations according to EU numbering. In some embodiments of any of the IgG1 modified Fe, the Fc comprises S267E and L328F mutations according to EU numbering. In some embodiments of any of the IgG1 modified Fe, the Fc comprises S267E mutations according to EU numbering. In some embodiments of any of the IgG1 modified Fe, the Fc comprises a substitute of the constant heavy 1 (CH1) and hinge region of IgG1 with CH1 and hinge region of IgG2 (amino acids 118-230 of IgG2 according to EU numbering) with a Kappa light chain.

In some embodiments of any of the IgG1 modified Fe, the Fe includes two or more amino acid substitutions that increase antibody clustering without activating complement as compared to a corresponding antibody having an Fc region that does not include the two or more amino acid substitutions. Accordingly, in some embodiments of any of the IgG1 modified Fe, the IgG1 modified Fe is an antibody comprising an Fc region, where the antibody comprises an amino acid substitution at position E430G and one or more amino acid substitutions in the Fc region at a residue position selected from: L234F, L235A, L235E, S267E, K322A, L328F, A330S, P331S, and any combination thereof according to EU numbering. In some embodiments, the IgG1 modified Fc comprises an amino acid substitution at positions E430G, L243A, L235A, and P331S according to EU numbering. In some embodiments, the IgG1 modified Fc comprises an amino acid substitution at positions E430G and P331S according to EU numbering. In some embodiments, the IgG1 modified Fc comprises an amino acid substitution at positions E430G and K322A according to EU numbering. In some embodiments, the IgG1 modified Fc comprises an amino acid substitution at positions E430G, A330S, and P331S according to EU numbering. In some embodiments, the IgG1 modified Fc comprises an amino acid substitution at positions E430G, K322A, A330S, and P331S according to EU numbering. In some embodiments, the IgG1 modified Fc comprises an amino acid substitution at positions E430G, K322A, and A330S according to EU numbering. In some embodiments, the IgG1 modified Fc comprises an amino acid substitution at positions E430G, K322A, and P331S according to EU numbering.

In some embodiments of any of the IgG1 modified Fe, the IgG1 modified Fe may further comprise herein may be combined with an A330L mutation (Lazar et al. Proc Natl Acad Sci USA, 103:4005-4010 (2006)), or one or more of L234F, L235E, and/or P331S mutations (Sazinsky et al. Proc Natl Acad Sci USA, 105:20167-20172 (2008)), according to the EU numbering convention, to eliminate complement activation. In some embodiments of any of the IgG1 modified Fe, the IgG1 modified Fe may further comprise one or more of A330L, A330S, L234F, L235E, and/or P331S according to EU numbering. In some embodiments of any of the IgG1 modified Fe, the IgG1 modified Fe may further comprise one or more mutations to enhance the antibody half-life in human serum (e.g., one or more (including all) of M252Y, S254T, and T256E mutations according to the EU numbering convention). In some embodiments of any of the IgG1 modified Fe, the IgG1 modified Fe may further comprise one or more of E430G, E430S, E430F, E430T, E345K, E345Q, E345R, E345Y, S440Y, and/or S440W according to EU numbering.

Other aspects of the present disclosure relate to antibodies having modified constant regions (i.e., Fc regions). An antibody dependent on binding to FcgR receptor to activate targeted receptors may lose its agonist activity if engineered to eliminate FcgR binding (see, e.g., Wilson et al. Cancer Cell 19:101-113 (2011); Armour at al. Immunology 40:585-593 (2003); and White et al. Cancer Cell 27:138-148 (2015)). As such, it is thought that an anti-Sortlin antibody of the present disclosure with the correct epitope specificity can activate the target antigen, with minimal adverse effects, when the antibody has an Fc domain from a human IgG2 isotype (CH1 and hinge region) or another type of Fc domain that is capable of preferentially binding the inhibitory FcgRIIB r receptors, or a variation thereof.

In some embodiments of any of the antibodies provided herein, the modified antibody Fe is an IgG2 modified Fe. In some embodiments, the IgG2 modified Fc comprises one or more modifications. For example, in some embodiments, the IgG2 modified Fc comprises one or more amino acid substitutions (e.g., relative to a wild-type Fc region of the same isotype). In some embodiments of any of the IgG2 modified Fe, the one or more amino acid substitutions are selected from V234A (Alegre et al. Transplantation 57:1537-1543 (1994); Xu et al. Cell Immunol, 200:16-26 (2000)); G237A (Cole et al. Transplantation, 68:563-571 (1999)); H268Q, V309L, A330S, P331S (US 2007/0148167; Armour et al. Eur J Immunol 29: 2613-2624 (1999); Armour et al. The Haematology Journal 1 (Suppl. 1):27 (2000); Armour et al. The Haematology Journal 1 (Suppl. 1):27 (2000)), C219S, and/or C220S (White et al. Cancer Cell 27, 138-148 (2015)); S267E, L328F (Chu et al. Mol Immunol, 45:3926-3933 (2008)); and M252Y, S254T, and/or T256E according to the EU numbering convention. In some embodiments of any of the IgG2 modified Fe, the Fc comprises an amino acid substitution at positions V234A and G237A according to EU numbering. In some embodiments of any of the IgG2 modified Fe, the Fc comprises an amino acid substitution at positions C219S or C220S according to EU numbering. In some embodiments of any of the IgG2 modified Fe, the Fc comprises an amino acid substitution at positions A330S and P331S according to EU numbering. In some embodiments of any of the IgG2 modified Fe, the Fc comprises an amino acid substitution at positions S267E and L328F according to EU numbering.

In some embodiments of any of the IgG2 modified Fe, the Fc comprises a C127S amino acid substitution according to the EU numbering convention (White et al., (2015) Cancer Cell 27, 138-148; Lightle et al. Protein Sci. 19:753-762 (2010); and WO 2008/079246). In some embodiments of any of the IgG2 modified Fe, the antibody has an IgG2 isotype with a Kappa light chain constant domain that comprises a C214S amino acid substitution according to the EU numbering convention (White et al. Cancer Cell 27:138-148 (2015); Lightle et al. Protein Sci. 19:753-762 (2010); and WO 2008/079246).

In some embodiments of any of the IgG2 modified Fe, the Fc comprises a C220S amino acid substitution according to the EU numbering convention. In some embodiments of any of the IgG2 modified Fe, the antibody has an IgG2 isotype with a Kappa light chain constant domain that comprises a C214S amino acid substitution according to the EU numbering convention.

In some embodiments of any of the IgG2 modified Fe, the Fc comprises a C219S amino acid substitution according to the EU numbering convention. In some embodiments of any of the IgG2 modified Fe, the antibody has an IgG2 isotype with a Kappa light chain constant domain that comprises a C214S amino acid substitution according to the EU numbering convention.

In some embodiments of any of the IgG2 modified Fe, the Fe includes an IgG2 isotype heavy chain constant domain 1(CH1) and hinge region (White et al. Cancer Cell 27:138-148 (2015)). In certain embodiments of any of the IgG2 modified Fe, the IgG2 isotype CH1 and hinge region comprise the amino acid sequence of 118-230 according to EU numbering. In some embodiments of any of the IgG2 modified Fe, the antibody Fc region comprises a S267E amino acid substitution, a L328F amino acid substitution, or both, and/or a N297A or N297Q amino acid substitution according to the EU numbering convention.

In some embodiments of any of the IgG2 modified Fe, the Fc further comprises one or more amino acid substitution at positions E430G, E430S, E430F, E430T, E345K, E345Q, E345R, E345Y, S440Y, and S440W according to EU numbering. In some embodiments of any of the IgG2 modified Fe, the Fe may further comprise one or more mutations to enhance the antibody half-life in human serum (e.g., one or more (including all) of M252Y, S254T, and T256E mutations according to the EU numbering convention). In some embodiments of any of the IgG2 modified Fe, the Fe may further comprise A330S and P331S.

In some embodiments of any of the IgG2 modified Fe, the Fe is an IgG2/4 hybrid Fe. In some embodiments, the IgG2/4 hybrid Fc comprises IgG2 aa 118 to 260 and IgG4 aa 261 to 447. In some embodiments of any IgG2 modified Fe, the Fc comprises one or more amino acid substitutions at positions H268Q, V309L, A330S, and P331S according to EU numbering.

In some embodiments of any of the IgG1 and/or IgG2 modified Fe, the Fc comprises one or more additional amino acid substitutions selected from A330L, L234F; L235E, or P331S according to EU numbering; and any combination thereof.

In certain embodiments of any of the IgG1 and/or IgG2 modified Fe, the Fc comprises one or more amino acid substitutions at a residue position selected from C127S, L234A, L234F, L235A, L235E, S267E, K322A, L328F, A330S, P331S, E345R, E430G, S440Y, and any combination thereof according to EU numbering. In some embodiments of any of the IgG1 and/or IgG2 modified Fe, the Fc comprises an amino acid substitution at positions E430G, L243A, L235A, and P331S according to EU numbering. In some embodiments of any of the IgG1 and/or IgG2 modified Fe, the Fc comprises an amino acid substitution at positions E430G and P331S according to EU numbering. In some embodiments of any of the IgG1 and/or IgG2 modified Fe, the Fc comprises an amino acid substitution at positions E430G and K322A according to EU numbering. In some embodiments of any of the IgG1 and/or IgG2 modified Fe, the Fc comprises an amino acid substitution at positions E430G, A330S, and P331S according to EU numbering. In some embodiments of any of the IgG1 and/or IgG2 modified Fe, the Fc comprises an amino acid substitution at positions E430G, K322A, A330S, and P331S according to EU numbering. In some embodiments of any of the IgG1 and/or IgG2 modified Fe, the Fc comprises an amino acid substitution at positions E430G, K322A, and A330S according to EU numbering. In some embodiments of any of the IgG1 and/or IgG2 modified Fe, the Fc comprises an amino acid substitution at positions E430G, K322A, and P331S according to EU numbering. In some embodiments of any of the IgG1 and/or IgG2 modified Fe, the Fc comprises an amino acid substitution at positions S267E and L328F according to EU numbering. In some embodiments of any of the IgG1 and/or IgG2 modified Fe, the Fc comprises an amino acid substitution at position C127S according to EU numbering. In some embodiments of any of the IgG1 and/or IgG2 modified Fe, the Fc comprises an amino acid substitution at positions E345R, E430G and S440Y according to EU numbering.

In some embodiments of any of the antibodies provided herein, the modified antibody Fe is an IgG4 modified Fe. In some embodiments, the IgG4 modified Fc comprises one or more modifications. For example, in some embodiments, the IgG4 modified Fc comprises one or more amino acid substitutions (e.g., relative to a wild-type Fc region of the same isotype). In some embodiments of any of the IgG4 modified Fe, the one or more amino acid substitutions are selected from L235A, G237A, S229P, L236E (Reddy et al. J Immunol 164:1925-1933(2000)), S267E, E318A, L328F, M252Y, S254T, and/or T256E according to the EU numbering convention. In some embodiments of any of the IgG4 modified Fe, the Fe may further comprise L235A, G237A, and E318A according to the EU numbering convention. In some embodiments of any of the IgG4 modified Fe, the Fe may further comprise S228P and L235E according to the EU numbering convention. In some embodiments of any of the IgG4 modified Fe, the IgG4 modified Fe may further comprise S267E and L328F according to the EU numbering convention.

In some embodiments of any of the IgG4 modified Fe, the IgG4 modified Fc comprises may be combined with an S228P mutation according to the EU numbering convention (Angal et al. Mol Immunol. 30:105-108 (1993)) and/or with one or more mutations described in (Peters et al. J Biol Chem. 287(29):24525-33 (2012)) to enhance antibody stabilization.

In some embodiments of any of the IgG4 modified Fe, the IgG4 modified Fe may further comprise one or more mutations to enhance the antibody half-life in human serum (e.g., one or more (including all) of M252Y, S254T, and T256E mutations according to the EU numbering convention).

In some embodiments of any of the IgG4 modified Fe, the Fc comprises L235E according to EU numbering. In certain embodiments of any of the IgG4 modified Fe, the Fc comprises one or more amino acid substitutions at a residue position selected from C127S, F234A, L235A, L235E, S267E, K322A, L328F, E345R, E430G, S440Y, and any combination thereof, according to EU numbering. In some embodiments of any of the IgG4 modified Fe, the Fc comprises an amino acid substitution at positions E430G, L243A, L235A, and P331S according to EU numbering. In some embodiments of any of the IgG4 modified Fe, the Fc comprises an amino acid substitution at positions E430G and P331S according to EU numbering. In some embodiments of any of the IgG4 modified Fe, the Fc comprises an amino acid substitution at positions E430G and K322A according to EU numbering. In some embodiments of any of the IgG4 modified Fe, the Fc comprises an amino acid substitution at position E430 according to EU numbering. In some embodiments of any of the IgG4 modified Fe, the Fe region comprises an amino acid substitution at positions E430G and K322A according to EU numbering. In some embodiments of any of the IgG4 modified Fe, the Fe comprises an amino acid substitution at positions S267E and L328F according to EU numbering. In some embodiments of any of the IgG4 modified Fe, the Fe comprises an amino acid substitution at position C127S according to EU numbering. In some embodiments of any of the IgG4 modified Fe, the Fe comprises an amino acid substitution at positions E345R, E430G and S440Y according to EU numbering.

### Nucleic Acids, Vectors, and Host Cells

Anti-Sortilin antibodies of the present disclosure may be produced using recombinant methods and compositions, e.g., as described in U.S. Pat. No. 4,816,567. In some embodiments, isolated nucleic acids having a nucleotide sequence encoding any of the anti-Sortilin antibodies of the present disclosure are provided. Such nucleic acids may encode an amino acid sequence comprising the V_{L} and/or an amino acid sequence comprising the V_{H} Of the anti-Sortilin antibody (e.g., the light and/or heavy chains of the antibody). In some embodiments, one or more vectors (e.g., expression vectors) comprising such nucleic acids are provided. In some embodiments, a host cell comprising such nucleic acid is also provided. In some embodiments, the host cell comprises (e.g., has been transduced with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-Sortilin antibody of the present disclosure are provided. In some embodiments, the method includes culturing a host cell of the present disclosure comprising a nucleic acid encoding the anti-Sortilin antibody, under conditions suitable for expression of the antibody. In some embodiments, the antibody is subsequently recovered from the host cell (or host cell culture medium).

For recombinant production of an anti-Sortilin antibody of the present disclosure, a nucleic acid encoding the anti-Sortilin antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-Sortilin antibodies of the present disclosure, or cell-surface expressed fragments or polypeptides thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Suitable host cells for cloning or expression of antibody-encoding vectors include prokaryotic or eukaryotic cells. For example, anti-Sortilin antibodies of the present disclosure may be produced in bacteria, in particular when glycosylation and Fe effector function are not needed. For expression of antibody fragments and polypeptides in bacteria (e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross Nat. Biotech. 22:1409-1414 (2004); and Li et al. Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al. Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### Biomarkers

In some embodiments, administration of an anti-Sortilin antibody of the present disclosure increases the level (e.g., in whole blood, plasma, and/or CSF) of one or more lysosomal markers, such as CTSB, by any of at least 10%, at least 20%, at least 30%, at least 40%, at least 50%, at least 60%, at least 70%, at least 80%, at least 90%, at least 100%, or more, compared to the baseline level (e.g., in whole blood, plasma, and/or CSF) of the one or more lysosomal markers, such as CTSB. In some embodiments, administration of an anti-Sortilin antibody of the present disclosure increases the level of CTSB (e.g., in whole blood, plasma, and/or CSF) by at least about 20% compared to the baseline level of CTSB (e.g., in whole blood, plasma, and/or CSF). Another non-limiting example of a lysosomal marker is N-acetylglucosamine kinase (NAGK). In some embodiments, administration of an anti-Sortilin antibody of the present disclosure increases the level of NAGK (e.g., in whole blood, plasma, and/or CSF) by any of at least 10%, at least 20%, at least 30%, at least 40%, at least 50%, at least 60%, at least 70%, at least 80%, at least 90%, at least 100%, or more, compared to the baseline level of NAGK (e.g., in whole blood, plasma, and/or CSF).

In some embodiments, administration of an anti-Sortilin antibody of the present disclosure decreases the level (e.g., in whole blood, plasma, and/or CSF) of one or more inflammatory markers, such as SPP1, by any of at least 10%, at least 20%, at least 30%, at least 40%, at least 50%, at least 60%, at least 70%, at least 80%, at least 90%, or 100% compared to the baseline level (e.g., in whole blood, plasma, and/or CSF) of the one or more inflammatory markers, such as SPP1. In some embodiments, administration of an anti-Sortilin antibody of the present disclosure decreases the level of SPP1 (e.g., in whole blood, plasma, and/or CSF) by at least about 10% compared to the baseline level of SPP1 (e.g., in whole blood, plasma, and/or CSF). Other examples of inflammatory markers include, without limitation, YWHAE (14-3-3 protein epsilon), allograft inflammatory factor 1 (AIF1), colony stimulating factor 1 (CSF1), chitinase 1 (CHIT1), lymphocyte antigen 86 (LY86), and CD86. In some embodiments, administration of an anti-Sortilin antibody of the present disclosure decreases the level (e.g., in whole blood, plasma, and/or CSF) of one or more inflammatory markers, such as YWHAE (14-3-3 protein epsilon), allograft inflammatory factor 1 (AIF1), colony stimulating factor 1 (CSF1), chitinase 1 (CHIT1), lymphocyte antigen 86 (LY86), or CD86, by any of at least 10%, at least 20%, at least 30%, at least 40%, at least 50%, at least 60%, at least 70%, at least 80%, at least 90%, or 100% compared to the baseline level (e.g., in whole blood, plasma, and/or CSF) of the one or more inflammatory markers, such as YWHAE (14-3-3 protein epsilon), allograft inflammatory factor 1 (AIF1), colony stimulating factor 1 (CSF1), chitinase 1 (CHIT1), lymphocyte antigen 86 (LY86), or CD86.

Also provided herein are methods of monitoring the treatment of an individual being administered an anti-Sortilin antibody of the present disclosure.

In some embodiments, the methods comprise measuring the level of one or more proteins in a sample from the individual before and after the individual has received one or more doses of an anti-Sortilin antibody, wherein the one or more proteins are CTSB and/or SPP1. In some embodiments, the method further comprises a step of assessing the activity of the anti-Sortilin antibody in the individual based on the level of the one or more proteins in the sample. In some embodiments, the sample is from the cerebrospinal fluid of the individual or the blood of the individual. In some embodiments, the sample is from the cerebrospinal fluid of the individual.

In some embodiments, the methods comprise measuring the level of one or more proteins in a sample from the individual before and after the individual has received one or more doses of an anti-Sortilin antibody, wherein the one or more proteins are selected from the group consisting of CTSB, SPP1, NAGK, YWHAE, AIF1, CSF1, CHIT1, LY86, and CD86. In some embodiments, the method further comprises assessing the activity of the anti-Sortilin antibody in the individual based on the level of the one or more proteins in the sample. In some embodiments, the sample is from the cerebrospinal fluid of the individual. In some embodiments, the sample is from the blood of the individual.

In some embodiments, the anti-Sortilin antibody is determined to be active in the individual if the level of CTSB in the cerebrospinal fluid after the individual has received one or more doses of the anti-Sortilin antibody is increased (e.g., by any of at least 10%, at least 20%, at least 30%, at least 40%, at least 50%, at least 60%, at least 70%, at least 80%, at least 90%, at least 100%, or more) compared to the level of CTSB in the cerebrospinal fluid before the individual received one or more doses of the anti-Sortilin antibody. In some embodiments, the anti-Sortilin antibody is determined to be active in the individual if the level of CTSB in the cerebrospinal fluid after the individual has received one or more doses of the anti-Sortilin antibody is increased by at least about 20% compared to the level of CTSB in the cerebrospinal fluid before the individual received one or more doses of the anti-Sortilin antibody.

In some embodiments, the anti-Sortilin antibody is determined to be active in the individual if the level of SPP1 in the cerebrospinal fluid after the individual has received one or more doses of the anti-Sortilin antibody is decreased (e.g., by any of at least 10%, at least 20%, at least 30%, at least 40%, at least 50%, at least 60%, at least 70%, at least 80%, at least 90%, or 100%) compared to the level of SPP1 in the cerebrospinal fluid before the individual has received one or more doses of the anti-Sortilin antibody. In some embodiments, the anti-Sortilin antibody is determined to be active in the individual if the level of SPP1 in the cerebrospinal fluid after the individual has received one or more doses of the anti-Sortilin antibody is decreased by at least about 10% compared to the level of SPP1 in the cerebrospinal fluid before the individual has received one or more doses of the anti-Sortilin antibody.

In some embodiments, the anti-Sortilin antibody is determined to be active in the individual if the level of NAGK in the cerebrospinal fluid after the individual has received one or more doses of the anti-Sortilin antibody is increased (e.g., by any of at least 10%, at least 20%, at least 30%, at least 40%, at least 50%, at least 60%, at least 70%, at least 80%, at least 90%, at least 100%, or more) compared to the level of NAGK in the cerebrospinal fluid before the individual has received one or more doses of the anti-Sortilin antibody.

In some embodiments, the anti-Sortilin antibody is determined to be active in the individual if the levels of one or more inflammatory proteins in the cerebrospinal fluid after the individual has received one or more doses of the anti-Sortilin antibody are decreased (e.g., by any of at least 10%, at least 20%, at least 30%, at least 40%, at least 50%, at least 60%, at least 70%, at least 80%, at least 90%, at least 100%, or more) compared to the levels of the one or more inflammatory proteins in the cerebrospinal fluid before the individual has received one or more doses of the anti-Sortilin antibody, wherein the one or more inflammatory proteins are selected from the group consisting of 14-3-3 protein epsilon (YWHAE), allograft inflammatory factor 1 (AIF1), colony stimulating factor 1 (CSF1), chitinase 1 (CHIT1), lymphocyte antigen 86 (LY86), and CD86.

In some embodiments, the sample is from the cerebrospinal fluid of the individual.

In some embodiments, the sample is from the blood of the individual.

In some embodiments, the levels of one or more proteins (e.g., one or more of CTSB, SPP1, NAGK, YWHAE, AIF1, CSF1, CHIT1, LY86, or CD86) may be measured in a sample obtained from the individual, such as a sample of whole blood, plasma, and/or CSF. Non-limiting examples of methods that may be used to measure the levels of one or more proteins (e.g., one or more of CTSB, SPP1, NAGK, YWHAE, AIF1, CSF1, CHIT1, LY86, or CD86) in a sample obtained from the individual include SOMASCAN assay (see, e.g., Candia et al. (2017) Sci Rep 7, 14248), Western blots, mass spectrometry, flow cytometry, and enzyme-linked immunosorbent assay (ELISA) assays.

### Pharmaceutical Compositions

Provided herein are pharmaceutical compositions and/or pharmaceutical formulations comprising the anti-Sortilin antibodies of the present disclosure and a pharmaceutically acceptable carrier.

In some embodiments, pharmaceutically acceptable carrier preferably are nontoxic to recipients at the dosages and concentrations employed. The antibodies described herein may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols. Pharmaceutically acceptable carriers can include, depending on the formulation desired, pharmaceutically-acceptable, non-toxic carriers of diluents, which are vehicles commonly used to formulate pharmaceutical compositions for animal or human administration. In certain embodiments, the pharmaceutical composition can comprise formulation materials for modifying, maintaining or preserving, for example, the pH, osmolarity, viscosity, clarity, color, isotonicity, odor, sterility, stability, rate of dissolution or release, adsorption or penetration of the composition.

In certain embodiments, pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Further examples of formulations that are suitable for various types of administration can be found in Remington: The Science and Practice of Pharmacy, Pharmaceutical Press 22nd ed. (2013). For a brief review of methods for drug delivery, see, Langer, Science 249:1527-1533 (1990).

Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Formulations may be optimized for retention and stabilization in the brain or central nervous system. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the antibody, such as an anti-Sortilin antibody of the present disclosure, in a biodegradable or bioerodible implant. The rate of release of the therapeutically active agent is controlled by the rate of transport through the polymeric matrix, and the biodegradation of the implant. Implants may be particles, sheets, patches, plaques, fibers, microcapsules and the like and may be of any size or shape compatible with the selected site of insertion. Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid.

### Kits/Articles of Manufacture

Provided herein are articles of manufacture (e.g., kit) comprising an anti-Sortilin antibody described herein. Article of manufacture may include one or more containers comprising an antibody described herein. Containers may be any suitable packaging including, but is not limited to, vials, bottles, jars, flexible packaging (e.g., sealed Mylar or plastic bags), and the like. The containers may be unit doses, bulk packages (e.g., multi-dose packages) or sub-unit doses.

In some embodiments, the kits may further include a second agent. In some embodiments, the second agent is a pharmaceutically-acceptable buffer or diluting agent including, but not limited to, such as bacteriostatic water for injection (BWFI), phosphate-buffered saline, Ringer's solution and dextrose solution. In some embodiments, the second agent is a pharmaceutically active agent.

In some embodiments of any of the articles of manufacture, the article of manufactures further include instructions for use in accordance with the methods of this disclosure. The instructions generally include information as to dosage, dosing schedule, and route of administration for the intended treatment. In some embodiments, these instructions comprise a description of administration of the isolated antibody of the present disclosure (e.g., an anti-Sortilin antibody described herein) to prevent, reduce risk, or treat an individual having a disease, disorder, or injury selected from dementia, frontotemporal dementia, Alzheimer's disease, gauche's disease, vascular dementia, seizures, retinal dystrophy, a traumatic brain injury, a spinal cord injury, atherosclerotic vascular diseases, undesirable symptoms of normal aging, amyotrophic lateral sclerosis (ALS), long-term depression, Parkinson's disease, Huntington's disease, Taupathy disease, multiple sclerosis, age related macular degeneration, glaucoma, degenerative disc disease (DDD), Creutzfeldt-Jakob disease, normal pressure hydrocephalus, Nasu-Hakola disease, stroke, acute trauma, chronic trauma, lupus, acute and chronic colitis, Crohn's disease, inflammatory bowel disease, ulcerative colitis, malaria, essential tremor, central nervous system lupus, Behcet's disease, mixed dementia, dementia with Lewy bodies, multiple system atrophy, Shy-Drager syndrome, progressive supranuclear palsy, cortical basal ganglionic degeneration, acute disseminated encephalomyelitis, granulomatous disorders, sarcoidosis, diseases of aging, retinitis pigmentosa, retinal degeneration, respiratory tract infection, sepsis, eye infection, systemic infection, lupus, arthritis, and wound healing, according to any methods of this disclosure. In some embodiments, the disease, disorder, or injury is frontotemporal dementia. In some embodiments, the instructions include instructions for use of the anti-Sortilin antibody and the second agent (e.g., second pharmaceutically active agent).

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Anti-Sortilin Antibody PK and PD in Non-Human Primates

In this Example, the pharmacokinetics (PK) and pharmacodynamics (PD) of intravenously (IV) administered anti-Sortilin antibody S-60-15.1 [N33T] LALAPS were determined in non-human primates.

### Materials and Methods

### Single Dose Pharmacokinetic and Pharmacodynamic Studies

For single dose pharmacokinetic studies, cynomolgus monkeys were administered anti-Sortilin antibody by single IV dose of 5 mg/kg, 20 mg/kg, 60 mg/kg, or 200 mg/kg on Day 0 (n=3 animals per dose). Blood and CSF were drawn from the animals at multiple time-points thereafter to obtain anti-Sortilin antibody concentrations in plasma and cerebrospinal fluid (CSF), which are measurements of anti-Sortilin antibody pharmacokinetics. Progranulin (PGRN) concentration and the levels of Sortilin (SORT1) on white blood cells (WBCs), which are measurements of pharmacodynamics, were also determined.

Anti-Sortilin antibody concentrations were assayed using an ELISA assay with anti-Sortilin antibody-specific anti-idiotypic antibodies. PGRN concentrations were assayed with a commercially-available ELISA kit. Levels of SORT1 on white blood cells were assayed using an ELISA assay, and normalized to protein concentration.

### Results

Table 2 provides the plasma mean Cₘₐₓ, mean AUC, and t_{1/2} for each of the tested anti-Sortilin antibody doses.

TABLE 2
Cₘₐₓ, mean AUC, and t_{1/2} for the indicated anti-
Sortilin antibody doses (n = 3 for each dose).

| Antibody | Mean Cₘₐₓ | Mean AUC | t½ |
|---|---|---|---|
| Dose | (μg/ml) | (μg × hr/ml) | hours |
| 5 mg/kg | 156 | 2,870 | 4.7 |
| 20 mg/kg | 697 | 26,500 | 13.3 |
| 60 mg/kg | 2,570 | 118,000 | 42 |
| 200 mg/kg | 7,910 | 366,000 | 73.6 |

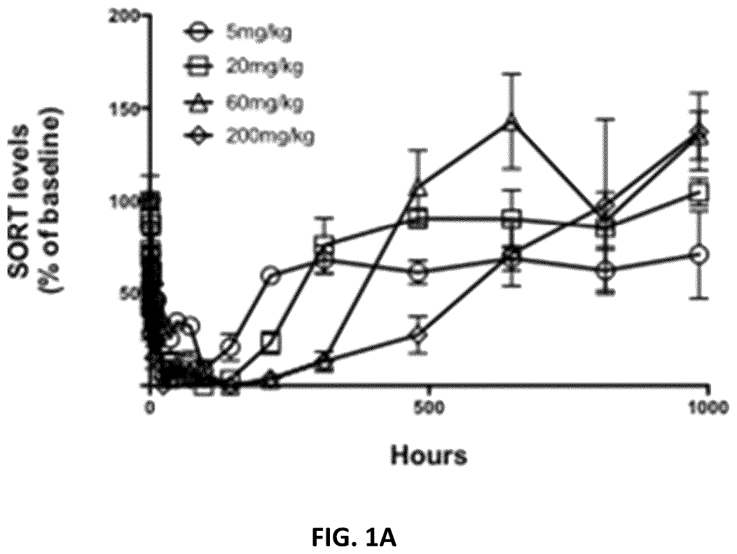

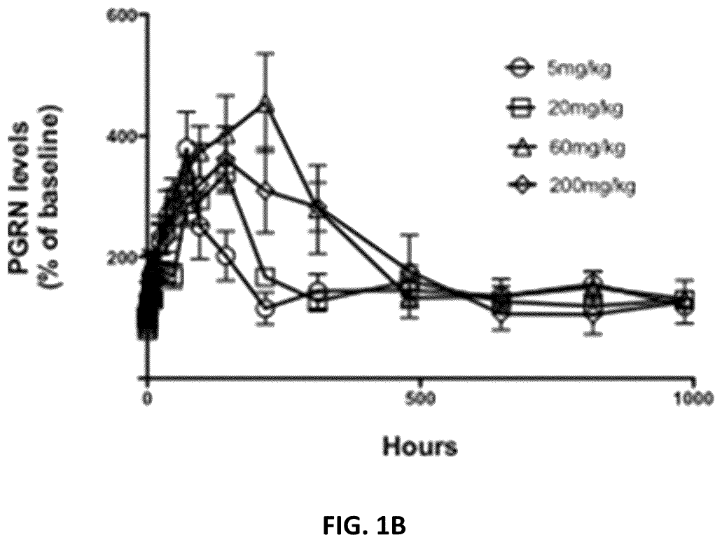

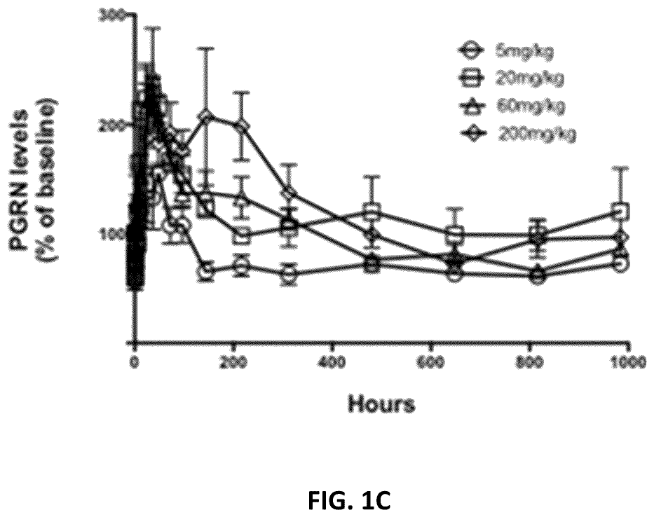

As shown in FIG. 1A, SORT1 expression levels in peripheral white blood cells decreased after treatment of non-human primates with any of the anti-Sortilin antibody doses tested. The higher anti-Sortilin antibody doses (60 mg/kg, 200 mg/kg) resulted in both an earlier and more prolonged decrease of SORT1 levels in peripheral white blood cells compared to lower anti-Sortilin antibody doses (5 mg/kg, 20 mg/kg).

The levels of PGRN increased in the plasma of non-human primates administered a single IV injection of anti-Sortilin antibody in a time- and dose-dependent manner (FIG. 1B). In particular, plasma PGRN levels increased 3- to 4-fold at Cₘₐₓ, compared to baseline levels, for all anti-Sortilin antibody doses tested. Plasma PGRN levels remained elevated for longer periods of time at the higher antibody doses. Additionally, increased plasma PGRN levels were correlated with decreased expression levels of SORT1 in peripheral white blood cells.

The levels of PGRN in CSF were also increased in non-human primates administered a single IV injection of anti-Sortilin antibody. As shown in FIG. 1C, CSF PGRN levels increased 2- to 3-fold above baseline in animals administered either 20 mg/kg, 60 mg/kg, or 200 mg/kg. As observed with plasma PGRN levels, CSF PGRN levels remained elevated over time in the higher antibody dose groups.

Table 3 provides the CSF mean Cₘₐₓ, mean AUC, and t_{1/2} for each of the tested anti-Sortilin antibody doses in non-human primates. Anti-Sortilin antibody CSF concentrations were on average around 0.1% the amount observed in plasma.

TABLE 3
Anti-Sortilin antibody CSF PK parameters and
estimated half-life in non-human primates.

| Dose | Cₘₐₓ | AUCₐₗₗ | CL | t_{1/2} hours | |
|---|---|---|---|---|---|
| Level | (μg/mL) | (h*μg/mL) | (mL/h/kg) | (days) | |
| 5 mg/kg | 20 | 184 | 20692 | 32.3 | (1.34) |
| 20 mg/kg | 2243 | 35717 | 745 | 23.8 | (1) |
| 60 mg/kg | 6842 | 113573 | 623 | 38.3 | (1.6) |
| 200 mg/kg | 4595 | 349187 | 1037 | 72.4 | (3.02) |

### Repeat Dose Pharmacokinetic and Pharmacodynamic Studies

Further pharmacokinetic and pharmacodynamic studies were performed in non-human primates administered anti-Sortilin antibody following a repeat-dose regimen. In these studies, animals (2 males and 2 females) were administered anti-Sortilin antibody at a dose of 60 mg/kg once per week for four weeks. At various timepoints thereafter, SORT1 expression levels in peripheral white blood cells were determined. In addition, plasma and CSF levels of the anti-Sortilin antibody were determined.

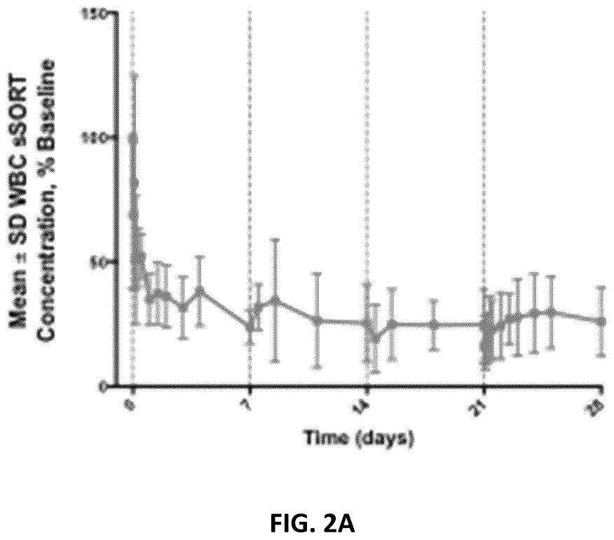

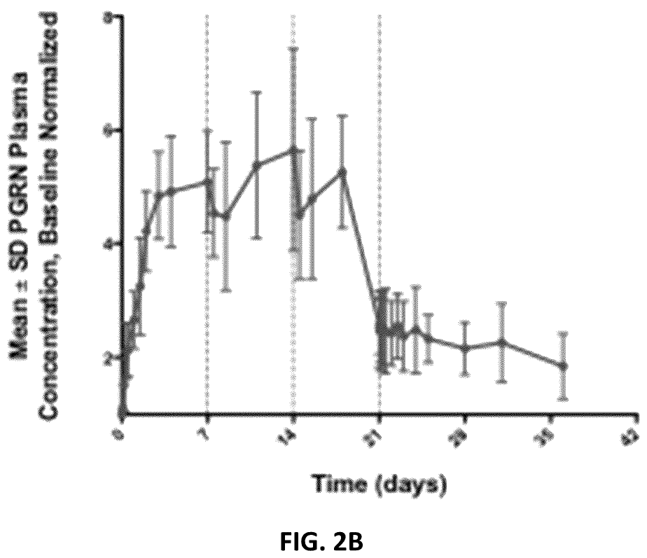

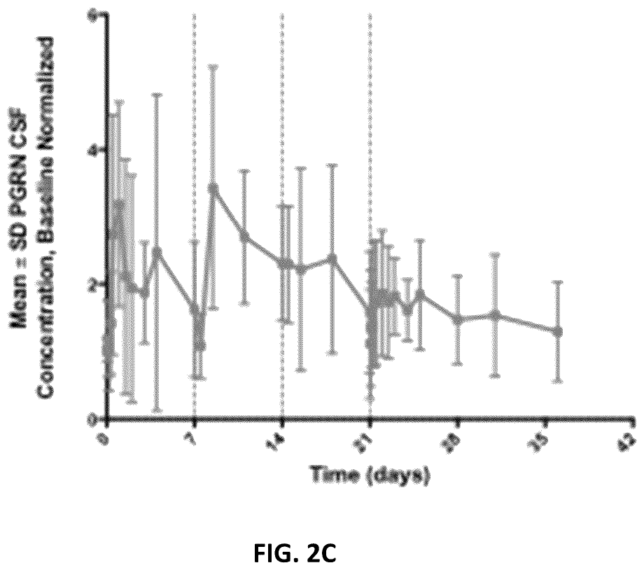

As shown in FIG. 2A, SORT1 levels in peripheral white blood cells remained decreased throughout the duration of the study. Plasma PGRN levels increased to 5- to 6-fold above baseline at peak levels (FIG. 2B). A decrease in plasma PGRN was observed following the fourth and final administration of anti-Sortilin antibody; however, the plasma PGRN levels remained elevated by 2-fold above baseline. Additionally, CSF PGRN levels were increased 3- to 4-fold above baseline (FIG. 2C).

The systemic anti-Sortilin antibody exposure, assessed by mean Cmax and AUC₀₋₁₆₈, was 2100 μg/mL and 114,000 μg/mL×hr on Day 1, and 3020 μg/mL and 174,000 μg/mL×hr on Day 22. These results showed that exposure was higher on Day 22 compared to Day 1, indicating some accumulation of the antibody.

CSF concentration of anti-Sortilin antibody in these animals ranged from 0.03% to 0.12% of that observed in plasma, consistent with the distribution of other antibodies in the CSF (Pestalozzi et al., (2000) J Clin Oncol 18(11):2349-51; Petereit et al., (2009) Mult Scler 15(2):189-92).

### Example 2: Anti-Sortilin Antibody PK and PD in Human Healthy Volunteers

In this Example, the pharmacokinetics and pharmacodynamics of intravenously administered anti-Sortilin antibody S-60-15.1 [N33T] LALAPS in humans were investigated.

### Materials and Methods

To investigate the pharmacokinetics and pharmacodynamics of intravenously administered anti-Sortilin antibody in humans, the following human Phase 1a clinical study was performed:

Six cohorts of both male and female healthy volunteers, aged 18-65, were included in these studies and were administered a single-dose of anti-Sortilin antibody (or placebo control) as an IV infusion over approximately one hour. Each cohort included at least 8 healthy volunteer subjects, with at least 6 subjects administered anti-Sortilin antibody and at least 2 subjects administered placebo control. Antibody dose levels used for the six cohorts were 2 mg/kg, 6 mg/kg, 15 mg/kg, 30 mg/kg, and 60 mg/kg. Two separate cohorts were studied at 60 mg/kg to investigate cerebrospinal fluid (CSF) effects at different post-dose time points, as described below.

Blood was drawn from the human subjects at multiple time-points to obtain anti-Sortilin antibody concentrations in plasma and a lumbar puncture was performed to collect CSF, both for measurements of pharmacokinetics; to obtain SORT1 expression levels on white blood cells (WBCs), a measurement of pharmacodynamics; and to obtain PGRN concentrations, a measurement of pharmacodynamics. For CSF measurements, lumbar punctures were performed on human subjects administered antibody doses of 15 mg/kg or higher. Anti-Sortilin antibody concentrations (PK) and PGRN concentrations (PD) were determined in the CSF samples.

Anti-Sortilin antibody concentrations were assayed using an ELISA assay with anti-Sortilin antibody-specific anti-idiotypic antibodies. PGRN concentrations were assayed with a commercially-available ELISA kit, and levels of SORT1 on white blood cells were assayed using an ELISA assay, and normalized to protein concentration.

In all healthy volunteer cohorts, anti-Sortilin antibody or placebo was administered on Study Day 1, and blood samples were taken from the subjects on Study Days 1, 2, 3, 6, 8, 13, 18, 30, 43, 57, 85, and 113 for PK and PD determinations. CSF samples were obtained on Study Days 1 (pre-dose), 2, and 13 for three cohorts (15 mg/kg, 30 mg/kg, 60 mg/kg cohort). CSF samples were obtained from a second cohort of subjects administered 60 mg/kg on Study Days 1 (pre-dose), 25 and 43.

### Results

A total of fifty healthy volunteers were administered a single dose of anti-Sortilin antibody S-60-15.1 [N33T] LALAPS.

### Pharmacokinetics in Plasma

Plasma PK data from ascending dose cohorts in healthy volunteers, including at least 30—days of post-dose data for all cohorts, are provided in Table 4. Anti-Sortilin antibody administered to healthy volunteers displayed an approximate dose-proportional Cₘₐₓ (i.e., 47.2 g/mL at 2 mg/kg; 1540 g/mL at 60 mg/kg). The results also showed that upon increasing dose levels of anti-Sortilin antibody from 2 mg/kg to 60 mg/kg, plasma clearance of the antibody decreased, plasma half-life increased, and total plasma exposure (calculated as AUC_{0-inf}) increased in a non-linear fashion. Notably, plasma terminal half-life of anti-Sortilin antibody was short at all doses tested, ranging from 29.6 hours (1.2 days) at the 2 mg/kg dose to 190 hours (7.9 days) at the 60 mg/kg dose.

TABLE 4
Plasma pharmacokinetics of anti-Sortilin antibody administered as a
single dose (mean values presented for each dose level).

| | | AUC_{0-inf} | | t_{1/2} hours |
|---|---|---|---|---|
| Dose Level | Cₘₐₓ (μg/mL) | (h*μg/mL) | CL (L/h) | (days) |
| 2 mg/kg | 47.2 | 2,700 | 0.0531 | 29.6 hours |
| | | | | (1.2 days) |
| 6 mg/kg | 140 | 13,200 | 0.0424 | 51.2 hours |
| | | | | (2.1 days) |
| 15 mg/kg | 412 | 52,400 | 0.0232 | 86.4 hours |
| | | | | (3.6 days) |
| 30 mg/kg | 830 | 135,000 | 0.0181 | 119 hours |
| | | | | (5 days) |
| 60 mg/kg #1 | 1540 | 307,000 | 0.0138 | 190 hours |
| | | | | (7.9 days) |

Cmax = maximum concentration;
AUC_{0-inf} = AUC from time 0 extrapolated to infinity;
CL = clearance;
t_{1/2}: terminal half-life.

A further analysis of the plasma PK results in Table 4 is provided in Table 5.

TABLE 5
Further analysis of plasma pharmacokinetics of
anti-Sortilin antibody administered as a single dose
(mean values presented for each dose level).

| | | Cₘₐₓ | AUC_{inf} | CL | t_{1/2} |
|---|---|---|---|---|---|
| Dose Level (n) | | (μg/mL) | (h*μg/mL) | (mL/h) | (days) |
| 2 | mg/kg (7) | 46.8 | 2,640 | 51.5 | 1.2 |
| 6 | mg/kg (6) | 128 | 12,900 | 40.6 | 2.0 |
| 15 | mg/kg (6) | 409 | 52,100 | 23.0 | 3.5 |
| 30 | mg/kg (6) | 819 | 134,000 | 18.0 | 4.8 |
| 60 | mg/kg (12) | 1640 | 327,000 | 13.4 | 6.7 |

Geometric means provided.

Taken together, these results indicated that that at the doses tested, anti-Sortilin antibody is cleared more rapidly that other therapeutic antibodies of similar class, thus demonstrating that, unexpectedly, anti-Sortilin antibody showed a shorter half-life of this antibody compared to others of similar class (Ovacik, M and Lin, L, (2018) Clin Transl Sci 11, 540-552). The short half-life of the antibody suggested that it may not be useful therapeutically.

### Pharmacokinetics in CSF

Preliminary CSF PK data for the three single-ascending dose healthy human volunteer cohorts for which CSF was collected are shown below in Table 6.

CSF concentrations of anti-Sortilin antibody showed a decrease over time from 30-hours post-dose to 12—days post-dose in both the 15 mg/kg and 30 mg/kg cohorts (Table 6). These results indicated that anti-Sortilin antibody concentration in CSF peaked at a time prior to 12—days post-dose in healthy volunteers administered either 15 mg/kg or 30 mg/kg antibody. In contrast, CSF concentrations of anti-Sortilin antibody increased from 30-hours post-dose to 12—days post-dose in the 60 mg/kg cohorts (Table 6).

TABLE 6
CSF concentrations of anti-Sortilin antibody
(ng/mL) administered as a single dose
(mean values presented for each level).

| | | 30-hours | 12-days |
|---|---|---|---|
| | | Post-Dose | Post-Dose |
| | | Nominal Time | Nominal Time |
| Dose Level | Pre-Dose | (μg/ml) | (μg/ml) |
| 15 mglkg | 0 (N/A) | 42.4 | 35.6 |
| 30 mg/kg | 0 (N/A) | 264 | 214 |
| 60 mmg/kg #1 | 0 (N/A) | 587 | 973 |

Additionally, CSF concentrations of the antibody from a second 60 mg/kg cohort of healthy volunteers were measured at 24—days and 42—days post-dose, revealing that anti-Sortilin antibody was present in the CSF as much as 42—days post-dose (Table 7).

TABLE 7
CSF concentrations of anti-Sortilin antibody (ng/mL) administered
as a single dose (mean values presented for each level).

| | | 24-days Post-Dose | 42-days Post-Dose |
|---|---|---|---|
| | | Nominal Time | Nominal Time |
| Dose Level | Pre-Dose | (μg/ml) | (μg/ml) |
| 60 mg/kg #2 | 0 (N/A) | 243.0 | 37.8 |

The ratio of the percentage of CSF concentration to plasma concentration of anti-Sortilin antibody for the 15 mg/kg, 30 mg/kg, and 60 mg/kg doses was determined, and the results are provided in Table 8.

As shown in Table 8, anti-Sortilin antibody concentrations in CSF at 12 days post-dose were 0.09% of that observed in plasma at the 15 mg/kg dose, 0.12% of that observed in plasma at the 30 mg/kg dose, and 0.26% of that observed in plasma at the 60 mg/kg dose. These results indicated that higher central nervous system penetrance of anti-Sortilin antibody was observed with increased doses.

TABLE 8
Percentage of CSF concentration to plasma concentration of
anti-Sortilin antibody administered as a single dose (mean
values presented for each level).

| | 30-hours Post-Dose | 12-days Post-Dose |
|---|---|---|
| Dose Level | Nominal Time | Nominal Time |
| 15 mg/kg | 0.01% | 0.09% |
| 30 mg/kg | 0.04% | 0.12% |
| 60 mg/kg #1 | 0.05% | 0.26% |

A further analysis of the percentage of CSF concentration to plasma concentration of anti-Sortilin antibody for the 15 mg/kg, 30 mg/kg, and 60 mg/kg doses is provided in Table 9.

TABLE 9
Further analysis of the percentage of CSF concentration to plasma
concentration of anti-Sortilin antibody administered as a single
dose mean values presented for each level).

| Dose Level | Day 2 | Day 13 |
|---|---|---|
| 15 mg/kg | 0.01 | 0.07 |
| 30 mg/kg | 0.04 | 0.12 |
| 60 mg/kg | 0.05 | 0.27 |

Taken together, these results indicated that anti-Sortilin antibody entered the CSF in a similar proportion to other IgG antibodies, displaying a % CSF PK to plasma PK consistent with other therapeutic monoclonal antibodies.

### Pharmacodynamics in Blood

The effect of anti-Sortilin antibody on SORT1 levels on peripheral white blood cells and on plasma PGRN concentration levels was determined. In these studies, SORT1 and PGRN levels were determined from 5 healthy volunteer cohorts (2 mg/kg, 6 mg/kg, 15 mg/kg, 30 mg/kg, and 60 mg/kg).

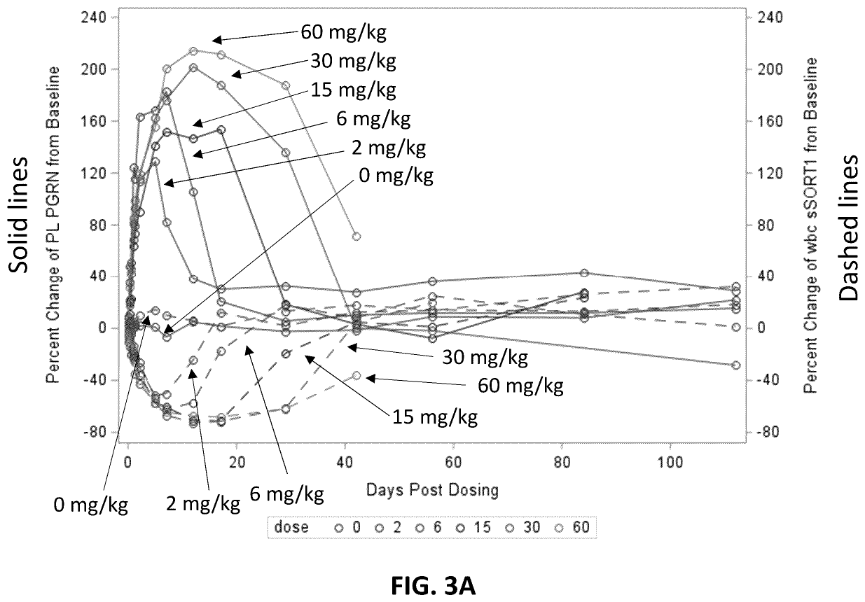

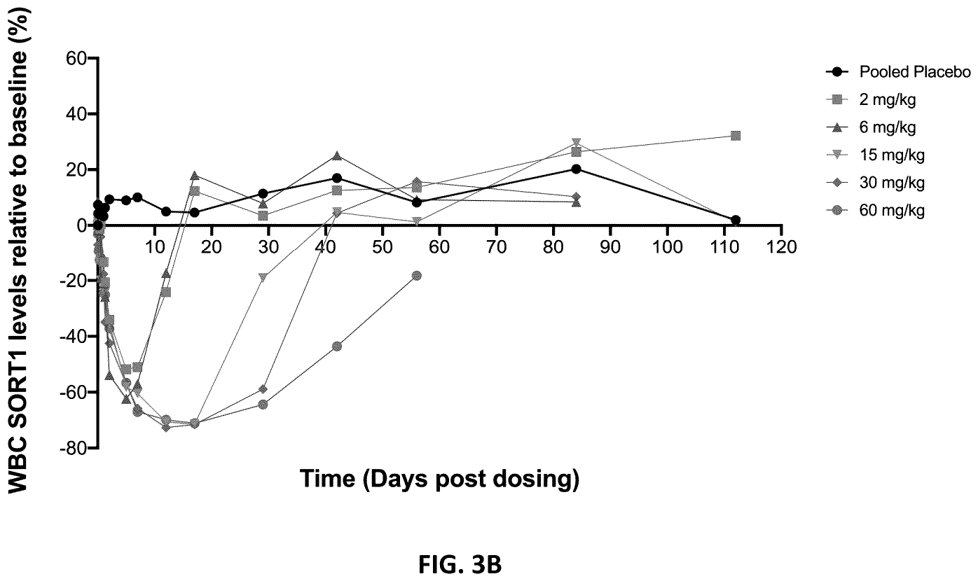

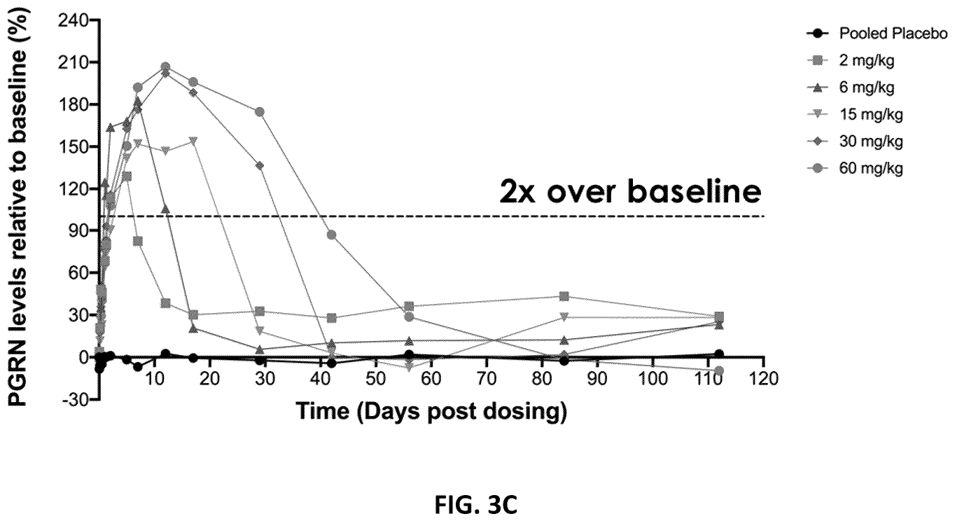

As shown in FIG. 3A (dashed lines), administration of anti-Sortilin antibody to human subjects resulted in a decrease in SORT1 expression levels on peripheral white blood cells.

For example, subjects administered an anti-Sortilin antibody dose of 2 mg/kg showed a maximum decrease in SORT1 expression levels on peripheral white blood cells of approximately 50% from baseline levels at 5-7 days post antibody administration. Subjects administered an anti-Sortilin antibody dose of 6 mg/kg, 15 mg/kg, 30 mg/kg, or 60 mg/kg showed a maximum decrease in SORT1 expression levels on peripheral white blood cells of approximately 70% from baseline levels at 12-17 days post antibody administration. The decreases in SORT1 expression levels on peripheral white blood cells were sustained for longer periods of time following antibody administration with each increased dose of anti-Sortilin antibody. The longest sustained decrease in SORT1 expression levels occurred more than 40 days after antibody administration in the 60 mg/kg group.

A further analysis of SORT1 expression levels on peripheral white blood cells following administration of anti-Sortilin antibody to human subjects is provided in FIG. 3B.

Additionally, as shown in FIG. 3A (solid lines), administration of anti-Sortilin antibody to human subjects resulted in an increase in plasma PGRN levels.

For example, increased plasma PGRN concentration levels were observed in all human subjects administered a single IV dose of anti-Sortilin antibody. As shown in FIG. 3A, increased plasma PGRN concentration levels were observed in subjects at all anti-Sortilin antibody doses. Maximum concentrations of plasma PGRN were seen at 5 to 12 days following antibody administration. The maximum increase in percent change from baseline levels was statistically significant compared to pooled placebo samples for each of the 5 cohorts; increases in plasma PGRN concentration levels ranged from 1.29 to 2.14-fold above baseline (a 1-fold increase from baseline corresponds to a 100% increase from baseline). Plasma PGRN levels remained elevated for increasingly longer durations after anti-Sortilin antibody administration in a dose-dependent manner. The duration of increased plasma PGRN levels ranged from 40 days to 42 days or more at anti-Sortilin antibody doses of 30 mg/kg and 60 mg/kg, indicating that the observed increases in plasma PGRN levels were more sustained at the highest antibody dose levels.

A further analysis of plasma PGRN levels following administration of anti-Sortilin antibody to human subjects is provided in FIG. 3C.

### Pharmacodynamics in CSF

The effect of anti-Sortilin antibody on PGRN concentration levels in CSF was also determined. Pharmacodynamic data for CSF PGRN concentration levels were obtained from 4 cohorts of healthy volunteers dosed at 15 mg/kg, 30 mg/kg, or 60 mg/kg. For three of the cohorts (15 mg/kg, 30 mg/kg, and 60 mg/kg), CSF samples were collected from human subjects at pre-dose, and then at approximately 30-hours (on day 2) and 12—days after antibody administration (on day 13). In these three cohorts, six subjects received placebo and CSF samples were obtained from them at approximately 30-hours and 12—days following placebo administration. A fourth cohort was dosed at 60 mg/kg and CSF samples were obtained from these subjects at pre-dose and on day 25 and day 43. Two subjects in this fourth cohort received placebo and CSF samples were obtained from them at pre-dose and on day 25 and day 43. This additional cohort of 60 mg/kg was added to the study to further assess the duration of the effect of the anti-Sortilin antibody on CSF PGRN concentration levels.

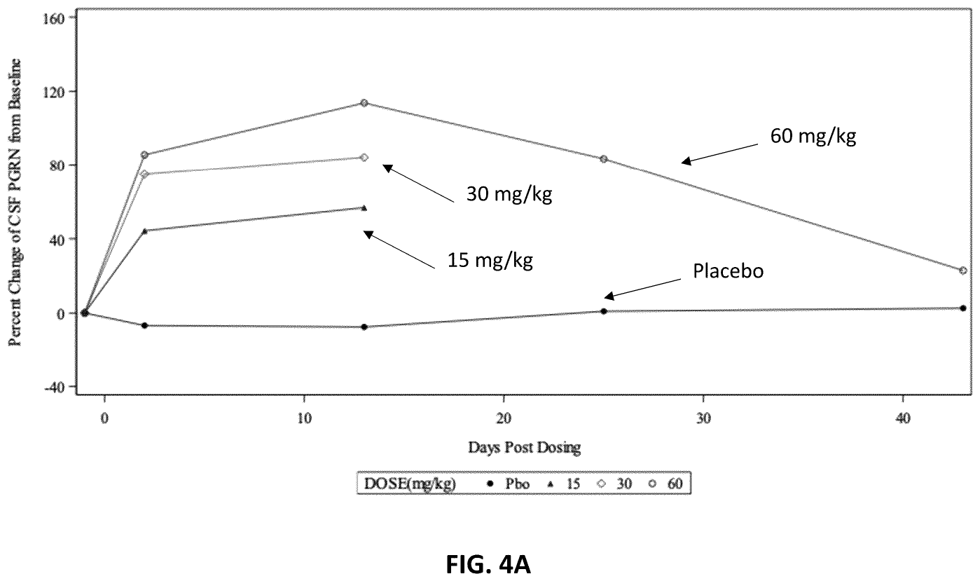

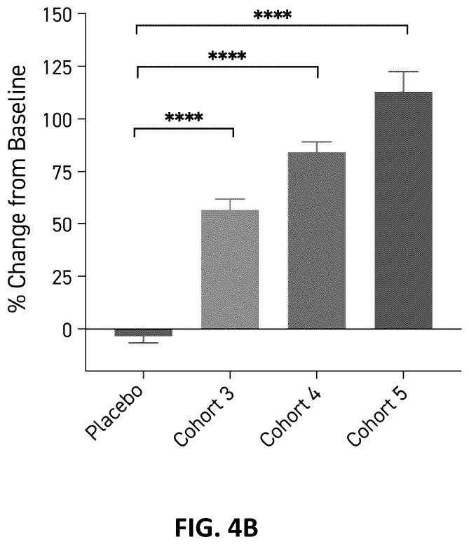

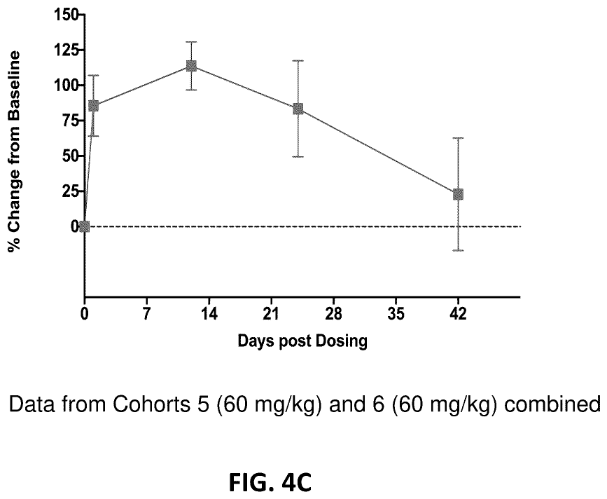

As shown in FIG. 4A, a statistically significant increase in CSF PGRN concentration levels (compared to PGRN concentration levels observed at baseline) was seen at both examined post-dose time points (30—hours and 12—days) for the first three cohorts. A maximum increase in CSF PGRN levels was observed 12—days post anti-Sortilin antibody administration. At 12—days post anti-Sortilin antibody administration, CSF PGRN concentration levels increased 0.57-fold for the 15 mg/kg dose, 0.84-fold for the 30 mg/kg dose, and 1.13-fold for the 60 mg/kg dose compared to baseline (a 1-fold increase from baseline corresponds to a 100% increase from baseline). A bar graph showing the percent change from baseline in CSF PGRN levels for the 15 mg/kg, 30 mg/kg, and 60 mg/kg cohorts is provided in FIG. 4B.

As stated above, CSF samples were obtained from subjects from the fourth cohort (60 mg/kg) at pre-dose and at days 25 and 43 (i.e., 24 and 42 days after antibody administration). Mean increases of 0.83-fold and 0.23-fold in CSF PGRN concentration levels compared to baseline were observed on day 25 and day 43, respectively. These results are shown in FIG. 4A as the percent change from baseline at day 25 and day 43 for 60 mg/kg dose and for placebo.

In addition, PGRN levels were analyzed in CSF samples obtained from subjects in both 60 mg/kg cohorts from pre-dose to 42—days post dose. These results are shown in FIG. 4C as the percent change from baseline.

These results showed that anti-Sortilin antibody administration increased concentration levels of CSF PGRN in humans, and that the increased concentration levels of CSF PGRN were sustained for at least 24—days after a single IV dose of anti-Sortilin antibody at 60 mg/kg.

In summary, in spite of its having a short half-life in plasma, administration of S-60-15.1 [N33T]LALAPS showed promising pharmacodynamics effects in humans such as a reduction of SORT1 expression on white blood cells and increases in PGRN levels in plasma and in the CSF. Unexpectedly, these therapeutic effects were sustained for a long duration in the human subjects. Thus, further study of the antibody in humans was pursued.

### Safety Summary

Anti-Sortilin antibody S-60-15.1 [N33T] LALAPS was generally safe and well-tolerated at all of the administered doses. No dose-limiting adverse effects, drug-related serious adverse events (SAEs), or dose limiting toxicities (DLTs) were observed. Most of the treatment emergent adverse events (TEAEs) were of mild or moderate severity. There were no apparent dose-dependent trends in adverse events. The most common TEAEs were post lumbar puncture syndrome (lumbar punctures were performed starting at the 15 mg/kg dose level), puncture site pain, headache, anemia, and vomiting. Table 10 displays the observed adverse events in the Phase 1 study.

TABLE 10
Safety analysis of Anti-Sortilin antibody S-60-15.1 [N33T] LALAPS administered
at the indicated doses.

| | Placebo | 2 mg/kg | 6 mg/kg | 15 mg/kg | 30 mg/kg | 60 mg/kg |
|---|---|---|---|---|---|---|
| | n (%) [E] | n (%) [E] | n (%) [E] | n (%) [E] | n (%) [E] | n (%) [E] |
| Healthy | 12 | 7 | 6 | 6 | 6 | 13 |
| Volunteers | | | | | | |
| Exposed | | | | | | |
| Any TEAE | 8 (66.7) [18] | 2 (28.6) | 5 (83.3) | 4 (66.7) | 5 (83.3) [16] | 8 (61.5) [15] |
| | | [5] | [9] | [7] | | |
| Any | 1 (8.3) [1] | 0 | 0 | 0 | 1 (16.7) [1] | 0 |
| Treatment- | | | | | | |
| Related | | | | | | |
| TEAE | | | | | | |

Severity of TEAEs

| | | | | | | |
|---|---|---|---|---|---|---|
| Mild (WHO | 0 [4] | 0 [1] | 2 (33.3) | 1 (16.7) | 0 [6] | 3 (231) [4] |
| Grade 1) | | | [4] | [4] | | |
| Moderate | 7 (58.3) [13] | 2 (28.6) | 3 (50.0) | 3 (50.0) | 5 (83.3) [10] | 4 (30.8) [10] |
| (WHO | | [4] | [5] | [3] | | |
| Grade 2) | | | | | | |
| Severe | 0 | 0 | 0 | 0 | 0 | 0 |
| (WHO | | | | | | |
| Grade 3) | | | | | | |
| Life | 1 (8.3) [1] | 0 | 0 | 0 | 0 | 1 (7.7) [1] |
| Threatening | | | | | | |
| (WHO | | | | | | |
| Grade 4) | | | | | | |

Most common TEAE

| | | | | | | |
|---|---|---|---|---|---|---|
| Post lumbar | 2 (16.7) [4] | 0 | 0 | 1 (16.7) | 3 (50.0) [3] | 3 (23.1) [3] |
| puncture | | | | [1] | | |
| syndrome* | | | | | | |
| Puncture site | 2 (16.7) [2] | 0 | 0 | 0 | 0 | 3 (23.1) [3] |
| pain | | | | | | |
| Headache | 0 | 0 | 0 | 0 | 3 (50.0) [3] | 1 (7.7) [1] |
| Anemia | 1 (8.3) [1] | 0 | 0 | 1 (16.7) | 1 (16.7) [1] | 0 |
| | | | | [1] | | |
| Vomiting | 0 | 1 (14.3) | 0 | 0 | 1 (16.7) [1] | 1 (7.7) [1] |
| | | [1] | | | | |

### Phase 1b Study

In an ongoing open label Phase 1b study, asymptomatic carriers of Granulin mutations (aFTD-GRN) were administered a single dose of anti-Sortilin antibody S-60-15.1 [N33T] LALAPS at 60 mg/kg. The CSF was sampled pre-dose and at 12 days and 24 days post-dose (on study day 1 (pre-dose) and at study days 13 and 25). Symptomatic carriers of Granulin mutations (FTD-GRN) were administered three doses of anti-Sortilin antibody S-60-15.1 [N33T] LALAPS at 30 mg/kg, q2w (every two weeks). The CSF was sampled pre-dose and 56 days post-dose (on study day 1 (pre-dose) and on study day 57), or about 4 weeks after the last dose. Plasma samples were obtained at several timepoints during the study to analyze PGRN levels. The objectives of this study were to assess safety and tolerability, pharmacokinetics, and pharmacodynamics in Granulin mutation carriers and Granulin mutation FTD patients. The exploratory objectives of this study included analysis of biomarkers.

### Results

### Study Subjects

Three aFTD-GRN subjects were administered a single IV dose of anti-Sortilin antibody S-60-15.1 [N33T] LALAPS at 60 mg/kg.

Six FTD-GRN patients were administered three IV doses of anti-Sortilin antibody S-60-15.1 [N33T] LALAPS at 30 mg/kg, q2w (every two weeks).

Anti-Sortilin antibody S-60-15.1 [N33T] LALAPS was generally safe and well-tolerated in GRN carriers.

### Plasma PGRN Levels

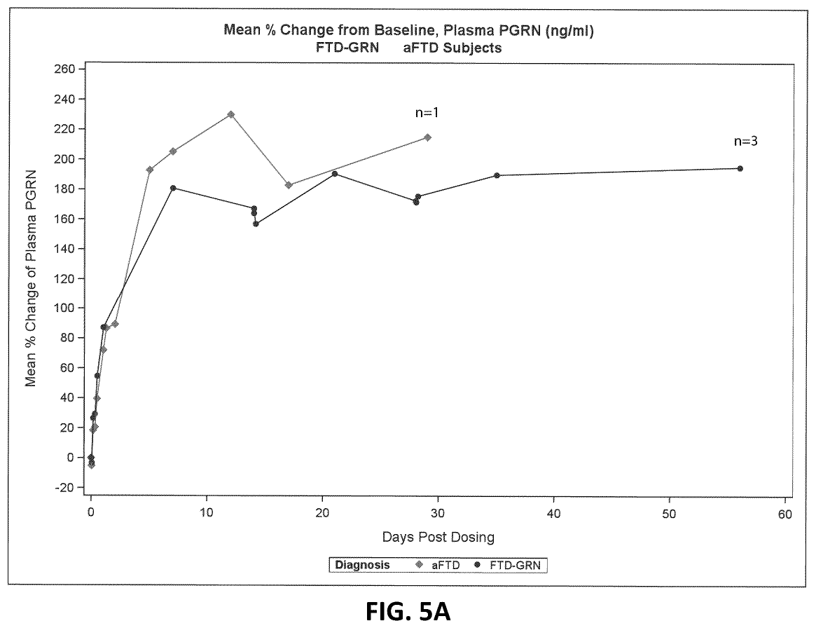

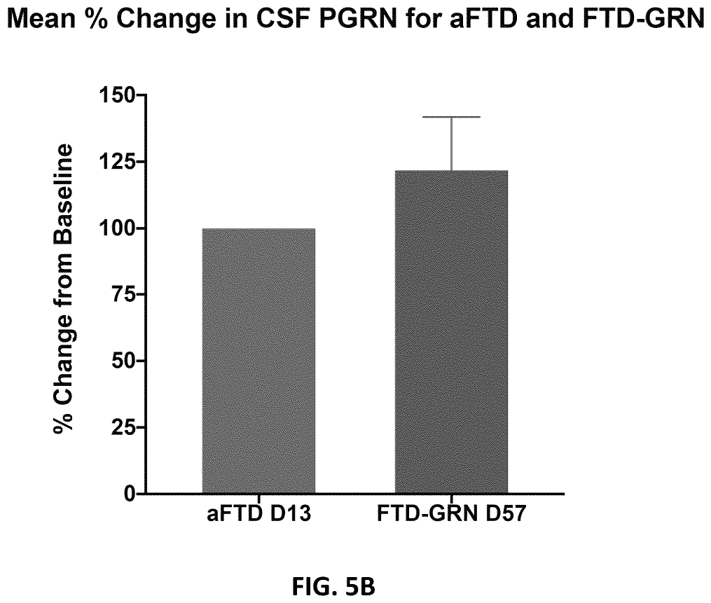

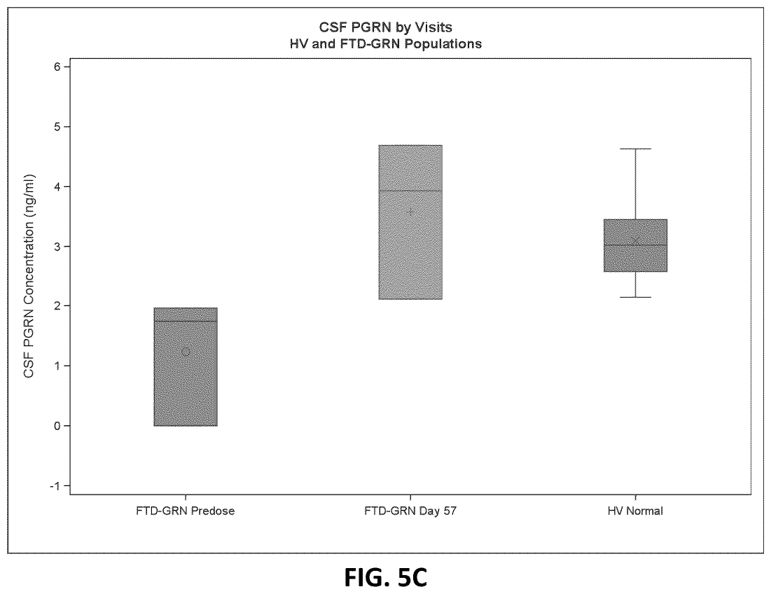

The percent change in plasma PGRN levels at the indicated days post-dosing are provided in FIG. 5A for one aFTD-GRN subject and three FTD-GRN patients.

### CSF PGRN Levels

The percent change in CSF PGRN levels in one aFTD-GRN subject (study day 13) and three FTD-GRN patients (study day 57) are provided in FIG. 5B.

The concentration of PGRN in CSF (ng/mL) from normal healthy volunteers and from three FTD-GRN patients pre-dose and on study day 57 are provided in FIG. 5C.

### Conclusions

The results thus far of this ongoing Phase 1b study show that anti-Sortilin antibody S-60-15.1 [N33T] LALAPS is generally safe and well tolerated up to the highest dose level of 60 mg/kg. In addition, the results show that anti-Sortilin antibody 5-60-15.1 [N33T] LALAPS causes dose-dependent and long lasting increases in PGRN levels in both plasma and CSF of GRN mutation carriers (FIGS. 5A-5B). Moreover, anti-Sortilin antibody S-60-15.1 [N33T] LALAPS restored PGRN levels in the CSF of FTD-GRN patients to levels comparable to the normal range exhibited by normal healthy volunteers (FIG. 5C).

### Example 3: Phase 2 Study to Evaluate Anti-Sortilin Antibody in Heterozygous Carriers of Granulin or C9orf72 Mutations Causative of Frontotemporal Dementia

This Example describes a Phase 2, multicenter, open-label study to evaluate the safety, tolerability, pharmacokinetics, and pharmacodynamics of anti-Sortilin antibody 5-60-15.1 [N33T] LALAPS in heterozygous carriers of Granulin or C9orf72 mutations causative of frontotemporal dementia (FTD).

### Study Objectives

### Primary Objective

The primary objective of this study is to evaluate the safety and tolerability of intravenous (IV) administration of anti-Sortilin antibody over up to 48 weeks in asymptomatic and symptomatic carriers of a GRN mutation causative of FTD and in symptomatic carriers of a C9orf72 mutation causative of FTD.

### Secondary Objectives

The secondary objectives of this study are to evaluate the effect of IV administration of anti-Sortilin antibody over up to 48 weeks in asymptomatic and symptomatic carriers of a GRN mutation causative of FTD and in symptomatic carriers of a C9orf72 mutation causative of FTD based on the following:
Pharmacokinetics (PK).
Pharmacodynamics (PD) biomarkers:
Longitudinal plasma and CSF PGRN concentration levels.
Longitudinal levels of SORT1 on white blood cells (WBCs) and soluble SORT1 (sSORT1) levels in CSF.

### Exploratory Objectives

The exploratory objectives of this study are to assess the effect of IV administration of anti-Sortilin antibody over up to 48 weeks in asymptomatic and symptomatic carriers of a GRN mutation causative of FTD and in symptomatic carriers of a C9orf72 mutation causative of FTD based on the following:
PD biomarkers:
Longitudinal blood, plasma, and CSF concentration levels of exploratory biomarkers of neurodegeneration, lysosomal function, and microglial activity.
Magnetic Resonance Imaging (MRI) measures to evaluate changes in the brain.
Brain microglial activation.
   Correlations among exploratory fluid PD biomarkers, imaging PD measures, and Clinical Outcome Assessments (COAs).

The Exploratory Clinical Objective of this study is clinical progression as measured by COAs.

### Study Participants

Approximately 32 participants in two cohorts are enrolled in this study:
GRN Cohort (up to 24 asymptomatic and symptomatic participants; specifically, about 6 asymptomatic participants and about 18 symptomatic participants), including:
Asymptomatic and symptomatic participants in a previous Phase 1 study of anti-Sortilin antibody in healthy volunteers and heterozygous GRN mutation carriers (hereinafter referred to as “the previous Phase 1 study of anti-Sortilin antibody”).
New symptomatic GRN mutation carriers.
   C9orf72 Cohort (up to 8 symptomatic patients).

Participants are assigned to study treatment only if they meet all of the inclusion criteria and none of the exclusion criteria.

### Inclusion Criteria

Each participant meets all of the following criteria to be enrolled in this study:

### Key Inclusion Criteria

Participant Category 1: GRN Mutation Carriers, Symptomatic, from the previous Phase 1 study of anti-Sortilin antibody:
Patient completed the previous Phase 1 study of anti-Sortilin antibody through the Day 57 visit and did not experience adverse events (AEs) that the investigator deems would prevent safe participation in this study.
   All patients from the previous Phase 1 study of anti-Sortilin antibody are rescreened and meet all inclusion/exclusion criteria applicable to this study.
   Patient meets diagnostic criteria for possible behavioral variant FTD (bvFTD) or probable bvFTD (Rascovsky et al., (2011) Brain 134(9):2456-2477) or primary progressive aphasia (PPA) (Gorno et al., (2011) Neurology 76(11):1006-1014). Patients with mild symptomatology, not significantly affecting activities of daily living (e.g., mild cognitive impairment, mild behavioral impairment), bvFTD patients if they have 1 or more of the 6 behavioral/cognitive symptoms required for a diagnosis of possible bvFTD (Rascovsky et al., (2011) Brain 134(9):2456-2477). bvFTD or PPA patients with concomitant motor neuron disease.

Participant Category 2: GRN Mutation Carriers, Asymptomatic, from the previous Phase 1 study of anti-Sortilin antibody:
Participant completed the previous Phase 1 study of anti-Sortilin antibody through the Day 43 visit and did not experience AEs that the investigator deems would prevent safe participation in this study.
All participants from the previous Phase 1 study of anti-Sortilin antibody are rescreened and meet all inclusion/exclusion criteria applicable to this study.

Participant Category 3: GRN Mutation Carriers, Symptomatic, New:
Patient is a carrier of a loss-of-function GRN mutation causative of FTD-GRN and knows their mutation status.
Patient meets diagnostic criteria for possible bvFTD or probable bvFTD (Rascovsky et al., (2011) Brain 134(9):2456-2477) or PPA (Gorno et al., (2011) Neurology 76(11):1006-1014). Patients with mild symptomatology, not significantly affecting activities of daily living (e.g., mild cognitive impairment, mild behavioral impairment). bvFTD patients if they have 1 or more of the 6 behavioral/cognitive symptoms required for a diagnosis of possible bvFTD (Rascovsky et al., (2011) Brain 134(9):2456-2477). bvFTD or PPA patients with concomitant motor neuron disease.
Patient is of mild severity, as defined by:
A Clinical Dementia Rating Scale (CDR) global score of 1 or less, and
A box score of 1 or less on both the Language domain, and the Behavior, Comportment and Personality domain of the Frontotemporal Dementia Clinical Rating Scale (FCRS).

Participant Category 4: C9orf72 Mutation Carriers, Symptomatic, New
Patient is a carrier of a hexanucleotide repeat expansion C9orf72 mutation causative of FTD-C9orf72 and knows their mutation status.
Patient meets diagnostic criteria for possible bvFTD or probable bvFTD (Rascovsky et al., (2011) Brain 134(9):2456-2477) or PPA (Gorno et al., (2011) Neurology 76(11):1006-1014). Patients with mild symptomatology, not significantly affecting activities of daily living (e.g., mild cognitive impairment, mild behavioral impairment). bvFTD patients if they have 1 or more of the 6 behavioral/cognitive symptoms required for a diagnosis of possible bvFTD (Rascovsky et al., (2011) Brain 134(9):2456-2477). bvFTD or PPA patients with concomitant motor neuron disease.
Patient is of mild severity, as defined by:
A CDR global score of 1 or less, and
A box score of 1 or less on both the Language domain, and the Behavior, Comportment and Personality domain of the FCRS.

### General Inclusion Criteria

Each participant also meets all of the following criteria to be enrolled in this study:
Participants are 18 to 80 years of age, inclusive, at screening.
At screening, female participants are non-pregnant and non-lactating, and at least one of the following conditions applies:
Participant is not a woman of childbearing potential (WOCBP).
Participant is a WOCBP and using an acceptable contraceptive method from screening until 90 days after the follow-up visit.
A WOCBP has a serum pregnancy test conducted at screening.
Male participants, if not surgically sterilized, agree to use acceptable contraception and not donate sperm from Day 1 until 90 days after the follow-up visit.
Participant is in good physical health on the basis of no clinically significant findings from medical history, physical exams (PEs), laboratory tests, electrocardiogram (ECGs), and vital signs.
Participant is willing and able to comply with the study protocol.
Participant has availability of a person (“study partner”) who has frequent and sufficient contact with the patient (e.g., ≥10 hours per week of in-person contact), can provide accurate information regarding the participant's cognitive and functional abilities, agrees to provide information at site visits that require partner input for COA completion, and signs the necessary consent form.

### Exclusion Criteria

Participants meeting any of the following criteria are excluded from the study:
Participant has a known history of severe allergic, anaphylactic, or other hypersensitivity reactions to chimeric, human, or humanized antibodies or fusion proteins.
Participant has history of alcohol or substance use disorder (according to the DSM-5, American Psychiatric Association, 2013) within the past 2 years.
   Nicotine use is allowed.
   Participant has donated or lost more than 100 mL of blood within 30 days prior to Day 1.
   Participant has had a blood transfusion within 30 days prior to screening.
   Participant has had clinically significant and/or acute illness within 5 days prior to drug administration that may affect safety assessments.
   Participant had surgery, hospitalization, or clinically significant infection requiring oral or IV antibiotics during the 30 days prior to screening.
   Participant has planned procedure or surgery during the study that interferes with the ability to perform study assessments.
   Participant has past history of seizures, with the exception of childhood febrile seizures.
   Participant has clinically significant systemic immunocompromised condition because of continuing effects of immune-suppressing medication.
   Participant has major depressive disorder (unless in remission and treated at enrollment and throughout the study) or history of schizophrenia, schizoaffective disorder, or bipolar disorder (regardless of current or past treatment).
   Participant has history of cancer except:
   If clinically cured.
   Is not being actively treated with anticancer therapy or radiotherapy and is not likely to require treatment in the ensuing 3 years.
   Is considered to have low probability of recurrence.
   Participant has history or presence of intracranial tumor that is clinically relevant (e.g., glioma, cerebral metastasis).
   Participant has any clinically significant medical condition or laboratory abnormality that precludes the participant's safe participation in and completion of the study.
   Participant is positive for hepatitis B surface antigen, hepatitis C virus antibodies, or human immunodeficiency virus-1 and -2 antibodies or antigen, or has history of spirochetal infection of the CNS (e.g., syphilis or borreliosis).
   Participant has significant kidney disease as indicated by a screening for creatinine clearance result of <30 m/min, as calculated by the central laboratory using the Cockcroft-Gault formula, and which remains <30 m/min if retested.
   Participant has impaired hepatic function as indicated by a screening for aspartate aminotransferase (AST) or alanine aminotransferase (ALT) with a result of ≥2× the upper limit of normal (ULN) or total bilirubin ≥1.5×ULN, and which remains above either of these limits if retested; other abnormalities in synthetic function that are clinically significant.
   The participant has, within the last 2 years, had unstable or clinically significant cardiovascular disease (e.g., myocardial infarction, angina pectoris, New York Heart Association Class III or more cardiac failure).
   Participant has uncontrolled hypertension (e.g., blood pressure (BP) generally >140 mm Hg systolic or >90 mm Hg diastolic).
   Participant has history or presence of an abnormal ECG that is clinically significant, including complete left bundle branch block, second- or third-degree heart block, or evidence of prior myocardial infarction.
   Participant has QT interval corrected using Fridericia formula (QTcF) that is >450 ms for male participants and >470 ms for female participants, as demonstrated by at least 2 ECGs 5 minutes apart.
   Participant has history of ventricular dysrhythmias or risk factors for ventricular dysrhythmias such as structural heart disease (e.g., severe left ventricular systolic dysfunction, left ventricular hypertrophy), coronary heart disease (symptomatic or with ischemia demonstrated by diagnostic testing), clinically significant electrolyte abnormalities (e.g., hypokalemia, hypomagnesemia, hypocalcemia), or family history of sudden unexplained death or long QT syndrome.
   Participant has contraindication to lumbar dural puncture, including coagulopathy, concomitant anticoagulation (except for a platelet inhibitor such as aspirin), thrombocytopenia, or other factor that precludes safe lumbar puncture.
   Participant has dementia or a milder, symptomatic syndrome (e.g., mild cognitive impairment, mild behavioral impairment, or mild motor impairment) due to a condition other than FTD, including, but not limited to, Alzheimer's disease, Parkinson's disease, dementia with Lewy bodies, Huntington disease, or vascular dementia.
   Participant has history or presence of clinically evident vascular disease potentially affecting the brain (e.g., clinically significant carotid or vertebral artery stenosis or plaque; aortic aneurysm; intracranial aneurysm; cerebral hemorrhage; arteriovenous malformation) that has the potential to affect cognitive function.
   Participant has history or presence of symptomatic cerebral ischemia within the past 2 years, or documented history within the last 6 months of an acute event consistent with a transient ischemic attack.
   Participant has history of severe, clinically significant (persistent neurologic deficit or structural brain damage) CNS trauma (e.g., cerebral contusion).
   Participant has any other severe or unstable medical condition that could be expected to progress, recur, or change to such an extent that it could put the participant at special risk, bias the assessment of the clinical or mental status of the participant to a significant degree, interfere with the participant's ability to complete the study assessments, or would require the equivalent of institutional or hospital care.
   Participant is unable to tolerate MRI procedures or has a contraindication to MRI, including, but not limited to, the presence of pacemakers, aneurysm clips, artificial heart valves, ear implants, or foreign metal objects in the eyes, skin, or body that would contraindicate an MRI scan; or any other clinical history or examination finding that would pose a potential hazard in combination with MRI.

### Medication-Related Criteria

The following medications are prohibited for a pre-specified duration prior to study start, as indicated, and during the entire period of study participation (participants who start these medications during the study are withdrawn from study treatment):
Any continuous use of medications known to impair consciousness or cognition unless these medications are necessary for the treatment of medical conditions with the approval of the medical monitor. Intermittent or short-term use (<1 week) of these medications may be allowed but must be stopped 2 days or 5 half-lives, whichever is longer, prior to any cognitive or behavioral assessment, with the exception of the Winterlight Lab Speech Assessment (WLA) or Summerlight Lab Speech Assessment (SLA). Use of cannabinoids is prohibited within 24 hours prior to any cognitive or behavioral assessment, with the exception of the WLA or SLA.
Any investigational active immunotherapy (vaccine) that is under evaluation to prevent or postpone cognitive decline.
Any passive immunotherapy (immunoglobulin) or other long-acting biologic agent that is under evaluation to prevent or postpone cognitive decline within 1 year of screening. Participation in the previous Phase 1 study of anti-Sortilin antibody discussed above does not apply to this criterion.
A drug from a clinical trial (other than the previous Phase 1 study of anti-Sortilin antibody) within 30 days prior to drug administration in this study (Day 1); use of any experimental oral therapy within 30 days or 5 half-lives prior to Day 1, whichever is greater; use of any biologic therapy within 12 weeks or 5 half-lives prior to Day 1, whichever is greater; or any other investigational treatment within 5 half-lives or 3 months of screening, whichever is longer. Participants who received an experimental therapy that has no half-life, like a vaccine, completed that therapy at least 12 weeks prior to Day 1.
Typical antipsychotic or neuroleptic medication within 6 months of screening except as brief treatment for a nonpsychiatric indication (e.g., emesis).
Anti-coagulation (coumadin, heparinoids, apixaban) medications within 3 months of screening.
   Anti-platelet treatments (e.g., aspirin, dipyridamol) are permitted.
   Systemic immunosuppressive therapy or anticipated to be needed during the study.
   Use of prednisone of ≤10 mg/day or equivalent corticosteroid is allowed if stable for at least 3 months prior to enrollment and hemoglobin is >9 g/dL, white blood cell count is >3000/mm³, absolute neutrophil count is >1500/mm³, and platelet count is >100 000/mm³.
   Chronic use of opiates or opioids (including long-acting opioid medication) within 3 months of screening.
   Intermittent short-term use (<1 week) of short-acting opioid medications for pain is permitted except within 2 days or 5 half-lives (whichever is longer) prior to any neurocognitive assessment.
   Any stimulant medications (amphetamine, methylphenidate preparations, or modafinil) within 1 month of screening and throughout the study.
   Chronic use of barbiturates, or hypnotics from 3 months before screening.
   Intermittent short-term (<1 week) use of buspirone or short-acting hypnotic medication for sleep or anxiety is allowed except within 2 days or 5 half-lives (whichever is longer) prior to any neurocognitive assessment.

### Study Design

This Phase 2, multicenter, open-label study will evaluate the safety, tolerability, PK, PD, and effect on COAs of anti-Sortilin antibody in asymptomatic carriers and symptomatic patients who are heterozygous for a loss-of-function GRN mutation causative of FTD, and in symptomatic patients with C9orf72 hexanucleotide repeat expansion mutations causative of FTD.

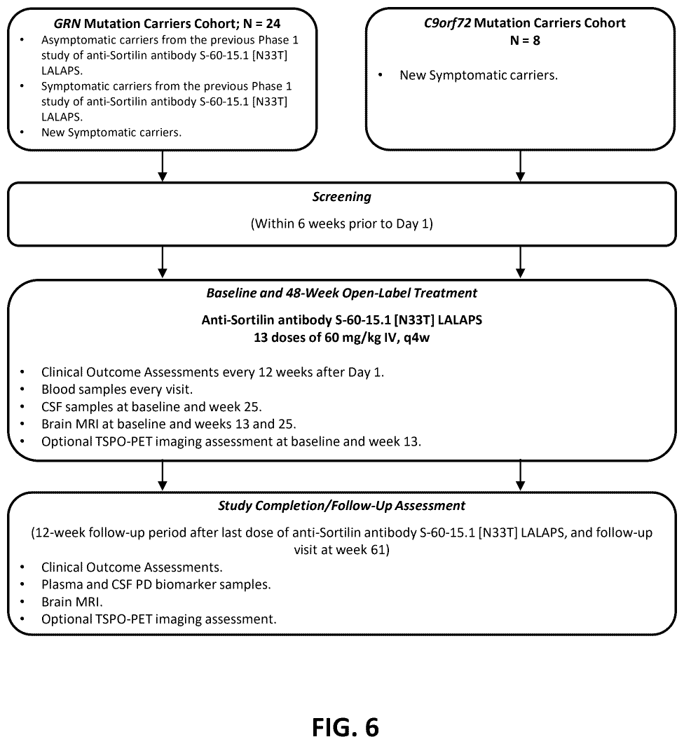

As shown in FIG. 6, the study includes a screening period (within 6 weeks prior to Day 1), a treatment period (48 weeks), and a follow-up period (12 weeks after the last dose of anti-Sortilin antibody) with a follow-up visit at week 61 (study completion).

### Study Treatment and Follow-Up

All enrolled participants undergo baseline evaluation with magnetic resonance imaging (MRI), optional TSPO-PET imaging, biofluid sampling for PD biomarker measurement, lumbar puncture for CSF collection, safety assessments, and several COAs.

Patients in the GRN Cohort and the C9orf72 Cohort (see the “Study Participants” section, above) are administered anti-Sortilin antibody intravenously at a dose of 60 mg/kg on day 1 and every four weeks (q4w) thereafter for a total of 13 doses (48—week treatment period), up to and including week 49. Anti-Sortilin antibody is administered IV over approximately 60 minutes. Participants are followed up at least 60 minutes after the end of IV infusion and completion of all activities scheduled for that visit day. Dosing solution preparation instructions are provided separately in a Pharmacy Manual.

Cognitive and functional testing including the participant and study partner are performed during screening, every 12 weeks after the baseline assessment (i.e., at weeks 13, 25, and 37), and at the study completion visit at week 61 (or an early termination visit).

Imaging is performed during screening, at week 13, week 25, and at the study completion visit at week 61 (or an early termination visit). Lumbar puncture for CSF collection is performed at screening, week 25, and at the study completion visit at week 61.

Participants are required to complete a follow-up assessment visit after the end of the 48—week treatment period and 12 weeks after the last dose (week 61), except for those participants who withdraw consent for study participation. If a participant is discontinued because of an AE, the event is followed up until it is resolved. Additionally, serious AEs (SAEs) considered related to study drug or radiotracer which occur at any time during the study are reported until resolution, participant withdrawal of consent, loss to follow up, or death, whichever is applicable.

Evaluations of MRI, optional TSPO-PET imaging, biofluid sampling for PD biomarker measurement, lumbar puncture for CSF collection, and several COAs occur during the treatment and follow-up periods.

### Optional TSPO-PET Imaging Assessment

An optional exploratory assessment to evaluate brain microglial activation as measured by TSPO-PET imaging is carried out to evaluate changes in brain microglial activation after IV dosing with anti-Sortilin antibody. A baseline TSPO-PET scan is performed prior to anti-Sortilin antibody dosing only after a patient has demonstrated eligibility for study participation, based on completion of all other screening assessments, at Week 13 and the study completion visit at Week 61.

### Study Drug

The anti-Sortilin antibody (study drug) is provided as a liquid solution formulated at a concentration of 50 mg/mL in an aqueous solution containing anti-Sortilin antibody in 20 mM histidine/histidine HCL, 7.5% (w/v) sucrose and 0.02 (w/v) polysorbate-80 at pH 5.5.

### Concomitant and Prior Therapy

During the course of the study, participants continue the use of accepted prescribed medications identified during the screening procedures, in accordance with study inclusion and exclusion criteria. Participants are advised against taking any new medication, both prescribed and over the counter, without consulting the investigator, unless the new medication is required for emergency use.

Any concomitant medication deemed necessary for the welfare of the participant during the study is given at the discretion of the investigator.

Any restricted medication is stopped as required by the study inclusion and exclusion criterion; participants who start these medications during the study are withdrawn from study treatment at the discretion of the sponsor's medical monitor.

### Patient Withdrawal

A participant is discontinued from study drug or study treatment at any time if it is not in the participant's best interest to continue. The following is a list of possible reasons for study drug or study treatment discontinuation:
Participant is noncompliant with the protocol.
Participant is lost to follow-up.
Participant withdraws consent.
Participant has a serious or intolerable AE that, in the investigator's opinion, requires withdrawal from the study treatment.
Occurrence of an intercurrent illness that, in the investigator's opinion, affects assessments of clinical status or safety to a significant degree.
Use of a non-permitted concomitant medication.
Pregnancy.
Discretion of the investigator.
Death

If a participant discontinues because of an AE or SAE, the event is followed up until it is resolved.

Participants who withdraw from the study are replaced in consultation with the investigator.

### Study Assessments

### Study Endpoints

### Primary Safety Endpoints

To assess the potential effect of cumulative exposure on the safety profile of anti-Sortilin antibody, the following is evaluated by dose, such as by using tertiles of the actual dose (normalized to weight) received:
Incidence, nature, and severity of AEs and SAEs.
Incidence of treatment discontinuations and study discontinuations due to AEs.
Physical examination abnormalities.
Neurological examination abnormalities.
Changes in vital signs from baseline over time.
Changes in ECGs from baseline over time.
MRI abnormalities after dosing relative to baseline.
Changes in clinical laboratory tests from baseline over time.
Sheehan Suicidality Tracking Scale (Sheehan-STS).
Incidence of ADAs to anti-Sortilin antibody.

### Secondary PK Endpoints

The secondary PK endpoints of this study are:
Serum concentration of anti-Sortilin antibody at specified time points.
Anti-Sortilin antibody PK parameters.
Cₘₐₓ.
C_{trough}.
AUCₛₛ.

The secondary PD biomarker endpoints of this study are:
The overall change from baseline of PGRN in CSF.
The overall change from baseline of PGRN in plasma.
The overall change from baseline of SORT1 on WBCs and sSORT1 in CSF.

The exploratory PD biomarker endpoints of this study are:
The overall change from baseline in exploratory biomarkers of neurodegeneration, lysosomal function, and microglial activity in blood, plasma, and CSF.
Global and regional brain MRI atrophy measures.
Neuroinflammation assessed by TSPO-PET (for participants who agree to participate in the optional imaging assessment only).
Correlations among exploratory fluid biomarkers, imaging measures, and COAs.

The exploratory clinical endpoints of this study are:
The overall change from baseline on the scores of the instruments in the COAs.
FTD Clinical Rating Scale (FCRS).
Frontotemporal Dementia Rating Scale (FRS).
Clinician's Global Impression-Improvement (CGI-I).
Neuropsychiatric Inventory (NPI).
Color Trails Test (CTT) Part 2.
Repeatable Battery for the Assessment of Neuropsychological Status (RBANS).
Delis-Kaplan Executive Function System (D-KEFS; Color-Word Interference only).
Interpersonal Reactivity Index.
Winterlight and Summerlight Lab Speech Assessments (WLA and SLA; for participants who agree to participate in these optional assessments only).

### Analysis Populations

Enrolled Population: The enrolled population consists of all participants who signed the Informed Consent Form and are eligible to participate in the study. The enrolled population is used for study population and COA summaries.

Safety Analysis Population: The safety analysis population consists of all participants who receive at least 1 dose of anti-Sortilin antibody. The safety analysis population is used for safety summaries.

PK Analysis Population: The PK analysis population includes all participants in the safety population who have adequate assessments for determination of at least 1 PK parameter. The PK analysis population is used for PK summaries.

PD Analysis Population: The PD analysis population includes all participants in the safety analysis population who have both a baseline and at least 1 post-dose PD assessment. The PD analysis population is used for summaries of PD activities.

Biomarker Population: The biomarker population consists of all participants in the safety population who have both a baseline and at least 1 post-dose measurement for at least 1 PD biomarker parameter. The PD biomarker population is used for exploratory PD biomarker summaries.

Descriptive statistics are used to assess clinically significant associated findings (for example, study-drug related AEs leading to study drug discontinuation or study-drug related SAEs).

Except for safety endpoints, all other study endpoints specified above are summarized by GRN mutation carriers versus C9orf72 mutation carriers.

### Statistical Analysis Methodology

The statistical analysis is performed using SAS software Version 9.4 or later (SAS Institute Inc., Cary, North Carolina, USA). For categorical variables, frequencies and percentages are presented. Continuous variables are summarized using descriptive statistics (number of participants, mean, standard deviation [SD], median, minimum, maximum, and 95% confidence interval [CI] where applicable). All CIs are 2-sided and performed using a 5% significance level except for PK parameters, for which 90% CI and geometric mean is used.

All summaries are presented by participant status and dementia type at baseline (aFTD-GRN vs. FTD-GRN bvFTD vs. FTD-GRN PPA vs. FTD-C9orf72 bvFTD vs. FTD-C9orf72 PPA) if the size of the subtype is at least 2 participants.

Baseline is defined as the last non-missing assessment, including repeated and unscheduled measurements, prior to the start of first study drug administration.

### Participant Demographics, Medical History, and Baseline Characteristics

Demographic information is recorded at screening.

All relevant medical history, including history of current disease, other pertinent history, and information regarding underlying diseases is recorded at screening prior to study drug administration. A diagnostic characterization form is completed at screening for symptomatic participants only. A diagnostic characterization form is also completed for any asymptomatic participant who becomes symptomatic during the course of the study; for these participants, the diagnostic characterization form is completed only at the first visit in which they exhibit clinical symptomatology.

Demographics (including but not limited to age, gender, and race) and baseline and background characteristics are presented in summary tables. Qualitative data (e.g., medical history, diagnostic characterization) is summarized in contingency tables. Quantitative data (e.g., age) is summarized using quantitative descriptive statistics. All genotype data is presented in a summary table.

### Study Drug and Prior/Concomitant Medications

Study drug administration data is summarized by number of doses received and total dose received. The overall treatment compliance is calculated based on dose interruptions/discontinuations.

Prior and concomitant medications are coded using the WHO-DD, March 2019 or later. All prior and concomitant medications data are summarized by anatomical therapeutic chemical classes and generic names. Separate summaries are presented for prior and concomitant medications.

### Safety Assessments

All safety summaries are presented using the safety analysis population.

Adverse events, regardless of relationship to study drug, radiotracer (¹⁸F-PBR06 or ¹¹C-PBR28) or TSPO-PET optional assessment procedure are recorded. AEs are coded to system organ class and preferred term according to MedDRA, version 21.1 or later. The following AE summaries are reported by system organ class, preferred term, participant status, and dementia type at baseline:
Treatment emergent AEs (TEAEs).
Treatment-related TEAEs.
TEAEs by relationship to study drug.
TEAEs by severity.
SAEs.
TEAEs leading to study drug discontinuation.
TEAEs leading to study discontinuation.

For safety reporting, any clinically significant changes in MRI after dosing relative to baseline are evaluated by the investigator and are included as AEs. A separate analysis of AEs identified through MRI is not conducted.

### Physical and Neurological Examinations

Complete neurological examinations are performed, including evaluation of consciousness, orientation, cranial nerves, motor and sensory system, coordination and gait, and reflexes. Changes from baseline abnormalities and changes from previous neurological examinations are recorded at each subsequent neurologic examination. New or worsened abnormalities are recorded as AEs if considered clinically significant.

Complete physical examinations (PE) are performed, including evaluation of the head, eyes, ears, nose, and throat, and the cardiovascular, dermatological, musculoskeletal, respiratory, and gastrointestinal systems. A limited, symptom-directed examination is performed at all other specified time points, prior to study drug administration (if applicable), or as clinically indicated. Abnormalities observed at baseline, as well as new or worsened clinically significant abnormalities at all other visits are recorded. New abnormal PE findings are followed up at the next scheduled visit. New or worsened abnormalities are recorded as AEs if considered clinically significant. Height (cm) is measured at screening.

Separate shift tables for physical and neurological examinations are generated by the categorical interpretation of findings and are presented by body system.

Supine systolic and diastolic blood pressure (BP), pulse, body temperature, and respiratory rate are recorded after the participant has been resting for ≥5 minutes in the supine position. Body temperature and respiratory rate are measured subsequently. Abnormalities observed at baseline, and new or worsened clinically significant abnormalities in subsequent visits are recorded. New or worsened abnormalities are recorded as AEs if considered clinically significant. Weight (kg) is collected at the same visits that vital signs are taken.

Actual values and changes from baseline for vital signs and weight are summarized at each time point using descriptive statistics.

Triplicate 12-lead ECGs are obtained after the patient has been in the supine position for ≥5 minutes. All ECGs are analyzed from a clinical safety basis (without intensive QT analysis). The clinical significance of ECG changes are determined by the investigator after review of the ECG report in relation to the participant's medical history, PE, and concomitant medications.

Actual values and changes from baseline for quantitative ECG results are summarized at each time point using descriptive statistics. A shift table is generated for the categorical interpretation of ECGs. Any grade 3 or higher QTcF prolongation is listed.

### Clinical Laboratory Analysis

Blood and urine samples are collected for clinical safety laboratory tests (chemistry, coagulation, hematology, urinalysis, serology, and pregnancy testing).

Actual values and changes from baseline for clinical laboratory test results are summarized at each time point using descriptive statistics. Shift tables are generated for clinical laboratory test results.

### Suicidality Tracking Scale

The Sheehan Suicidality Tracking Scale is a brief scale designed to assess and monitor over time the core phenomena of suicidality. An AE is recorded if the investigator makes an evaluation and deems there to be suicidal ideation or behavior.

A summary table for Sheehan-STS Total Score is presented by time point using descriptive statistics.

### Immunogenicity Analysis

Blood serum samples are collected for determination of anti-drug antibodies (ADAs). Additional ADA samples are collected in participants with signs and symptoms of infusion-related reactions. A corresponding additional PK sample is obtained at the same time point, and a plasma sample for cytokine analysis.

Immunogenicity test results for ADA to anti-Sortilin antibody are summarized by time point.

### Pharmacokinetic and Pharmacodynamic Assessments

### Sample Collection

Blood serum samples are collected for assessment of serum concentrations of anti-Sortilin antibody. All PK samples are collected from the arm that is not used for the infusion on day of study drug administration.

Blood PGRN plasma samples are collected for evaluation of levels of PGRN.

Whole blood samples are collected for evaluation of levels of SORT1 on WBCs and for evaluation of other analytes.

Cerebrospinal fluid samples are assessed for concentration of anti-Sortilin antibody. Cerebrospinal fluid samples are also evaluated for levels of PGRN and sSORT1. Cerebrospinal fluid samples are collected via lumbar puncture prior to study drug administration (if applicable) at Screening, Week 25, and Study Completion/Early Termination to evaluate PK, PD, and exploratory PD biomarker measures. The Week 25 lumbar puncture is adjusted based on review of exploratory PD biomarkers.

Exploratory whole blood, plasma, and CSF PD biomarker samples are collected for evaluation of neurodegeneration (i.e., neurofilament-light [Nfl], tau, pTau), lysosomal function (i.e., cathepsins), and microglial activity (i.e., YKL-40, interleukin-6), evaluation of messenger ribonucleic acid (mRNA) expression in peripheral cells, and to potentially evaluate levels of other analytes relevant to disease biology and response to anti-Sortilin antibody.

### Analysis of Secondary Pharmacokinetic Endpoints

All PK summaries are presented using the PK analysis population.

Individual and mean serum anti-Sortilin antibody concentration-time data is tabulated and plotted by study day and mutation carriers. As applicable, the serum PK of anti-Sortilin antibody is summarized by estimation of maximum observed concentration (Cmax), trough concentration (Ctrough), and area under the concentration-time curve (AUCss) on the basis of results obtained following multiple doses of anti-Sortilin antibody by study day and cohort.

The individual serum concentration versus actual time data for anti-Sortilin antibody is used to derive PK parameters by standard non-compartmental methods using Phoenix® WinNonlin® (Certara USA Inc., Princeton, NJ, USA) version 6.4 or higher. The individual PK parameters are presented in listings. PK parameters are summarized in tables using the following descriptive statistics: n, arithmetic mean, SD, coefficient of variation (CV), geometric mean, geometric mean CV, minimum, median, and maximum. Geometric mean and geometric mean, 90% CI, and CV are only included for Cmax, Ctrough, and AUCss.

Potential correlations of relevant PK parameters with demographics, safety (including QT changes), and PD measures are explored, as data allow. Additional modeling, including population PK analysis, to characterize these correlations is performed.

### Analysis of Secondary and Exploratory Pharmacodynamic Endpoints

All PD summaries are presented using the PD analysis population.

PD endpoints are described and summarized by study day and mutation carriers at baseline and each time point specified, as well as the percent change from baseline for each PD endpoint. Pharmacodynamic endpoints evaluated in plasma and CSF samples include but are not limited to PGRN, SORT1, sSORT1, and Nfl.

Summary statistics for PD endpoints and their corresponding changes from baseline (i.e., percent change from baseline) are tabulated by study day and cohort. The time course of PD endpoints is presented graphically for both observed values and percent change from baseline values. In addition, a mixed model of repeated measures (MMRM) is used to summarize the mean percent changes in PD endpoints from baseline with 95% CIs. Association between PD endpoints and clinical response are also explored.

The PK-PD relationship is modeled by population PK/PD model using nonlinear mixed effects modeling. Baseline exploratory PD biomarkers are also explored as potential predictors of response to anti-Sortilin antibody, including baseline serum or CSF PGRN levels and PGRN genotyping.

### Analysis of Exploratory Pharmacodynamic Biomarker Endpoints

All exploratory PD biomarker summaries are presented using the biomarker population.

Correlations among fluid PD biomarker levels, imaging PD measures, and clinical outcome measures are evaluated.

### Magnetic Resonance Imaging (MRI)

MRI scans of the brain are performed and centrally reviewed for assessment of safety, and for evaluation of global and regional brain volumes, volume of white matter hyperintensities, brain perfusion (measured by arterial spin labeling MRI), fractional anisotropy, mean diffusivity, axial diffusivity, and radial diffusivity (measured by diffusion tensor imaging), and functional brain activity (measured by functional MRI).

Actual results and percent change from baseline values for quantitative MRI parameters are summarized by visit using descriptive statistics. Mean percent change from baseline values, plus or minus the SD, are also presented in a plot.

### Translocator Protein Positron Emission Tomography (TSPO-PET)

Due to a polymorphism (rs6971) in the TSPO affecting the amino acid at position 147, approximately 10% of the population exhibits low-affinity binding of TSPO radioligands to the TSPO mitochondrial protein. As part of the screening visit and prior to traveling to the imaging site, if the participant agrees and provides consent, the participant is prescreened for the optional TSPO-PET imaging assessment. An optional blood sample is collected at the clinical site to genotype the rs6971 TSPO polymorphism to determine whether they are high-affinity binders (Ala/Ala amino acid at position 147), medium-affinity binders (Ala/Thr), or low-affinity binders (Thr/Thr). All individuals who are low-affinity binders (Thr/Thr) are excluded from participation in the optional TSPO-PET imaging assessment. High- and medium-affinity binders are eligible to participate in the in the optional TSPO-PET imaging assessment.

Participants in the optional TSPO-PET imaging undergo TSPO-PET imaging prior to anti-Sortilin antibody dosing, and at Week 13 and the Study Completion Visit (Week 61). Baseline optional TSPO-PET imaging is performed prior to anti-Sortilin antibody dosing only after a participant has demonstrated eligibility for study participation, based on completion of all screening assessments.

The overall goal of TSPO-PET analysis is to evaluate [¹¹C]PBR28 and [¹⁸F]PBR06 as radiotracer pharmacodynamic (PD) biomarkers of microglial activation in the brain of study patients prior to and following treatment with anti-Sortilin antibody.

In addition, TSPO-PET analyses are carried out to evaluate changes in brain microglial activation after IV dosing with anti-Sortilin antibody. During TSPO-PET analyses, MRI and [11C]PBR28 and [¹⁸F]PBR06 PET images are co-aligned for anatomy-based definition of regions of interest (ROI) for analysis of regional [¹¹C]PBR28 and [¹⁸F]PBR06 binding/uptake. An anatomic template is used to define brain sub-structures in both the MRI and PET scan.

### Other Exploratory Pharmacodynamic Biomarkers

Actual results and change from baseline values for other exploratory PD biomarker parameters are summarized by visit using descriptive statistics. Mean change from baseline values, plus or minus the SD, are also presented in a plot.

### Analysis of Exploratory Clinical Outcome Assessment Endpoints

The following neurocognitive and functional tests are performed. Neurocognitive and functional tests are performed prior to study drug administration (if applicable) and prior to any stressful procedures (e.g., blood collections, imaging).
Frontotemporal Dementia Clinical Rating Scale (FCRS).
Frontotemporal Dementia Rating Scale (FRS).
Clinical Global Impression-Improvement (CGI-I).
Neuropsychiatric Inventory (NPI).
Color Trails Test (CTT) Part 2.
Repeatable Battery for the Assessment of Neuropsychological Status (RBANS).
Delis-Kaplan Executive Function System Color-Word Interference Test.
Interpersonal Reactivity Index.
Winterlight Lab Speech Assessment (WLA) and Summerlight Lab Speech Assessment (SLA) (US, UK, and Canadian participants who are proficient in English and who agree and are eligible to participate in the optional assessments only).

All clinical outcome assessment (COA) summaries are presented using the enrolled population. The full details of analysis for COA include total and subscale scores.

Actual results and change from baseline values for COA total and/or subscale scores are summarized by visit using descriptive statistics. Mean change from baseline values, plus or minus the SD, are also presented in a plot.

The COA endpoint is assessed using MMRM methodology. The dependent variable is the change from baseline score to each post-baseline visit assessment. The fixed effects include the participant mutation type and dementia type, and time point is the repeated measure. Covariates including but not limited to baseline PGRN level, gender, and age are explored.

### Pharmacogenomic Assessments

A blood sample is collected at screening for DNA extraction to enable analysis via whole genome sequencing to identify common and rare genetic variants that are predictive of response to study drug, are associated with progression to a more severe disease state, are associated with susceptibility to developing AEs, or can increase the knowledge and understanding of disease biology.

### Example 4: A Phase 2 Clinical Study to Evaluate Anti-Sortilin Antibody in Amyotrophic Lateral Sclerosis (ALS)

This Example describes a Phase 2 study to evaluate the safety, tolerability, pharmacokinetics, and pharmacodynamics of anti-Sortilin antibody S-60-15.1 [N33T] LALAPS in ALS patients.

### Study Objectives

### Primary Objective

The primary objective of this study is to determine whether treatment with anti-Sortilin antibody impacts the pathophysiology of ALS and exerts a clinical benefit.

### Secondary Objectives

The secondary objectives of this study are:
Assess the safety and tolerability of anti-Sortilin antibody in ALS patients.
Assess the pharmacokinetics and pharmacodynamics of anti-Sortilin antibody in ALS patients.

### Study Population

Patients meeting any one of the following criteria are included in this study:
Familial or sporadic ALS patients that exhibit accumulation of TDP-43 or another TDP-43-related pathology.
Familial or sporadic ALS patients that carry TDP-43 mutations.
Familial or sporadic ALS patients that carry a C9orf72 hexanucleotide repeat expansion.

### Study Treatment

ALS patients are administered anti-Sortilin antibody intravenously at a dose of 60 mg/kg on day 1 and every four weeks (q4w) thereafter. Anti-Sortilin antibody is administered IV over approximately 60 minutes. Dosing solution preparation instructions are provided separately in a Pharmacy Manual.

### Study Assessments

### Pharmacokinetics Assessments

Target engagement in the blood is assessed by measuring free SORT1 levels in white blood cells using an immunoassay.

### Pharmacodynamic Assessments

The following pharmacodynamic markers are measured in both serum and CSF:
Progranulin (Adipogen immunoassay).
Markers of neurodegeneration, e.g., neurofilament light chain (Quanterix or Roche Diagnostics).
Markers of glial activation, e.g., YKL-40 (CHI3L), IL-6, GFAP (Roche Diagnostics).
Markers of TDP-43 pathology.

In addition, MRI studies are used to assess the effect of anti-Sortilin antibody on brain atrophy (structural MRI), connectivity, and free water/inflammation (DTI).

### Example 5: A Phase 1B Study to Evaluate the Effect of a Single Intravenous Dose of Anti-Sortilin Antibody in Asymptomatic GRN Mutation Carriers (aFTD-GRN) and Multiple Intravenous Doses of Anti-Sortilin Antibody in FTD-GRN Patients

This Example provides results of the Phase 1b open label study described in Example 2, which evaluated asymptomatic carriers of Granulin mutations (aFTD-GRN) administered a single dose of anti-Sortilin antibody S-60-15.1 [N33T] LALAPS at 60 mg/kg, as well as symptomatic carriers of Granulin mutations (FTD-GRN patients) administered three doses of anti-Sortilin antibody S-60-15.1 [N33T]LALAPS at 30 mg/kg, q2w (every two weeks).

### Results

### Safety Outcomes

No serious adverse events (SAEs) were observed. All adverse events were mild. No drug or study discontinuations occurred.

### Plasma PGRN Levels

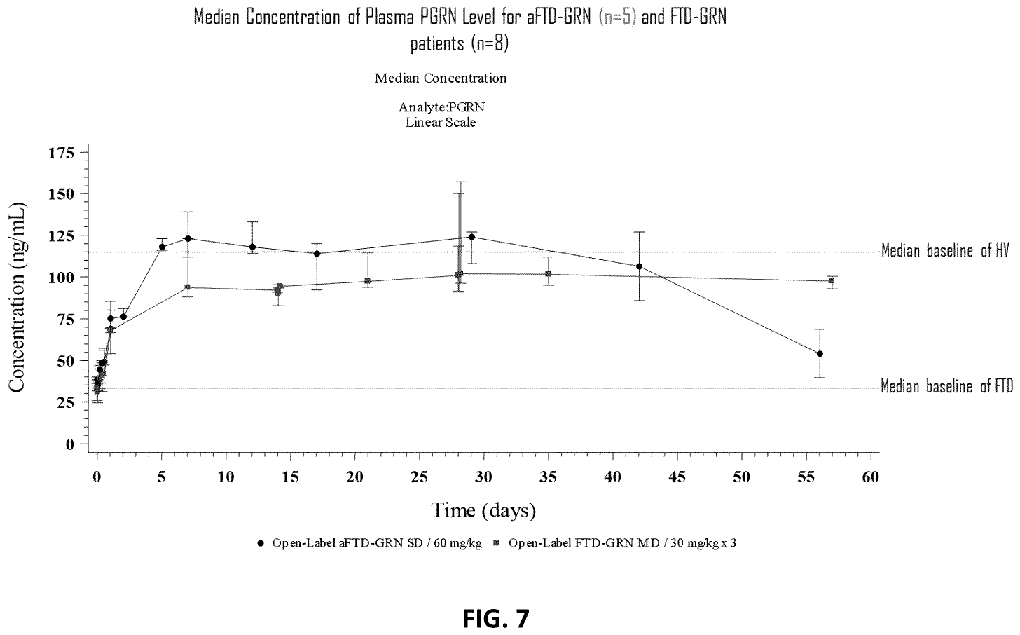

As shown in FIG. 7, administration of anti-Sortilin antibody 5-60-15.1 [N33T] LALAPS increased plasma PGRN concentrations in aFTD-GRN carriers (circles) and FTD-GRN patients (squares). The median plasma PGRN concentrations of healthy volunteers (HV) and FTD patients are provided in FIG. 7 for comparison. In FTD-GRN patients, PGRN levels were increased throughout the study, reaching up to 3-fold higher than the median baseline levels observed in FTD-GRN patients and achieving levels comparable to healthy volunteers. This effect was sustained up to day 56 post-dose (about 4 weeks after the last dose). Similar results were observed in aFTD-GRN carriers, with peak levels of PGRN reaching up to 4-fold higher than median baseline levels.

### CSF PGRN Levels

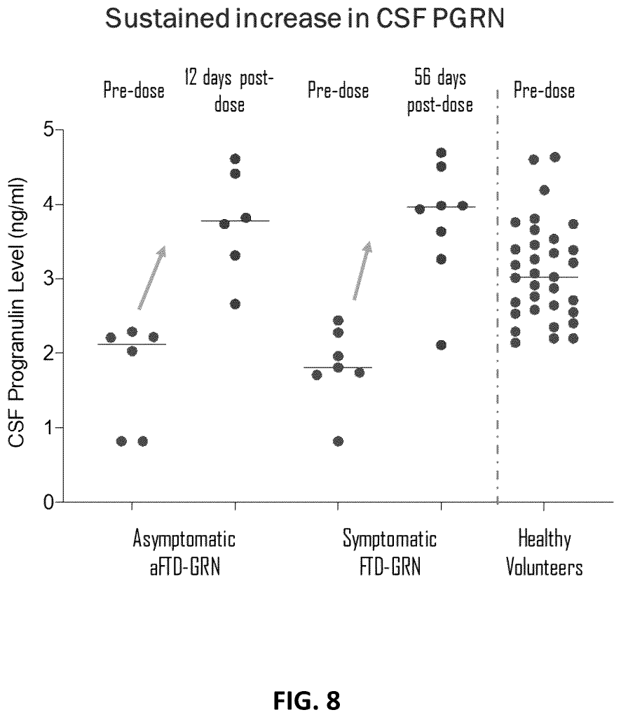

As shown in FIG. 8, the concentration of PGRN in CSF was increased after administration of anti-Sortilin antibody S-60-15.1 [N33T] LALAPS to either FTD-GRN patients (symptomatic) or aFTD-GRN carriers (asymptomatic). For example, at 56 days after administration of anti-Sortilin antibody S-60-15.1 [N33T] LALAPS to FTD-GRN patients, the median PGRN concentration (ng/ml) in CSF was about 2-fold higher than the baseline (pre-dose) PGRN level. Similarly, 12 days after administration of anti-Sortilin antibody 5-60-15.1 [N33T] LALAPS to aFTD-GRN carriers, the median PGRN concentration in CSF was about 2-fold higher than the baseline (pre-dose) PGRN level. The concentration of PGRN in healthy volunteers (HV) is provided in FIG. 8 for comparison.

### Disease Protein Signature in FTD-GRN Patients

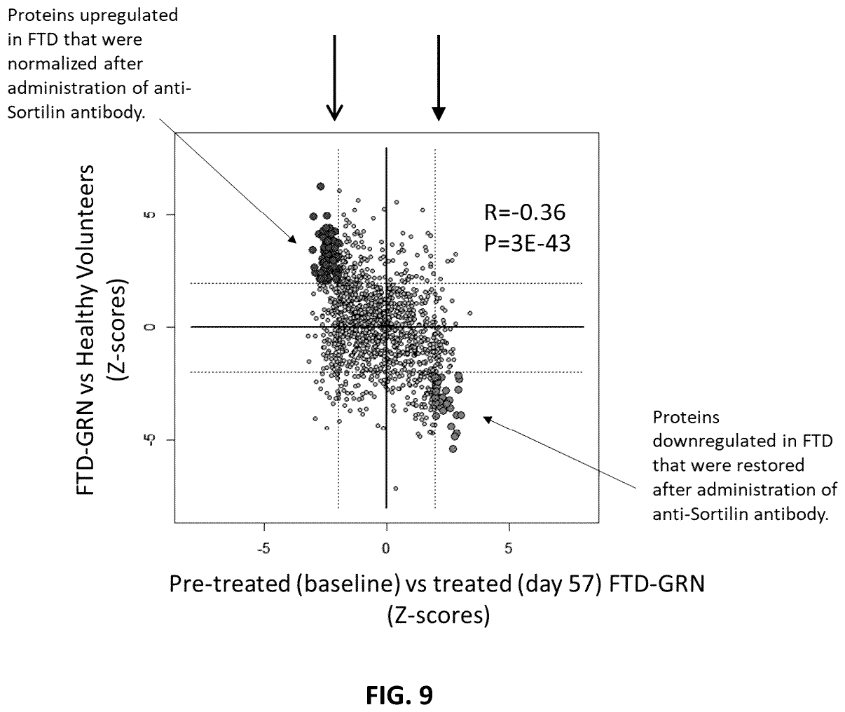

The SOMASCAN aptamer-based proteomics assay (see, e.g., Candia et al. (2017) Sci Rep 7, 14248) was used to generate a protein signature profile from the CSF of FTD-GRN patients. In this assay, the relative abundance of over 1,000 proteins in the CSF of FTD-GRN patients was analyzed before and after treatment (day 57) with S-60-15.1 [N33T] LALAPS to identify relevant biomarkers. FIG. 9 shows the results using a four-way restoration plot, with each data point representing an individual protein. All data points above the highest horizontal line are proteins that are upregulated in FTD. All data points below the lowest horizontal line are proteins that are downregulated in FTD. All data points to the right of the vertical line indicated by the closed arrow are proteins that are upregulated by S-60-15.1 [N33T] LALAPS. All data points to the left of the vertical line indicated by the open arrow are proteins that are downregulated by S-60-15.1 [N33T] LALAPS. Thus, the lower right quadrant shows that S-60-15.1 [N33T] LALAPS upregulates proteins that are downregulated in FTD, and the upper left quadrant shows that S-60-15.1 [N33T] LALAPS downregulates proteins that are upregulated in FTD. Therefore, S-60-15.1 [N33T] LALAPS counteracts the protein signature of the disease state as indicated in these quadrants, and this effect is highly statistically significant in view of the lower amount of data points (proteins) in the upper right and lower left quadrants.

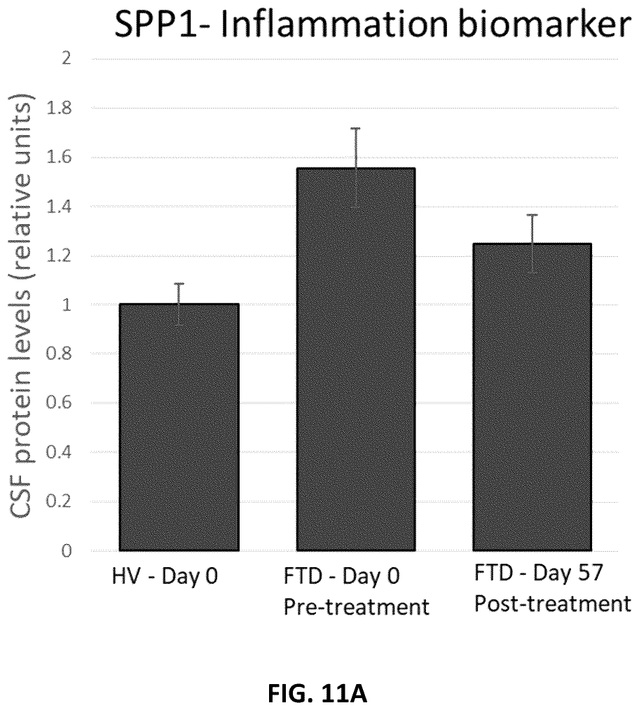

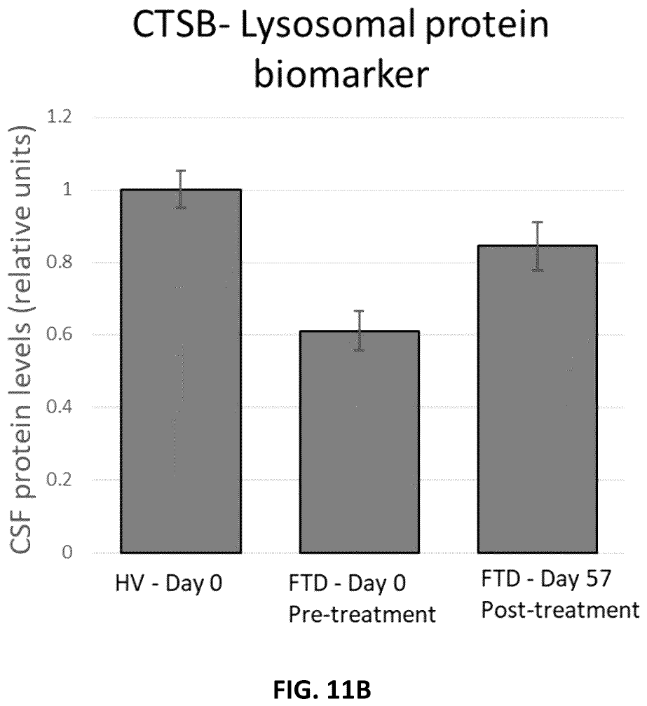

The proteins in the lower right quadrant, which are upregulated by 5-60-15.1 [N33T] LALAPS, include lysosomal proteins that are downregulated in FTD, including Granulin and Cathepsin B (CTSB). The proteins in the upper left quadrant, which are downregulated by S-60-15.1 [N33T] LALAPS, include inflammatory proteins that are upregulated in FTD, such as Osteopontin (SPP1). Therefore, the reversal of the disease protein signature is consistent with the function that these proteins would otherwise play in the normal state. FIGS. 11A-11B show in detail the levels of CTSB and SPP1 proteins in the cerebrospinal fluid (CSF) of healthy volunteers and FTD-GRN patients before and after treatment (day 57) with S-60-15.1 [N33T] LALAPS. As FIG. 11A shows, SPP1 is upregulated in untreated FTD patients (“FTD-Day 0 Pre-treatment”) relative to healthy volunteers (“HV-Day 0”), and treatment with S-60-15.1 [N33T] LALAPS significantly decreased SPP1 levels in FTD patients (“FTD-Day 57 Post-treatment”). Conversely, as FIG. 11B shows, CTSB is downregulated in untreated FTD patients (“FTD-Day 0 Pre-treatment”) relative to healthy volunteers (“HV-Day 0”), and treatment with S-60-15.1 [N33T] LALAPS significantly increased CTSB levels in FTD patients (“FTD-Day 57 Post-treatment”).

Additional proteins in the upper left quadrant of the four-way restoration plot shown in FIG. 9 were identified and include the following inflammatory proteins: YWHAE (14-3-3 protein epsilon), allograft inflammatory factor 1 (AIF1), colony stimulating factor 1 (CSF1), chitinase 1 (CHIT1), lymphocyte antigen 86 (LY86), and CD86. These results showed that certain inflammatory proteins that are upregulated in FTD are downregulated or normalized following administration of S-60-15.1 [N33T] LALAPS.

An additional protein in the lower right quadrant of the four-way restoration plot shown in FIG. 9 was identified as N-acetylglucosamine kinase (NAGK). NAGK is a lysosomal protein that is downregulated in FTD. These results showed that certain lysosomal proteins that are downregulated in FTD are upregulated or normalized following administration of S-60-15.1 [N33T] LALAPS.

### Neurofilament Light (NfL) Levels

Neurofilament light chain (NfL) is a biomarker for neurodegenerative diseases, including FTD. NfL levels are five- to seven-fold elevated in FTD-GRN patients compared to controls. (Meeter et al. (2016) Ann. Clin. Transl. Neurol. 3(8):623-636). Conversely, NfL levels have been shown to decrease after ˜6 months of treatment with drugs that are effective in other neurodegenerative disorders. (Kuhle et al. (March 2019) Neurology 92 (10) e1007-e1015); Olsson et al. (2019) Journal of Neurology 266:2129-2136.) Accordingly, plasma NfL levels were examined in FTD-GRN patients after treatment with S-60-15.1 [N33T]LALAPS.

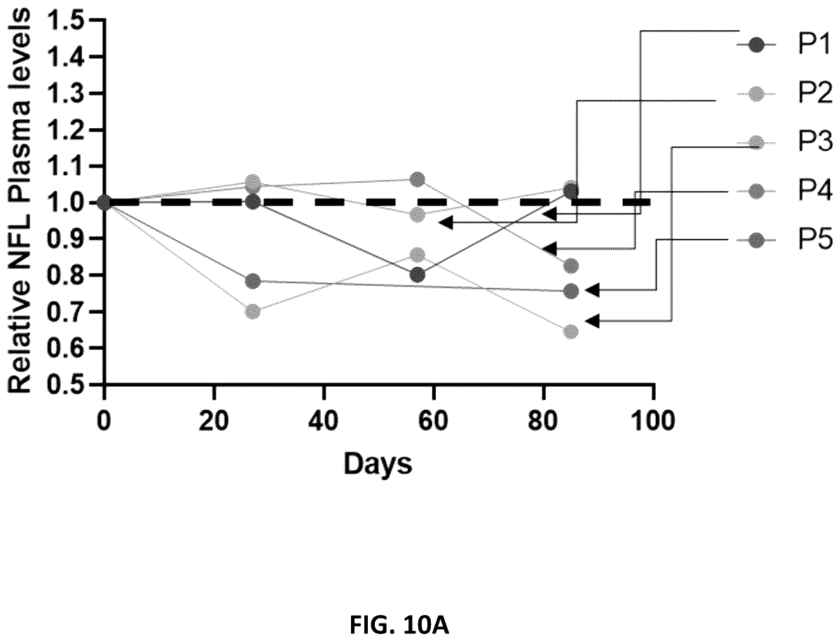

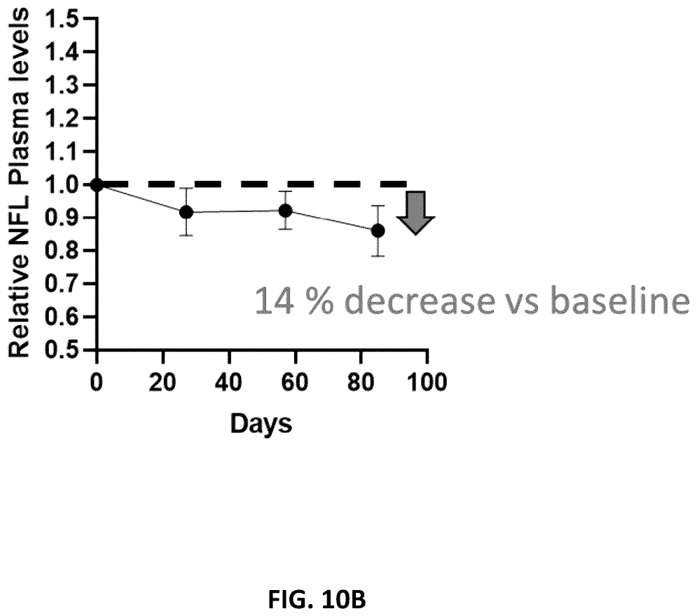

FIG. 10A shows preliminary data from five patients for which blood samples were available up through day 85, or about three months after the first dose. NfL plasma levels were measured using the SIMOA Nf-Light Advantage assay by Quinterix. In FIG. 10A, NfL plasma levels are indicated at various time points as a ratio to baseline level for each of the five patients. FIG. 10B shows the geometric mean of the data of FIG. 10A, suggesting an initial trend of about a 14% decrease in plasma NfL levels.

### CONCLUSIONS

The results of this ongoing Phase 1b study show that anti-Sortilin antibody S-60-15.1 [N33T]LALAPS was generally safe and well tolerated up to the highest dose level of 60 mg/kg. In addition, the results showed that anti-Sortilin antibody S-60-15.1 [N33T] LALAPS increased PGRN levels in the plasma and CSF of aFTD-GRN mutation carriers and FTD-GRN patients, restoring the levels to within the normal ranges observed in healthy volunteers. Furthermore, administration of anti-Sortilin antibody S-60-15.1 [N33T] LALAPS to FTD-GRN patients led to a normalization of protein signatures in the CSF.

TABLE 11
Heavy chain HVR H1 sequences of anti-SORT1 antibodies

| | | SEQ ID |
|---|---|---|
| Ab(s) | HVR H1 | NO: |
| S-60; S-60-10; S-60-11; S-60-12; S-60-13; S-60-14; S-60-15 | YSISSGYYWG | 1 |
| [N33(wt)]; S-60-15.1 [N33T]; S-60-15.2 [N33S]; S-60-15.3 | | |
| [N33G]; S-60-15.4 [N33R]; S-60-15.5 [N33D]; S-60-15.6 | | |
| [N33H]; S-60-15.7 [N33K]; S-60-15.8 [N33Q]; S-60-15.9 | | |
| [N33Y]; S-60-15.10 [N33E]; S-60-15.11 [N33W]; S-60-15.12 | | |
| [N33F]; S-60-15.13 [N33I]; S-60-15.14 [N33V]; S-60-15.15 | | |
| [N33A]; S-60-15.16 [N33M]; S-60-15.17 [N33L]; S-60-16; | | |
| S-60-18; S-60-19; S-60-24 | | |

TABLE 12
Heavy chain HVR H2 sequences of anti-SORT1 antibodies

| Ab(s) | HVR H2 | SEQ ID NO: |
|---|---|---|
| S-60; S-60-10; S-60-11; S-60-12; S-60-15 [N33(wt)]; S-60- | TIYHSGSTYYNPSL | 2 |
| 15.1 [N33T]; S-60-15.2 [N33S]; S-60-15.3 [N33G]; S-60-15.4 | KS | |
| [N33R]; S-60-15.5 [N33D]; S-60-15.6 [N33H]; S-60-15.7 | | |
| [N33K]; S-60-15.8 [N33Q]; S-60-15.9 [N33Y]; S-60-15.10 | | |
| [N33E]; 5-60-15.11 [N33W]; S-60-15.12 [N33F]; S-60-15.13 | | |
| [N33I]; S-60-15.14 [N33V]; S-60-15.15 [N33A]; S-60-15.16 | | |
| [N33M]; S-60-15.17 [N33L]; S-60-16; S-60-18; S-60-19; S-60- | | |
| 24 | | |
| S-60-13; S-60-14 | TIYHSGSTYYNPSL | 3 |
| | ES | |
| Formula I | TIYHSGSTYYNPSL | 4 |
| | X₁S | |
| | X₁ is K or E | |

TABLE 13
Heavy chain HVR H3 sequences
of anti-SORT1 antibodies

| | | SEQ |
|---|---|---|
| | | ID |
| Ab(s) | HVR H3 | NO: |
| S-60-10; S-60-11; | ARQGSIQQGYYGM | 5 |
| S-60-12; S-60-13; | DV | |
| S-60-14; S-60-19 | | |
| S-60; | ARQGSIKQGYYGM | 6 |
| S-60-15 [N33 (wt)]; | DV | |
| S-60-15.1 [N33T]; | | |
| S-60-15.2 [N33S]; | | |
| S-60-15.3 [N33G]; | | |
| S-60-15.4 [N33R]; | | |
| S-60-15.5 [N33D]; | | |
| S-60-15.6 [N33H]; | | |
| S-60-15.7 [N33K]; | | |
| S-60-15.8 [N33Q]; | | |
| S-60-15.9 [N33Y]; | | |
| S-60-15.10 [N33E]; | | |
| S-60-15.11 [N33W]; | | |
| S-60-15.12 [N33F]; | | |
| S-60-I5.13 (N33I]; | | |
| S-60-15.14 [N33V]; | | |
| S-60-15.15 [N33A]; | | |
| S-60-15.16 [N33M]; | | |
| S-60-15.17 [N33L]; | | |
| S-60-16; S-60-18; | | |
| S-60-24 | | |
| Formula II | ARQGSIX₁QGYYGM | 7 |
| | DV | |
| | X₁ is Q or K | |

TABLE 14
Light chain HVR L1 sequences of anti-SORT1
antibodies

| | | SEQ |
|---|---|---|
| Ab(s) | HVR L1 | ID NO: |
| S-60-10; S-60-11; | RSSQSLLRSNGYNY | 8 |
| S-60-12; S-60-13; | LD | |
| S-60-14; S-60-15 | | |
| [N33 (wt)]; S-60-16; | | |
| S-60-18 | | |
| S-60-15.1 [N33T] | RSSQSLLRSTGYNYL | 9 |
| | D | |
| S-60-15.2 [N33S] | RSSQSLLRSSGYNYL | 10 |
| | D | |
| S-60-15.3 [N33G] | RSSQSLLRSGGYNY | 11 |
| | LD | |
| S-60-15.4 [N33R] | RSSQSLLRSRGYNY | 12 |
| | LD | |
| S-60-15.5 [N33D] | RSSQSLLRSDGYNY | 13 |
| | LD | |
| S-60-15.6 [N33H] | RSSQSLLRSHGYNY | 14 |
| | LD | |
| S-60-15.7 [N33K] | RSSQSLLRSKGYNY | 15 |
| | LD | |
| S-60-15.8 [N33Q] | RSSQSLLRSQGYNY | 16 |
| | LD | |
| S-60-15.9 [N33Y] | RSSQSLLRSYGYNY | 17 |
| | LD | |
| S-60-15.10 [N33E] | RSSQSLLRSEGYNYL | 18 |
| | D | |
| S-60-15.11 [N33W] | RSSQSLLRSWGYNY | 19 |
| | LD | |
| S-60-15.12 [N33F] | RSSQSLLRSFGYNYL | 20 |
| | D | |
| S-60-15.13 [N33I] | RSSQSLLRSIGYNYL | 21 |
| | D | |
| S-60-15.14 [N33V] | RSSQSLLRSVGYNY | 22 |
| | LD | |
| S-60-15.15 [N33A] | RSSQSLLRSAGYNY | 23 |
| | LD | |
| S-60-15.16 [N33M] | RSSQSLLRSMGYNY | 24 |
| | LD | |
| S-60-15.17 [N33L] | RSSQSLLRSLGYNYL | 25 |
| | D | |
| S-60; S-60-19 | RSSQSLLHSNGYNY | 26 |
| | LD | |
| S-60-24 | RSSQGLLRSNGYNY | 27 |
| | LD | |
| Formula III | RSSQX₁LLX₂SX₃GYN | 28 |
| | YLD | |
| | X₁ is S or G | |
| | X₂ is R or H | |
| | X₃ is N, T, S, | |
| | G, R, D, H, K, Q, | |
| | Y, E, W, F, I, | |
| | V, A, M, or L | |

TABLE 15
Light chain HVR L2 sequences of
anti-SORT1 antibodies

| | | SEQ |
|---|---|---|
| | | ID |
| Ab(s) | HVR L2 | NO: |
| S-60; S-60-10; S-60-11; S-60-13; | LGSNRAS | 29 |
| S-60-14; S-60-15 [N33 | | |
| (wt)]; S-60-15.1 [N33T]; | | |
| S-60-15.2 [N33S]; S-60-15.3 [N33G]; | | |
| S-60-15.4 [N33R]; S-60-15.5 [N33D]; | | |
| S-60-15.6 [N33H]; S-60-15.7 [N33K]; | | |
| S-60-15.8 [N33Q]; S-60-15.9 [N33Y]; | | |
| S-60-15.10 [N33E]; 5-60-15.11 [N33W]; | | |
| S-60-15.12 [N33F]; S-60-15.13 [N33I]; | | |
| S-60-15.14 [N33V]; S-60-15.15 [N33A]; | | |
| S-60-15.16 [N33M]; S-60-15.17 [N33L]; | | |
| S-60-16; S-60-18; S-60-19; S-60-24 | | |
| S-60-12 | LGSNRVS | 30 |
| Formula IV | LGSNRX₁S | 31 |
| | X₁ is A | |
| | or V | |

TABLE 16
Light chain HVR L3 sequences of anti-SORT1 antibodies

| Ab(s) | HVR L3 | SEQ ID NO: |
|---|---|---|
| S-60-10; S-60-11; S-60-13; S-60-14; S-60-15 [N33 (wt)]; S- | MQQQEAPLT | 32 |
| 60-15.1 [N33T]; S-60-15.2 [N33S]; S-60-.15.3 [N33G]; S-60- | | |
| 15.4 [N33R]; S-60-15.5 [N33D]; S-60-15.6 [N33H]; S-60-15.7 | | |
| [N33K]; S-60-15.8 [N33Q]; S-60-15.9 [N33Y]; S-60-15.10 | | |
| [N33E]; S-60-15.11 [N33W]; S-60-15.12 [N33F]; S-60-15.13 | | |
| [N33I]; S-60-15.14 [N33V]; S-60-15.15 [N33A]; S-60-15.16 | | |
| [N33M]; S-60-15.17 [N33L]; S-60-16; S-60-24 | | |
| S-60; S-60-12; S-60-18; S-60-19 | MQQQETPLT | 33 |
| Formula V | MQQQEXᵢPLT | 34 |
| | Xᵢ is A or T | |

TABLE 17
Heavy chain framework 1 sequences of
anti-SORT1 antibodies

| | | SEQ |
|---|---|---|
| | | ID |
| Ab(s) | VH FR1 | NO: |
| S-60-10; S-60-11; S-60-12; | QVQLQESGPGLVKP | 35 |
| S-60-13; S-60-14; S-60-15 | SETLSLTCAVSG | |
| [N33 (wt)]; S-60-15.1 [N33T]; | | |
| S-60-15.2 [N33S]; S-60-15.3 | | |
| [N33G]; S-60-15.4 [N33R]; | | |
| S-60-15.5 [N33D]; S-60-15.6 | | |
| [N33H]; S-60-15.7 [N33K]; | | |
| S-60-15.8 [N33Q]; S-60-15.9 | | |
| [N33Y]; S-60-15.10 [N33E]; | | |
| S-60-15.11 [N33W]; S-60-15.12 | | |
| [N33F]; S-60-15.13 [N33I]; | | |
| S-60-15.14 [N33V]; S-60-15.15 | | |
| [N33A]; S-60-15.16 [N33M]; | | |
| S-60-15.17 [N33L]; S-60-16; | | |
| S-60-18; S-60-19; S-60-24 | | |

TABLE 18
Heavy chain framework 2 sequences of
anti-SORT1 antibodies

| | | SEQ |
|---|---|---|
| | | ID |
| Ab(s) | VH FR2 | NO: |
| S-60-10; S-60-11; S-60-12; | WIRQPPGKGLEWIG | 36 |
| S-60-13; S-60-14; S-60-15 | | |
| [N33 (wt)]; S-60-15.1 [N33T]; | | |
| S-60-15.2 [N33S]; S-60-15.3 | | |
| [N33G]; S-60-15.4 [N33R]; | | |
| S-60-15.5 [N33D]; S-60-15.6 | | |
| [N33H]; S-60-15.7 [N33K]; | | |
| S-60-15.8 [N33Q]; S-60-15.9 | | |
| [N33Y]; S-60-15.10 [N33E]; | | |
| S-60-15.11 [N33W]; S-60-15.12 | | |
| [N33F]; S-60-15.13 [N33I]; | | |
| S-60-15.14 [N33V]; S-60-15.15 | | |
| [N33A]; S-60-15.16 [N33M]; | | |
| S-60-15.17 [N33L]; S-60-16; | | |
| S-60-18; S-60-19; S-60-24 | | |

TABLE 19
Heavy chain framework 3 sequences of anti-SORT1 antibodies

| Ab(s) | VH FR3 | SEQ ID NO: |
|---|---|---|
| S-60-10; S-60-11; S-60-12; S-60-19 | QVTISVDTSKNQFSLELSSVTAAD | 37 |
| | TAVYYC | |
| S-60-13; S-60-14; S-60-15 [N33 (wt)]; | RVTISVDTSKNQFSLKLSSVTAAD | 38 |
| S-60-15.1 [N33T]; S-60-15.2 [N33S]; | TAVYYC | |
| S-60-15.3 [N33G]; S-60-15.4 [N33R]; | | |
| S-60-15.5 [N33D]; S-60-15.6 [N33H]; | | |
| S-60-15.7 [N33K]; S-60-15.8 [N33Q]; | | |
| S-60-15.9 [N33Y]; S-60-15.10 [N33E]; | | |
| S-60-15.11 [N33W]; S-60-15.12 [N33F]; | | |
| S-60-15.13 [N33I]; S-60-15.14 [N33V]; | | |
| S-60-15.15 [N33A]; S-60-15.16 [N33M]; | | |
| S-60-15.17 [N33;]; S-60-15.18; S-60-24 | | |
| Formula VI | X₁VTISVDTSKNQFSLX₂LSSVTAA | 39 |
| | DTAVYYYC | |
| | X₁ is Q or R | |
| | X₂ is E or K | |

TABLE 20
Heavy chain framework 4 sequences
of anti-SORT1 antibodies

| | | SEQ |
|---|---|---|
| | | ID |
| Ab(s) | VH FR4 | NO: |
| S-60-10; S-60-11; | WGQGTTVTVSS | 40 |
| S-60-12; S-60-13; | | |
| S-60-14; | | |
| S-60-15 [N33 (wt)]; | | |
| S-60-15.1 [N33T]; | | |
| S-60-15.2 [N33S]; | | |
| S-60-15.3 [N33G]; | | |
| S-60-15.4 [N33R]; | | |
| S-60-15.5 [N33D]; | | |
| S-60-15.6 [N33H]; | | |
| S-60-15.7 [N33K]; | | |
| S-60-15.8 [N33Q]; | | |
| S-60-15.9 [N33Y]; | | |
| S-60-15.10 [N33E]; | | |
| S-60-15.11 [N33W]; | | |
| S-60-15.12 [N33F]; | | |
| S-60-15.13 [N33I]; | | |
| S-60-15.14 [N33V]; | | |
| S-60-15.15 [N33A]; | | |
| S-60-15.16 [N33M]; | | |
| S-60-15.17 [N33L]; | | |
| S-60-16; S-60-18; | | |
| S-60-19; S-60-24 | | |

TABLE 21
Light chain framework 1 sequences
of anti-SORT1 antibodies

| | | SEQ |
|---|---|---|
| | | ID |
| Ab(s) | VL FR1 | NO: |
| S-60-10: S-60-11; | DIVMTQSPLSLP | 41 |
| S-60-12; S-60-13; | VTPGEPASISC | |
| S-60-14; | | |
| S-60-15 [N33 (wt)]; | | |
| S-60-15.1 [N33T]; | | |
| S-60-15.2 [N33S]; | | |
| S-60-15.3 [N33G]; | | |
| S-60-15.4 [N33R]; | | |
| S-60-15.5 [N33D], | | |
| S-60-15.6 [N33H]; | | |
| S-60-15.7 [N33K]; | | |
| S-60-15.8 [N33Q]; | | |
| S-60-15.9 [N33Y]; | | |
| S-60-15.10 [N33E]; | | |
| S-60-15.11 [N33W]; | | |
| S-60-15.12 [N33F]; | | |
| S-60-15.13 [N33I]; | | |
| S-60-15.14 [N33V]; | | |
| S-60-15.15 [N33A]; | | |
| S-60-15.16 [N33M]; | | |
| S-60-15.17 [N33L]; | | |
| S-60-16; S-60-19 | | |
| S-60-18 | DIVMTQSPLSLP | 42 |
| | VTPGGPASISC | |
| S-60-24 | DIVMTQSPLSLP | 43 |
| | VTPGESASISC | |
| Formula VII | DIVMTQSPLSLP | 44 |
| | VTPGX₁X₂ASISC | |
| | X₁ is E or G | |
| | X₂ is P or S | |

TABLE 22
Light chain framework 2 sequences of anti-SORT1
antibodies

| | | SEQ |
|---|---|---|
| | | ID |
| Ab(s) | YL FR2 | NO: |
| S-60-10; S-60-11; S-60-13; S-60-14; | WYLQKP | 45 |
| S-60-15 [N33 (wt)]; S-60-15.1 [N33T]; | GQSPQL | |
| S-60-15.2 [N33S]; S-60-15.3 [N33G]; | LIY | |
| S-60-15.4 [N33R]; S-60-15.5 [N33D]; | | |
| S-60-15.6 [N33H]; S-60-15.7 [N33K]; | | |
| S-60-15.8 [N33Q]; S-60-15.9 [N33Y]; | | |
| S-60-15.10 [N33E]; S-60-15.11 [N33W]; | | |
| S-60-15.12 [N33F]; S-60-15.13 [N33I]; | | |
| S-60-15.14 [N33V]; S-60-15.15 [N33A]; | | |
| S-60-15.16 [N33M]; S-60-15.17 [N33L]; | | |
| S-60-16; S-60-18; S-60-19; S-60-24 | | |
| S-60-12 | WYLQKP | 46 |
| | GQPPQL | |
| | LIY | |
| Formula VIII | WYLQKP | 47 |
| | GQX₁PQ | |
| | LLIY | |
| | X₁ is | |
| | S or P | |

TABLE 23
Light chain framework 3 sequences of anti-SORTI antibodies

| Ab(s) | VL FR3 | SEQ ID NO: |
|---|---|---|
| S-60-10; S-60-13; S-60-15 [N33(wt)]; S-60-15.1 [N33T]; S- | GVPDRFSGSGSGTD | 48 |
| 60-15.2 [N33S]; S-60-15.3 [N33G]; S-60-15.4 [N33R]; S-60- | FTLKISRAEAEDVGV | |
| 15.5 [N33D]; S-60-15.6 [N33H]; S-60-15.7 [N33K]; S-60- | YYC | |
| 15.8 [N33Q]; S-60-15.9 [N33Y]; S-60-15.10 [N33E]; S-60- | | |
| 15.11 [N33W]; S-60-15.12 [N33F]; S-60-15.13 [N33I]; S-60- | | |
| 15.14 [N33V]; S-60-15.15 [N33A]; S-60-15.16 [N33M]; S-60- | | |
| 15.17 [N33L] | | |
| S-60-11; 5-60-12; 5-60-14; 5-60-19; 5-60-24 | GVPDRFSGSGSGTD | 49 |
| | FTLKISRVEAEDVGV | |
| | YYC | |
| S-60-16 | GVPDRFSGSGSGTD | 50 |
| | FTLKISRVEAEDVGA | |
| | YYC | |
| S-60-18 | GVPDRLSGSGSGTD | 51 |
| | FTLKISRVEAEDVGV | |
| | YYC | |
| Formula IX | GVPDRX₁SGSGSGTD | 52 |
| | FTLKISRX₂EAEDVG | |
| | X₃YYC | |
| | X₁ is F or L | |
| | X₂ is A or V | |
| | X₃ is V or A | |

TABLE 24
Light chain framework 4 sequences
of anti-SORT1 antibodies

| | | SEQ |
|---|---|---|
| | | ID |
| Ab(s) | VL FR4 | NO: |
| S-60-10; S-60-11; S-60-12; | FGGGTKVEIK | 53 |
| S-60-13; S-60-14; S-60-15 | | |
| [N33(wt)]; S-60-15.1 [N33T]; | | |
| S-60-15.2 [N33S]; S-60-15.3 | | |
| [N33G]; S-60-15.4 [N33R]; | | |
| S-60-15.5 [N33D]; S-60-15.6 | | |
| [N33H]: S-60-15.7 [N33K]; | | |
| S-60-15.8 [N33Q]; S-60-15.9 | | |
| [N33Y]; S-60-15.10 [N33E]; | | |
| S-60-15.11 [N33W]; S-60-15.12 | | |
| [N33F]; S-60-15.13 [N33I]; | | |
| S-60-15.14 [N33V]; S-60-15.15 | | |
| [N33A]; S-60-15.16 [N33M]; | | |
| S-60-15.17 [N33L]; S-60-16; | | |
| S-60-18; S-60-19; S-60-24 | | |

TABLE 25
Heavy chain variable region sequences of anti-SORT1 antibodies

| Ab(s) | HCVR | SEQ ID NO: |
|---|---|---|
| S-60-10, S-60-11, S-60-12, S-60-19 | QVQLQESGPGLVKPSETLSLTCAVSG | 54 |
| | YSISSGYYWGWIRQPPGKGLEWIGTI | |
| | YHSGSTYYNPSLKSQVTISVDTSKNQ | |
| | FSLELSSVTAADTAVYYCARQGSIQQ | |
| | GYYGMDVWGQGTTVTVSS | |
| S-30-13, S-60-14 | QVQLQESGPGLVKPSETLSLTCAVSG | 55 |
| | YSISSGYYWGWIRQPPGKGLEWIGTI | |
| | YHSGSTYYNPSLESRIVTISVDTSKN | |
| | QFSLKLSSVTAADTAVYNCARQGSIQ | |
| | QGYYGMDVWGQGTTVTVSS | |
| S-60, S-60-15 [N33 (wt)], S-60-15.1 | QVQLQESGPGINKPSETLSLTCAVSG | 56 |
| [N33T], S-60-15.2 [N33S], S-60-15.3 | YSISSGYYWGWIRQPPGKGLENVIGI | |
| [N33G], S-60-15.4 [N33R], S-60-15.5 | TIYHSGSTYYNPSLKSRVT1SVDTSK | |
| [N33D], S-60-15.6 [N33H], S-60-15.7 | NQFSLKLSSVTAADTAVYWARQGSIK | |
| [N33K], S-60-15.8 [N33Q], 5-60-15.9 | QGYYGMDVWGOGTTNITVSS | |
| [N33Y], S-60-15.10 [N33E], 5-60-15.11 | | |
| [N33W], S-60-15.12 [N33F], S-60-15.13 | | |
| [N33I], S-60-15.14 [N33V], S-60-15.15 | | |
| [N33A], S-60-15.16 [N33M], S-60-15.17 | | |
| [N33L], S-60-16, S-60-18, S-60-24 | | |

TABLE 26
Light chain variable region sequences
of anti-SORT1 antibodies

| | | SEQ |
|---|---|---|
| | | ID |
| Ab(s) | LCVR | NO: |
| S-60-10; S-60-13; | DIVMTQSPLSLPVTPGEPASISCRS | 57 |
| S-60-15 | SQSLLRSNGYNYLDWYLQKPGQSPQ | |
| [N33 (wt)] | LLIYLGSNRASGVPDRFSGSGSGTD | |
| | FTLKISRAEAEDVGVYYCMQQQEAP | |
| | LTFGGGTKVEIK | |
| S-60-11; S-60-14 | DIVMTQSPLSLPVTPGEPASISCRS | 58 |
| | SQSLLRSNGYNYLDWYLQKPGQSPQ | |
| | LLIYLGSNRASGVPDRFSGSGSGTD | |
| | FTLKISRVEAEDVGVYYCMQQQEAP | |
| | LTFGGGTKVEIK | |
| S-60-12 | DIVMTQSPLSLPVTPGEPASISCRS | 59 |
| | SQSLLRSNGYNYLDWYLQKPGQPPQ | |
| | LLIYLGSNRVSGVPDRFSGSGSGTD | |
| | FTLKISRVEAEDVGVYYCMQQQETP | |
| | LTFGGGTKVEIK | |
| S-60-15.1 [N33T] | DIVMTQSPLSLPVTPGEPASISCRS | 60 |
| | SQSLLRSTGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.2 [N33S] | DIVMTQSPLSLPVTPGEPASISCRS | 61 |
| | SQSLLRSSGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.3 [N33G] | DIVMTQSPLSLPVTPGEPASISCRS | 62 |
| | SQSLLRSGGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.4 [N33R] | DIVMTQSPLSLPVTPGEPASISCRS | 63 |
| | SQSLLRSRGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.5 [N33D] | DIVMTQSPLSLPVTPGEPASISCRS | 64 |
| | SQSLLRSDGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.6 [N33H] | DIVMTQSPLSLPVTPGEPASISCRS | 65 |
| | SQSLLRSHGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.7 [N33K] | DIVMTQSPLSLPVTPGEPASISCRS | 66 |
| | SQSLLRSRGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.8 [N33Q] | DIVMTQSPLSLPVTPGEPASISCRS | 67 |
| | SQSLLRSQGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.9 [N33Y] | DIVMTQSPLSLPVTPGEPASISCRS | 68 |
| | SQSLLRSYGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.10 [N33E] | DIVMTQSPLSLPVTPGEPASISCRS | 69 |
| | SQSLLRSEGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.11 [N33W] | DIVMTQSPLSLPVTPGEPASISCRS | 70 |
| | SQSLLRSWGYNYLDWYLQKPGQSPQ | |
| | LLIYLGSNRASGVPDRFSGSGSGID | |
| | PTLKISRAEAEDVGVYYCMQQQEAP | |
| | LTFGGGTKVEIK | |
| S-60-15.12 [N33F] | DIVMTQSPLSLPVTPGEPASISCRS | 71 |
| | SQSLLRSFGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.13 [N33I] | DIVMTQSPLSLPVTPGEPASISCRS | 72 |
| | SQSLLRSIGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.14 [N33V] | DIVMTQSPLSLPVTPGEPASISCRS | 73 |
| | SQSLLRSVGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.15 [N33A] | DIVMTQSPLSLPVTPGEPASISCRS | 74 |
| | SQSLLRSAGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.16 [N33M] | DIVMTQSPLSLPVTPGEPASISCRS | 75 |
| | SQSLLRSMGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-15.17 [N33L] | DIVMTQSPLSLPVTPGEPASISCRS | 76 |
| | SQSLLRSLGYNYLDWYLQKPGQSPQ | |
| | LLIYLGSNRASGVPDRFSGSGSGTD | |
| | FTLKISRAEAEDVGVYYCMQQQEAP | |
| | LTFGGGTKVEIK | |
| S-60-16 | DIVMTQSPLSLPVTPGEPASISCRS | 77 |
| | SQSLLRSNGYNYLDWYLQKPGQSPQ | |
| | LLIYLGSNRASGVPDRFSGSGSGTD | |
| | FTLKISRVEAEDVGAYYCMQQQEAP | |
| | LTFGGGTKVEIK | |
| S-60-18 | DIVMTQSPLSLPVTPGGPASISCRS | 78 |
| | SQSLLRSNGYNYLDWYLQKPGQSPQ | |
| | LLIYLGSNRASGVPDRLSGSGSGTD | |
| | FTLKISRVEAEDVGVYYCMQQQETP | |
| | LTFGGGTKVEIK | |
| S-60, S-60-19 | DIVMTQSPLSLPVTPGEPASISCRS | 79 |
| | SQSLLHSNGYNYLDWYLQKPGQSPQ | |
| | LLIYLGSNRASGVPDRFSGSGSGTD | |
| | FTLKISRVEAEDVGVYYCMQQQETP | |
| | LTFGGGTKVEIK | |
| S-60-24 | DIVMTQSPLSLPVTPGESASISCRS | 80 |
| | SQGLLRSNGYNYLDWYLQKPGQSPQ | |
| | LLIYLGSNRASGVPDRFSGSGSGTD | |
| | FTLKISRVEAEDVGVYYCMQQQEAP | |
| | LTFGGGTKVEIK | |

TABLE 27
Sortilin amino acid sequences

| | | SEQ ID |
|---|---|---|
| Description | Sequence | NO |
| Human | 10 20 30 40 50 | 81 |
| Sortilin | MERPWGAADG LSRWPHGLGL LLLLQLLPPS TLSQDRLDAP PPPAAPLPRW | |
| | 60 70 80 90 100 | |
| | SGPIGVSWGL RAAAAGGAFP RGGRWRRSAP GEDEECGRVR DFVAKLANNT | |
| | 110 120 130 140 150 | |
| | HQHVFDDLRG SVSLSWVGDS TGVILVLTTF HVPLVIMTFG QSKLYRSEDY | |
| | 160 170 180 190 200 | |
| | GKNFKDITDL INNTFIRTEF GMAIGPENSG KVVLTAEVSG GSRGGRIFRS | |
| | 210 220 230 240 250 | |
| | SDFAKNFVQT DLPFHPLTQM MYSPQNSDYL LALSTENGLW VEKNFGGKWE | |
| | 260 270 280 290 300 | |
| | EIHKAVCLAK WGSDNTIFFT TYANGSCKAD LGALELWRTS DLGKSFKTIG | |
| | 310 320 330 340 350 | |
| | VKIYSFGLGG RFLFASVMAD KDTTRRIHVS TDQGDTWSMA QLPSVGQEQF | |
| | 360 370 380 390 400 | |
| | YSILAANDDM VFMHVDEPGD TGFGTIFTSD DRGIVYSKSL DRHLYTTTGG | |
| | 410 420 430 440 450 | |
| | ETDFTNVTSL RGVYITSVLS EDNSIQTMIT FDQGGPWTHL RKPENSECDA | |
| | 460 470 480 490 500 | |
| | TAKNKNECSL HIHASYSISQ KLNVPMAPLS EPNAVGIVIA HGSVGDAISV | |
| | 510 520 530 540 550 | |
| | MVPDVIISDD GGYSWTKMLE GPHYYTILDS GGIIVAIEHS SRPINVIKFS | |
| | 560 570 580 590 600 | |
| | TDEGQCWQTY TFTRDPIYFT GLASEPGARS MNISIWGFTE SFLTSQWVSY | |
| | 610 620 630 640 650 | |
| | TIDFKDILER NCEEKDYTIW LAHSTDPEDY EDGCILGYKE QFLRLRKSSV | |
| | 660 670 680 690 700 | |
| | CQNGRDYVVT KQPSICLCSL EDFLCDFGYY RPENDSKCVE QPELKGHDLE | |
| | 710 720 730 740 750 | |
| | FCLYGREEHL TTNGYRKIPG DKCQGGVNPV REVKDLKKKC TSNFLSPEKQ | |
| | 760 770 780 790 800 | |
| | NSKSNSVPII LAIVGLMLVT VVAGVLIVKK YVCGGRFLVH RYSVLQQHAE | |
| | 810 820 830 | |
| | ANGVDGVDAL DTASHTNKSG YHDDSDEDLL E | |
| Mouse | MERPRGAADG LLRWPLGLLL LLQLLPPAAV GQDRLDAPPP | 82 |
| Sortilin | PAPPLLRWAG PVGVSWGLRA AAPGGPVPRA GRWRRGAPAE | |
| | DQDCGRLPDF IAKLTNNTHQ HVFDDLSGSV SLSWVGDSTG | |
| | VILVLTTFQV PLVIVSFGQS KLYRSEDYGK NFKDITNLIN | |
| | NTFIRTEFGM AIGPENSGKV ILTAEVSGGS RGGRVFRSSD | |
| | FAKNFVQTDL PFHPLTQMMY SPQNSDYLLA LSTENGLWVS | |
| | KNFGEKWEEI HKAVCLAKWG PNNIIFFTTH VNGSCKADLG | |
| | ALELWRTSDL GKTFKTIGVK IYSFGLGGRF LFASVMADKD | |
| | TTRRIHVSTD QGDTWSMAQL PSVGQEQFYS ILAANEDMVF | |
| | MHVDEPGDTG FGTIFTSDDR GIVYSKSLDR HLYTTTGGET | |
| | DFTNVTSLRG VYITSTLSED NSIQSMFITD QGGRWEHLRK | |
| | PENSKCDATA KNKNECSLHI HASYSISQKL NVPMAPLSEP | |
| | NAVGIVIAHG SVGDAISVMV PDVYISDDGG YSWAKMLEGP | |
| | HYYTILDSGG IIVAIEHSNR PINVIKFSTD EGQCWQSYVF | |
| | TQEPIYFTGL ASEPGARSMN ISIWGFTESF ITRQWVSYTV | |
| | DFKDILERNC EEDDYTTWLA HSTDPGDYKD GCILGYKEQF | |
| | LRLRKSSVCQ NGRDYVVAKQ PSVCPCSLED FLCDFGYFRP | |
| | ENASECVEQP ELKGHELEFC LYGKEEHLTT NGYRKIPGDK | |
| | CQGGMNPARE VKDLKKKCTS NFLNPTKQNS KSNSVPIILA | |
| | IVGLMLVTVV AGVLIVKKYV CGGRFLVHRY SVLQQHAEAD | |
| | GVEALDSTSH AKSGYHDDSD EDLLE | |
| Rat | MERPRGAADG LLRWPLGLLL LLQLLPPAAV GQDRLDAPPP | 83 |
| Sortilin | PAPPLLRWAG PVGVSWGLRA AAPGGPVPRA GRWRRGAPAE | |
| | DQDCGRLPDF IAKLTNNTHQ HVFDDLSGSV SLSWVGDSTG | |
| | VILVLTTFQV PLVIVSFGQS KLYRSEDYGK NFKDITNLIN | |
| | NTFIRTEFGM AIGPENSGKV ILTAEVSGGS RGGRVFRSSD | |
| | FAKNFVQFTL PFHPLTQMMY SPQNSDYLLA LSTENGLWVS | |
| | KNFGEKWEEI HKAVCLAKWG PNNIIFFTTH VNGSCKADLG | |
| | ALELWRTSDL GKTEKTIGVK IYSFGLGGRF LFASVMADKD | |
| | TTRRIHWSTD QGDTWSMAQL PSVGQEQFYS ILAANDDMVF | |
| | MHVDEPGDTG FGTIFTSDDR GIVYSKSLDR HLYTTTGGET | |
| | DFTNVTSLRG VYITSTLSED NSIQSMITFD QGGRWEHLQK | |
| | PENSKCDATA KNKNECSLHI HASYSISQKL NVPMAPLSEP | |
| | NAVGIVIAHG SVGDAISVMV PDVYISDDGG YSWAKMLEGP | |
| | HYYTILDSGG IIVAIEHSNR PINVIKFSTD EGQCWQSYVF | |
| | SQEPVYFTGL ASEPGARSMN ISIWGFTESF LTRQWVSYTI | |
| | DFKDILERNC EENDYTTWLA HSTDPGDYKD GCILGYKEQF | |
| | LRLRKSSVCQ NGRDYVVAKQ PSICPCSLED FLCDFGYFRP | |
| | ENASECVEQP ELKGHELEFC LYGKEEHLTT NGYRKIPGDR | |
| | CQGGMNPARE VKDLKKKCTS NFLNPKKQNS KSSSVPIILA | |
| | IVGLMLVTVV AGVLIVKKYV CGGRFLVHRY SVLQQHAEAD | |
| | GVEALDTASH AKSGYHDDSD EDLLE | |

TABLE 28
Fc domain amino acid sequences

| | | SEQ ID |
|---|---|---|
| Description | Sequence | NO |
| huIgG1 | ASTKGPSVFPLAPSSKSTSGGTAALGCLVKDYFPEPVTVSWNSGALT | 84 |
| LALAPS Fc | SGVHTFPAVLQSSGLYSLSSVVTVPSSSLGTQTYICNVNHKPSNTKV | |
| with C- | DKKVEPKSCDKTHTCPPCPAPEAAGGPSVFLFPPKPKDTLMISRTPE | |
| terminal | VTCVVVDVSHEDPEVKFNWYVDGVEVHNAKTKPREEQYNSTYRV | |
| lysine | VSVLTVLHQDWLNGKEYKCKVSNKALPASIEKTISKAKGQPREPQV | |
| | YTLTPSRDELTKNQVSLTCLVKGFYPSDIAVEWESNGQPENNYKTT | |
| | PPVLDSDGSFFLYSKLTVDKSRWQQGNVFSCSVMHEALHNHYTQK | |
| | SLSLSPGK | |
| huIgG1 | ASTKGPSVFPLAPSSKSTSGGTAALGCLVKDYFPEPVTVSWNSGALT | 85 |
| LALAPS Fc | SGVHTFPAVLQSSGLYSLSSVVTVPSSSLGTQFYICNVNHKPSNTKV | |
| without C- | DKKVEPKSCDKTHTCPPCPAPEAAGGPSVFLFPPKPKDTLMISRTPE | |
| terminal | VTCVVVDVSHEDPEVKFNWYVDGVEVHNAKTKPREEQYNSTYRV | |
| lysine | VSVLTVLHQDWLNGKEYKCKVSNKALPASIEKTISKAKGQPREPQV | |
| | YTLPPSRDELTKNQVSLTCLVKGFYPSDIAVEWESNGQPENNYKTT | |
| | PPVLDSDGSFFLYSKLTVDKSRWQQGNVFSCSVMHEALHNHYTQK | |
| | SLSLSPG | |

TABLE 29
Full-length heavy chain amino acid sequences

| | | SEQ ID |
|---|---|---|
| Description | Sequence | NO |
| S-60-10, S-60-11, S-60-12, S- | QVQLQESGPGLVKPSETLSLTCAVSGYSISSGYYWGW | 86 |
| 60-19 with Fc LALAPS with | IRQPPGKGLEWIGTIYHSGSTYYNPSLKSQVTISVDTS | |
| C-terminal Lysine | KNQFSLELSSVTAADTAVYYCARQGSIQQGYYGMDV | |
| | WGQGTTVTVSSASTKGPSVFPLAPSSKSTSGGTAALG | |
| | CLVKDYFPEPVTVSWNSGALTSGVHTFPAVLQSSGLY | |
| | SLSSVVTVPSSSLGTQTYICNVNHKPSNTKVDKKVEP | |
| | KSCDKTHTCPPCPAPEAAGGPSVFLFPPKPKDTLMISR | |
| | TPEVTCVVVDVSHEDPEVKFNWYVDGVEVHNAKTK | |
| | PREEQYNSTYRVVSVLTVLHQDWLNGKEYKCKVSN | |
| | KALPASIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNH | |
| | YTQKSLSLSPGK | |
| S-60-10, S-60-11, S-60-12, S- | QVQLQESGPGLVKPSETLSLTCAVSGYSISSGYYWGW | 87 |
| 60-19 with Fc LALAPS | IRQPPGKGLEWIGTIYHSGSTYYNPSLKSQVTISVDTS | |
| without C-terminal Lysine | KNQFSLELSSVTAADTAVYYCARQGSIQQGYYGMDV | |
| | WGQGTTVTVSSASTKGPSVFPLAPSSKSTSGGTAALG | |
| | CLVKDYFPEPVTVSWNSGALTSGVHTFPAVLQSSGLY | |
| | SLSSVVTVPSSSLGTQTYICNVNHKPSNTKVDKKVEP | |
| | KSCDKTHTCPPCPAPEAAGGPSVFLFPPKPKDTLMISR | |
| | TPEVTCVVVDVSHEDPEVKFNWYVDGVEVHNAKTK | |
| | PREEQYNSTYRVVSVLTVLHQDWLNGKEYKCKVSN | |
| | KALPASIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNH | |
| | YTQKSLSLSPG | |
| S-60-13, S-60-14 with Fc | QVQLQESGPGLVKPSETLSLTCAVSGYSISSGYYWGW | 88 |
| LALAPS with C-terminal | IRQPPGKGLEWIGTIYHSGSTYYNPSLESRVTISVDTSK | |
| Lysine | NQFSLKLSSVTAADTAVYYCARQGSIQQGYYGMDV | |
| | WGQGTTVTVSSASTKGPSVFPLNPSSKSTSGGTAALG | |
| | CLVKDYFPEPVTVSWNSGALTSGVHTFPAVLQSSGLY | |
| | SLSSVVTVPSSSLGTQTYICNVNHKPSNTKVDKKVEP | |
| | KSCDKTHTCPPCPAPEAAGGPSVFLFPPKPKDTLMISR | |
| | TPEVTCVVVDVSHEDPEVKFNWYVDGVEVHNAKTK | |
| | PREEQYNSTYRVVSVLTVLHQDWLNGKEYKCKVSN | |
| | KALPASIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNH | |
| | YTQKSLSLSPGK | |
| S-60-13, S-60-14 with Fc | QVQLQESGPGLVKPSETLSLTCAVSGYSISSGYYWGW | 89 |
| LALAPS without C- | IRQPPGKGLEWIGTIYHSGSTYYNPSLESRVTISVDTSK | |
| terminal Lysine | NQFSLKLSSVTAADTAVYYCARQGSIQQGYYGMDV | |
| | WGQGTTVTVSSASTKGPSVFPLNPSSKSTSGGTAALG | |
| | CLVKDYFPEPVTVSWNSGALTSGVHTFPAVLQSSGLY | |
| | SLSSVVTVPSSSLGTQTYICNVNHKPSNTKVDKKVEP | |
| | KSCDKTHTCPPCPAPEAAGGPSVFLFPPKPKDTLMISR | |
| | TPEVTCVVVDVSHEDPEVKFNWYVDGVEVHNAKTK | |
| | PREEQYNSTYRVVSVLTVLHQDWLNGKEYKCKVSN | |
| | KALPASIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNH | |
| | YTQKSLSLSPG | |
| S-60, S-60-15 [N33 (wt)], S- | QVQLQESGPGLVKPSETLSLTCAVSGYSISSGYYWGW | 90 |
| 60-15.1 [N33T], S-60-15.2 | IRQPPGKGLEWIGTIYHSGSTYYNPSLESRVTISVDTS | |
| [N33S], S-60-15.3 [N33G], | KNQFSLKLSSVTAADTAVYYCARQGSIQQGYYGMD | |
| S-60-15.4 [N33R], S-60-15.5 | VWGQGTTVTVSSASTKGPSVFPLNPSSKSTSGGTAAL | |
| [N33D], S-60-15.6 [N33H], | GCLVKDYFPEPVTVSWNSGALTSGVHTFPAVLQSSGL | |
| 5-60-15.7 [N33K], S-60-15.8 | YSLSSVVTVPSSSLGTQTYICNVNHKPSNTKVDKKVE | |
| [N33Q], S-60-15.9 [N33Y], | PKSCDKTHTCPPCPAPEAAGGPSVFLFPPKPKDTLMIS | |
| S-60-15.10 [N33E], S-60- | RTPEVTCVVVDVSHEDPEVKFNWYVDGVEVHNAKT | |
| 15.11 [N33W], S-60-15.12 | KPREEQYNSTYRVVSVLTVLHQDWLNGKEYKCKVS | |
| [N33F], S-60-15.13 [N33I], | NKALPASIEKTISKAKGQPREPQVYTLPPSRDELTKNQ | |
| S-60-15.14 [N33V], S-60- | VSLTCLVKGFYPSDIAVEWESNGQPENNYKTTPPVLD | |
| 15.15 [N33A], S-60-15.16 | SDGSFFLYSKLTVDKSRWQQGNVFSCSVMHEALHNH | |
| [N33M], S-60-15.17 [N33L], | YTQKSLSLSPGK | |
| S-60-16, S-60-18, S-60-24 | | |
| with Fc LALAPS with C- | | |
| terminal Lysine | | |
| S-60, S-60-15 [N33 (wt)], S- | QVQLQESGPGLVKPSETLSLTCAVSGYSISSGYYWGW | 91 |
| 60-15.1 [N33T], S-60-15.2 | IRQPPGKGLEWIGTIYHSGSTYYNPSLESRVTISVDTS | |
| [N33S], S-60-15.3 [N33G], | KNQFSLKLSSVTAADTAVYYCARQGSIQQGYYGMD | |
| S-60-15.4 [N33R], S-60-15.5 | VWGQGTTVTVSSASTKGPSVFPLNPSSKSTSGGTAAL | |
| [N33D], S-60-15.6 [N33H], | GCLVKDYFPEPVTVSWNSGALTSGVHTFPAVLQSSGL | |
| S-60-15.7 [N33K], S-60-15.8 | YSLSSVVTVPSSSLGTQTYICNVNHKPSNTKVDKKVE | |
| [N33Q], S-60-15.9 [N33Y], | PKSCDKTHTCPPCPAPEAAGGPSVFLFPPKPKDTLMIS | |
| S-60-15.10 [N33E], S-60- | RTPEVTCVVVDVSHEDPEVKFNWYVDGVEVHNAKT | |
| 15.11 [N33W], S-60-15.12 | KPREEQYNSTYRVVSVLTVLHQDWLNGKEYKCKVS | |
| [N33F], S-60-15.13 [N33I], | NKALPASIEKTISKAKGQPREPQVYTLPPSRDELTKNQ | |
| S-60-15.14 [N33V], S-60- | VSLTCLVKGFYPSDIAVEWESNGQPENNYKTTPPVLD | |
| 15.15 [N33A], S-60-15.16 | SDGSFFLYSKLTVDKSRWQQGNVFSCSVMHEALHNH | |
| [N33M], S-60-15.17 [N331], | YTQKSLSLSPG | |
| S-60-16, S-60-18, S-60-24 | | |
| with Fc LALAPS without | | |
| C-terminal Lysine | | |

TABLE 30
Full-length light chain amino acid sequences

| Description | Sequence | SEQ ID NO |
|---|---|---|
| S-60-10; S-60-13; S- | DIVMTQSPLSLPVTPGEPASIS | 92 |
| 60-15 [N33 (wt)] | CRSSQSLLRSNGYNYLDWYL | |
| | QKPGQSPQLLIYIGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-11; S-60-14 | DIVMTQSPLSLPVTPGEPASIS | 93 |
| | CRSSQSLLRSNGYNYLDWYL | |
| | QKPGQSPQLLIYLGSNRASGV | |
| | PDRFSGSGSGTDFTLKISRVEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-12 | DIVMTQSPLSLPVTPGEPASIS | 94 |
| | CRSSQSLLRSNGYNYLDWYL | |
| | QKPGQPPQLLYLGSNRVSGV | |
| | PDRFSGSGSGTDFTLKISRVEA | |
| | EDVGVYYCMQQQETPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKEKVYACEVTHQGLSS | |
| | PVTKSFNRGEC | |
| S-60-15.1 [N33T] | DIVMTQSPLSLPVTPGEPASIS | 95 |
| | CRSSQSLLRSTGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.2 [N33S] | DIVMTQSPLSLPVTPGEPASIS | 96 |
| | CRSSQSLLRSSGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.3 [N33G] | DIVNMTQSPLSLPVTPGEPASIS | 97 |
| | CRSSQSLLRSGGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.4 [N33R] | DIVMTQSPLSLPVTPGEPASIS | 98 |
| | CRSSQSLLRSRGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.5 [N33D] | DIVMTQSPLSLPVTPGEPASIS | 99 |
| | CRSSQSLLRSDGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.6 [N33H] | DIVMTQSPLSLPVTPGEPASIS | 100 |
| | CRSSQSLLRSHGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.7 [N33K] | DIVMTQSPLSLPVTPGEPASIS | 101 |
| | CRSSQSLLRSKGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.8 [N33Q] | DIVMTQSPLSLPVTPGEPASIS | 102 |
| | CRSSQSLLRSQGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.9 [N33Y] | DIVMTQSPLSLPVTPGEPASIS | 103 |
| | CRSSQSLLRSYGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.10 [N33E] | DIVMTQSPLSLPVTPGEPASIS | 104 |
| | CRSSQSLLRSEGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.11 [N33W] | DIVMTQSPLSLPVTPGEPASIS | 105 |
| | CRSSQSLLRSWGYNYLLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.12 [N33F] | DIVMTQSPLSLPVTPGEPASIS | 106 |
| | CRSSQSLLRSFGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.13 [N33I] | DIVMTQSPLSLPVTPGEPASIS | 107 |
| | CRSSQSLLRSIGYNYLDWYLQ | |
| | KPGQSPQLLIYLGSNRASGVP | |
| | DRFSGSGSGTDFTLKISRAEAE | |
| | DVGVYYCMQQQEAPLTFGGG | |
| | TKVEIKRTVAAPSVFIFPPSDE | |
| | QLKSGTASVVCLLNNFYPREA | |
| | KVQWKVDNALQSGNSQESVT | |
| | EQDSKDSTYSLSSTLTLSKAD | |
| | YEKHKVYACEVTHQGLSSPV | |
| | TKSFNRGEC | |
| S-60-15.14 [N33V] | DIVMTQSPLSLPVTPGEPASIS | 108 |
| | CRSSQSLLRSVGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.15 [N33A] | DIVMTQSPLSLPVTPGEPASIS | 109 |
| | CRSSQSLLRSAGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.16 [N33M] | DIVMTQSPLSLPVTPGEPASIS | 110 |
| | CRSSQSLLRSMGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.17 [N33L] | DIVNTQSPLSLPVTPGEPASIS | 111 |
| | CRSSQSLLRSLGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-16 | DIVMTQSPLSLPVTPGEPASIS | 112 |
| | CRSSQSLLRSNGYNYLDWYL | |
| | QKPGQSPQLLIYLGSNRASGV | |
| | PDRFSGSGSGTDFTLKISRVEA | |
| | EDVGAYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-18 | DIVMTQSPLSLPVTPGGPASIS | 113 |
| | CRSSQSLLRSNGYNYLDWYL | |
| | QKPGQSPQLLIYLGSNRASGV | |
| | PDRLSGSGSGTDFTLKISRVEA | |
| | EDVGVYYCMQQQETPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60, S-60-19 | DIVMTQSPLSLPVTPGEPASIS | 114 |
| | CRSSQSLLHSNGYNYLDWYL | |
| | QKPGQSPQLLIYLGSNRASGV | |
| | PDRFSGSGSGTDFTLKISRVEA | |
| | EDVGVYYCMQQQETPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-24 | DIVMTQSPLSLPVTPGESASIS | 115 |
| | CRSSQGLLRSNGYNYLDWYL | |
| | QKPGQSPQLLIYLGSNRASGV | |
| | PDRFSGSGSGTDFTLKISRVEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |

1. A method of treating and/or delaying the progression of a disease or injury in an individual, comprising administering to the individual an anti-Sortilin antibody intravenously at a dose of at least about 30 mg/kg once every four weeks or more frequently, wherein the antibody comprises:
(i) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), and the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32);
(ii) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRVS (SEQ ID NO: 30), and the HVR-L3 comprising the amino acid sequence MQQQETPLT (SEQ ID NO: 33);
(iii) a heavy chain variable region comprising the HVR-H1 comprising the amino acid sequence YSISSGYYWG (SEQ ID NO: 1), the HVR-H2 comprising the amino acid sequence TIYHSGSTYYNPSLES (SEQ ID NO: 3), the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32);
(iv) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32);
(v) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSTGYNYLD (SEQ ID NO: 9), the HVR-L2 comprising the amino acid sequence LGSNRAS (SEQ ID NO: 29), and the HVR-L3 comprising the amino acid sequence MQQQEAPLT (SEQ ID NO: 32);
(vi) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQETPLT (SEQ ID NO: 33);
(vii) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIQQGYYGMDV (SEQ ID NO: 5); and a light chain variable region comprising the HVR-L1 comprising the amino acid sequence RSSQSLLHSNGYNYLD (SEQ ID NO: 26), the HVR-L2 comprising the amino acid sequence LGSNRAS (SEQ ID NO: 29), and the HVR-L3 comprising the amino acid sequence MQQQETPLT (SEQ ID NO: 33); or
(viii) a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQGLLRSNGYNYLD (SEQ ID NO: 27), the HVR-L2 comprising the amino acid sequence LGSNRAS (SEQ ID NO: 29), and the HVR-L3 comprising the amino acid sequence MQQQEAPLT (SEQ ID NO: 32).

2. The method of claim 1, wherein the dose is:
(i) at least about 35 mg/kg, at least about 40 mg/kg, at least about 45 mg/kg, at least about 50 mg/kg, at least about 55 mg/kg, or at least about 60 mg/kg;
(ii) between about 30 mg/kg and about 60 mg/kg; or
(iii) about 60 mg/kg.

3-4. (canceled)

5. The method of claim 1, wherein the anti-Sortilin antibody is administered once every two weeks, once every three weeks, or once every four weeks.

6-7. (canceled)

8. The method of claim 1, wherein the anti-Sortilin antibody is administered once every four weeks at a dose of about 60 mg/kg.

9. The method of claim 1, wherein;
(i) the heavy chain variable region comprises an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and the light chain variable region comprises an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); or
(ii) the heavy chain variable region comprises an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKOGYYGMDV (SEQ ID NO: 6); and the light chain variable region comprises an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

10. (canceled)

11. The method of claim 1, wherein the antibody comprises a heavy chain variable region comprising the amino acid sequence of SEQ ID NO: 54, and a light chain variable region comprising the amino acid sequence of SEQ ID NO: 57; a heavy chain variable region comprising the amino acid sequence of SEQ ID NO: 54, and a light chain variable region comprising the amino acid sequence of SEQ ID NO: 58; a heavy chain variable region comprising the amino acid sequence of SEQ ID NO: 54, and a light chain variable region comprising the amino acid sequence of SEQ ID NO: 59; a heavy chain variable region comprising the amino acid sequence of SEQ ID NO: 55, and a light chain variable region comprising the amino acid sequence of SEQ ID NO: 57; a heavy chain variable region comprising the amino acid sequence of SEQ ID NO: 55, and a light chain variable region comprising the amino acid sequence of SEQ ID NO: 58; a heavy chain variable region comprising the amino acid sequence of SEQ ID NO: 56, and a light chain variable region comprising the amino acid sequence of SEQ ID NO: 57; a heavy chain variable region comprising the amino acid sequence of SEQ ID NO: 56, and a light chain variable region comprising the amino acid sequence of SEQ ID NO: 77; a heavy chain variable region comprising the amino acid sequence of SEQ ID NO: 56, and a light chain variable region comprising the amino acid sequence of SEQ ID NO: 78; a heavy chain variable region comprising the amino acid sequence of SEQ ID NO: 54, and a light chain variable region comprising the amino acid sequence of SEQ ID NO: 79; or a heavy chain variable region comprising the amino acid sequence of SEQ ID NO: 56, and a light chain variable region comprising the amino acid sequence of SEQ ID NO: 80.

12. The method of claim 1, wherein the antibody comprises:
(i) a heavy chain variable region comprising the amino acid sequence of SEQ ID NO: 56, and a light chain variable region comprising the amino acid sequence of SEQ ID NO: 57; or
(ii) a heavy chain variable region comprising the amino acid sequence of SEQ ID NO: 56, and a light chain variable region comprising the amino acid sequence of SEQ ID NO: 60.

13. The method of claim 1, wherein the antibody is an IgG1 isotype and the Fc region comprises amino acid substitutions at positions L234A, L235A, and P331S, wherein the numbering of the residue position is according to EU numbering.

14. The method of claim 1, wherein the antibody comprises a heavy chain comprising the amino acid sequence of SEQ ID NO: 90 or SEQ ID NO: 91, and a light chain comprising the amino acid sequence of SEQ ID NO: 95.

15. The method of claim 1, wherein the disease or injury is selected from the group consisting of frontotemporal dementia, progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, amytrophic lateral sclerosis, and osteoarthritis.

16. (canceled)

17. The method of claim 1, wherein:
(i) the individual is heterozygous for a mutation in GRN; or
(ii) the individual is heterozygous for a C9orf72 hexanucleotide repeat expansion.

18. The method of claim 17, wherein the individual is heterozygous for a mutation in GRN, and wherein the mutation in GRN is a loss-of-function mutation.

19. (canceled)

20. The method of claim 17, wherein the individual shows symptoms of frontotemporal dementia.

21. The method of claim 17, wherein the individual does not show symptoms of frontotemporal dementia.

22. The method of claim 1, wherein:
(i) the level of PGRN protein in the plasma of the individual after administration of the anti-Sortilin antibody is at least two-fold, three-fold, or four-fold higher than the level of PGRN protein in the plasma of the individual before administration of the anti-Sortilin antibody;
(ii) the level of PGRN protein in the cerebrospinal fluid of the individual after administration of the anti-Sortilin antibody is at least two-fold higher than the level of PGRN protein in the cerebrospinal fluid of the individual before administration of the anti-Sortilin antibody; and/or
(iii) the expression level of SORT1 protein on peripheral white blood cells of the individual after administration of the anti-Sortilin antibody is reduced by at least 50% compared to the expression level of SORT1 protein on peripheral white blood cells of the individual before administration of the anti-Sortilin antibody.

23. (canceled)

24. The method of claim 22, wherein:
(i) the fold increase in the level of PGRN protein in the plasma of the individual is present at about 5 days, 28 days, 35 days, 42 days, 49 days, and/or 56 days after the last administration of the anti-Sortilin antibody;
(ii) the fold increase in the level of PGRN protein in the cerebrospinal fluid of the individual is present at about 12 days, 24 days, 28 days, 35 days, 42 days, 49 days, and/or 56 days after the last administration of the anti-Sortilin antibody; and/or
(iii) the reduction in the expression level of SORT1 on peripheral white blood cells of the individual is present at about twelve days or more after the last administration of the anti-Sortilin antibody.

25-35. (canceled)

36. The method of claim 1, wherein:
(i) the individual is treated for a treatment period of up to 48 weeks in length; and/or
(ii) the individual is treated for a treatment period of 48 weeks in length.

37. (canceled)

38. The method of claim 36, wherein:
(i) administration of the anti-Sortilin antibody occurs on the first day of the treatment period and every four weeks thereafter; and/or
(ii) the anti-Sortilin antibody is administered a total of 13 times during the treatment period.

39. (canceled)

40. The method of claim 1, wherein:
(i) the disease or injury is frontotemporal dementia (FTD), and wherein plasma neurofilament light chain (NfL) levels are reduced by at least 10% after administration of the anti-Sortilin antibody compared to the plasma neurofilament light chain (NfL) levels before administration of the anti-Sortilin antibody;
(ii) the protein levels of CTSB in the CSF of the individual are increased by at least about 20% after administration of the anti-Sortilin antibody compared to the protein levels of CTSB in the CSF of the individual before administration of the anti-Sortilin antibody;
(iii) the protein levels of SPP1 in the CSF of the individual are decreased by at least about 10% after administration of the anti-Sortilin antibody compared to the protein levels of SPP1 in the CSF of the individual before administration of the anti-Sortilin antibody;
(iv) the protein levels of N-acetylglucosamine kinase (NAGK) in the CSF of the individual are increased after administration of the anti-Sortilin antibody compared to the protein levels of NAGK in the CSF of the individual before administration of the anti-Sortilin antibody; and/or
(v) the protein levels of one or more inflammatory proteins in the CSF of the individual are decreased after administration of the anti-Sortilin antibody compared to the protein levels of the one or more inflammatory proteins in the CSF of the individual before administration of the anti-Sortilin antibody, wherein the one or more inflammatory proteins are selected from the group consisting of 14-3-3 protein epsilon (YWHAE), allograft inflammatory factor 1 (AIF1), colony stimulating factor 1 (CSF1), chitinase 1 (CHIT1), lymphocyte antigen 86 (LY86), and CD86.

41-44. (canceled)

45. A method of monitoring treatment of an individual being administered an anti-Sortilin antibody, comprising measuring the level of one or more proteins in a sample from the individual before and after the individual has received one or more doses of an anti-Sortilin antibody, wherein the one or more proteins are selected from the group consisting of CTSB, SPP1, NAGK, YWHAE, AIF1, CSF1, CHIT1, LY86, neurofilament light chain (NfL), and CD86.

46-53. (canceled)
